(19) United States
(12) Patent Application Publication
(10) Pub. No.: US 2025/0115634 A1
(43) Pub. Date: Apr. 10, 2025

(54) LIGAND, METHOD FOR PREPARING SAME, AND USE THEREOF

(71) Applicant: LNCTAC CO., LTD., Chengdu (CN)

(72) Inventors: Hailin ZHANG, Chengdu (CN); Jia MA, Chengdu (CN); Yibao JIANG, Chengdu (CN); Guigang DENG, Chengdu (CN); Xin CHEN, Chengdu (CN)

(21) Appl. No.: 18/832,561

(22) PCT Filed: Jan. 19, 2023
(86) PCT No.: PCT/CN2023/073154

§ 371 Date: Jul. 24, 2024

(30) Foreign Application Priority Data
Jan. 30, 2022 (CN) 202210114418.X
Feb. 18, 2022 (CN) 202210149606.6

(51) Int. Cl. C07H 15/26; C07H 15/08; C07H 15/18

(52) U.S. Cl. CPC C07H 15/26; C07H 15/08; C07H 15/18

(57) ABSTRACT

The present invention relates to a ligand, a method for preparing same, and a use thereof. The ligand has a structure represented by formula (I) or formula (II), wherein R₁, R₂, R₃, and R₄ are all linear structures with a glycosyl group at a terminal end; X and Y are both Y-shaped backbone structures connected with other chemical groups; and X₁, X₂, and Y are all Y-shaped backbone structures connected with other chemical groups.

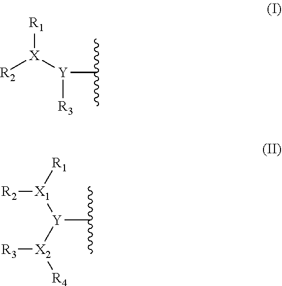

### TECHNICAL FIELD

The present disclosure belongs to the field of pharmaceutical chemistry, and specifically, relates to a ligand, a method for preparing the same, and use thereof.

### BACKGROUND

A ligand is a molecule or group that can bind to a target protein. A precisely designed ligand that can perfectly bind to a target protein is covalently linked to a certain functional molecule, which facilitates the delivery of such functional molecule to the target tissue and performance of its specific functions, and greatly reduces the toxic side effects caused by off-target. Nucleic acid drugs, as an emerging type of drugs in recent years, are of great significance for the treatment of major human diseases. However, nucleic acids per se are difficult to penetrate the cell membrane, and have non-targeting property and poor druggability, while ligand-linked nucleic acid drugs are endowed with tissue targeting specificity, and can enter cells smoothly through receptor-ligand mediated endocytosis, and play a role. Asialoglycoprotein receptor (ASGP-R), a transmembrane glycoprotein highly expressed on hepatocytes, can specifically recognize glycosyl residues such as galactose (Gal-) and N-acetylgalactosamine (GalNAc-) residues, and has a 50-fold higher affinity for GalNAc than for Gal. Therefore, a designed and constructed ligand with GalNAc glycosyl residues is very suitable for the liver-targeted delivery of functional molecules, especially nucleic acid drugs.

On this basis, the present disclosure is proposed.

### SUMMARY

Firstly, the present disclosure relates to a ligand having a structure of Formula I or Formula II:

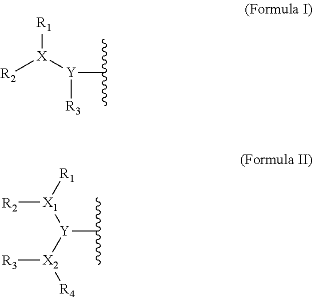

wherein,
R₁, R₂, R₃ and R₄ are each a chain structure having a terminal glycosyl group, and
X, X₁, X₂ and Y are each a backbone structure in a triply branched form for linking other chemical groups.

Further, in the ligand,
R₁ has a structure of

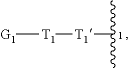

R₂ has a structure of

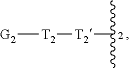

R₃ has a structure of and

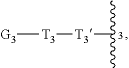

and
R₄ has a structure of

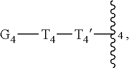

wherein,
T₁ to T₄ and T₁′ to T₄′ are each a structure comprising at least one chain, cyclic or branched chemical moiety, wherein T₁′ to T₄′ may be present or absent;
G₁ to G₄ are each a glycosyl group with or without a protective group on hydroxyl; preferably,
G₁ to G₄ are each independently selected from lactosyl, galactosyl, 2-aminogalactosyl, 2-formamidogalactosyl, 2-acetamidogalactosyl and 2-propionamidogalactosyl group, preferably, 2-acetamidogalactosyl group; more preferably, G₁ to G₄ are each independently selected from glycosyl groups having the following structures:

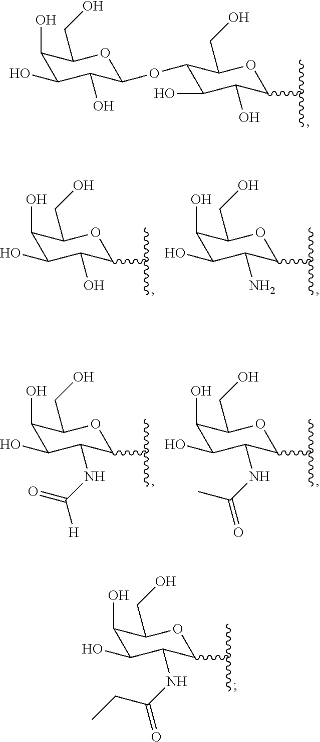

preferably, the protective group for hydroxyl is an acetyl group;
G₁ to G₄ are linked to T₁ to T₄ via an O-glycosidic bond, an S-glycosidic bond or an N-glycosidic bond, preferably via an O-glycosidic bond, respectively.

Further, in the ligand having a structure of Formula I,
X has a structure of

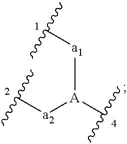

Y has a structure of

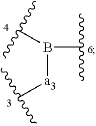

X is linked to Y at residue position 4, and A and B are each a backbone structure having a ring, chain or branch and in a triply branched form for linking other chemical groups;
a₁, a₂ and a₃ may be the same or different or absent;
where a₁, a₂ and a₃ are present, a₁ to a₃ are selected from O, S and NQ₁; wherein,
Q₁ is H, alkyl or acyl; preferably, Q₁ is selected from H, methyl, ethyl, n-propyl, isopropyl, formyl, acetyl, n-propionyl, isopropionyl and

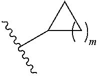

(m is an integer from 1 to 4);
preferably, a₁ to a₃ are each an O atom.

More further, in the ligand having a structure of Formula I, Y has a structure of:

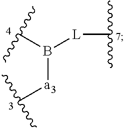

L is linked to B at residue position 5 by directly forming a bond with each other or by a linker (K), and K is a chain or ring with/without a branch, preferably, K is selected from

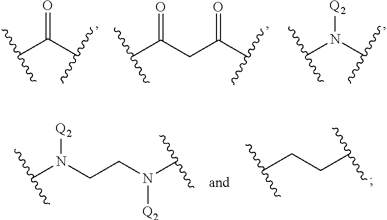

Q₂ is H or alkyl; preferably, Q₂ is selected from H, methyl, ethyl, n-propyl, isopropyl and

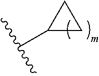

(m is an integer from 1 to 4);
L is a chain structure, a cyclic structure, or a chain comprising a cyclic structure, and is linked to B at residue position 5 in an appropriate manner;
preferably, L is selected from: M₁;

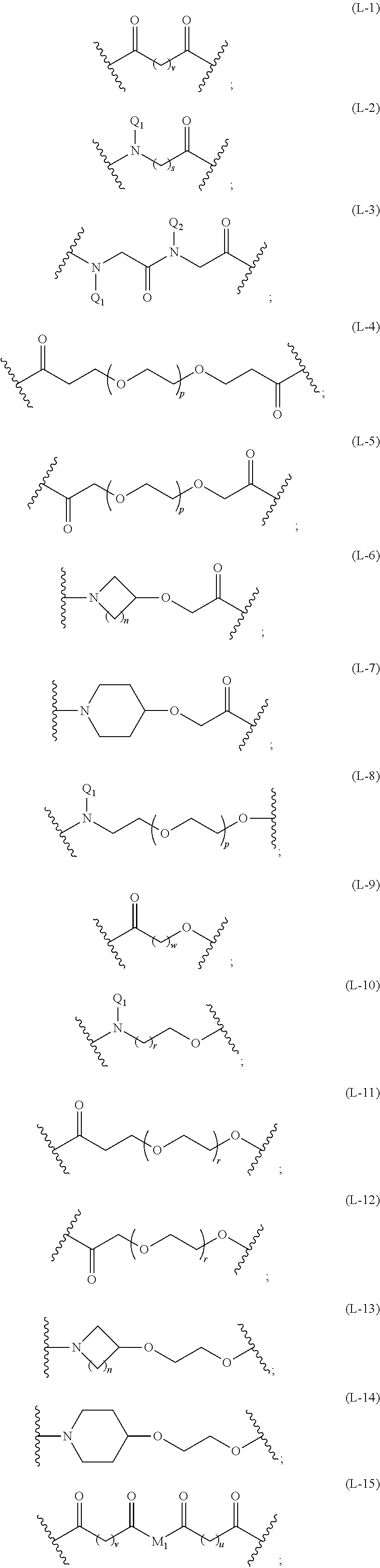

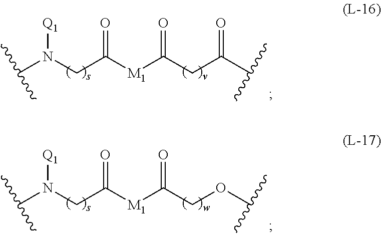

wherein,
M₁ is selected from:

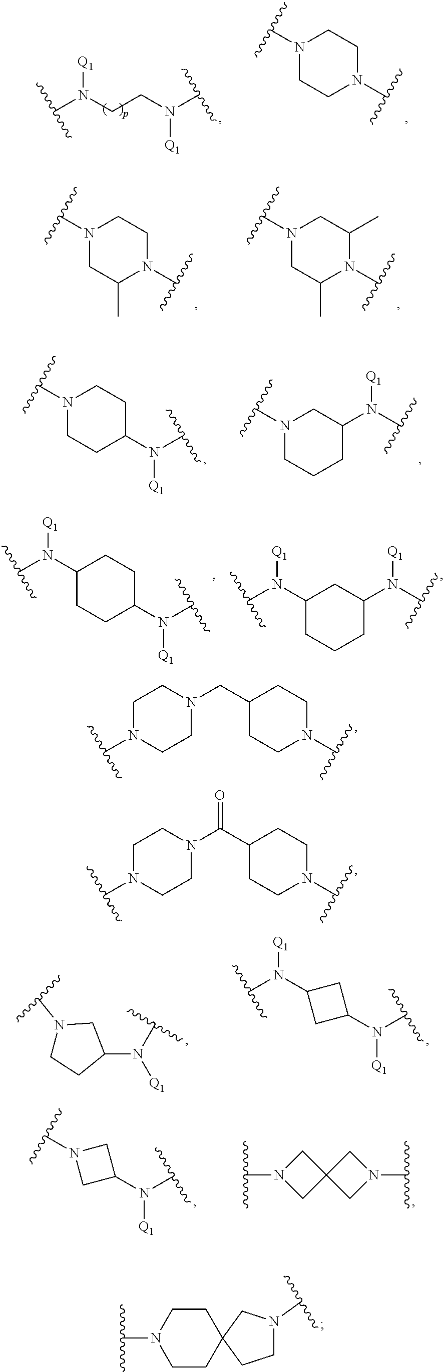

in the above formulas, e and f are each an integer from 1 to 2; m is an integer from 1 to 4; p and q are each an integer from 1 to 6; r is an integer from 1 to 11, preferably, from 1 to 5; s and t are each an integer from 1 to 17; v and u are each an integer from 1 to 18; and w is an integer from 1 to 19.

More further, in the ligand having a structure of Formula I, where a₁ and a₂ are present, A is selected from:

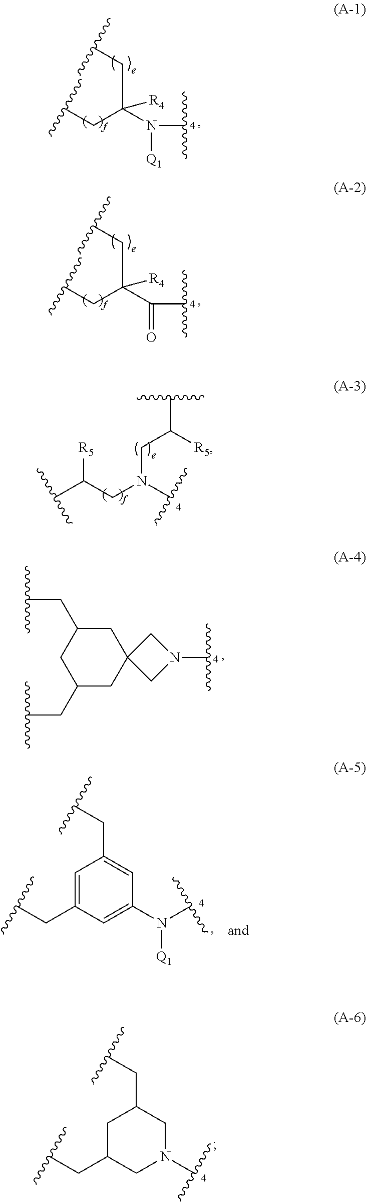

wherein,
R₄ is H, methyl or ethyl;
R₅ is H or methyl;
where both a₁ and a₂ are absent, A is selected from:

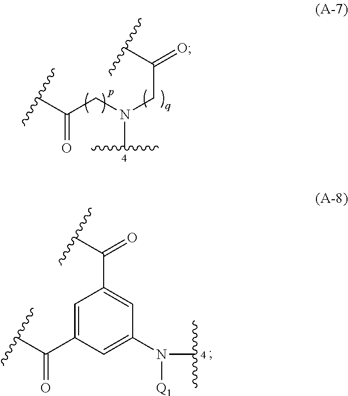

and
a nitrogen atom (A-9).

Where a₃ is present, B is selected from:

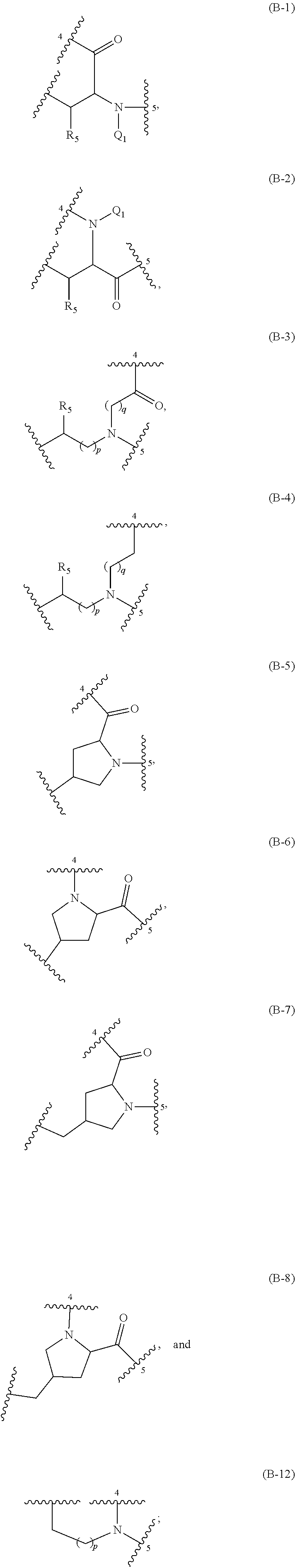

where a₃ is absent, B is selected from:

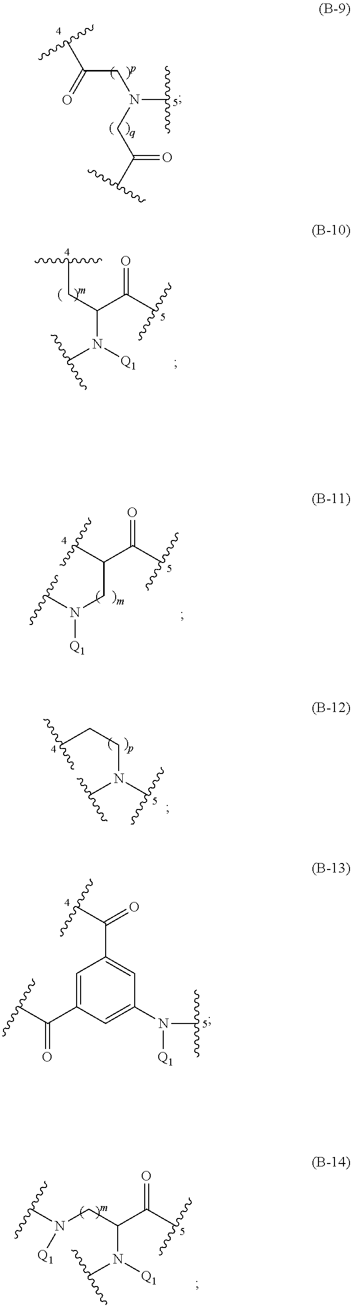

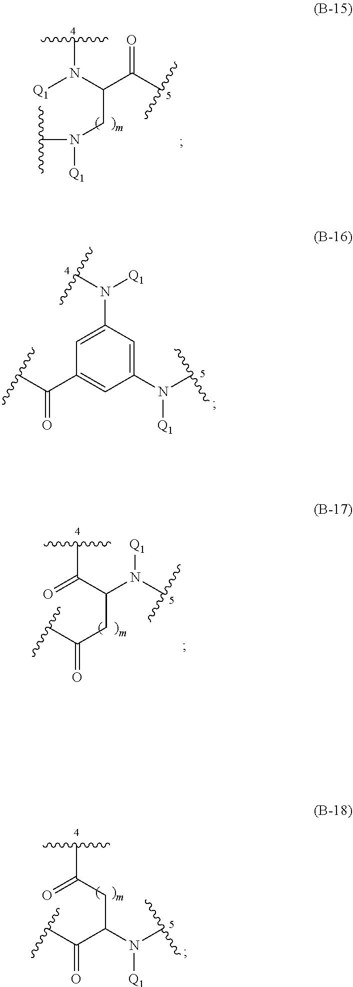

A is linked to B at residue position 4 by directly forming a bond with each other or by linker K; in the above formulas, e and f are each an integer from 1 to 2; m is an integer from 1 to 4; p and q are each an integer from 1 to 6; r is an integer from 1 to 11, preferably, from 1 to 5; s and t are each an integer from 1 to 17; v and u are each an integer from 1 to 18; w is an integer from 1 to 19.

Further, in the ligand having a structure of Formula I, T₁-T₃ may be the same or different, and T₁ to T₃ are each independently selected from:

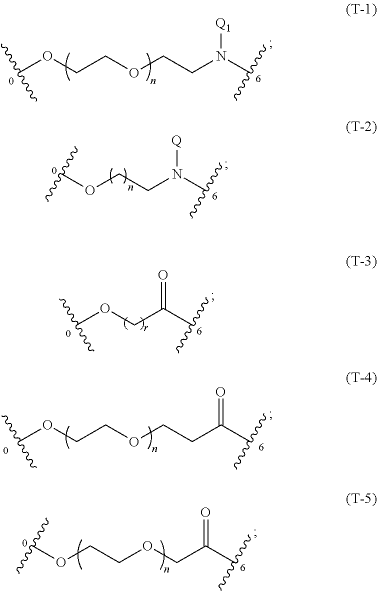

residue position 0 has a first atom/group that may be S or NQ₁;
T₁ to T₃ are linked to T₁′ to T₃′ at residue position 6 by directly forming a bond with each other or by linker K, respectively;
n is an integer from 1 to 3.

Further, in the ligand having a structure of Formula I,
T₁′ to T₃′ are linked to a₁ to a₃, or A or B at residue positions 1-3 by directly forming a bond with each other or by linker K, respectively; T₁′ to T₃′ are each independently selected from:

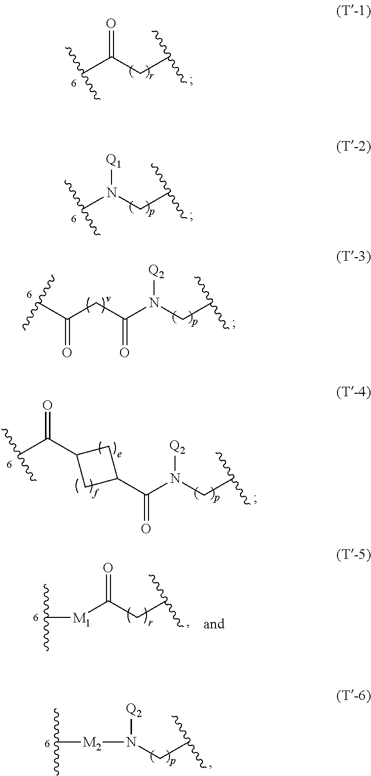

M₂ is selected from:

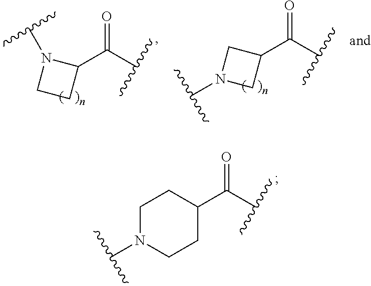

in the above formulas, e and f are each an integer from 1 to 2; m is an integer from 1 to 4; p and q are each an integer from 1 to 6; r is an integer from 1 to 11, preferably, from 1 to 5; s and t are each an integer from 1 to 17; v and u are each an integer from 1 to 18; w is an integer from 1 to 19.

Most preferably, the ligand having a structure of Formula I has the following structures:

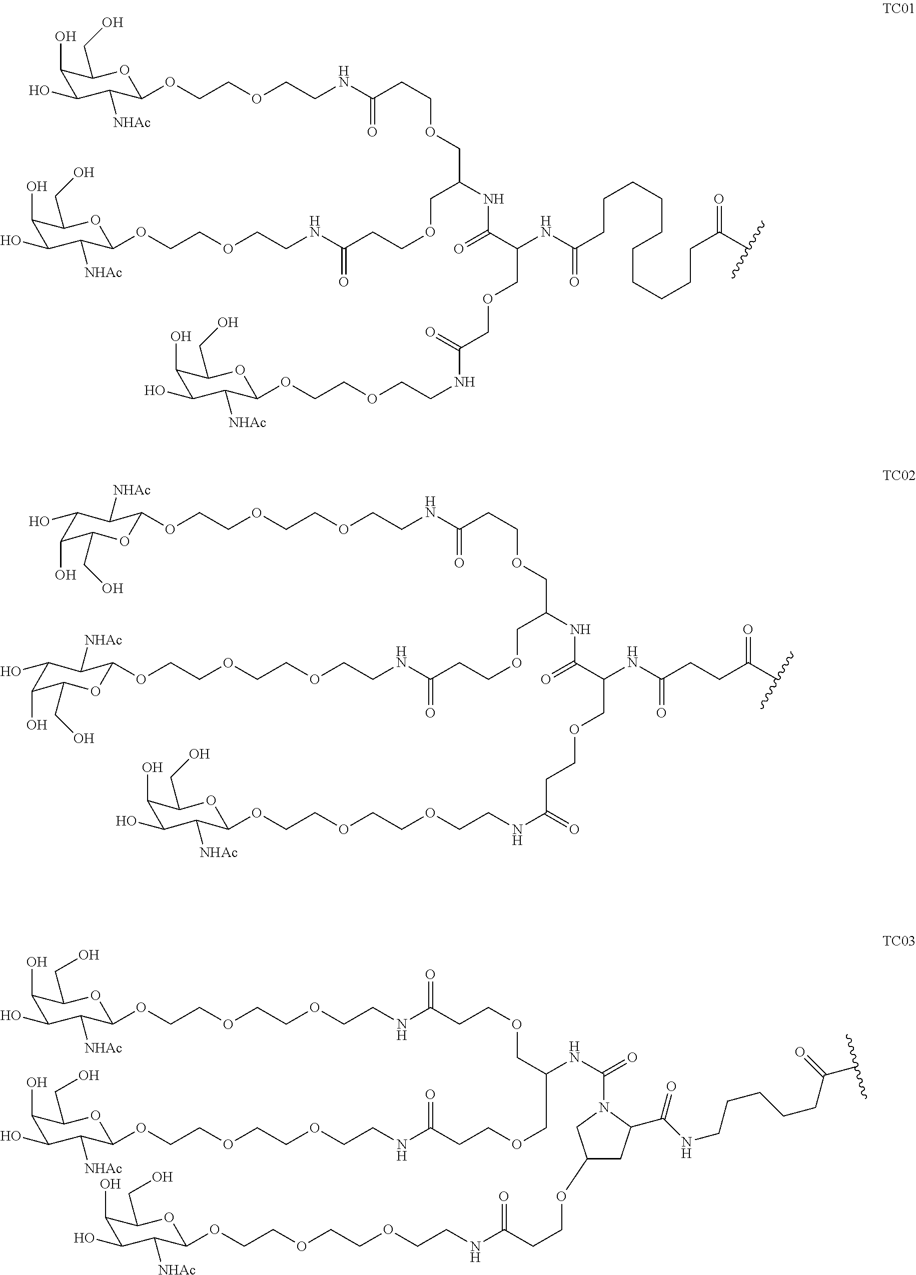 [SMILES: *C(=O)CCCCCCCCCCC(=O)NC(COCC(=O)NCCOCCOC1OC(CO)C(O)C(O)C1NC(C)=O)C(=O)NC(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCC(=O)NC(COCCC(=O)NCCOCCOCCOC1OC(CO)C(O)C(O)C1NC(C)=O)C(=O)NC(COCCC(=O)NCCOCCOCCOC1OC(CO)C(O)C(O)C1NC(C)=O)COCCC(=O)NCCOCCOCCOC1OC(CO)C(O)C(O)C1NC(C)=O ; *C(=O)CCCCCNC(=O)C1CC(OCCC(=O)NCCOCCOCCOC2OC(CO)C(O)C(O)C2NC(C)=O)CN1C(=O)NC(COCCC(=O)NCCOCCOCCOC1OC(CO)C(O)C(O)C1NC(C)=O)COCCC(=O)NCCOCCOCCOC1OC(CO)C(O)C(O)C1NC(C)=O]

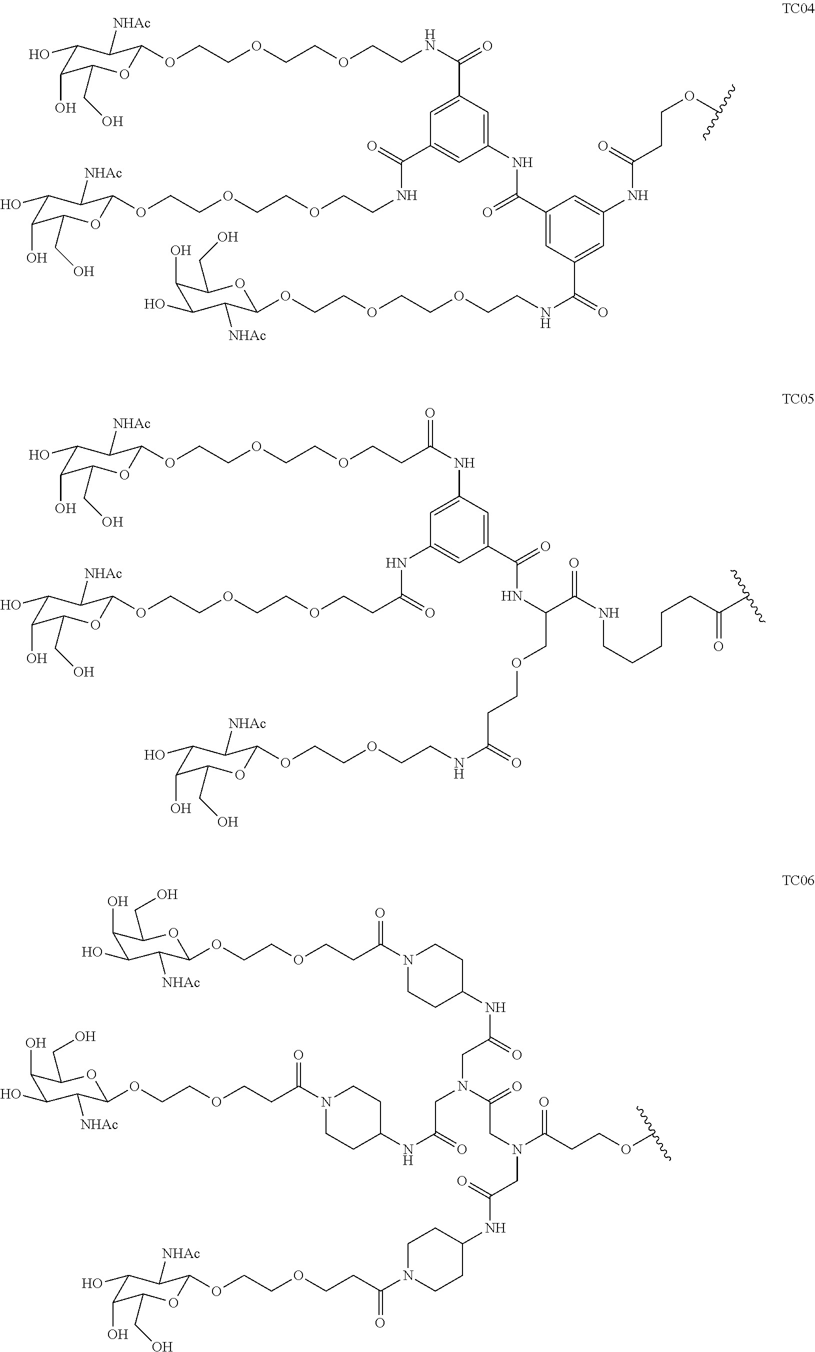

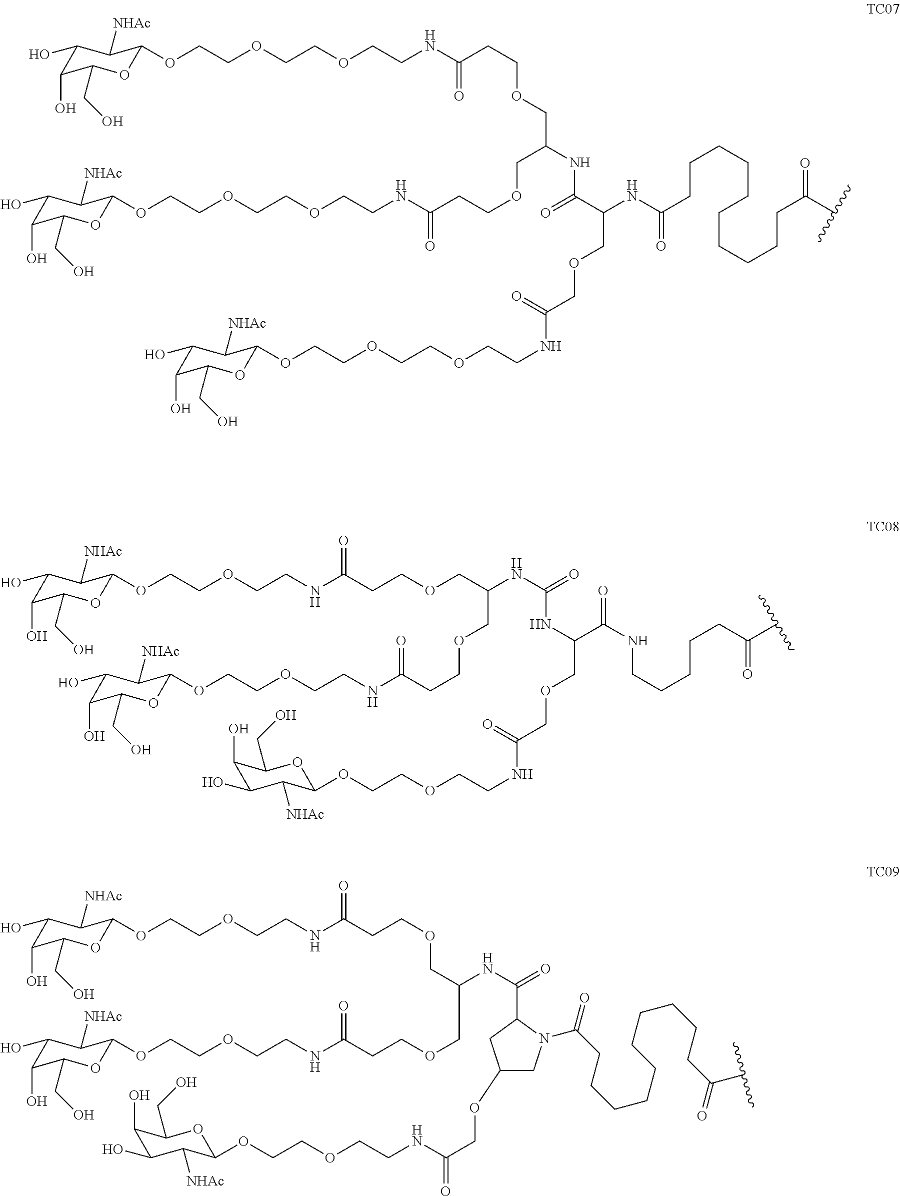 [SMILES: *C(=O)CCCCCCCCCCC(=O)NC(COCC(=O)NCCOCCOCCOC1OC(CO)C(O)C(O)C1NC(C)=O)C(=O)NC(COCCC(=O)NCCOCCOCCOC1OC(CO)C(O)C(O)C1NC(C)=O)COCCC(=O)NCCOCCOCCOC1OC(CO)C(O)C(O)C1NC(C)=O ; *C(=O)CCCCCNC(=O)C(COCC(=O)NCCOCCOC1OC(CO)C(O)C(O)C1NC(C)=O)NC(=O)NC(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCCCCCCCCCC(=O)N1CC(OCC(=O)NCCOCCOC2OC(CO)C(O)C(O)C2NC(C)=O)CC1C(=O)NC(COCCC(=O)NCCOCCOC1OC(CO)C(O)C(O)C1NC(C)=O)COCCC(=O)NCCOCCOC1OC(CO)C(O)C(O)C1NC(C)=O]

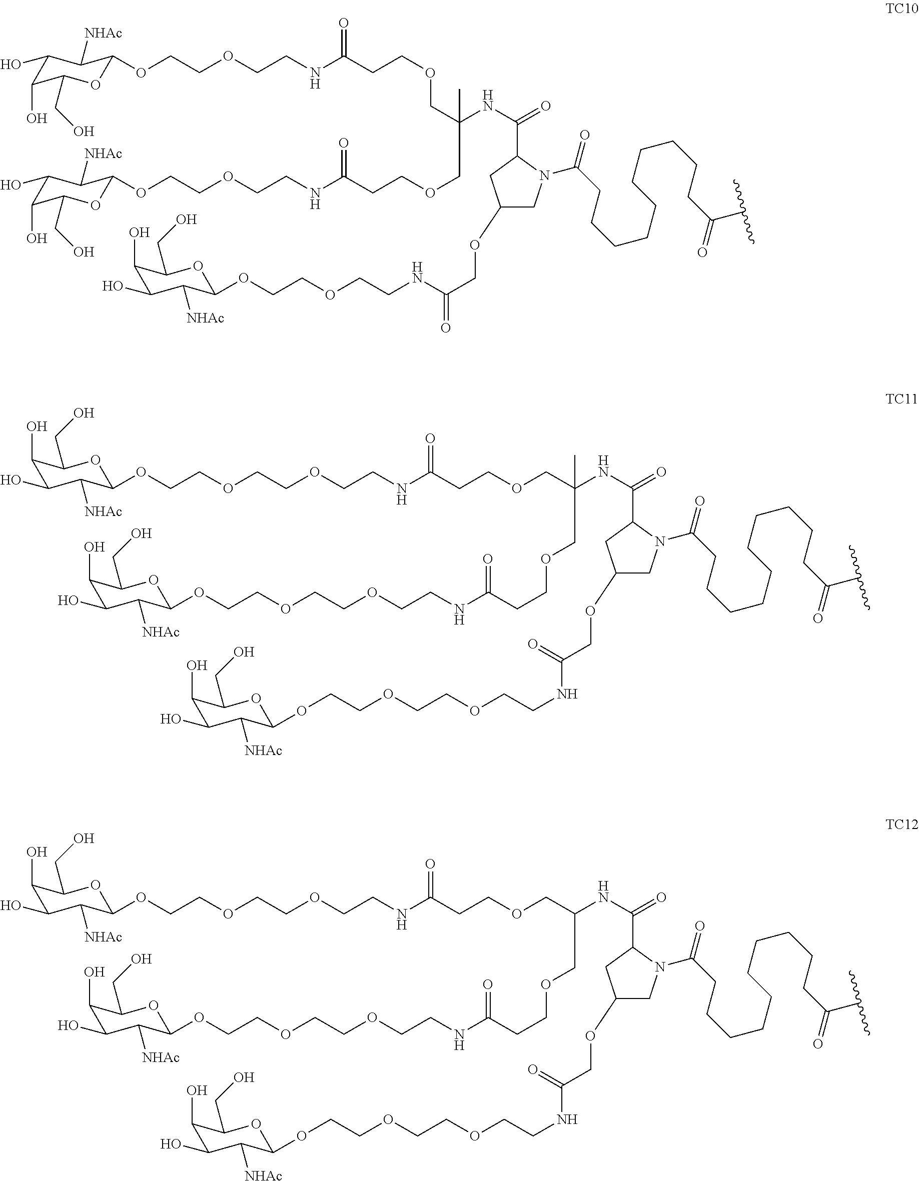

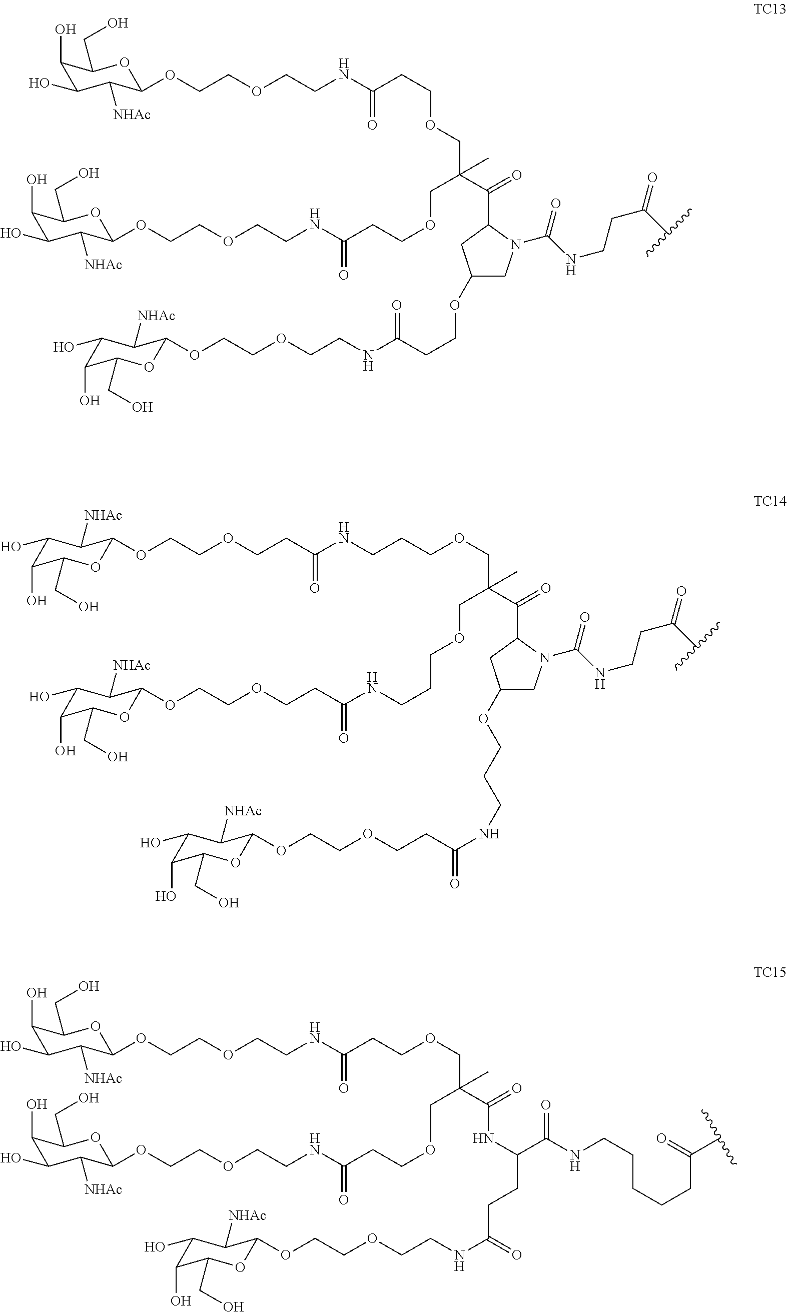 [SMILES: *C(=O)CCNC(=O)N1CC(OCCC(=O)NCCOCCOC2OC(CO)C(O)C(O)C2NC(C)=O)CC1C(=O)C(C)(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCNC(=O)N1CC(OCCCNC(=O)CCOCCOC2OC(CO)C(O)C(O)C2NC(C)=O)CC1C(=O)C(C)(COCCCNC(=O)CCOCCOC1OC(CO)C(O)C(O)C1NC(C)=O)COCCCNC(=O)CCOCCOC1OC(CO)C(O)C(O)C1NC(C)=O ; *C(=O)CCCCCNC(=O)C(CCC(=O)NCCOCCOC1OC(CO)C(O)C(O)C1NC(C)=O)NC(=O)C(C)(COCCC(=O)NCCOCCOC1OC(CO)C(O)C(O)C1NC(C)=O)COCCC(=O)NCCOCCOC1OC(CO)C(O)C(O)C1NC(C)=O]

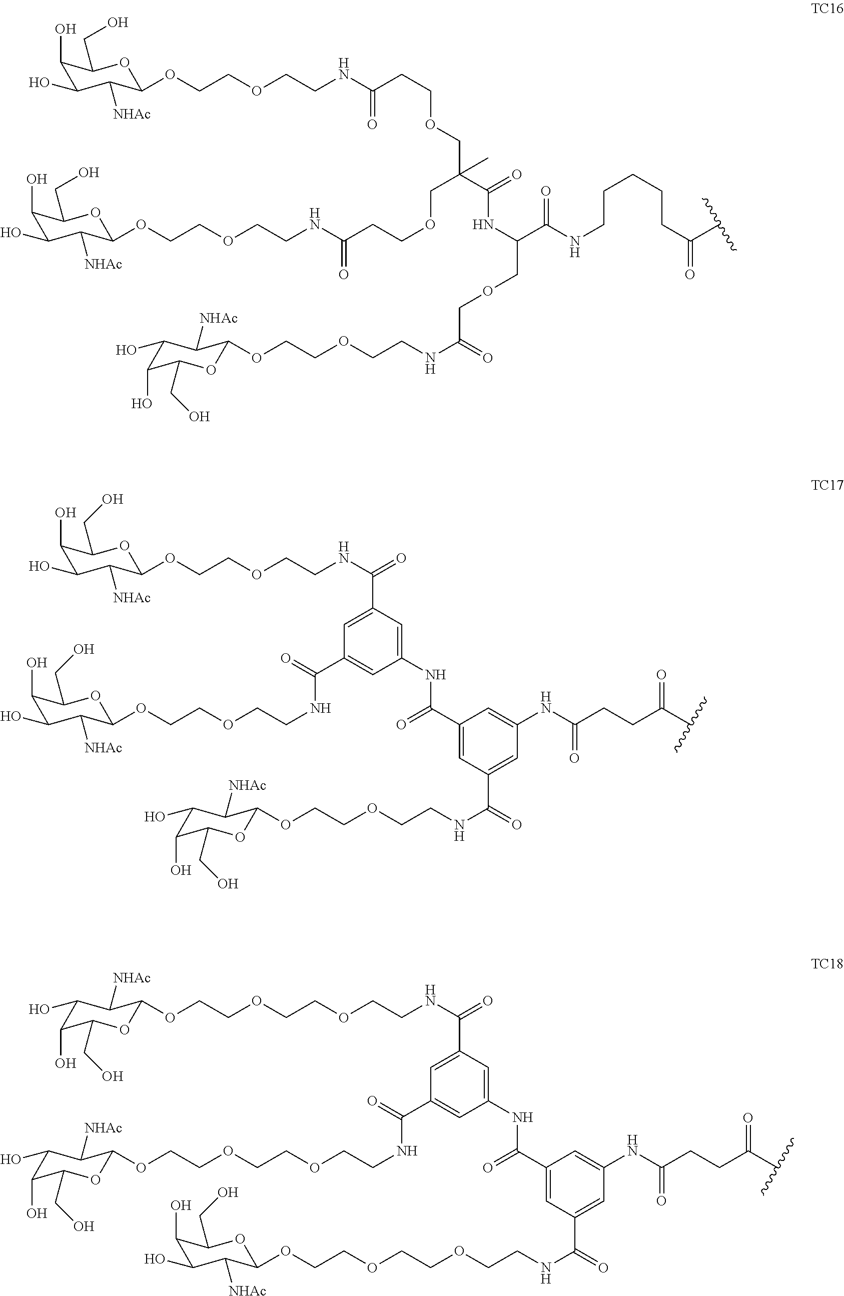 [SMILES: *C(=O)CCCCCNC(=O)C(COCC(=O)NCCOCCOC1OC(CO)C(O)C(O)C1NC(C)=O)NC(=O)C(C)(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCC(=O)Nc1cc(C(=O)NCCOCCOC2OC(CO)C(O)C(O)C2NC(C)=O)cc(C(=O)Nc2cc(C(=O)NCCOCCOC3OC(CO)C(O)C(O)C3NC(C)=O)cc(C(=O)NCCOCCOC3OC(CO)C(O)C(O)C3NC(C)=O)c2)c1 ; *C(=O)CCC(=O)Nc1cc(C(=O)NCCOCCOCCOC2OC(CO)C(O)C(O)C2NC(C)=O)cc(C(=O)Nc2cc(C(=O)NCCOCCOCCOC3OC(CO)C(O)C(O)C3NC(C)=O)cc(C(=O)NCCOCCOCCOC3OC(CO)C(O)C(O)C3NC(C)=O)c2)c1]

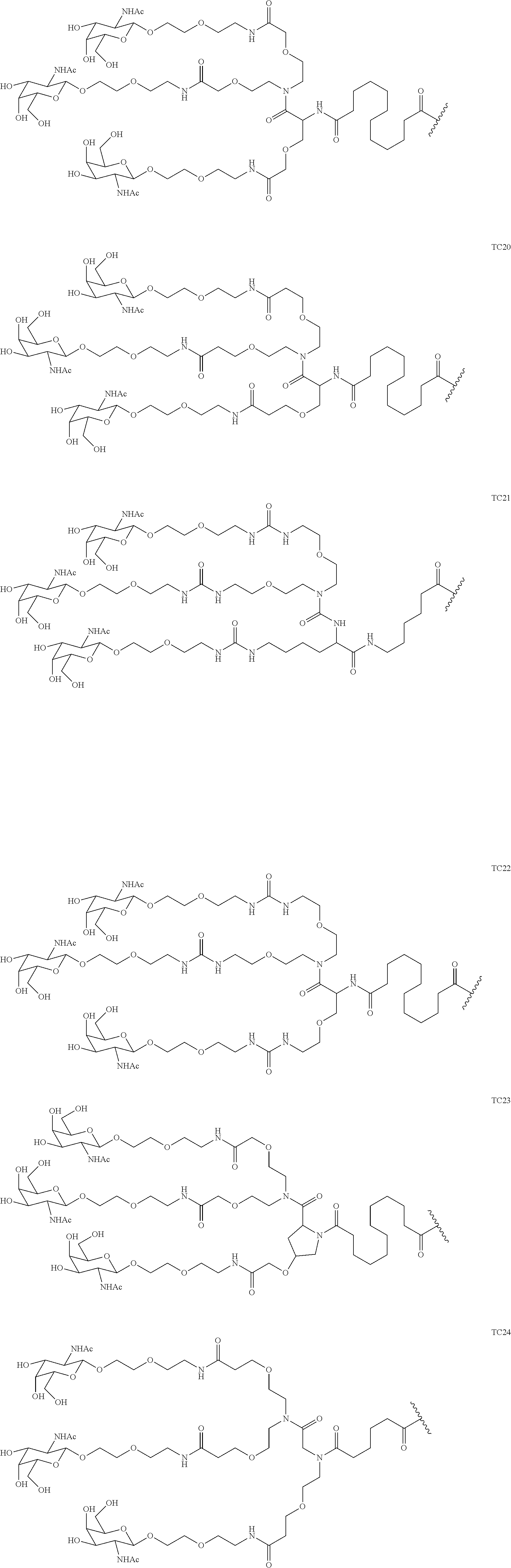

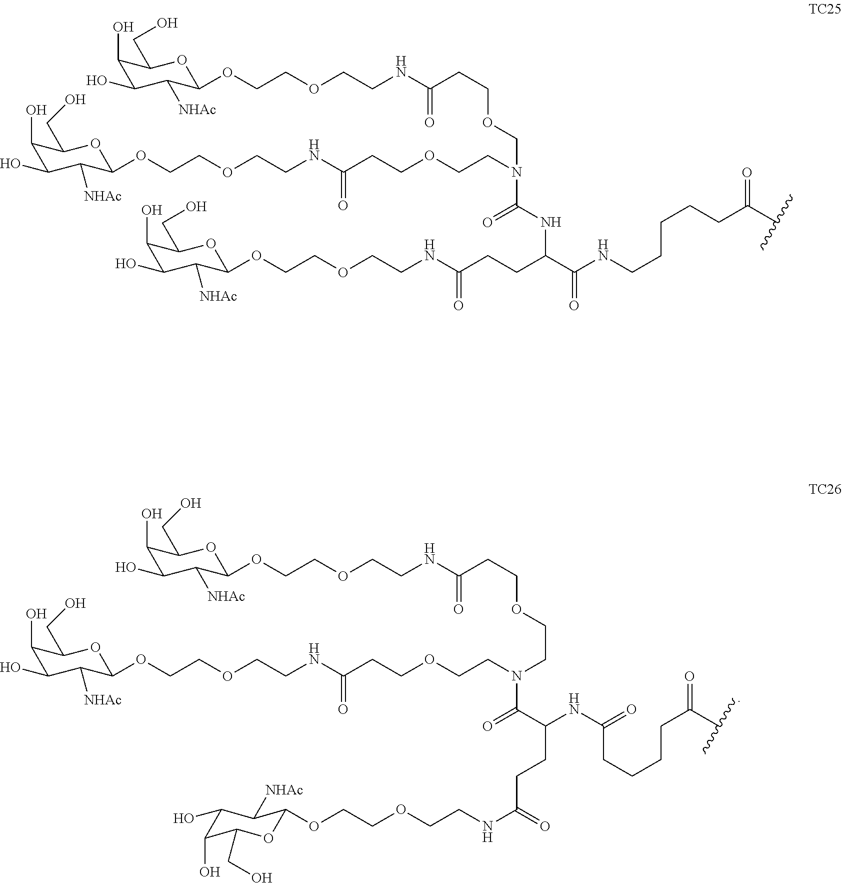 [SMILES: *C(=O)CCCCCNC(=O)C(CCC(=O)NCCOCCOC1OC(CO)C(O)C(O)C1NC(C)=O)NC(=O)N(CCOCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCCCC(=O)NC(CCC(=O)NCCOCCOC1OC(CO)C(O)C(O)C1NC(C)=O)C(=O)N(CCOCCC(=O)NCCOCCOC1OC(CO)C(O)C(O)C1NC(C)=O)CCOCCC(=O)NCCOCCOC1OC(CO)C(O)C(O)C1NC(C)=O]

In another aspect of the present disclosure, in the ligand having a structure of Formula II,
X₁ has a structure of

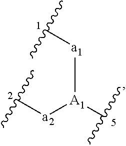

X₂ has a structure of

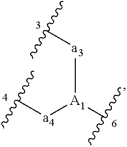

Y has a structure of

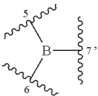 [SMILES: *B(*)*]

X₁ is linked to Y at residue position 5, X₂ is linked to Y at residue position 6, and A₁, A₂ and B are each a backbone structure having a ring, chain or branch and in a triple-branched form for linking other chemical groups;
a₁, a₂, a₃ and a₄ may be the same or different or absent;
where a₁, a₂, a₃ and a₄ are present, a₁ to a₄ are selected from O, S and NQ₁; wherein,
Q₁ is H, alkyl or acyl; preferably, Q₁ is selected from H, methyl, ethyl, n-propyl, isopropyl, formyl, acetyl, n-propionyl, isopropionyl and

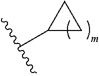

(m is an integer from 1 to 4);
preferably, a₁ to a₄ are each an O atom.

Further, in the ligand having a structure of Formula II, Y has a structure of

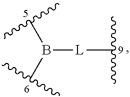

L is linked to B at residue position 7 by directly forming a bond with each other or by a linker (K), and K may be a chain or ring with/without a branch, and preferably, K is selected from

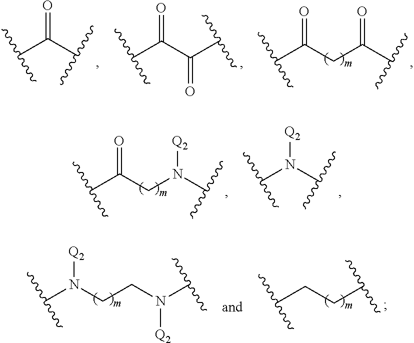

Q₂ is H or alkyl; preferably, Q₂ is selected from H, methyl, ethyl, n-propyl, isopropyl and

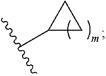

m is an integer from 1 to 4;
L is a chain structure, a cyclic structure, or a chain comprising a cyclic structure, and is linked to
B at residue position 7 in an appropriate manner;
preferably, L is selected from: M₁;

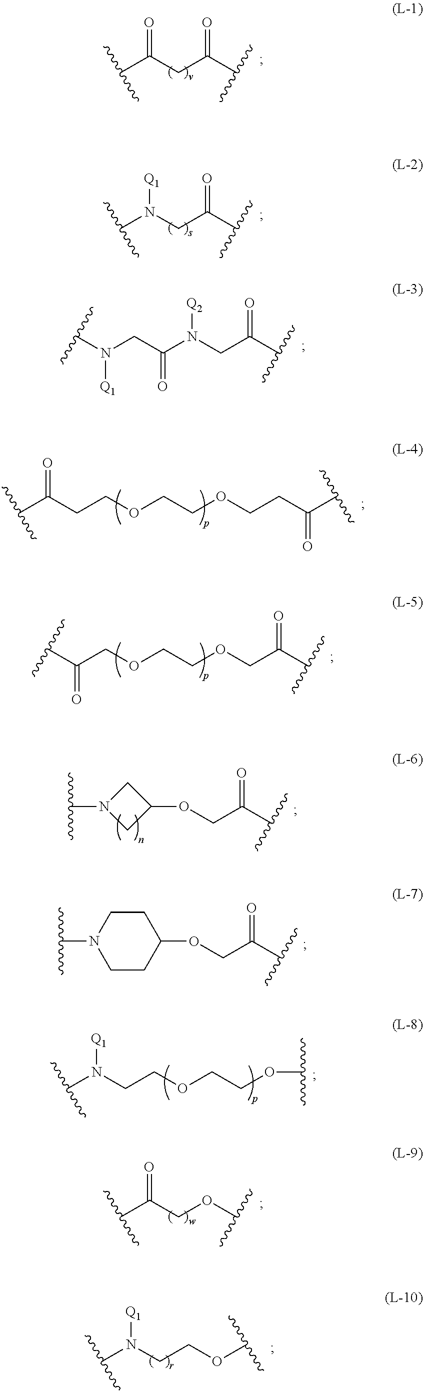

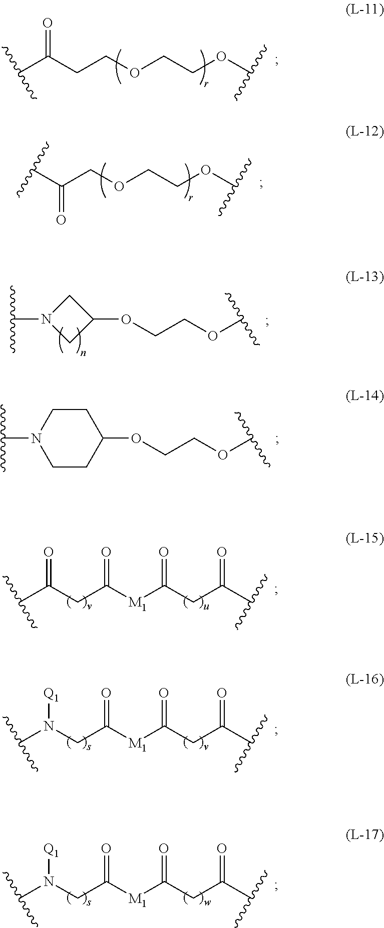

wherein,
M₁ is selected from:

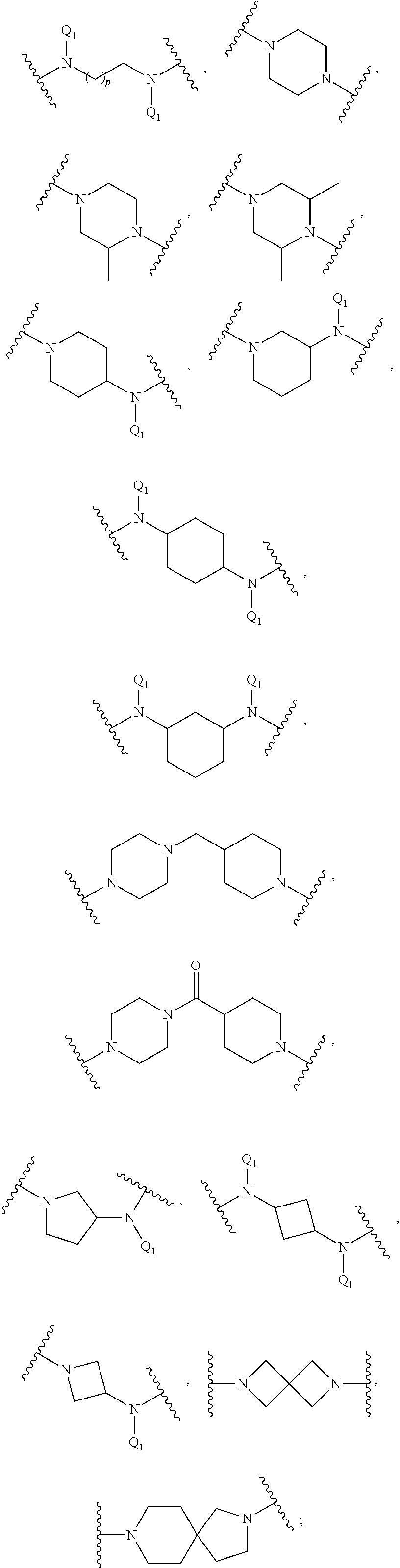

in the above formulas, e and f are each an integer from 1 to 2; n is an integer from 1 to 3; m is an integer from 1 to 4; j is an integer from 1 to 5; p and q are each an integer from 1 to 6; r is an integer from 1 to 11, preferably, from 1 to 5; s and t are each an integer from 1 to 17; v and u are each an integer from 1 to 18; w is an integer from 1 to 19.

More further, in the ligand having a structure of Formula II, where a₁, a₂, a₃ and a₄ are all present, A₁ and A₂ are each independently selected from:

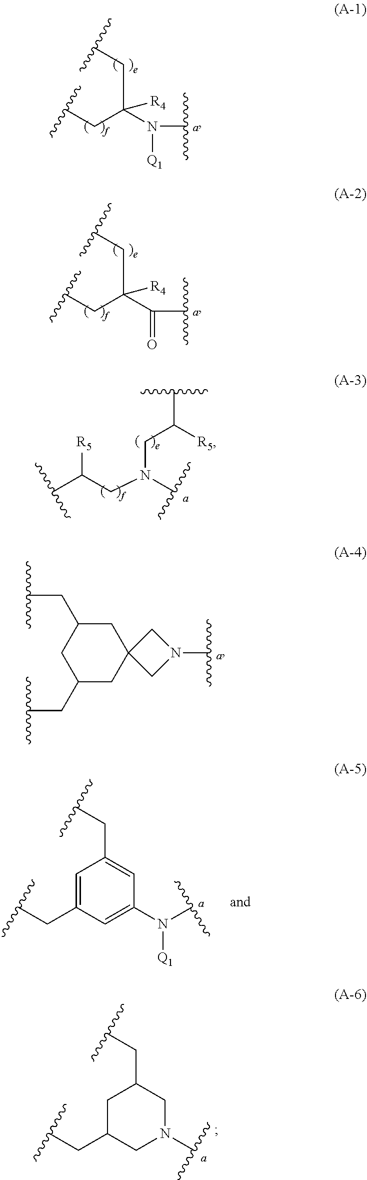

wherein,
R₄ is H, methyl or ethyl;
R₅ is H or methyl;
where a₁-a₄ are all absent, A₁ and A₂ are each independently selected from:

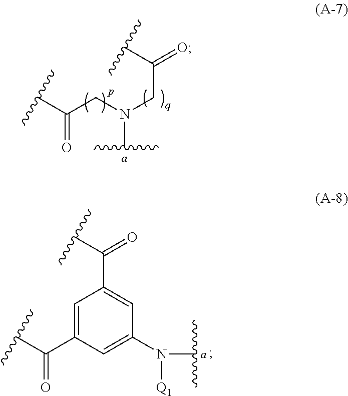

and
a nitrogen atom (A-9);
where a₁ and a₂ are present and a₃ and a₄ are absent, A₁ is selected from (A-1) to (A-6), and A₂ is selected from (A-7) to (A-9);
where a₃ and a₄ are present and a₁ and a₂ are absent, A₁ is selected from (A-7) to (A-9), and A₂ is selected from (A-1) to (A-6);
residue position a corresponds to residue position 5 of X₁ or residue position 6 of X₂.

More further, in the ligand having a structure of Formula II, B is selected from:

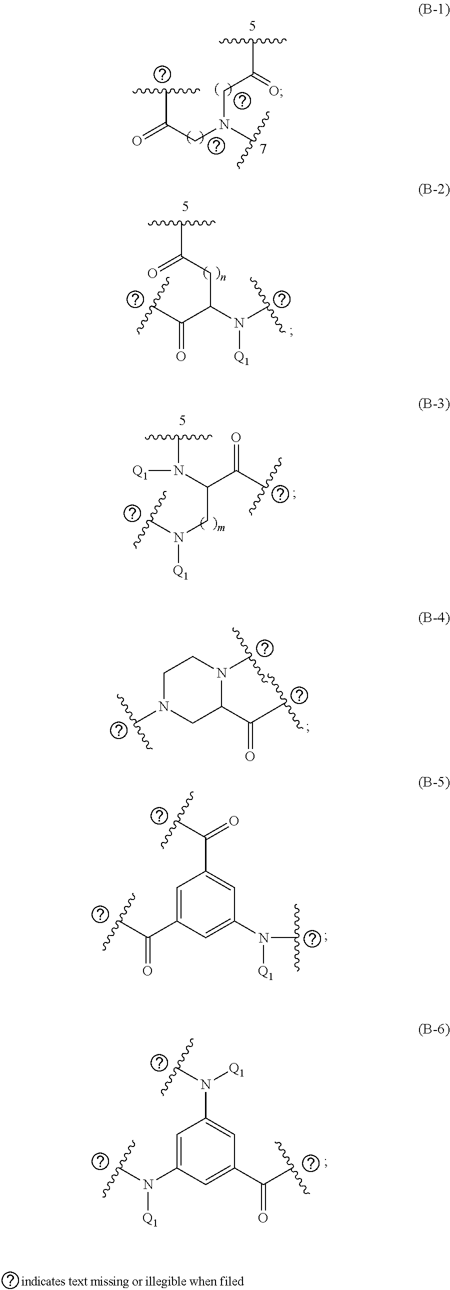

A₁ is linked to B at residue position 5, and A₂ is linked to B at residue position 6, by directly forming a bond with each other or by linker K;
in the above formulas, e and f are each an integer from 1 to 2; n is an integer from 1 to 3; m is an integer from 1 to 4; j is an integer from 1 to 5; p and q are each an integer from 1 to 6; r is an integer from 1 to 11, preferably, from 1 to 5; s and t are each an integer from 1 to 17; v and u are each an integer from 1 to 18; w is an integer from 1 to 19.

More further, in the ligand having a structure of Formula II, T₁ to T₄ may be the same or different, and T₁ to T₄ are each independently selected from:

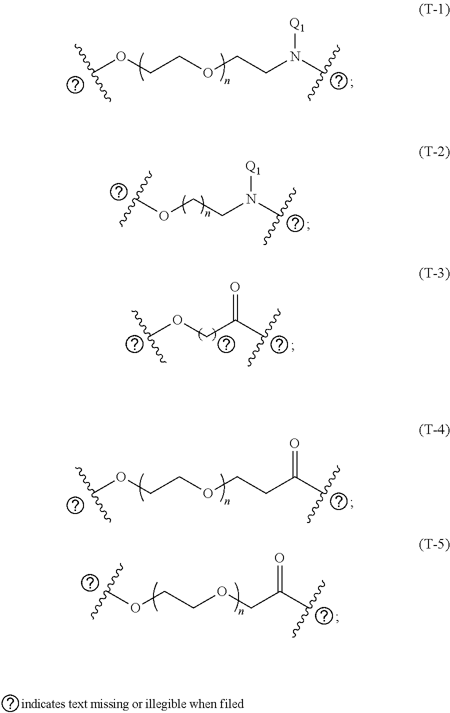

residue position 0 has a first atom/group that may be S or NQ₁;
T₁ to T₄ is linked to T₁′ to T₄′ at residue position 8, respectively, by directly forming a bond with each other or by linker K;
n is an integer from 1 to 3.

More further, in the ligand having a structure of Formula II,
T₁′ to T₄′ are linked to a₁ to a₄, or A₁ or A₂ at residue positions 1-4, by directly forming a bond with each other or by linker K; T₁′ to T₄′ are independently selected from:

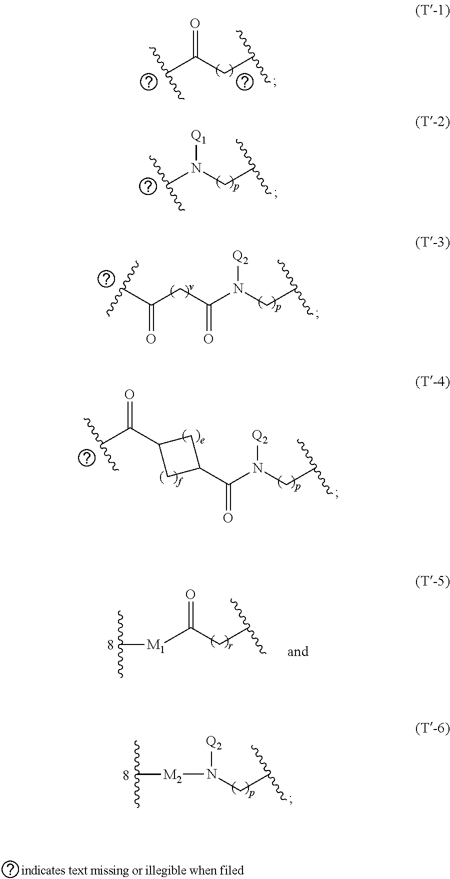

M₂ is selected from:

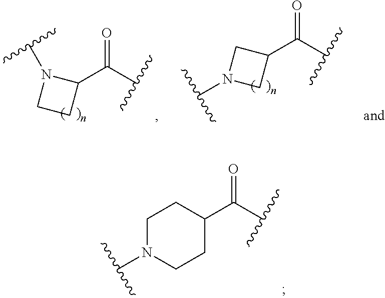 [SMILES: *C(=O)C1CCN1* ; *C(=O)C1CN(*)C1 ; *C(=O)C1CCN(*)CC1]

in the above formulas, e and f are each an integer from 1 to 2; n is an integer from 1 to 3; m is an integer from 1 to 4; j is an integer from 1 to 5; p and q are each an integer from 1 to 6; r is an integer from 1 to 11, preferably, from 1 to 5; s and t are each an integer from 1 to 17; v and u are each an integer from 1 to 18; w is an integer from 1 to 19.

Most preferably, the ligand having a structure of Formula II has any one of the following structures:

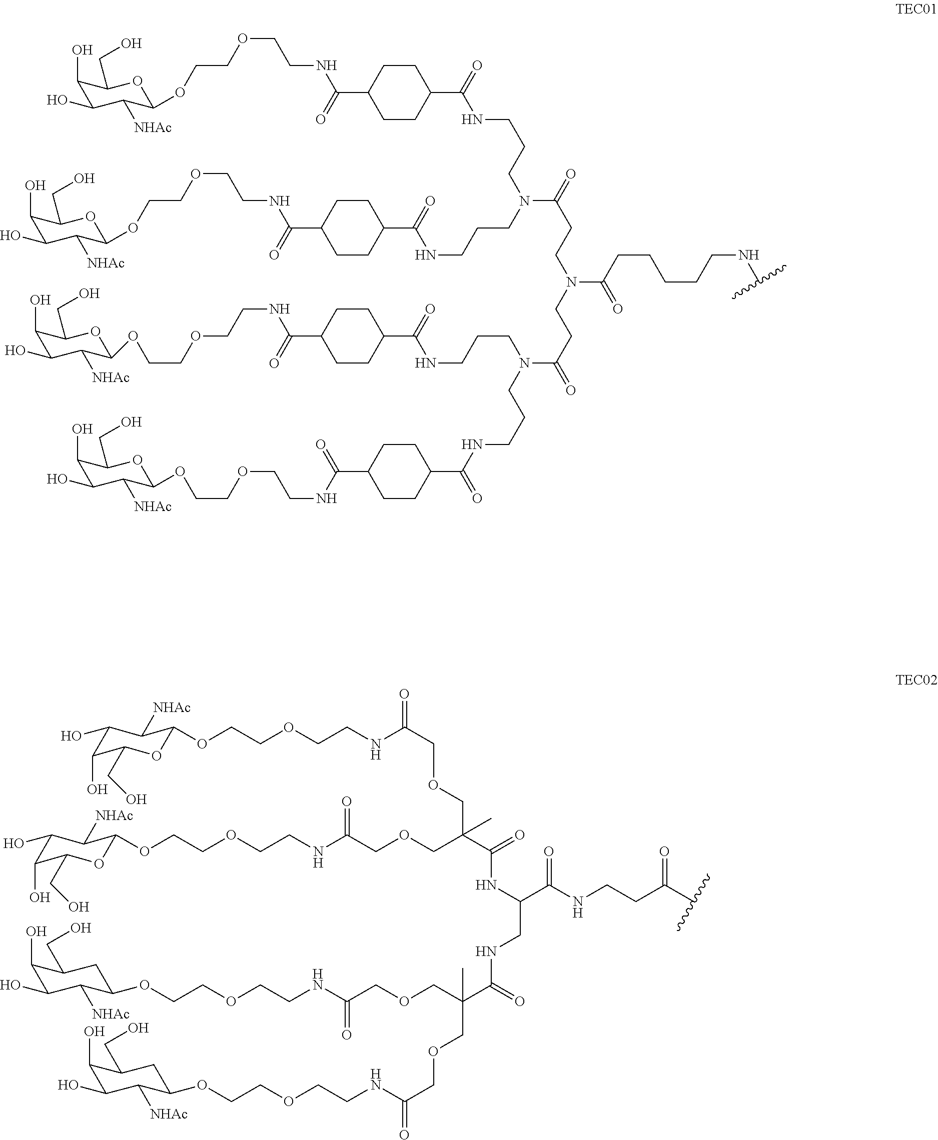 [SMILES: *NCCCCCC(=O)N(CCC(=O)N(CCCNC(=O)C1CCC(C(=O)NCCOCCOC2OC(CO)C(O)C(O)C2NC(C)=O)CC1)CCCNC(=O)C1CCC(C(=O)NCCOCCOC2OC(CO)C(O)C(O)C2NC(C)=O)CC1)CCC(=O)N(CCCNC(=O)C1CCC(C(=O)NCCOCCOC2OC(CO)C(O)C(O)C2NC(C)=O)CC1)CCCNC(=O)C1CCC(C(=O)NCCOCCOC2OC(CO)C(O)C(O)C2NC(C)=O)CC1 ; *C(=O)CCNC(=O)C(CNC(=O)C(C)(COCC(=O)NCCOCCOC1CC(CO)C(O)C(O)C1NC(C)=O)COCC(=O)NCCOCCOC1CC(CO)C(O)C(O)C1NC(C)=O)NC(=O)C(C)(COCC(=O)NCCOCCOC1OC(CO)C(O)C(O)C1NC(C)=O)COCC(=O)NCCOCCOC1OC(CO)C(O)C(O)C1NC(C)=O]

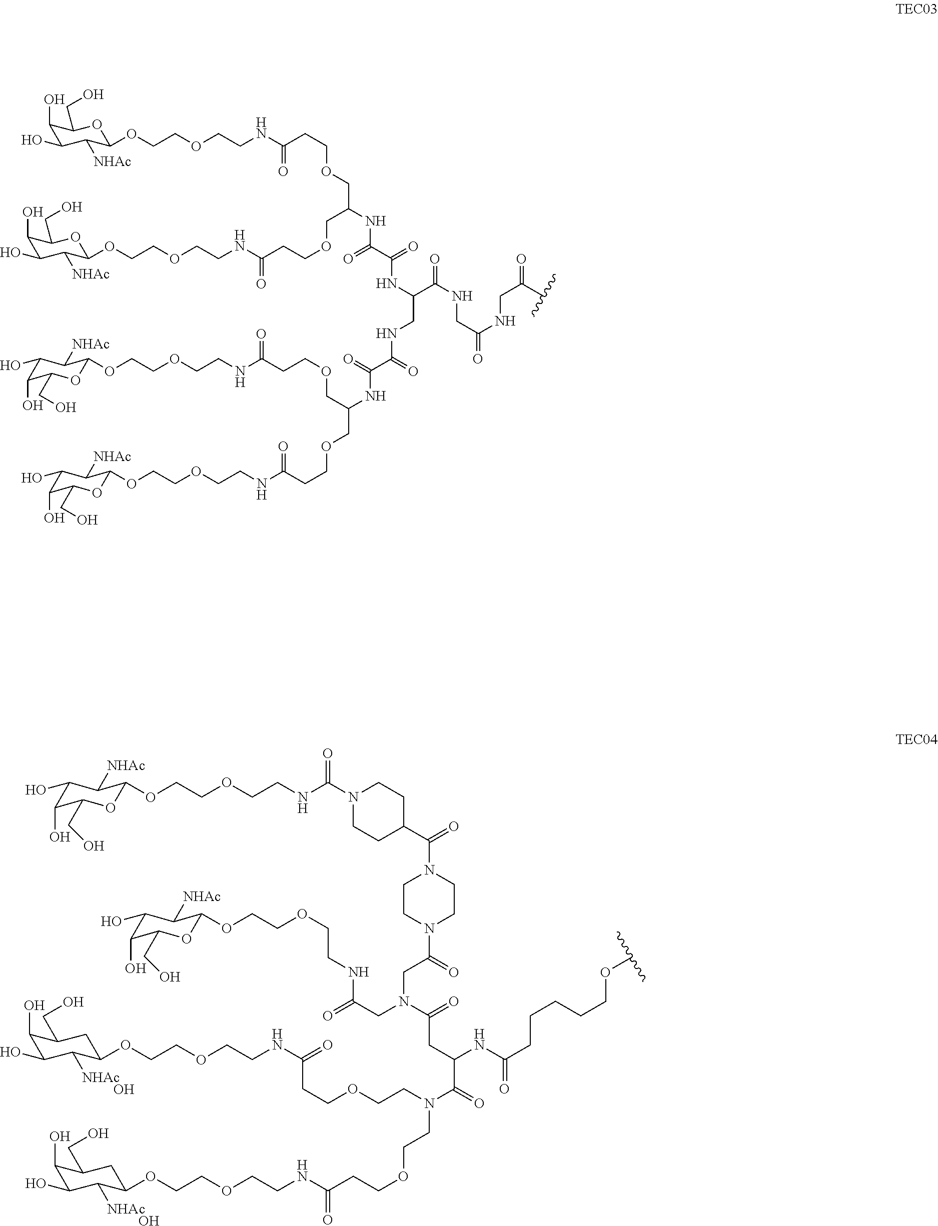

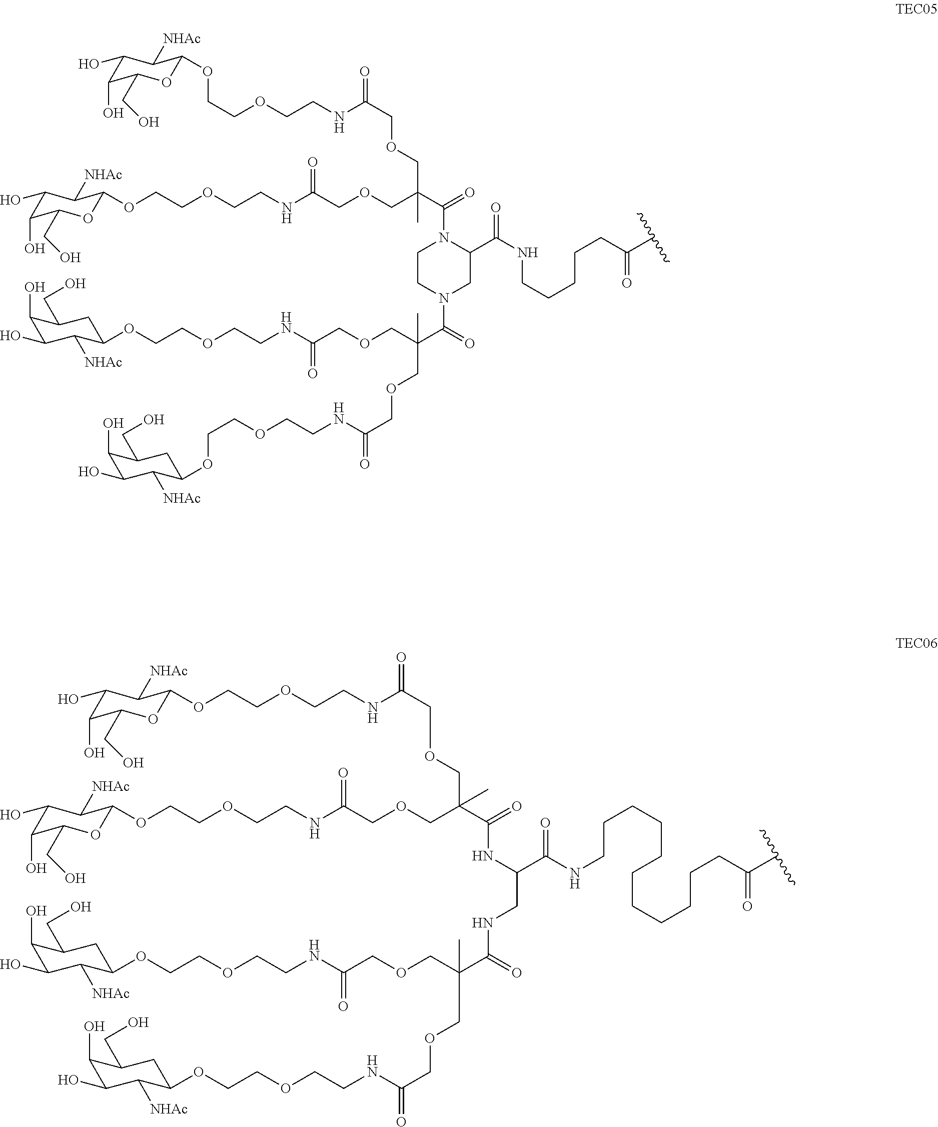 [SMILES: *C(=O)CCCCCNC(=O)C1CN(C(=O)C(C)(COCC(=O)NCCOCCOC2CC(CO)C(O)C(O)C2NC(C)=O)COCC(=O)NCCOCCOC2CC(CO)C(O)C(O)C2NC(C)=O)CCN1C(=O)C(C)(COCC(=O)NCCOCCOC1OC(CO)C(O)C(O)C1NC(C)=O)COCC(=O)NCCOCCOC1OC(CO)C(O)C(O)C1NC(C)=O ; *C(=O)CCCCCCCCCCCNC(=O)C(CNC(=O)C(C)(COCC(=O)NCCOCCOC1CC(CO)C(O)C(O)C1NC(C)=O)COCC(=O)NCCOCCOC1CC(CO)C(O)C(O)C1NC(C)=O)NC(=O)C(C)(COCC(=O)NCCOCCOC1OC(CO)C(O)C(O)C1NC(C)=O)COCC(=O)NCCOCCOC1OC(CO)C(O)C(O)C1NC(C)=O]

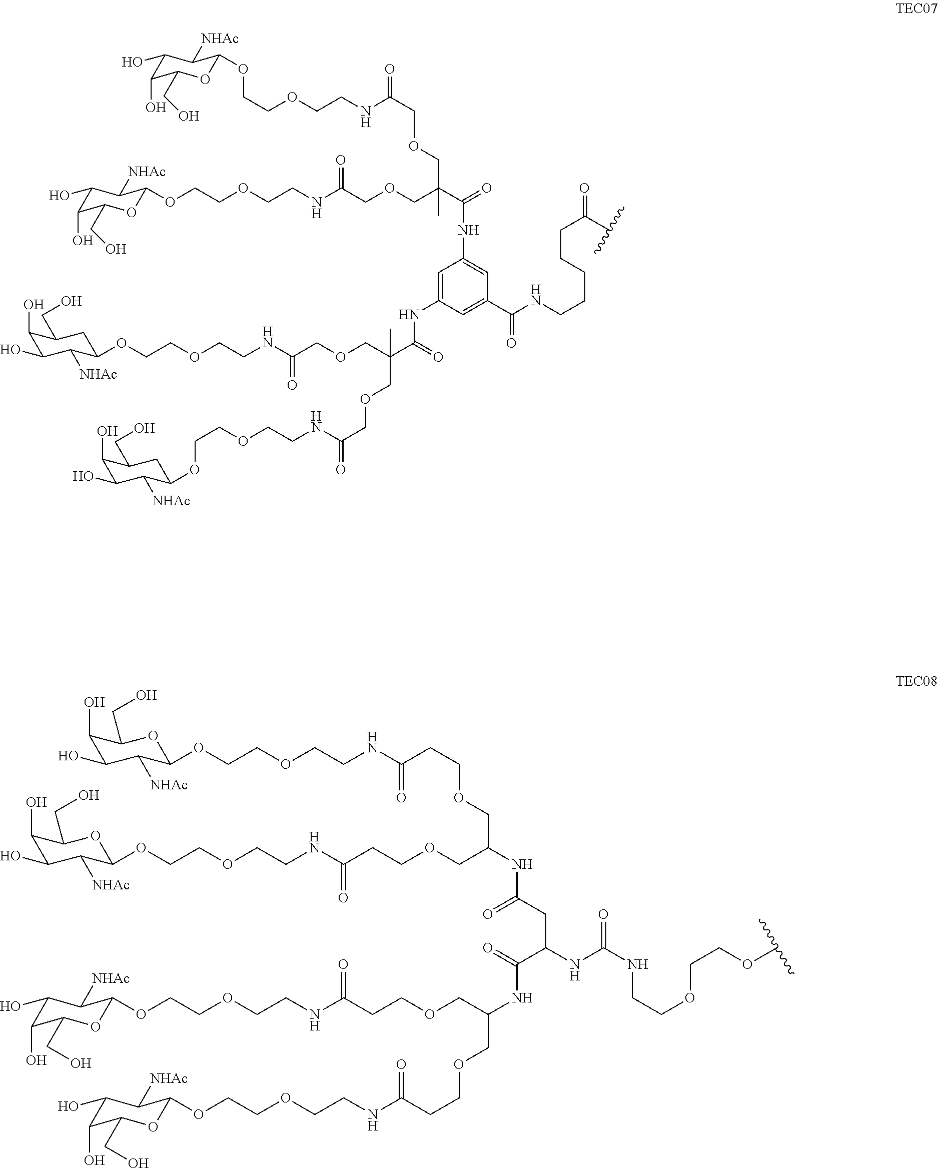

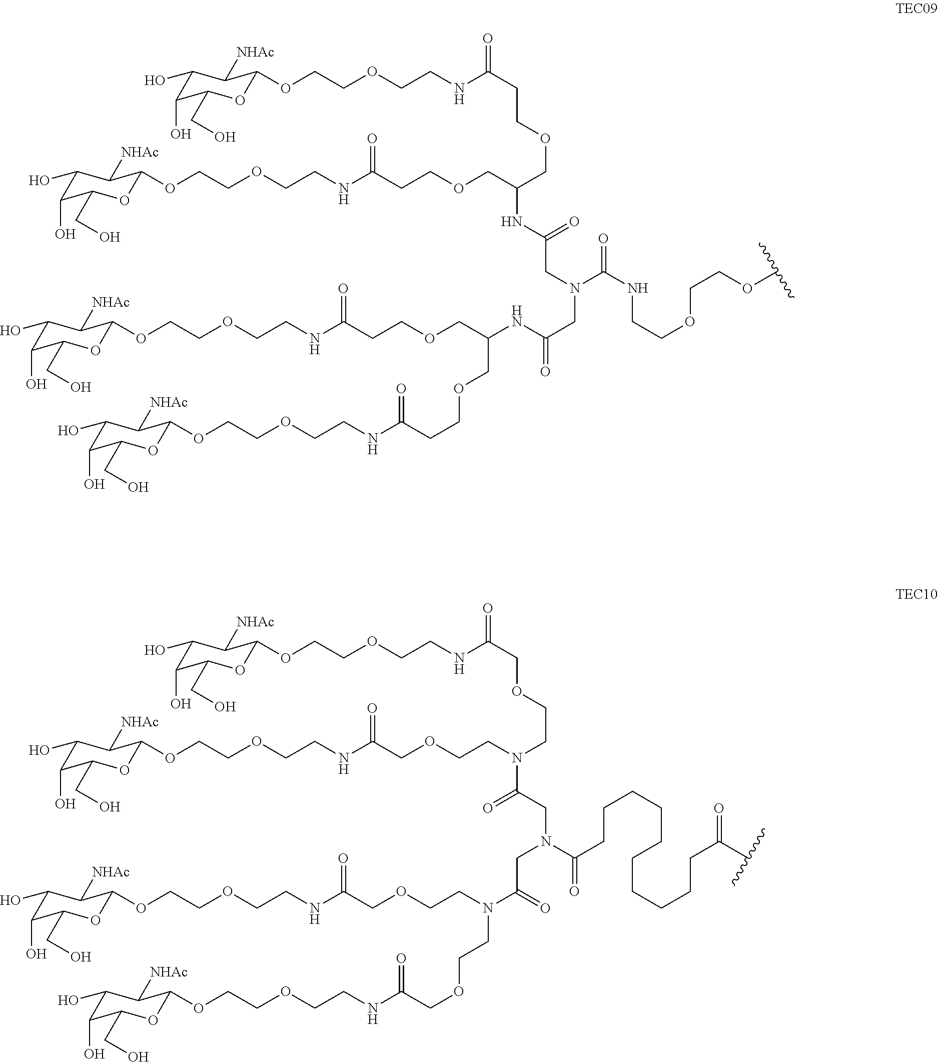 [SMILES: *OCCOCCNC(=O)N(CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O)CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCCCCCCCCCC(=O)N(CC(=O)N(CCOCC(=O)NCCOCCOC1OC(CO)C(O)C(O)C1NC(C)=O)CCOCC(=O)NCCOCCOC1OC(CO)C(O)C(O)C1NC(C)=O)CC(=O)N(CCOCC(=O)NCCOCCOC1OC(CO)C(O)C(O)C1NC(C)=O)CCOCC(=O)NCCOCCOC1OC(CO)C(O)C(O)C1NC(C)=O]

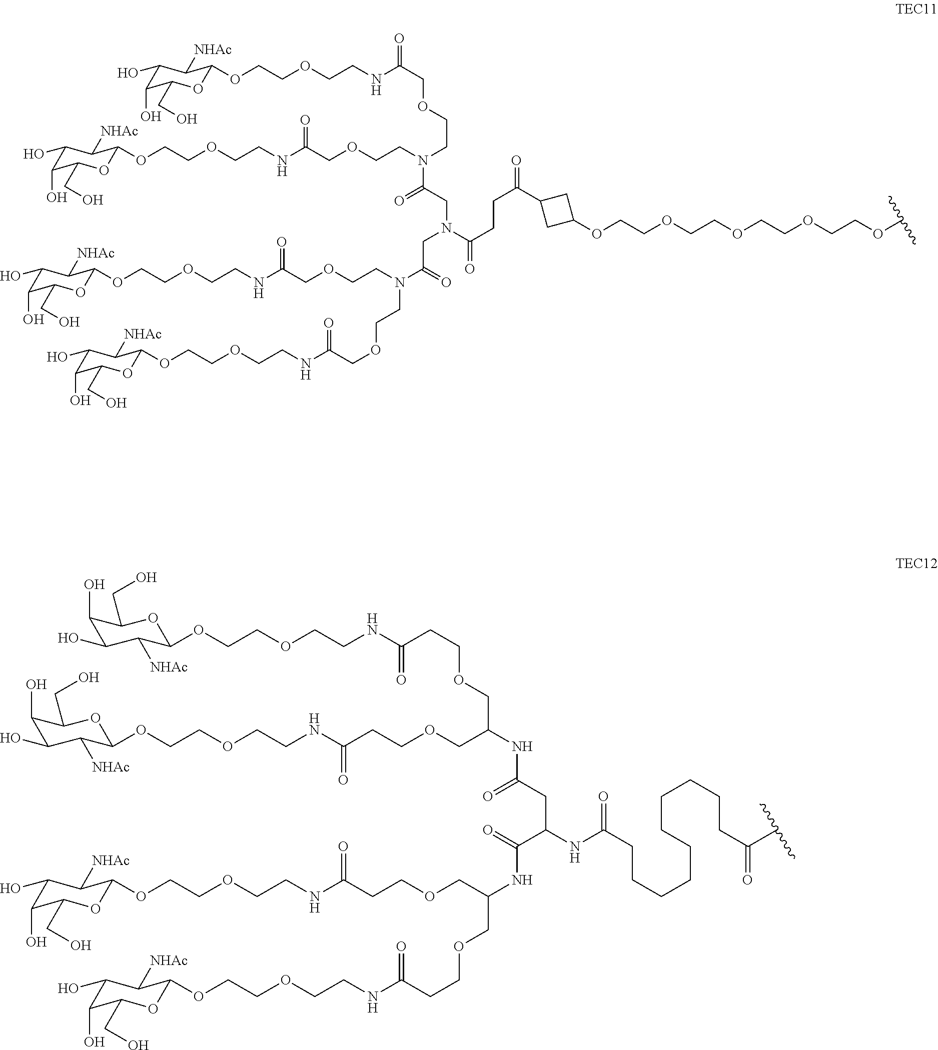

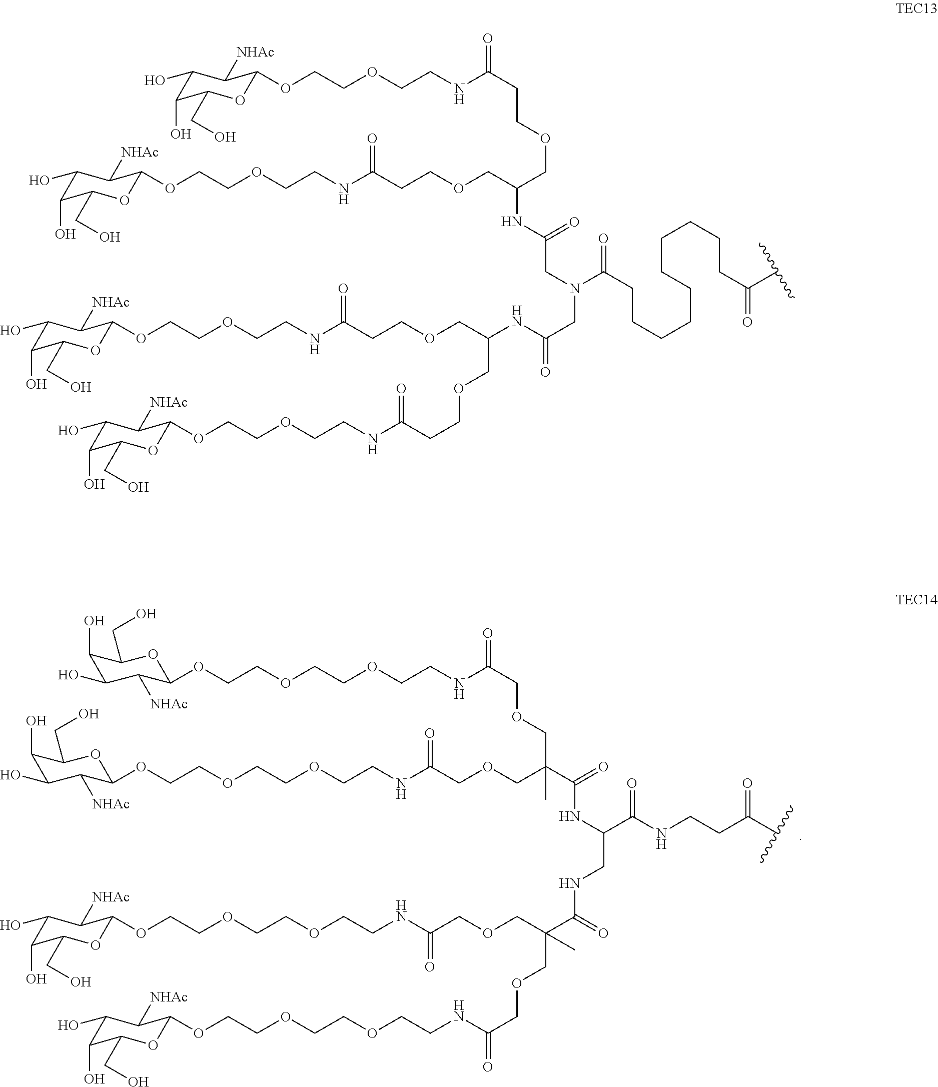 [SMILES: *C(=O)CCCCCCCCCCC(=O)N(CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O)CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCNC(=O)C(CNC(=O)C(C)(COCC(=O)NCCOCCOCCOC1OC(CO)C(O)C(O)C1NC(C)=O)COCC(=O)NCCOCCOCCOC1OC(CO)C(O)C(O)C1NC(C)=O)NC(=O)C(C)(COCC(=O)NCCOCCOCCOC1OC(CO)C(O)C(O)C1NC(C)=O)COCC(=O)NCCOCCOCCOC1OC(CO)C(O)C(O)C1NC(C)=O]

The present disclosure further relates to an activated/modified ligand having an activation/modification group obtained by activating/modifying the ligand, wherein the activation/modification group includes but is not limited to:

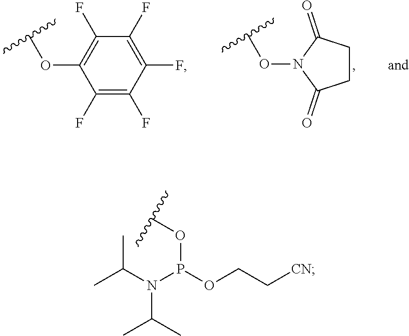 [SMILES: *Oc1c(F)c(F)c(F)c(F)c1F ; *ON1C(=O)CCC1=O ; *OP(OCCC#N)N(C(C)C)C(C)C]

and
the activation/modification is taken place at:
(1) residue position 5 of B terminus or residue position 7 of L terminus of the ligand of Formula I; or
(2) residue position 7 of B terminus or residue position 9 of L terminus of the ligand of Formula II.

The present disclosure further relates to use of the ligand and the activated/modified ligand in linking a functional compound; preferably, the functional compound is a drug; more preferably, the drug is a nucleic acid drug.

Further, the (activated/modified) ligand is used for in vivo delivery of the drug; preferably, the in vivo delivery is liver-targeted delivery.

The present disclosure further relates to a conjugate formed by coupling the ligand with a nucleic acid or a fluorescent compound, wherein,
(1) the conjugate formed by coupling the ligand of Formula I with a nucleic acid or a fluorescent compound has a structure of Formula III:

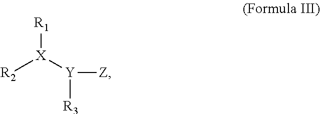

or
(2) the conjugate formed by coupling the ligand of Formula II with a nucleic acid or a fluorescent compound has a structure of Formula IV:

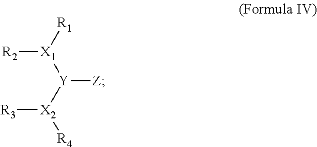

in Formula III and/or Formula IV, Z is the nucleic acid or the fluorescent compound;
the conjugate has a bond, including, but not limited to an amide bond, a phosphodiester bond and a phosphorothioate bond, between Y and Z.

The present disclosure further relates to a method for preparing the ligand of Formula V,

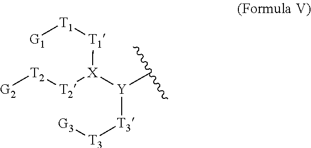

comprising the steps of:
(1) synthesis of a chain with sugar group comprising

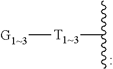

a glycoside comprising a chain of T₁-T₃ linked to position 1 of glycosyl and a protective group on saccharide hydroxyl is obtained by subjecting saccharide G₁-G₃ without a protective group to protection reaction (generally acetylation) to obtain a saccharide having a protective group on saccharide hydroxyl, which is then subjected to glycosylation reaction with a chain of T₁-T₃ having a terminal protective group at one end and a chemically reactive group at the other end, and further to deprotection reaction to remove the terminal protective group from T₁-T₃;
(2) synthesis of a chain with sugar group comprising

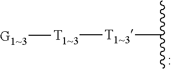

the saccharide chain comprising

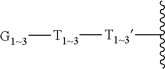

is obtained by subjecting the saccharide chain comprising

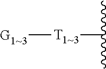

to condensation or substitution reaction with a compound comprising T₁′-T₃′, which may be deprotected first and then reacted with T₁-T₃;
(3) synthesis of a compound comprising

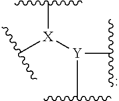

where a₁-a₃ are present, a X-comprising compound is firstly subjected to protection reaction to obtain a X-comprising compound in which a₁-a₃ have a protective group, which is then subjected to condensation or substitution reaction with a Y-comprising compound; a₁-a₃ may not be protected, and the X-comprising compound is directly linked to the Y-comprising compound by condensation or substitution reaction; if X and/or Y have a protective group at residue position 4, the linking reaction is conducted after the protective group is removed;
(4) synthesis of a compound comprising

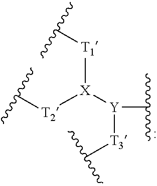

the compound comprising

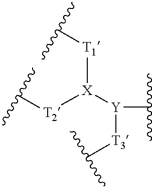

is obtained by subjecting the compound comprising

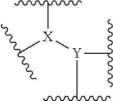

to addition, condensation or substitution reaction with a chain or a precursor compound comprising T₁′-T₃′, or alternatively by subjecting a compound comprising

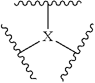

and a compound comprising

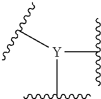

to addition, condensation or substitution reaction with the chain or the precursor compound comprising T₁′-T₃′ to obtain a compound comprising

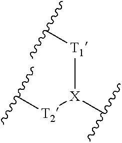

and a compound comprising

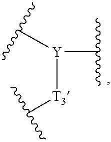

respectively, which are then subjected to condensation or substitution reaction with each other at residue position 4;
(5) synthesis of a compound comprising

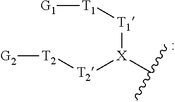

the compound comprising

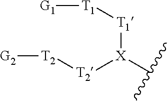

is obtained by subjecting the compound comprising

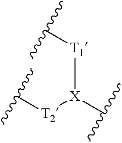

to deprotection reaction at T₁′-T₂′ terminus, and then to condensation or substitution reaction with the saccharide chain comprising

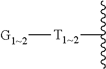

with protected glycosyl and unprotected T₁-T₂ terminus, or alternatively by subjecting a compound comprising

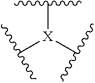

to condensation or substitution reaction with the saccharide chain comprising

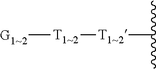

with protected glycosyl and unprotected T₁′-T₂′ terminus;
(6) synthesis of a compound comprising

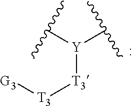

the compound comprising

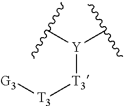

is obtained by subjecting the compound comprising

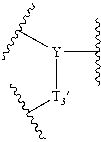

to deprotection reaction to remove the protective group at T₃′ terminus and then to condensation or substitution reaction with the saccharide chain comprising

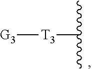

or alternatively by subjecting a compound comprising

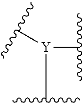

to condensation or substitution reaction with the saccharide chain comprising

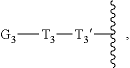

which are similar to the protocols in step (5);
(7) synthesis of a compound comprising

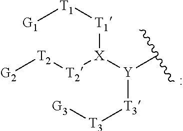

the compound comprising

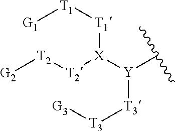

is obtained by subjecting the saccharide chain comprising

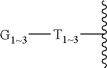

and the compound comprising

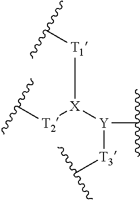

to deprotection reaction to remove their respective reactive groups, and then to condensation or substitution reaction with each other at residue positions 1-3, or alternatively by subjecting the saccharide chain comprising

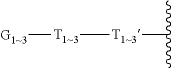

to reaction with the compound comprising

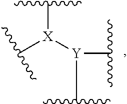

or alternatively by subjecting the compound comprising

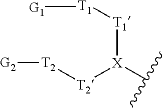

and the compound comprising

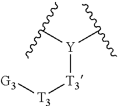

to deprotection reaction at residue position 4 and then to condensation or substitution reaction.

Further, the present disclosure relates to a method for further modifying the ligand of Formula III, comprising the steps of:
(8) linkage of L as defined to the ligand: a ligand compound comprising a naked chemically reactive group at L terminus is obtained by subjecting the L-comprising compound to condensation or substitution reaction with B at residue position 5 in any of steps (3)-(7), with or without final removal of the protective group from L terminus; and/or
(9) introduction of linker K as defined into the ligand: linker K is introduced either prior to or during the linkage of two segment at a specified position.

The present disclosure further relates to a method for preparing a ligand of Formula VI,

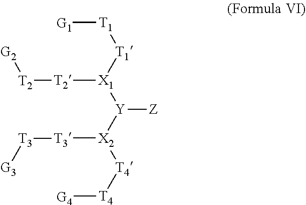

comprising the steps of:
(1) synthesis of a chain with sugar group comprising

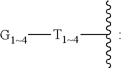

a glycoside comprising a chain of T₁-T₄ linked to position 1 of glycosyl and a protective group on saccharide hydroxyl is obtained by subjecting saccharide G₁-G₄ without a protective group to protection reaction (generally acetylation) to obtain a saccharide having a protective group on saccharide hydroxyl, which is then subjected to glycosylation reaction with a chain of T₁-T₄ having a protective group at one end and a chemically reactive group at the other end;
(2) synthesis of a chain with sugar group comprising

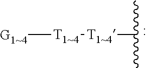

the saccharide chain comprising

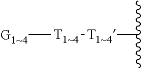

is obtained by subjecting the saccharide chain comprising

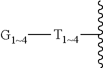

to deprotection reaction at T₁-T₄ terminus and then to condensation or substitution reaction with a compound comprising T₁′-T₄′; the compound comprising T₁′-T₄′ may be deprotected first and then react with T₁-T₄;
(3) synthesis of a compound comprising

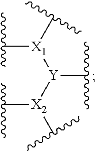

where a₁-a₄ are present, a compound comprising

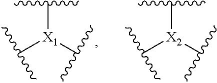

is firstly subjected to protection reaction to obtain a compound comprising

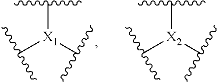

in which a₁-a₄ have a protective group, which is then subjected to condensation or substitution reaction with a compound comprising

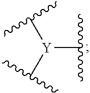

a₁-a₄ may not be protected, and the compound comprising

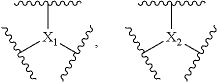

is directly linked to the compound comprising

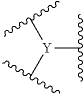

by condensation or substitution reaction; if

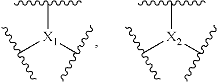

and/or

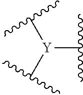

have a protective group at residue position 5/6, the linking reaction is conducted after the protective group is removed; where only a₁ and a₂ are absent, or where only a₃ and a₄ are absent, or where a₁-a₄ are all absent, the compound comprising

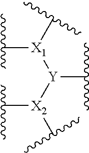

is synthesized by following the above-described method with a₁-a₄ protection/deprotection involved or not;
(4) synthesis of a compound comprising

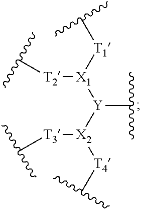

the compound comprising

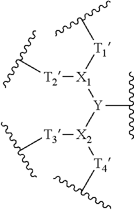

is obtained by subjecting the compound comprising

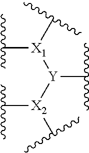

to addition, condensation or substitution reaction with a chain or precursor compound comprising T₁′-T₄′, or alternatively by subjecting the compound comprising

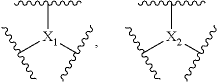

to addition, condensation or substitution reaction with the chain or precursor compound comprising T₁′-T₄′ to obtain a compound comprising

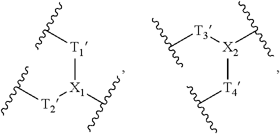

which is then subjected to condensation or substitution reaction with a compound comprising

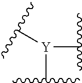

at residue position 5/6;
(5) synthesis of a compound comprising

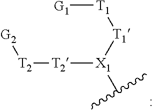

the compound comprising

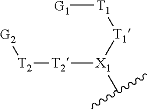

is obtained by subjecting the compound comprising

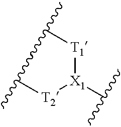

to deprotection reaction at T₁′-T₂′ terminus, and then to condensation or substitution reaction with the saccharide chain comprising

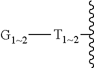

with protected glycosyl and unprotected T₁-T₂ terminus, or alternatively by subjecting a compound comprising

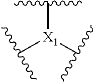

to condensation or substitution reaction with the saccharide chain comprising

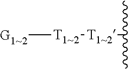

with protected glycosyl and unprotected T₁′-T₂′ terminus;
(6) synthesis of a compound comprising

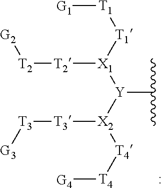

the compound comprising

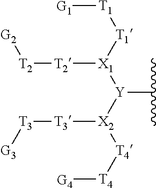

is obtained by subjecting the saccharide chain comprising

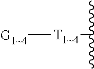

and the compound comprising

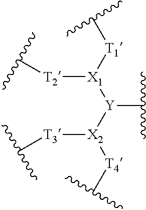

to deprotection reaction to remove their respective reactive groups, and then to condensation or substitution reaction with each other at residue positions 1-4, or alternatively by subjecting the saccharide chain comprising

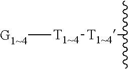

to reaction with the compound comprising

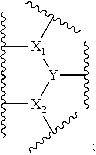

(7) introduction of L: a ligand compound having a naked chemically reactive group at L terminus is obtained by subjecting a L-comprising compound to condensation or substitution reaction with B at terminal residue position 7 in any of steps (3)-(6), with or without final removal of the protecting group from L terminus;
(8) introduction of linker K between any two of the abovementioned linked moieties: linker K is introduced either prior to or during the linkage of the two moieties at a specified position; and optionally, (9) modification of the ligand: the modified ligand is obtained by modifying the ligand having a chemically reactive group at L terminus with by an active ester or phosphoramidite.

### Term Definition

Chain structure refers to a chemical structure that morphologically extends in one dimension, formed by linking atom(s)/group(s) that can bond to each other in sequence, without strict limitation on the number of atom(s)/group(s). Backbone structure refers to a structure that serves as the starting point or core of a compound's structure, allows the molecule to extend outward by linking other chemical groups or structures, and plays a role similar to backbone. Triple-branched form refers to a branched structure which has three branches extending outward morphologically or a branched structure which can form a triple-branched compound by conveniently linking to other groups through three chemically modifiable sites. Chemically reactive group refers to a group that has sufficient chemical reactivity to carry out the desired chemical reaction under reaction conditions as described or relatively easy to achieve.

O-glucosidic, S-glucosidic and N-glucosidic bonds refer to oxygen-, sulfur- and nitrogen-glucosidic bonds, respectively, and O, S and N represent oxygen, sulfur and nitrogen atoms, respectively.

Receptor is a biological macromolecule that can bind to a substance such as a drug or a signaling molecule and cause changes in biological functions.

Ligand is a molecule that can specifically bind to a receptor.

In the present disclosure, the ligand is a chemical structure with a certain molecular backbone, and may comprise one or more chiral centers and thus may comprise optical enantiomers and diastereomers of the structure, since chiral change of other constituent moieties other than glycosyl in the structure generally does not affect the chemical construction mode, synthesis process and preparation method of the structure. There is a part

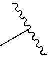

in the ligand structure, indicating that the group at that site is variable, can be a chemically acceptable group, and can be linked to or combined with other chemical groups, structures, supports, etc., in a covalent or non-covalent manner.

In the present disclosure, the synthesis of a certain compound or intermediate is described in a concise manner. Starting material refers to main raw material for synthesis of a compound or intermediate, and comprises the main structural moieties that constitute the compound or intermediate.

Substitution reaction refers to a reaction in which a compound having a leaving group such as halogen is subjected to substitution by a nucleophilic group such as an amino/amine, hydroxyl or sulfhydryl group under the action of alkali to liberate one molecule of hydrogen halide and generate a N—C, O—C or S—C bond.

Amidation condensation reaction or amidation reaction refers to a reaction in which a carboxyl group reacts with an amino/amine group in presence of a condensating agent to liberate one molecule of water and form an amide bond; it also comprises a reaction in which an amino/amine group reacts with acyl chloride, anhydride and active ester to form an amide bond.

Urea condensation reaction refers to a reaction in which two molecules of amino/amine groups react with one molecule of carbonyl donor reagent to form urea.

9-Fluorenylmethoxycarbonylation protection reaction refers to a reaction in which an acylation reagent such as 9-fluorenylmethoxycarbonyl chloride reacts with an amino/amine group to produce 9-fluorenylmethoxycarbonyl amino/substituted amino group.

Benzylation protection reaction refers to a reaction in which benzyl bromide or chloride reacts with a hydroxy, amino/amine or carbonyl group to produce benzyl ether of hydroxyl, benzylamine or benzyl carboxylate.

Benzyloxycarbonylation protection reaction refers to a reaction in which an acylation reagent such as benzyloxycarbonyl chloride with an amino/amine group to produce benzyloxycarbonyl amino/substituted amino group.

Debenzylation reaction refers to a reaction in which a hydroxyl, amino/amine or carboxyl group protected by a benzyl group is subjected to breakage and removal of the benzyl group to produce a naked hydroxyl, amino/amine or carboxyl group in the presence of Pd/C catalysts, and hydrogen or hydrogen donor reagents.

De-benzyloxycarbonylation reaction refers to a reaction in which an amino/amine group protected by a benzyloxycarbonyl group is subjected to breakage and removal of the benzyloxycarbonyl group to produce a naked amino/amine group under the action of Pd/C catalysts, and hydrogen or hydrogen donor reagents.

De-tert-butylation reaction refers to a reaction in which a carboxyl group protected by a tert-butyl group (tert-butyl ester) is subjected to removal of the tert-butyl group or decomposition/hydrolysis of the tert-butyl ester to produce a naked carboxyl group under acidic conditions.

De-tert-butoxycarbonylation reaction refers to a reaction in which an amino/amine group protected by a tert-butoxycarbonyl group is subjected to decomposition of the tert-butoxycarbonyl group to produce a naked amino/amine group (acid addition salts of ammonia/amine) under acidic conditions.

De-9-fluorenylmethoxycarbonylation reaction refers to a reaction in which an amino/amine group protected by a 9-fluorenylmethoxycarbonyl group is subjected to breakage and removal of the 9-fluorenylmethoxycarbonyl group to produce a naked amino/amine under the action of a base (generally a secondary amine or tertiary amine) or fluorine ion.

Demethylation reaction refers to a reaction in which a carboxyl group protected by methyl ester is subjected to hydrolysis of methyl ester to produce a naked carboxyl group/carboxylate under alkaline conditions.

Addition reaction refers to the nucleophilic addition of a compound having a nucleophilic group such as hydroxyl, sulfhydryl, primary amino group or secondary amino group to β-carbon atom of an α,β-unsaturated carbonyl or cyano compound under base catalysis to produce a saturated carbonyl or cyano compound having a O—C bond (an ether), S—C bond (a thioether) or N—C bond at β-carbon atom. Preferably, the nucleophilic group is a hydroxyl group, and the corresponding compound is a primary alcohol, secondary alcohol or tertiary alcohol, preferably a primary alcohol or secondary alcohol. The base is generally an alkali metal hydroxide (sodium hydroxide, potassium hydroxide or lithium hydroxide), a hydride (sodium hydride, potassium hydride or lithium hydride) or an alkoxide (potassium/sodium alkoxide, generally methoxide, ethoxide or tert-butoxide), or an alkali metal carbonate (potassium carbonate, sodium carbonate, cesium carbonate, lithium carbonate, etc.), or a basic amine (triethylamine, diisopropylethylamine, etc.), or an alkali amide (e.g., sodium amide, lithium diisopropylamide or potassium/sodium/lithium hexamethyldisilylamide). The α,β-unsaturated carbonyl compound is generally an acrylate (preferably tert-butyl acrylate, methyl acrylate, ethyl acrylate or benzyl acrylate), and the α,β-unsaturated cyano compound is generally acrylonitrile.

### BRIEF DESCRIPTION OF THE DRAWINGS

FIG. 1 shows binding test results of the ligand synthesized according to the present disclosure with primary mouse liver cells.

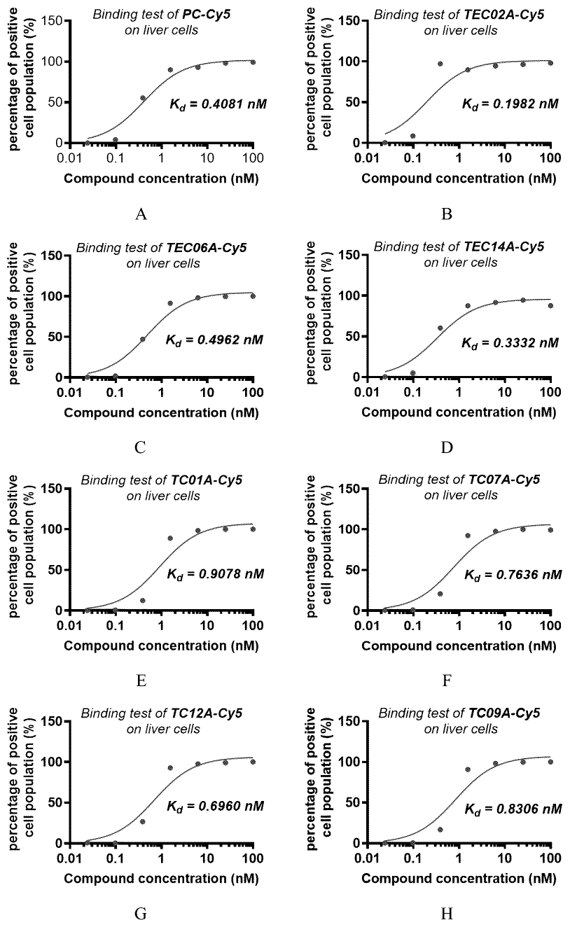

FIG. 2 shows binding test results of the ligand synthesized according to the present disclosure with HepG2 cells.

### DETAILED DESCRIPTION

### Reagents and Materials

Unless otherwise specified, the raw materials, reagents and solvents used in the present disclosure for synthesis are analytically pure.

### Main Reagents, Solvents, Abbreviations and CAS Numbers

DMF: N,N-dimethylformamide, 68-12-2
THF: tetrahydrofuran, 109-99-9
DCM: dichloromethane, 75-09-2
EtOAc: ethyl acetate, 141-78-6
1,4-Dioxane: 1,4-dioxane, 123-91-1
MTBE: methyl tert-butyl ether, 1634 Apr. 4
DMSO: dimethyl sulfoxide, 67-68-5
TBAI: tetrabutylammonium iodide, 311-28-4
TBAB: tetrabutylammonium bromide, 1643-19-2
TBAF: tetrabutylammonium fluoride, 429-41-4
Pd/C: palladium (Pd) catalyst on activated carbon, with 10 wt % of palladium, 7440 May 3
EDCI: 1-ethyl-(3-dimethylaminopropyl) carbodiimide hydrochloride, 25952-53-8
HOBt: 1-hydroxybenzotriazole, 2592-95-2
PyBOP: 1H-benzotriazol-1-yl-oxytripyrrolidinophosphonium hexafluorophosphate, 128625-52-5
DMT-MM: 4-(4,6-dimethoxy-1,3,5-triazin-2-yl)-4-methyl morpholinium chloride, 3945-69-5
EEDQ: 2-ethoxy-1-ethoxycarbonyl-1,2-dihydroquinoline, 16357-59-8
CDI: N,N′-carbonyldiimidazole, 530-62-1
DCC: N,N′-dicyclohexylcarbodiimide, 538-75-0
DMAP: 4-dimethylaminopyridine, 1122-58-3
NMM: N-methylmorpholine, 109-02-4
DIEA: N,N′-diisopropylethylamine, 7087-68-5
TEA: triethylamine, 121-44-8
2-cyanoethyl-N,N-diisopropylchlorophosphoramidite, 89992-70-1
N-hydroxysuccinimide, 6066-82-6
4-nitrophenyl chloroformate, 7693-46-1
isobutyl chloroformate, 543-27-1
TFA-Pfp: pentafluorophenyl trifluoroacetate, 14533-84-7
NaI: sodium iodide, 7681-82-5
LiOH: lithium hydroxide, 1310-65-2
NaH: sodium hydride, 7646-69-7
BnBr: benzyl bromide, 100-39-0
CbzCl: benzyl chloroformate, 501-53-1
FmocCl: 9-fluorenylmethoxycarbonyl chloride, 28920-43-6
Boc₂O: di-tert-butyl dicarbonate, 24424-99-5
TFA: trifluoroacetic acid, 76-05-1
0.5 N hydrochloric acid: an aqueous hydrochloric acid solution with a concentration of 0.5 mol/L
of hydrogen chloride
Boc: tert-butoxycarbonyl
Fmoc: 9-fluorenylmethoxycarbonyl
Cbz: benzyloxycarbonyl
^{t}Bu: tert-butyl
ⁱBu: isobutyl
Bn: benzyl
Pfp: pentafluorophenyl
OSu: succinimide
TLC: thin-layer chromatography on silica gel
LCMS: liquid chromatography-mass spectrometry
prep-HPLC: preparative high-performance liquid chromatography
monohydrate: a conjugate of compound molecules and water molecules in a ratio of 1:1

TABLE 1
Main raw materials

| ID | Name | CAS |
|---|---|---|
| SM1 | bis(hydroxymethyl)propionic acid | 4767-03-7 |
| SM2 | serinol | 534-03-2 |
| SM2-2 | 2-amino-2-methyl-1,3-propanediol | 115-69-5 |
| SM3 | diethanolamine | 111-42-2 |
| SM4 | N-Boc-L-serine monohydrate | 3262-72-4 |
| SM4-2 | L-serine benzyl ester hydrochloride | 60022-62-0 |
| SM5 | N-Boc-trans-4-hydroxy-L-proline methyl ester | 74844-91-0 |
| SM5-2 | trans-4-hydroxy-L-proline benzyl ester | 62147-27-7 |
| | hydrochloride | |
| SM5-3 | trans-N-Cbz-4-hydroxy-L-proline | 13504-85-3 |
| SM6 | N-Cbz-L-glutamic acid-5-tert-butyl ester | 3886-08-6 |
| SM7 | L-glutamic acid-5-tert-butyl ester | 2419-56-9 |
| SM8 | Nε-Boc-L-lysine | 2418-95-3 |
| SM9 | N-Cbz-L-serine | 1145-80-8 |
| SM10 | trans-4-hydroxy-L-proline methyl ester | 40216-83-9 |
| | hydrochloride | |

TABLE 2
Main raw materials

| Name | CAS | Name | CAS |
|---|---|---|---|
| tert-butyl bromoacetate | 5292-43-3 | Boc-β-alanine | 3303-84-2 |
| methyl bromoacetate | 96-32-2 | 3-amino-L-alanine | 1482-97-9 |
| | | hydrochloride | |
| tert-butyl 3-bromopropionate | 55666-43-8 | 12-aminododecanoic acid | 693-57-2 |
| methyl 3-bromopropionate | 3395-91-3 | glycylglycine | 556-50-3 |
| 3-hydroxypropionic acid | 503-66-2 | oxalyl chloride | 79-37-8 |
| tert-butyl acrylate | 1663-39-4 | N-Boc-6-aminocaproic | 6404-29-1 |
| | | acid | |
| succinic anhydride | 108-30-5 | ε-caprolactone | 502-44-3 |
| N-Boc-L-serine monohydrate | 204191-40-2 | (R)-1,4-bis-Boc-piperazine- | 173774-48-6 |
| | | 2-carboxylic acid | |
| N-Boc-bromoethylamine | 39684-80-5 | Boc-L-aspartic acid | 13726-67-5 |
| 3-(N-Boc-amino)propyl | 83948-53-2 | N-Fmoc-D-aspartic | 112883-39-3 |
| bromide | | acid-4-tert-butyl ester | |
| benzyl 6-aminohexanoate | 5515-01-5 | diglycolamine | 929-06-6 |
| 6-aminocaproic acid | 60-32-2 | N-Boc-minodiacetic acid | 56074-20-5 |
| monobenzyl dodecanediate | 88353-04-2 | 1-Cbz-3-hydroxyazetidine | 128117-22-6 |
| dimethyl 5-aminoisophthalate | 99-27-4 | tetraethylene glycol | 77544-60-6 |
| | | monotosylate | |
| methyl 5-nitroisophthalate | 1955-46-0 | benzylamine | 100-46-9 |
| 3,5-diaminobenzoic acid | 535-87-5 | chloroacetyl chloride | 79-04-9 |
| 2-Benzylaminoethanol | 104-63-2 | piperazine | 110-85-0 |
| Nα-Cbz-Nε-Boc-D-lysine | 2389-60-8 | N-Boc-piperidine-4- | 84358-13-4 |
| | | carboxylic acid | |
| trans-1,4-cyclohexanedicarboxylic | 15177-67-0 | 1-Boc-4-amino piperidine | 87120-72-7 |
| acid monomethyl ester | | | |

### Example 1. Synthesis Process and Molecular Identification of Ligands (Compounds of Formula I, Activated Esters and Fluorescent Conjugates Thereof)

### 1. Synthesis of Intermediates CC-1A, CC-1B, CC-2A and CC-2B

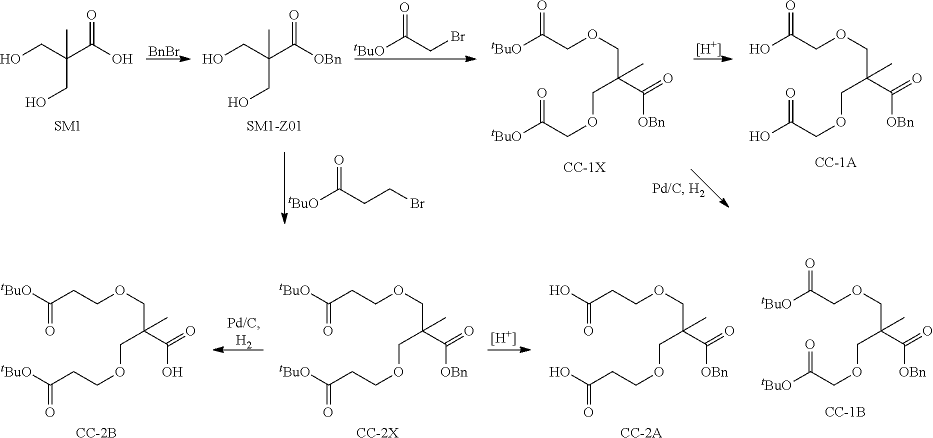

### (1) Synthesis of SM1-Z01 (Benzylation Protection Reaction)

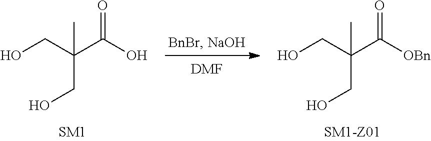

SM1 (13.4 g, 99.9 mmol) and sodium hydroxide (4 g, 100 mmol) were added to a flask containing N,N-dimethylformamide (74 mL), heated to 100° C. and stirred until the reaction mixture became clear. Next, with the temperature maintained 100° C. and under constant stirring, the reaction solution was dropwise added with benzyl bromide (20.5 g, 119.9 mmol) in 20 min, and then the reaction was carried out under stirring for 20 h when TLC showed that the conversion of the raw materials was complete (dichloromethane/methanol, 10/1, R_{f} of the product: 0.4). The reaction solution was slowly diluted with pure water (190 mL) and extracted with ethyl acetate (45 mL×3). The obtained organic phases were combined, washed with 26% aqueous sodium chloride solution (35 mL×2), dried over anhydrous sodium sulfate, filtered and concentrated under reduced pressure to obtain a colorless solid (a crude product). The crude product was purified by silica gel column chromatography (200-300 meshes, petroleum ether/ethyl acetate, 4/1 to pure ethyl acetate) to obtain 9.9 g of a colorless transparent liquid with a yield of 44.2%.

MS (ESI): m/z [M+Na]⁺, theoretical 247.2, found 247.2.

### (2) Synthesis of CC-1X (Substitution Reaction)

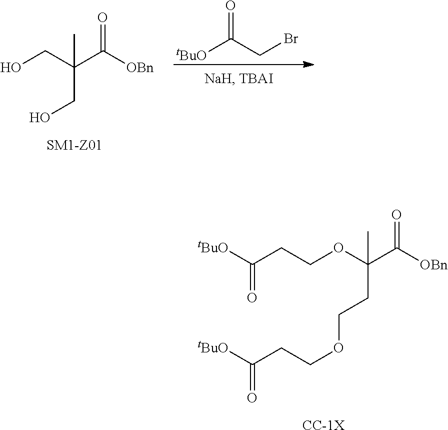

Under N₂ atmosphere and at room temperature, sodium hydride (60%, 19.2 g, 479.5 mmol) was slowly added to tert-butyl bromoacetate (109.1 g, 559.4 mmol) and tetrabutylammonium iodide (2.9 g, 8.0 mmol) in tetrahydrofuran (300 mL) in batches, and stirred at room temperature for 0.5 h. The reaction was then dropwise added with SM1-Z01 (17.9 g, 79.9 mmol) dissolved in tetrahydrofuran (60 mL) within 0.5 h and then carried out under stirring for 20 h when LCMS showed that the reaction was complete. The reaction solution was slowly added to 25% aqueous ammonium chloride solution (1000 mL) which was cooled in an ice bath of 0-5° C. and constantly stirred to quench the reaction, and concentrated under reduced pressure to remove about 300 mL of the solvent, and the residue was extracted with ethyl acetate (200 mL×3). The obtained organic phases were combined, washed with 26% aqueous sodium chloride solution (200 mL), dried over anhydrous sodium sulfate and concentrated to obtain a yellow liquid (a crude product). The crude product was purified by silica gel column chromatography (200-300 meshes, petroleum ether/ethyl acetate, 9/1) to obtain 8.0 g of a colorless transparent liquid with a yield of 22.2%.

MS (ESI): m/z [M+Na]⁺, theoretical 475.2, found 475.2.

¹H NMR (400 MHz, DMSO-d₆) δ 7.39-7.28 (m, 5H), 5.12 (s, 2H), 3.96 (s, 4H), 3.59 (dd, J=13.1, 8.7 Hz, 4H), 1.41 (br s, 19H), 1.18 (s, 3H).

### (3) Synthesis of CC-1A (De-Tert-Butylation Reaction)

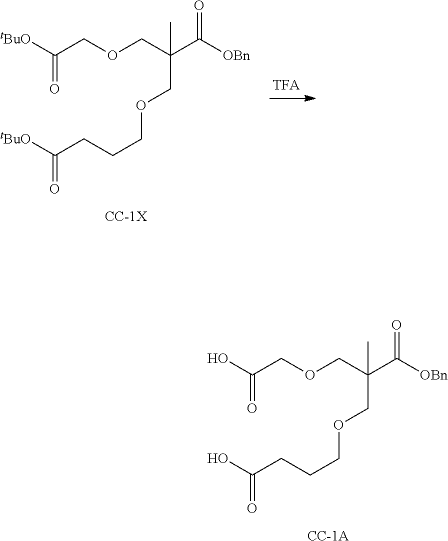

CC-1X (6 g, 13.3 mmol) and dichloromethane (30 mL) were successively added to a flask, and under constant stirring and at room temperature, trifluoroacetic acid (12.1 g, 106.2 mmol) was slowly added, and then the reaction was carried out for 16 h when LCMS showed the reaction was complete. The reaction solution was concentrated under reduced pressure to be oily, and dried under vacuum at 50° C. for 1 h to obtain 4.7 g of a yellow oily liquid product with a yield of 104% (the product contained some residual TFA and could be directly used in subsequent reaction without purification).

MS (ESI): m/z [M−H]⁻, theoretical 339.1, found 339.1.

### (4) Synthesis of CC-1B (Debenzylation Reaction)

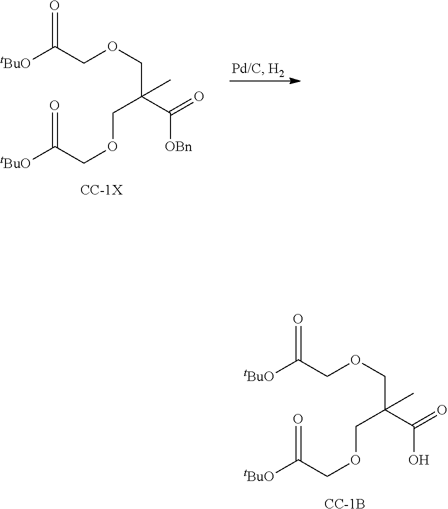

CC-1X (1.0 g, 2.2 mmol) was dissolved in tetrahydrofuran (6 mL) and added with Pd/C (0.1 g, 10 wt %). The reaction system was replaced with hydrogen for three times and then the reaction was carried out under stirring and under hydrogen atmosphere (about 10-20 kPa higher than the standard atmospheric pressure) for 2 h as LCMS showed that there was no residual raw material. The obtained reaction mixture was filtered to remove the insoluble solid, and the filtrate was concentrated and dried under vacuum to obtain 0.74 g of a white solid product with a yield of 93%.

MS (ESI): m/z [M+H]⁺, theoretical 363.2, found 363.3.

### (5) Synthesis of CC-2X (Substitution Reaction)

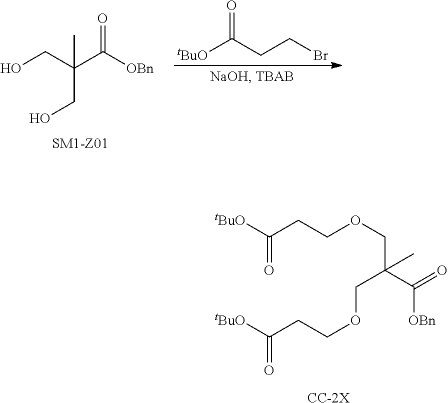

SM1-Z01 (4.0 g, 17.8 mmol) was added to a flask and dissolved with dichloromethane (40 mL), followed by successive addition of 37% aqueous sodium hydroxide solution (45 mL), tetrabutylammonium bromide (2.87 g, 8.9 mmol) and tert-butyl 3-bromopropionate (11.2 g, 53.4 mmol) under stirring at room temperature. The reaction was then carried out under stirring for 16 h when TLC showed the reaction was completed (petroleum ether/ethyl acetate, 4/1, R_{f} of the product: 0.3). The reaction solution was diluted with pure water (50 mL) and extracted with dichloromethane (30 mL×3). The obtained organic phases were combined, dried over anhydrous sodium sulfate, filtered, and concentrated under reduced pressure, and the residue was purified by silica gel column chromatography (200-300 meshes, petroleum ether/ethyl acetate, 10/1) to obtain 2.74 g of a colorless oily product with a yield of 32%.

MS (ESI): m/z [M+Na]⁺, theoretical 503.3, found 503.1.

¹H NMR (400 MHz, DMSO-d₆) δ 7.37-7.26 (m, 5H), 5.11 (s, 2H), 3.69-3.62 (m, 4H), 3.36 (dd, J=10.6, 7.2 Hz, 4H), 2.54 (t, J=5.4 Hz, 4H), 1.43 (br s, 19H), 1.16 (s, 3H).

### (6) Synthesis of CC-2A (De-Tert-Butylation Reaction)

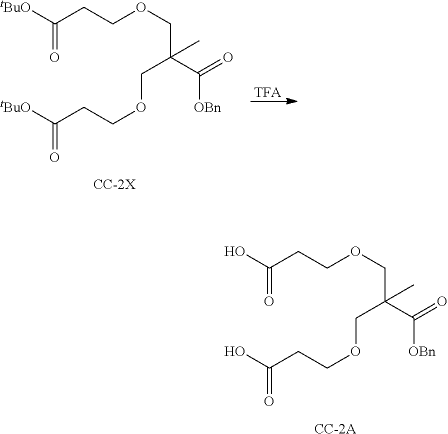

CC-2X (6 g, 12.5 mmol) and dichloromethane (20 mL) was successively added to a flask, and under constant stirring and at room temperature, trifluoroacetic acid (9.1 g, 80.0 mmol) was slowly added, and then the reaction was carried out for 6 h when LCMS showed that the reaction was completed. The reaction solution was concentrated under reduced pressure to be oily, and dried under vacuum at 50° C. for 1 h to obtain 5.1 g of a yellow oily liquid product with a yield of 110% (the product contained some residual TFA and could be directly used in subsequent reaction without purification).

MS (ESI): m/z [M−H]⁻, theoretical 367.2, found 367.1.

### (7) Synthesis of CC-2B (Debenzylation Reaction)

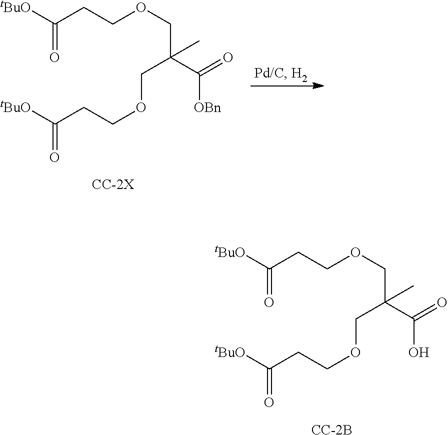

CC-2X (0.8 g, 1.67 mmol) was dissolved in tetrahydrofuran (4 mL) and added with Pd/C (80 mg, 10 wt %). The reaction system was replaced with hydrogen for three times and then the reaction was carried out under stirring and under hydrogen atmosphere (about 10-20 kPa higher than the standard atmospheric pressure) for 2 h when LCMS showed that there was no residual raw material. The obtained mixture was filtered to remove the insoluble solid, and the filtrate was concentrated and dried under vacuum to obtain 0.65 g of a white solid product with a yield of 99%.

MS (ESI): m/z [M+H]⁺, theoretical 391.2, found 391.0.

### 2. Synthesis of Intermediates CN-1A and CN-1B

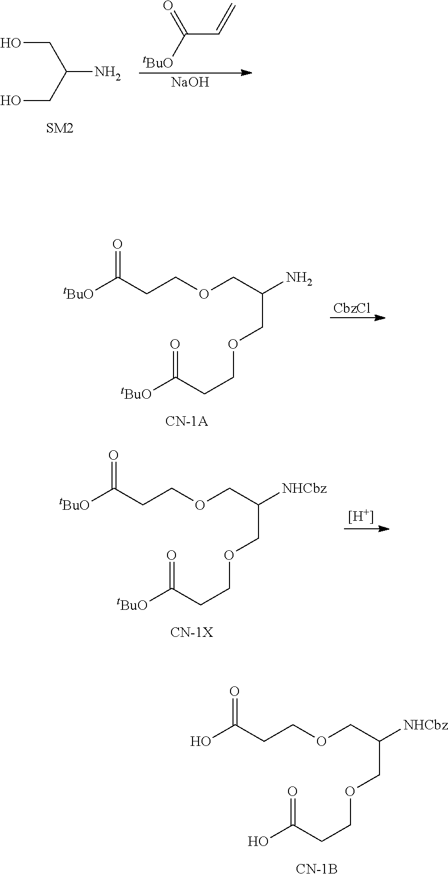

### (1) Synthesis of CN-1A (Addition Reaction)

SM2 (20 g, 219.8 mmoL) and tert-butyl acrylate (61.9 g, 483.6 mmoL) were successively added to a flask containing DMSO (60 mL) at room temperature and stirred until the reaction mixture became clear. The reaction was then added with sodium hydroxide (0.9 g, 22.5 mmoL) dissolved in pure water (4 mL) and carried out under stirring at room temperature for 20 h when LCMS showed that the reaction was substantially complete. The reaction solution was diluted with pure water (100 mL) and extracted with ethyl acetate (60 mL×3). The obtained organic phases were combined, washed with 26% aqueous sodium chloride solution (50 mL×3), dried over anhydrous sodium sulfate, filtered, and concentrated under reduced pressure to obtain a yellow liquid crude product. The crude product was purified by silica gel column chromatography (200-300 meshes, dichloromethane/methanol, 10/1) to obtain 38 g of a yellow oily liquid product with a yield of 50%.

MS (ESI): m/z [M+Na]⁺, theoretical 370.2, found 370.1.

### (2) Synthesis of CN-1X (Benzyloxycarbonylation Protection Reaction)

At room temperature, CN-1A (2.5 g, 7.2 mmol) was added to a flask containing dichloromethane (22 mL), and added with sodium bicarbonate (0.73 g, 8.7 mmol) dissolved in pure water (8 mL), and under constant stirring, benzyl chloroformate (1.23 g, 7.2 mmol) was dropwise added, and then the reaction was carried out for 24 h when LCMC showed that the reaction was completed.

The reaction solution was concentrated under reduced pressure to obtain a light green solid-liquid mixture, which was added with pure water (25 mL), mixed well and extracted with dichloromethane (20 mL×3). The organic phases were combined, washed with 26% aqueous sodium chloride solution (20 mL), dried over anhydrous sodium sulfate, filtered, concentrated and purified by silica gel column chromatography (200-300 meshes, petroleum ether/ethyl acetate, 10/1 to 7/2) to obtain 2.7 g of a light yellow oily product with a yield of 79%.

MS (ESI): m/z [M+Na]⁺, theoretical 504.3, found 504.5.

### (3) Synthesis of CN-1B (De-Tert-Butylation Reaction)

CN-1X (1.05 g, 2.18 mmol) was dissolved in dichloromethane (4 mL), and added with trifluoroacetic acid (4 mL) under constant stirring. The reaction was then carried out under stirring at 35° C. for 6 h when LCMS showed that the reaction was completed. The reaction mixture was subjected to distillation under reduced pressure to remove the solvent, and the obtained oily crude product was purified by silica gel column chromatography (100-200 meshes, dichloromethane/methanol, 10/1) to obtain 0.68 g of a yellow oily liquid product with a yield of 84%.

MS (ESI): m/z [M−H]⁻, theoretical 368.1, found 368.2.

¹H NMR (400 MHz, DMSO-d₆) δ 12.21 (br s, 2H), 7.40-7.28 (m, 5H), 7.18 (d, J=8.3 Hz, 1H), 5.01 (s, 2H), 3.77-3.66 (m, 1H), 3.57 (t, J=6.3 Hz, 4H), 3.34 (d, J=6.0 Hz, 4H), 2.43 (t, J=6.3 Hz, 4H).

### 3. Synthesis of Intermediates CN-2A and CN-2B

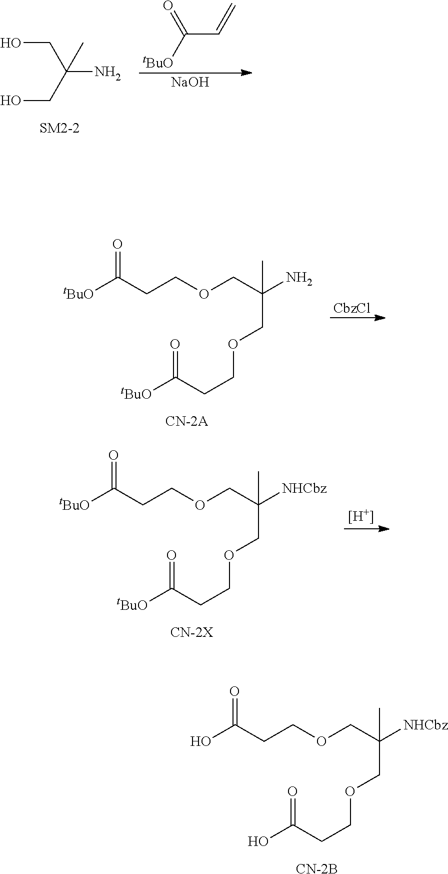

### (1) Synthesis of CN-2A (Addition Reaction)

At room temperature, SM2-2 (10.5 g, 100 mmol) and tert-butyl acrylate (33.3 g, 260 mmol) were successively added to a flask containing DMSO (140 mL) and stirred until the reaction mixture became clear. The reaction was then added with sodium hydroxide (0.8 g, 20 mmoL) dissolved in pure water (4 mL) and then carried out under stirring at room temperature for 48 h when LCMS showed that the reaction was complete. The reaction solution was diluted with pure water (200 mL) and extracted with ethyl acetate (150 mL×3). The obtained organic phases were combined, washed with 26% aqueous sodium chloride solution (100 mL), dried over anhydrous sodium sulfate, filtered and concentrated under reduced pressure to obtain a yellow liquid crude product. The crude product was purified by silica gel column chromatography (200-300 meshes, dichloromethane/methanol, 9/1) to obtain 15.4 g of a yellow oily liquid product with a yield of 42.7%.

MS (ESI): m/z [M+H]⁺, theoretical 362.3, found 362.1.

### (2) Synthesis of CN-2X (Benzyloxycarbonylation Protection Reaction)

At room temperature, CN-2A (5.1 g, 14.1 mmol) was added to a flask containing dichloromethane (30 mL), and then added with sodium bicarbonate (1.43 g, 17.0 mmol) dissolved in pure water (15 mL), and under constant stirring, benzyl chloroformate (2.4 g, 14.1 mmol) was slowly dropwise added, and then the reaction was carried out for 16 h when LCMS showed the reaction was completed. The reaction solution was concentrated under reduced pressure, mixed with pure water (30 mL) and extracted with dichloromethane (25 mL×3). The obtained organic phases were combined, washed with 26% aqueous sodium chloride solution (20 mL), dried over anhydrous sodium sulfate, filtered, and concentrated, and the crude product was purified by silica gel column chromatography (200-300 meshes, dichloromethane/methanol, 10/1 to 7/2) to obtain 6.36 g of a light yellow oily product with a yield of 91%.

MS (ESI): m/z [M+H]⁺, theoretical 496.3, found 496.2.

¹H NMR (400 MHz, DMSO-d₆) δ 7.38-7.25 (m, 5H), 7.14 (s, 1H), 4.98 (s, 2H), 3.73-3.60 (m, 8H), 2.52 (t, J=6.4 Hz, 4H), 1.40 (br s, 18H), 1.25 (s, 3H).

### (3) Synthesis of CN-2B (De-Tert-Butylation Reaction)

CN-2X (3.3 g, 6.66 mmol) was dissolved in dichloromethane (10 mL) and added with trifluoroacetic acid (6 mL) under constant stirring. The reaction was then carried out under stirring at 35° C. for 6 h when LCMS showed that the reaction was completed. The reaction mixture is subjected to distillation under reduced pressure to remove the solvent, and the obtained crude product was purified by silica gel column chromatography (100-200 meshes, dichloromethane/methanol, 10/1) to obtain 1.97 g of a colorless oily liquid product with a yield of 77%.

MS (ESI): m/z [M−H]⁻, theoretical 382.2, found 382.3.

### 4. Synthesis of Intermediates CN-3A, CN-3B, CN-4A, and CN-4B

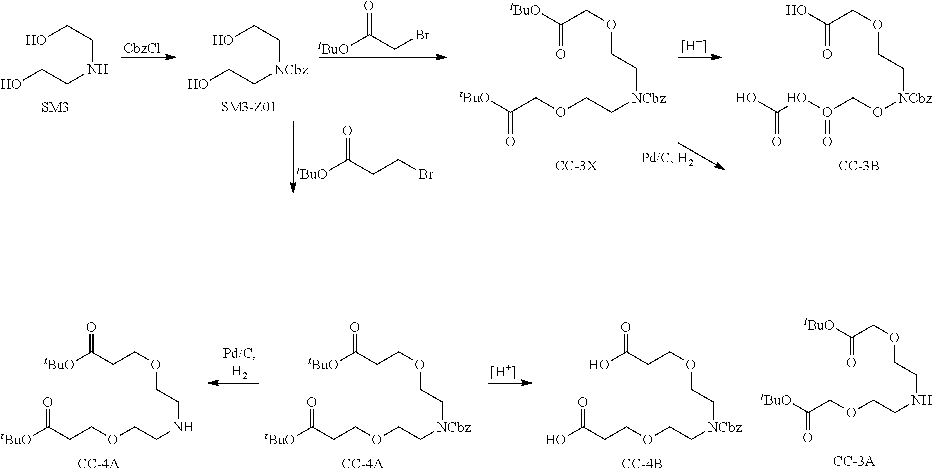

### (1) Synthesis of SM3-Z01 (Benzyloxycarbonylation Protection Reaction)

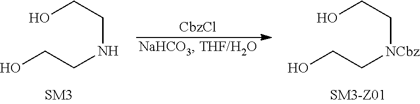 [SMILES: "CbzCl" ; OCCNCCO ; "NaHCO3, THF/H2O" ; O=C(OCc1ccccc1)N(CCO)CCO]

SM3 (2.1 g, 20 mmol) was added to a flask and dissolved with 60 mL of tetrahydrofuran, and under stirring, the reaction was successively added with pure water (20 mL) and sodium bicarbonate (2.0 g, 23.8 mmol), and slowly dropwise added with benzyl chloroformate (3.4 g, 20 mmol) dissolved in tetrahydrofuran (10 mL) within 0.5 h, and the reaction was then carried out for 2 h when TLC showed that the raw materials were completely converted (dichloromethane/methanol, 10/1, R_{f} of the product: 0.5). The reaction solution was diluted with pure water (50 mL) and extracted with dichloromethane (50 mL×3). The obtained organic phases were combined, dried over anhydrous sodium sulfate and filtered. The filtrate was subjected to distillation under reduced pressure to remove the solvent, and the residue was purified by silica gel column chromatography (200-300 meshes, dichloromethane/methanol, 9/1) to obtain 3.92 g of a yellow oily product with a yield of 82%.

MS (ESI): m/z [M+Na]⁺, theoretical 262.1, found 262.1.

### (2) Synthesis of CN-3X (Substitution Reaction)

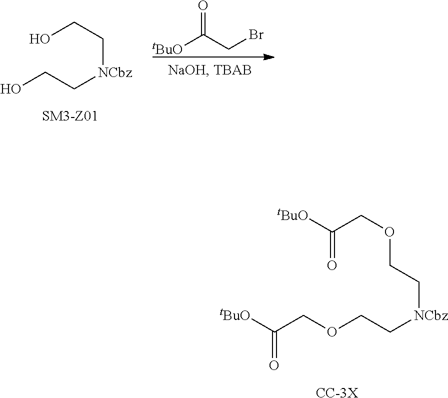

SM3-Z01 (2.39 g, 9.99 mmol) was added to a flask and dissolved with dichloromethane (40 mL), followed by successive addition of 37% aqueous sodium hydroxide solution (40 mL), tetrabutylammonium bromide (3.54 g, 8.4 mmol) and tert-butyl bromoacetate (15.6 g, 80.0 mmol) under stirring at room temperature. Then the reaction was carried out under stirring for 20 h when TLC showed that the reaction was completed (petroleum ether/ethyl acetate, 4/1, R_{f} of the product: 0.3). The reaction solution was diluted with pure water (50 mL) and extracted with dichloromethane (30 mL×3). The obtained organic phases were combined, dried over anhydrous sodium sulfate, filtered, and concentrated under reduced pressure, and the residue was purified by silica gel column chromatography (200-300 meshes, petroleum ether/ethyl acetate, 4/1) to obtain 1.82 g of a colorless oily product with a yield of 39%.

MS (ESI): m/z [M+Na]⁺, theoretical 490.3, found 490.2.

¹H NMR (400 MHz, CDCl₃) δ 7.35-7.25 (m, 5H), 5.05 (s, 2H), 3.95 (s, 4H), 3.69 (t, J=5.8 Hz, 4H), 3.32 (t, J=5.9 Hz, 4H), 1.38 (s, 18H).

### (3) Synthesis of CN-3A (De-Benzyloxycarbonylation Reaction)

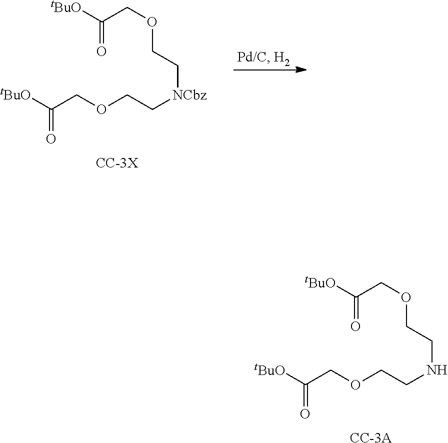

CN-3X (1.2 g, 2.57 mmol) was dissolved in methanol (25 mL) and added with Pd/C (0.12 g, 10 wt %). The reaction system was replaced with hydrogen for three times and then the reaction was carried out under stirring and under hydrogen atmosphere (about 10-20 kPa higher than the standard atmospheric pressure) for 2 h when LCMS showed that the reaction was complete. The obtained mixture was filtered to remove the solid, and the filtrate was concentrated under reduced pressure to obtain 0.8 g of a colorless oily liquid with a yield of 93%.

MS (ESI): m/z [M+H]⁺, theoretical 334.2, found 334.2.

### (4) Synthesis of CN-3B (De-Tert-Butylation Reaction)

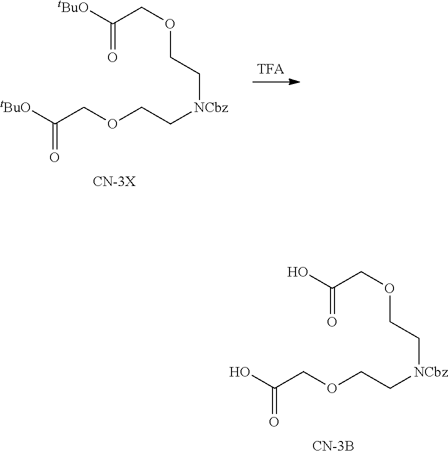

CN-3X (233 mg, 0.5 mmol) and dichloromethane (2 mL) were successively added to a flask, and under constant stirring at room temperature, trifluoroacetic acid (1.54 g, 13.5 mmol) was slowly added, and the reaction was then carried out for 4 h when LCMS showed that the reaction was completed. The reaction solution was concentrated under reduced pressure to be oily, and dried under vacuum at 50° C. for 1 h to obtain 204 mg of a light yellow oily product with a yield of 115% (the product contained some residual TFA and could be directly used in subsequent reaction without purification).

MS (ESI): m/z [M−H]⁻, theoretical 354.1, found 354.2.

### (5) Synthesis of CN-4X (Substitution Reaction)

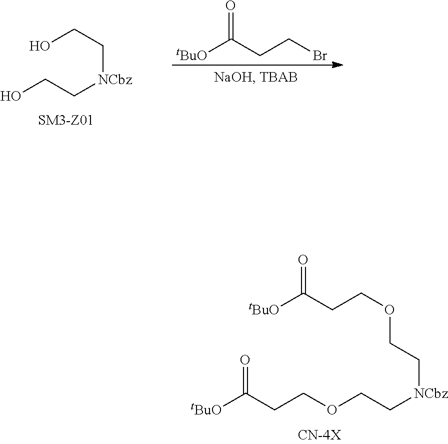 [SMILES: O=C(OCc1ccccc1)N(CCO)CCO ; CC(C)(C)OC(=O)CCBr ; CC(C)(C)OC(=O)CCOCCN(CCOCCC(=O)OC(C)(C)C)C(=O)OCc1ccccc1]

SM3-Z01 (1.3 g, 5.4 mmol) was added to a flask and dissolved with dichloromethane (20 mL), followed by successive addition of 37% aqueous sodium hydroxide solution (22 mL), tetrabutylammonium bromide (1.46 g, 4.54 mmol) and tert-butyl 3-bromopropionate (4.5 g, 21.6 mmol) under stirring at room temperature. The reaction was then carried out under stirring for 20 h when TLC showed that the reaction was completed (petroleum ether/ethyl acetate, 4/1, R_{f} of the product: 0.4). The reaction solution was diluted with pure water (30 mL) and extracted with dichloromethane (20 mL×3). The obtained organic phases were combined, dried over anhydrous sodium sulfate, filtered, and concentrated under reduced pressure, and the residue was purified by silica gel column chromatography (200-300 meshes, petroleum ether/ethyl acetate, 4/1) to obtain 0.78 g of a colorless oily product with a yield of 29%.

MS (ESI): m/z [M+Na]⁺, theoretical 518.3, found 518.4.

### (6) Synthesis of CN-4A (De-Benzyloxycarbonylation Reaction)

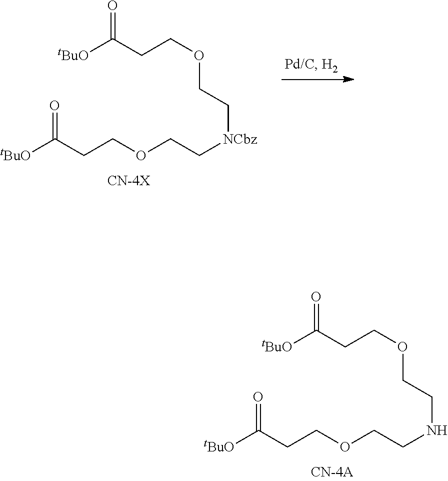 [SMILES: CC(C)(C)OC(=O)CCOCCN(CCOCCC(=O)OC(C)(C)C)C(=O)OCc1ccccc1 ; CC(C)(C)OC(=O)CCOCCNCCOCCC(=O)OC(C)(C)C]

CN-4X (0.52 g, 1.05 mmol) was dissolved in methanol (6 mL) and added with Pd/C (50 mg, 10 wt %). The reaction system was replaced with hydrogen for three times, and the reaction was then carried out under stirring and under hydrogen atmosphere (about 10-20 kPa higher than the standard atmospheric pressure) for 2 h when LCMS showed that the reaction was complete. The reaction mixture was filtered to remove the insoluble solid, and the filtrate was concentrated under reduced pressure to obtain 0.37 g of a colorless oily liquid with a yield of 97%.

MS (ESI): m/z [M+H]⁺, theoretical 362.3, found 362.5.

### (7) Synthesis of CN-4B (De-Tert-Butylation Reaction)

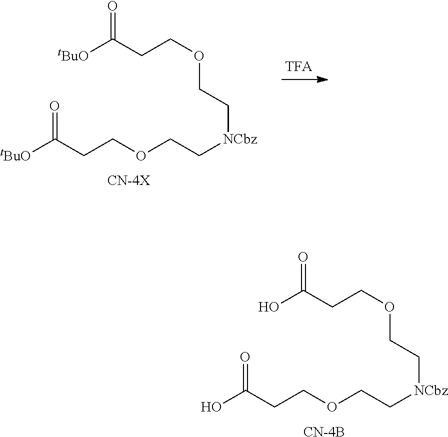

CN-4X (0.2 g, 0.404 mmol) and dichloromethane (2 mL) were successively added to a flask, and under constant stirring at room temperature, trifluoroacetic acid (1 mL) was slowly added, and the reaction was then carried out for 4 h when LCMS showed that the reaction was completed. The reaction solution was concentrated under reduced pressure to be oily, and the residue was dried under vacuum at 50° C. for 1 h to obtain 0.167 g of a light yellow oily product with a yield of 108% (the product contained some residual TFA and could be directly used in subsequent reaction without purification).

MS (ESI): m/z [M−H]⁻, theoretical 382.2, found 382.2.

### 5. Synthesis of Intermediates CC-3A and CC-3B

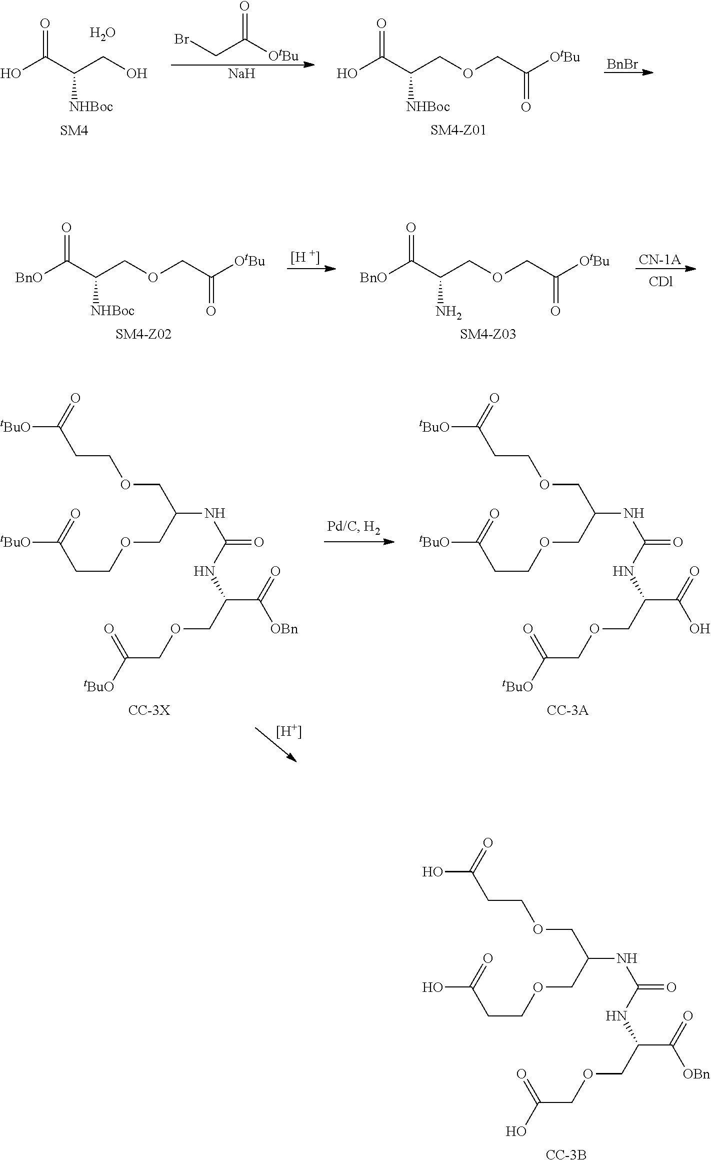

### (1) Synthesis of SM4-Z01 (Substitution Reaction)

SM4 monohydrate (10 g, 44.8 mmol), 150 mL of tetrahydrofuran and 20 g of anhydrous sodium sulfate were added to a flask and allowed to reaction under stirring at room temperature for 0.5 h, followed by suction filtration. The filtrate was added to a flask and added with sodium hydride (60%, 5.9 g, 147.5 mmol) in batches in an ice bath. The reaction was then increased to room temperature, carried out under stirring for 0.5 h, then added with tert-butyl bromoacetate (14.4 g, 73.8 mmol) dissolved in 20 mL of tetrahydrofuran and carried out under stirring at room temperature for 20 h when TLC showed that the conversion of raw materials was completed (dichloromethane/methanol, 10/1, R_{f} of the product: 0.4). The reaction was quenched with 100 mL of water, adjusted to about pH7 with 0.5 N hydrochloric acid, subjected to distillation under reduced pressure to remove most of tetrahydrofuran and extracted with dichloromethane (100 mL×3). The obtained organic phases were combined, dried over anhydrous sodium sulfate, filtered, and concentrated under reduced pressure, and the crude product was purified by silica gel column chromatography to obtain 3.96 g of a yellow oily product with a yield of 27.7%.

MS (ESI): m/z [M+H]⁺, theoretical 320.2, found 320.3.

### (2) Synthesis of SM4-Z02 (Benzylation Protection Reaction)

SM4-Z01 (2.65 g, 8.3 mmol) and potassium carbonate (1.26 g, 9.1 mmol) were added to a flask containing N,N-dimethylformamide (15 mL), and under stirring and at room temperature, benzyl bromide (1.56 g, 9.1 mmol) was dropwise added within 20 min, and then the reaction was carried out for 16 h when TLC showed disappearance of raw material points and appearance of a new product point (petroleum ether/ethyl acetate, 1/1, R_{f} of the product: 0.6). The reaction solution was added with pure water (30 mL) and extracted with ethyl acetate (15 mL×3). The obtained organic phases were combined, washed with 26% saturated saline solution (20 mL), dried over anhydrous sodium sulfate, filtered and concentrated to obtain a colorless liquid crude product. The crude product was purified by silica gel column chromatography (200-300 meshes, petroleum ether/ethyl acetate, 10/1 to 2/1) to obtain 2.86 g of a colorless oily liquid product with a yield of 84%.

MS (ESI): m/z [M+Na]⁺, theoretical 432.2, found 432.1.

### (3) Synthesis of SM4-Z03 (De-Tert-Butoxycarbonylation Reaction)

SM4-Z02 (2.5 g, 6.1 mmol) was added to a flask containing 1,4-dioxane (5 mL), followed by 4 M HCl/1,4-dioxane solution (5 mL), and the reaction was then carried out under stirring for 1 h when TLC showed disappearance of raw material points and appearance of a new product point (adjusted to about pH7, petroleum ether/ethyl acetate, 1/1, R_{f} of the product: 0.6). The reaction solution was concentrated under reduced pressure, and the crude product was dissolved in ethyl acetate (30 mL), washed with 8% aqueous sodium bicarbonate solution (10 mL×3), dried over anhydrous sodium sulfate, filtered, concentrated and dried under vacuum to obtain 1.8 g of a yellow oily product with a yield of 95%.

MS (ESI): m/z [M+H]⁺, theoretical 310.2, found 310.2.

### (4) Synthesis of CC-3X (Urea Condensation Reaction)

Under N₂ atmosphere, SM4-Z03 (1.5 g, 4.85 mmol) and triethylamine (0.5 g, 4.94 mmol) were added to a flask containing dichloromethane (18 mL), and under stirring, N,N′-carbonyldiimidazole (0.801 g, 4.94 mmol) was added slowly, and the reaction was then carried out for 1 h. The reaction was then added with CN-1A (1.72 g, 4.94 mmol) in batches and carried out under stirring at room temperature for 16 h when LCMS showed that the reaction was complete. The reaction solution was added with pure water (25 mL) and extracted with dichloromethane (15 mL×3). The obtained organic phases were combined, washed with 26% saturated saline solution (20 mL), dried over anhydrous sodium sulfate, filtered and concentrated. The obtained crude product was purified by silica gel column chromatography (200-300 meshes, petroleum ether/ethyl acetate, 2/1) to obtain 2.15 g of a white foamy solid product with a yield of 65%.

MS (ESI): m/z [M+H]⁺, theoretical 683.4, found 683.6.

¹H NMR (400 MHz, DMSO-d₆) δ 7.51 (d, J=7.3 Hz, 1H), 7.39-7.24 (m, 5H), 7.09 (d, J=6.8 Hz, 1H), 5.12 (s, 2H), 4.45-4.36 (m, 1H), 4.08 (dd, J=14.8, 4.3 Hz, 2H), 3.81-3.70 (m, 21), 3.73-3.63 (m, 5H), 3.55-3.48 (m, 4H), 2.65-2.55 (m, 4H), 1.41 (br s, 27H).

### (5) Synthesis of CC-3A (Debenzylation Reaction)

CC-3X (1.1 g, 1.61 mmol) was dissolved in tetrahydrofuran (8 mL) and added with Pd/C (0.1 g, 10 wt %). The reaction system was replaced with hydrogen for three times and the reaction was then carried out under stirring and under hydrogen atmosphere (about 10-20 kPa higher than the standard atmospheric pressure) for 2 h when LCMS showed that the reaction was complete. The reaction mixture was filtered to remove the insoluble solid, and the filtrate was concentrated under reduced pressure and dried under vacuum to obtain 0.89 g of a white foamy solid product with a yield of 93%.

MS (ESI): m/z [M+H]⁺, theoretical 593.3, found 593.4.

### (6) Synthesis of CC-3B (De-Tert-Butylation Reaction)

CC-3X (0.95 g, 1.39 mmol) and dichloromethane (6 mL) were successively added to a flask, and under constant stirring and at room temperature, trifluoroacetic acid (3 mL) was added, and the reaction was then carried out for 4 h when LCMS showed that the reaction was complete. The reaction solution was concentrated under reduced pressure to be oily and dried under vacuum at 50° C. for 1 h to obtain 0.75 g of a light yellow oily product with a yield of 105% (the product contained some residual TFA and could be directly used in subsequent reaction without purification).

MS (ESI): m/z [M−H]⁻, theoretical 513.2, found 513.1.

### 6. Synthesis of Intermediates CC-5A, CC-5B, CC-6A and CC-6B

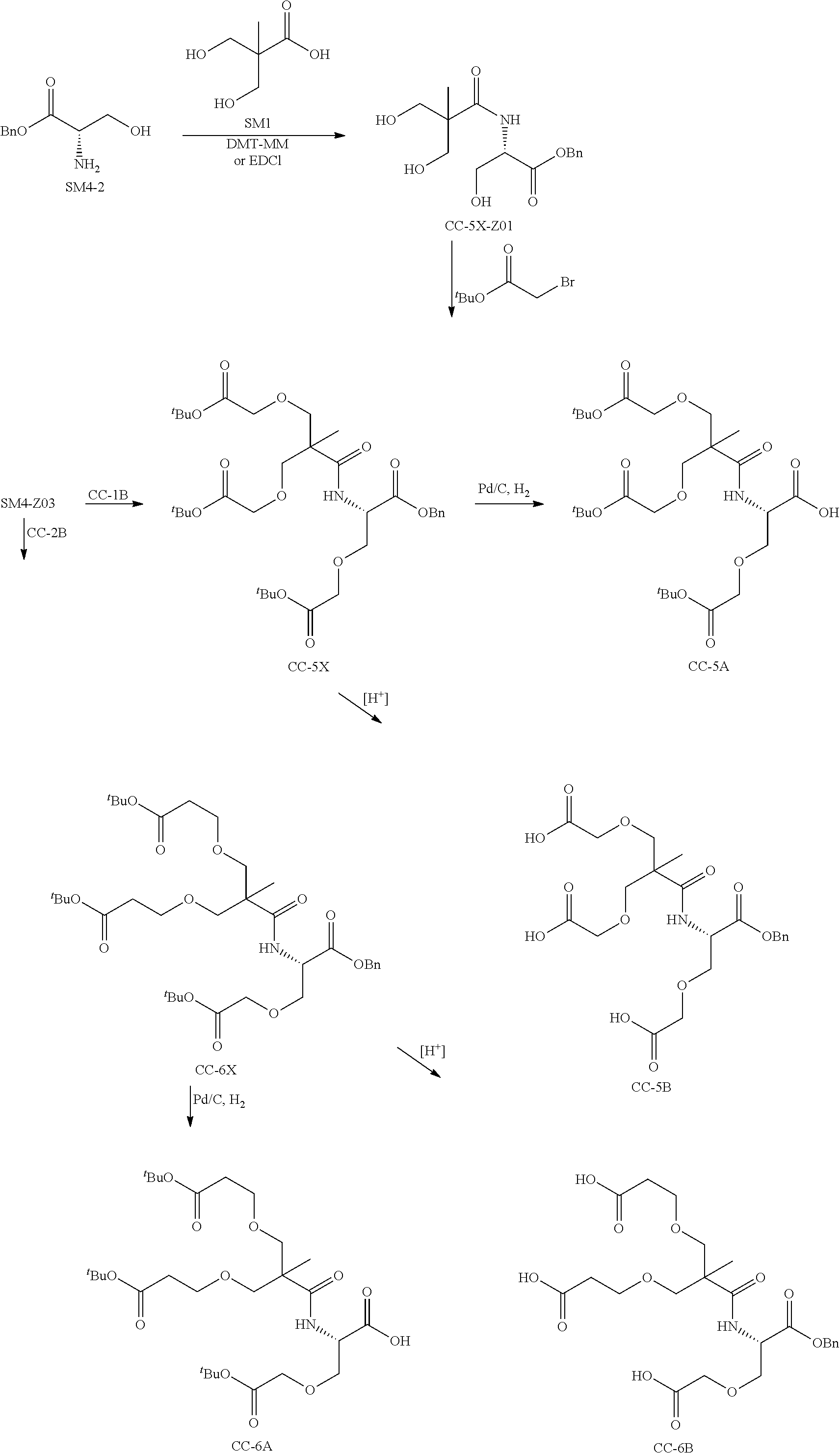

### (1) Synthesis of CC-5X-Z01 (Amidation Condensation Reaction)

Under N₂ atmosphere, SM4-2 (2.2 g, 11.3 mmol), SM1 (1.5 g, 11.3 mmol), EDCI (2.37 g, 12.4 mmol), HOBt (1.53 g, 11.3 mmol) and triethylamine (2.9 g, 22.6 mmol) were added to a flask containing N,N-dimethylformamide (30 mL). The reaction was carried out under stirring at room temperature for 6 h when LCMS showed that the reaction was complete. The reaction solution was concentrated under reduced pressure, and the crude product was purified directly by silica gel column chromatography (200-300 meshes, dichloromethane/methanol, 30/1) to obtain 1.72 g of a white solid product with a yield of 49%.

MS (ESI): m/z [M+H]⁺, theoretical 312.1, found 312.3.

### (2) Synthesis of CC-5X (Amidation Condensation Reaction)

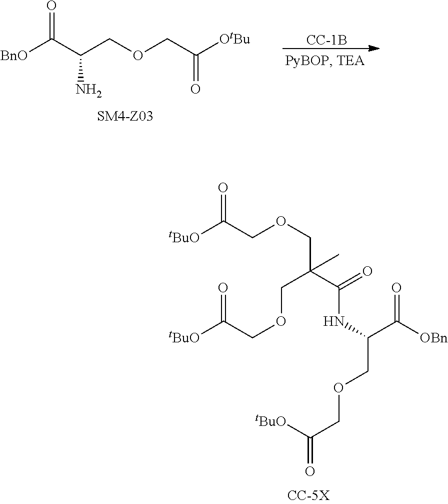 [SMILES: CC(C)(C)OC(=O)COC[C@H](N)C(=O)OCc1ccccc1 ; "CC-1B" ; CC(C)(C)OC(=O)COC[C@H](NC(=O)C(C)(COCC(=O)OC(C)(C)C)COCC(=O)OC(C)(C)C)C(=O)OCc1ccccc1]

Under N₂ atmosphere, SM4-Z03 (1.0 g, 3.2 mmol), CC-1B (1.41 g, 3.9 mmol) and triethylamine (0.98 g, 9.7 mmol) were added to a flask containing dichloromethane (30 mL) and stirred until the reaction mixture became clear. The reaction was then added with PyBOP (2.19 g, 4.2 mmol) and carried out under stirring at room temperature for 12 h when TLC showed the disappearance of raw material points and the appearance of a new product point (petroleum ether/ethyl acetate, 3/2, R_{f} of the product: 0.6). The reaction solution was concentrated under reduced pressure. The obtained crude product was purified by silica gel column chromatography (200-300 meshes, petroleum ether/ethyl acetate, 2/1) to obtain 1.49 g of a colorless viscous oily product with a yield of 71%.

MS (ESI): m/z [M+H]⁺, theoretical 654.3, found 654.5.

### (3) Synthesis of CC-5A (Debenzylation Reaction)

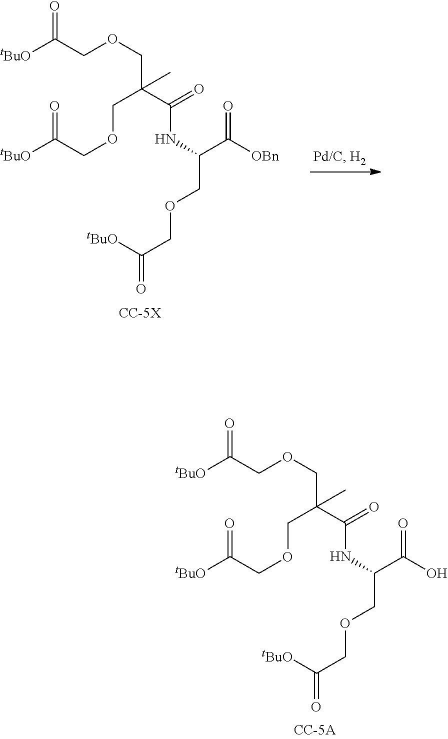 [SMILES: CC(C)(C)OC(=O)COC[C@H](NC(=O)C(C)(COCC(=O)OC(C)(C)C)COCC(=O)OC(C)(C)C)C(=O)OCc1ccccc1 ; CC(C)(C)OC(=O)COC[C@H](NC(=O)C(C)(COCC(=O)OC(C)(C)C)COCC(=O)OC(C)(C)C)C(=O)O]

CC-5X (0.58 g, 0.89 mmol) was dissolved in tetrahydrofuran (9 mL) and added with Pd/C (0.11 g, 10 wt %). The reaction system was replaced with hydrogen gas for three times and the reaction was carried out under stirring and under hydrogen atmosphere (about 10-20 kPa higher than the standard atmospheric pressure) for 2 h when LCMS showed no residual raw materials. The reaction mixture was filtered to remove the solid, and the filtrate was concentrated and purified by silica gel column chromatography (200-300 meshes, dichloromethane/methanol, 10/1) to obtain 0.44 g of a white solid product with a yield of 88%.

MS (ESI): m/z [M+H]⁺, theoretical 564.3, found 564.3.

¹H NMR (400 MHz, DMSO-d₆) δ 7.80 (d, J=7.3 Hz, 1H), 4.28 (dt, J=8.1, 4.5 Hz, 1H), 4.06-3.93 (m, 6H), 3.83 (dd, J=9.9, 5.1 Hz, 1H), 3.70 (dd, J=10.0, 3.9 Hz, 1H), 3.64-3.51 (m, 4H), 1.42 (s, 27H), 1.10 (s, 3H).

### (4) Synthesis of CC-5B (De-Tert-Butylation Reaction)

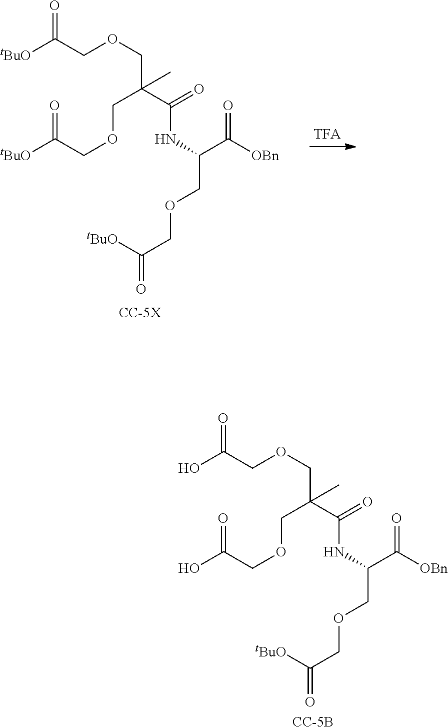

CC-5X (0.5 g, 0.76 mmol) was dissolved in trifluoroacetic acid (5 mL) and then the reaction was carried out under stirring at room temperature for 6 h when LCMS showed no residual raw materials. The reaction solution was subjected to distillation under reduced pressure to remove trifluoroacetic acid, and the crude product was purified by silica gel column chromatography (200-300 meshes, dichloromethane/methanol, 10/1) to obtain 0.27 g of a white solid product with a yield of 74%.

MS (ESI): m/z [M+H]⁺, theoretical 486.2, found 486.1.

¹H NMR (400 MHz, DMSO-d₆) δ 8.33 (d, J=7.1 Hz, 1H), 7.40-7.28 (m, 5H), 5.12 (dd, J=19.8, 12.7 Hz, 2H), 4.54-4.46 (m, 1H), 4.07-3.93 (m, 8H), 3.89 (dd, J=9.9, 5.3 Hz, 1H), 3.75 (dd, J=9.9, 4.4 Hz, 1H), 3.67-3.52 (m, 4H), 1.08 (s, 3H).

### (5) Synthesis of CC-6X (Amidation Condensation Reaction)

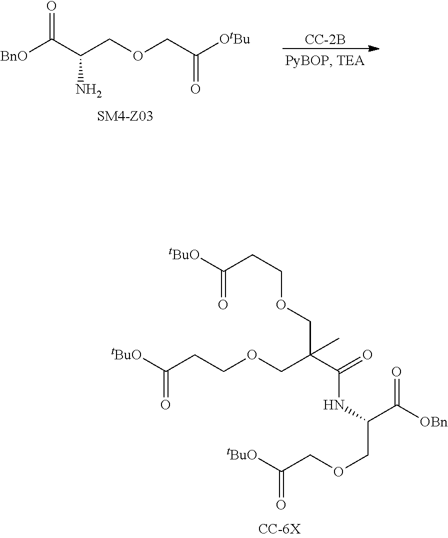 [SMILES: CC(C)(C)OC(=O)COC[C@H](N)C(=O)OCc1ccccc1 ; "CC-2B" ; CC(C)(C)OC(=O)CCOCC(C)(COCCC(=O)OC(C)(C)C)C(=O)N[C@@H](COCC(=O)OC(C)(C)C)C(=O)OCc1ccccc1]

CC-6X was obtained by subjecting SM4-Z03 and CC-2B as starting materials to the same amidation condensation reaction steps for synthesis of CC-5X.

MS (ESI): m/z [M+H]⁺, theoretical 682.4, found 682.4.

¹H NMR (400 MHz, DMSO-d₆) δ 7.75 (d, J=7.8 Hz, 1H), 7.39-7.24 (m, 5H), 3.15 (s, 2H), 4.45-4.35 (m, 1H), 4.08 (dd, J=34.8, 3.9 Hz, 2H), 3.78 (d, J=5.0 Hz, 2H), 3.75-3.65 (m, 4H), 3.57 (s, 4H), 2.62 (t, J=5.4 Hz, 4H), 1.41 (br s, 27H), 1.15 (s, 3H).

### (6) Synthesis of CC-6A (De-Benzylation Reaction)

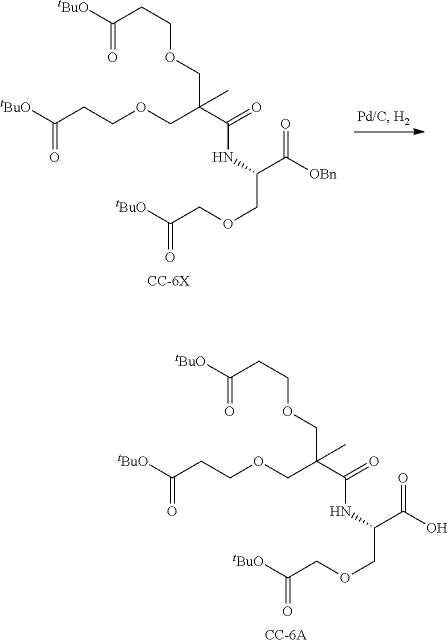 [SMILES: CC(C)(C)OC(=O)CCOCC(C)(COCCC(=O)OC(C)(C)C)C(=O)N[C@@H](COCC(=O)OC(C)(C)C)C(=O)OCc1ccccc1 ; CC(C)(C)OC(=O)CCOCC(C)(COCCC(=O)OC(C)(C)C)C(=O)N[C@@H](COCC(=O)OC(C)(C)C)C(=O)O]

CC-6A was obtained by subjecting CC-6X as a starting material to the same debenzylation reaction steps for synthesis of CC-5A.

MS (ESI): m/z [M+H]⁺, theoretical 592.3, found 592.4.

### (7) Synthesis of CC-6B (De-Tert-Butylation Reaction)

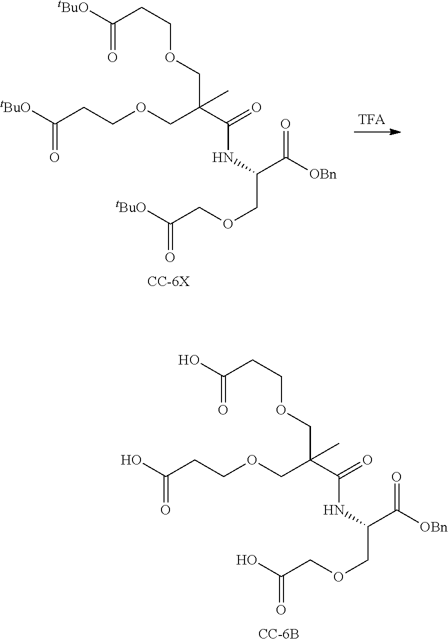 [SMILES: CC(C)(C)OC(=O)CCOCC(C)(COCCC(=O)OC(C)(C)C)C(=O)N[C@@H](COCC(=O)OC(C)(C)C)C(=O)OCc1ccccc1 ; CC(COCCC(=O)O)(COCCC(=O)O)C(=O)N[C@@H](COCC(=O)O)C(=O)OCc1ccccc1 ; "CC-6B"]

CC-6B was obtained by subjecting CC-6X as a starting material to the same de-tert-butylation reaction steps for synthesis of CC-5B.

MS (ESI): m/z [M−H]⁻, theoretical 512.2, found 512.1.

### 7. Synthesis of Intermediate CC-4A and CC-4B

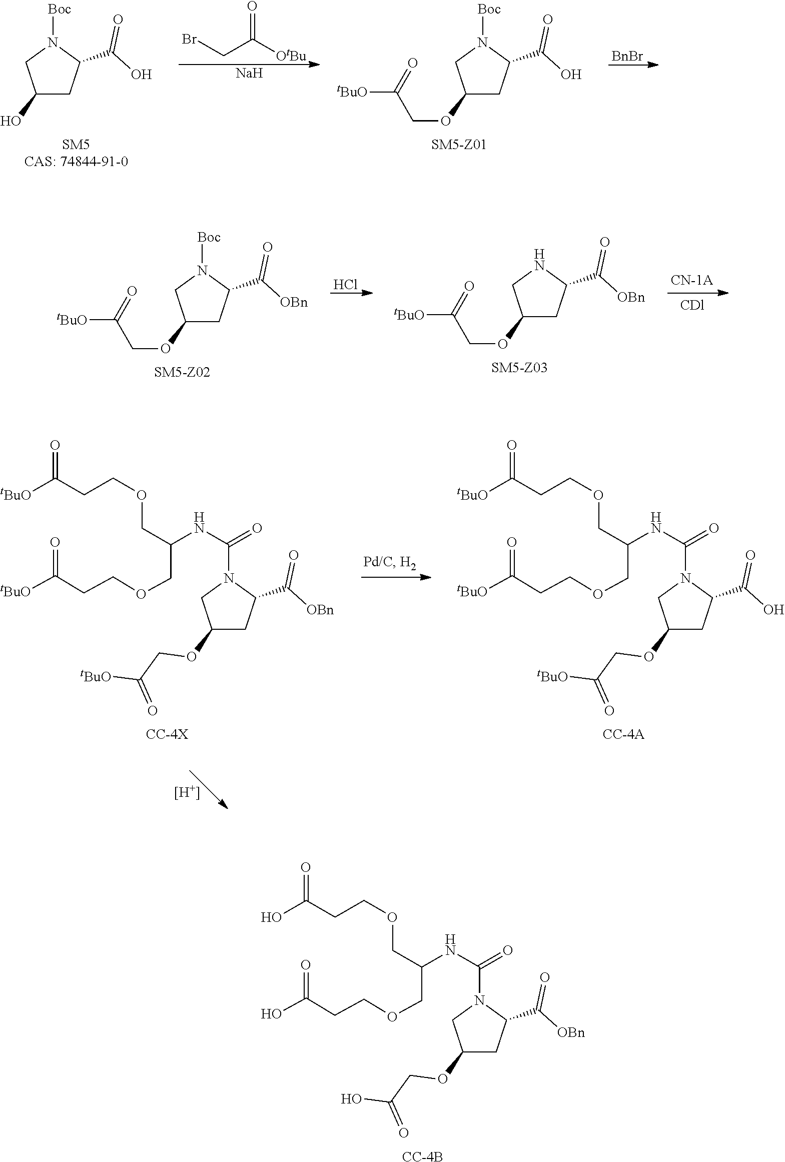

### (1) Synthesis of SM5-Z01

SM5-Z01 was obtained by subjecting SM5 as a starting material to the same substitution reaction steps for synthesis of SM4-Z01.

MS (ESI): m/z [M+H]⁺, theoretical 346.2, found 346.3.

### (2) Synthesis of SM5-Z02

SM5-Z02 was obtained by subjecting SM5-Z01 as a starting material to the same benzylation protection reaction steps for synthesis of SM4-Z02.

MS (ESI): m/z [M+H]⁺, theoretical 436.2, found 436.4.

### (3) Synthesis of SM5-Z03

SM5-Z03 was obtained by subjecting SM5-Z02 as a starting material to the same de-tert-butoxycarbonylation reaction steps for synthesis of SM4-Z03.

MS (ESI): m/z [M+H]⁺, theoretical 336.2, found 336.3.

### (4) Synthesis of CC-4X

CC-4X was obtained by subjecting SM5-Z03 and CN-1A as starting materials to the same urea condensation reaction steps of the synthesis of CC-3X.

MS (ESI): m/z [M+Na]⁺, theoretical 731.4, found 731.4.

¹H NMR (400 MHz, DMSO-d₆) δ 7.34-7.25 (m, 5H), 6.75 (d, J=7.5 Hz, 1H), 5.12 (s, 2H), 4.54-4.46 (m, 1H), 4.33-4.24 (m, 1H), 4.20 (dd, J=14.6, 9.2 Hz, 2H), 3.78-3.71 (m, 1H), 3.67 (L J=6.5 Hz, 4H), 3.57-3.40 (m, 6H), 2.60 (m, 4H), 2.38-2.29 (m, 1H), 2.28-2.18 (m, 1H), 1.41 (br s, 27H).

### (5) Synthesis of CC-4A

CC-4A was obtained by subjecting CC-4X as a starting material to the same debenzylation reaction steps for synthesis of CC-3A.

MS (ESI): m/z [M+H]⁺, theoretical 619.3, found 619.5.

### (6) Synthesis of CC-4B

CC-4B was obtained by subjecting CC-4X as a starting material to the same de-tert-butylation reaction steps for synthesis of CC-3B.

MS (ESI): m/z [M−H]⁻, theoretical 539.2, found 539.3.

### 8. Synthesis of Intermediates CC-7A, CC-7B, CC-8A, CC-8B, CC-13A, and CC-13B

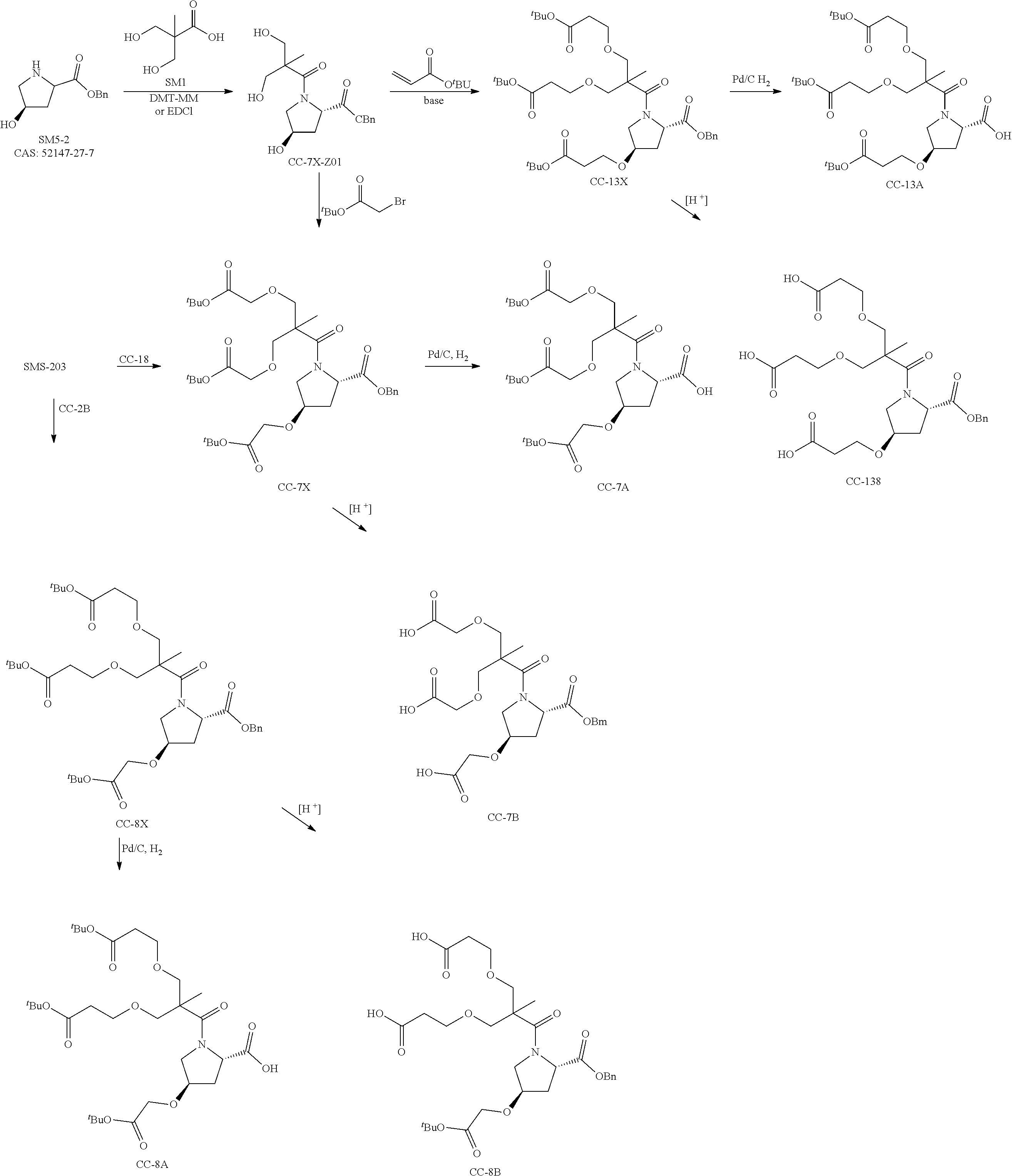

### (1) Synthesis of CC-7X-Z01 (Amidation Condensation Reaction)

SM5-2 (2.8 g, 12.7 mmol) and SM1 (1.7 g, 12.7 mmol) were successively added to a flask containing methanol (40 mL), followed by addition of DMT-MM monohydrate (5.6 g, 19.1 mmol) in batches under stirring. The reaction was then carried out under stirring at room temperature for 16 h when LCMS showed that the reaction was complete. The reaction solution was concentrated under reduced pressure, and the obtained crude product was purified directly by silica gel column chromatography (200-300 meshes, dichloromethane/methanol, 30/1) to obtain 2.96 g of a white solid product with a yield of 69%.

MS (ESI): m/z [M+H]⁺, theoretical 338.2, found 338.3.

### (2) Synthesis of CC-7X

CC-7X was obtained by subjecting SM5-Z03 and CC-1B as starting materials to the same amidation condensation reaction steps for synthesis of CC-5X.

MS (ESI): m/z [M+H]⁺, theoretical 680.4, found 680.3.

### (3) Synthesis of CC-7A

CC-7A was obtained by subjecting CC-7X as a starting material to the same debenzylation reaction steps for synthesis of CC-5A.

MS (ESI): m/z [M+H]⁺, theoretical 590.3, found 590.3.

### (4) Synthesis of CC-7B

CC-7B was obtained by subjecting CC-7X as a starting material to the same de-tert-butylation reaction steps for synthesis of CC-5B.

MS (ESI): m/z [M−H]⁻, theoretical 510.2, found 510.4.

### (5) Synthesis of CC-8X

CC-8X was obtained by subjecting SM5-Z03 and CC-2B as starting materials to the same amidation condensation reaction steps for synthesis of CC-6X.

MS (ESI): m/z [M+Na]⁺, theoretical 730.4, found 730.3.

### (6) Synthesis of CC-8A

CC-8A was obtained by subjecting CC-8X as a starting material to the same debenzylation reaction steps for synthesis of CC-6A.

MS (ESI): m/z [M+H]⁺, theoretical 618.3, found 618.3.

### (7) Synthesis of CC-8B

CC-8B was obtained by subjecting CC-8X as a starting material to the same de-tert-butylation reaction steps for synthesis of CC-6B.

MS (ESI): m/z [M+H]⁺, theoretical 540.2, found 540.2.

### (8) Synthesis of CC-13X

CC-13X was obtained by subjecting CC-7X-Z01 as a starting material to the same addition reaction steps for synthesis of CN-1A.

MS (ESI): m/z [M+H]⁺, theoretical 722.4, found 722.6.

### (9) Synthesis of CC-13A

CC-13A was obtained by subjecting CC-13X as a starting material to the same debenzylation reaction steps for synthesis of CC-7A.

MS (ESI): m/z [M+H]⁺, theoretical 632.4, found 632.4.

### (10) Synthesis of CC-13B

CC-13B was obtained by subjecting CC-13X as a starting material to the same de-tert-butylation reaction steps for synthesis of CC-7B.

MS (ESI): m/z [M−H]⁻, theoretical 552.2, found 552.0.

### 9. Synthesis of Intermediates CC-9, CC-10, CC-11, CC-12 and CC-14

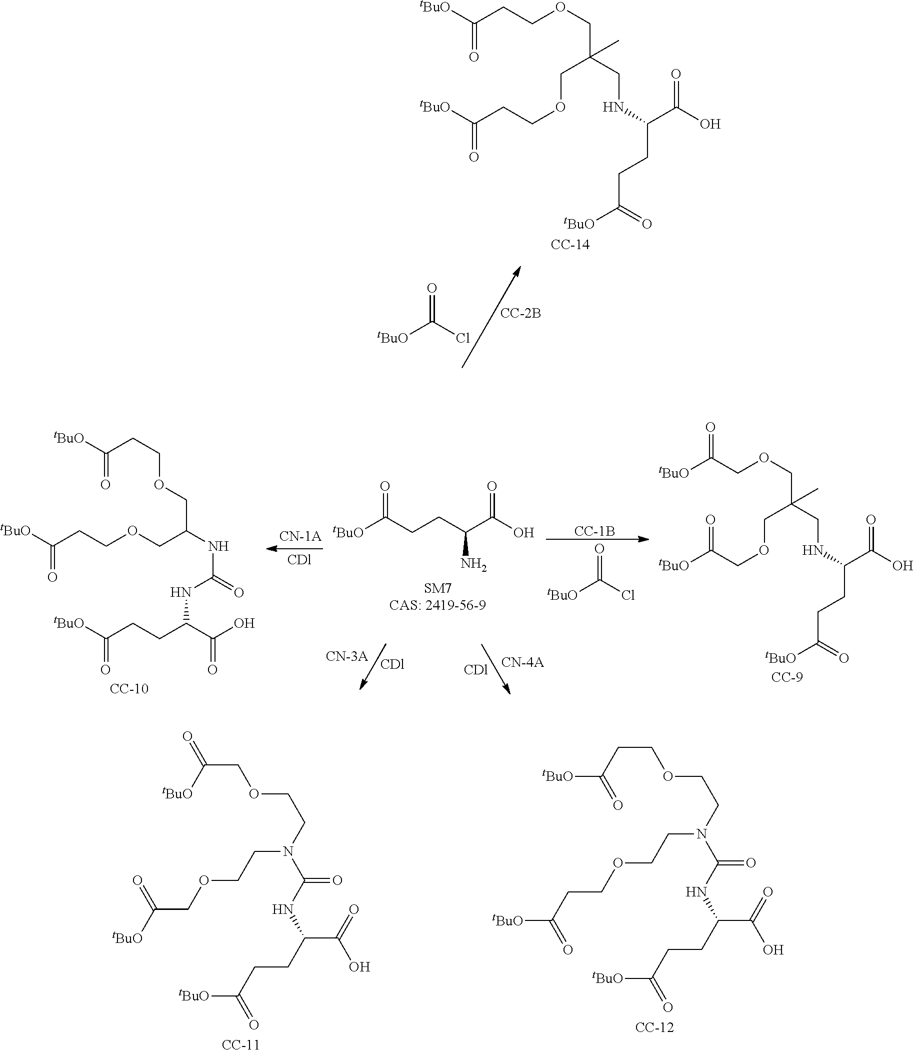

### (1) Synthesis of CC-9 (Amidation Condensation Reaction)

Under N₂ atmosphere, CC-1B (1.4 g, 3.86 mmol) and N-methylmorpholine (0.78 g, 7.73 mmol) were successively added to a flask containing dichloromethane (12 mL), cooled to 0-5° C. in an ice bath, and then slowly dropwise added with isobutyl chloroformate (0.54 g, 3.94 mmol). The reaction was maintained at the temperature and carried out under stirring for 1 h. The reaction was added with SM7 (0.8 g, 3.94 mmol) and then carried out under stirring at room temperature for 2 h when LCMS showed that the reaction was complete. The reaction solution was diluted with water (20 mL), adjusted to about pH4 with 0.5 N hydrochloric acid, and extracted with dichloromethane (10 mL×3). The obtained organic phases were combined, washed with 26% aqueous sodium chloride solution (20 mL), dried over anhydrous sodium sulfate, filtered and concentrated under reduced pressure. The crude product was crystallized with methyl tert-butyl ether to obtain 1.9 g of a light yellow solid product with a yield of 90%.

MS (ESI): m/z [M+H]⁺, theoretical 548.3, found 548.2.

### (2) Synthesis of CC-10

CC-10 was obtained by subjecting SM7 and CN-1A as starting materials to the same urea condensation reaction steps for synthesis of CC-3X.

MS (ESI): m/z [M+H]⁺, theoretical 577.3, found 577.5.

### (3) Synthesis of CC-11

CC-11 was obtained by subjecting SM7 and CN-3A as starting materials to the same urea condensation reaction steps for synthesis of CC-10.

MS (ESI): m/z [M+H]⁺, theoretical 563.3, found 563.4.

### (4) Synthesis of CC-12

CC-12 was obtained by subjecting SM7 and CN-4A as starting materials to the same urea condensation reaction steps for synthesis of CC-10.

MS (ESI): m/z [M+H]⁺, theoretical 577.3, found 577.5.

### (5) Synthesis of CC-14

CC-14 was obtained by subjecting SM7 and CN-2B as starting materials to the same amidation condensation reaction steps for synthesis of CC-9.

MS (ESI): m/z [M+H]⁺, theoretical 591.3, found 591.4.

### 10. Synthesis of Intermediates CN-5A, CN-5B, CN-7A, and CN-7B

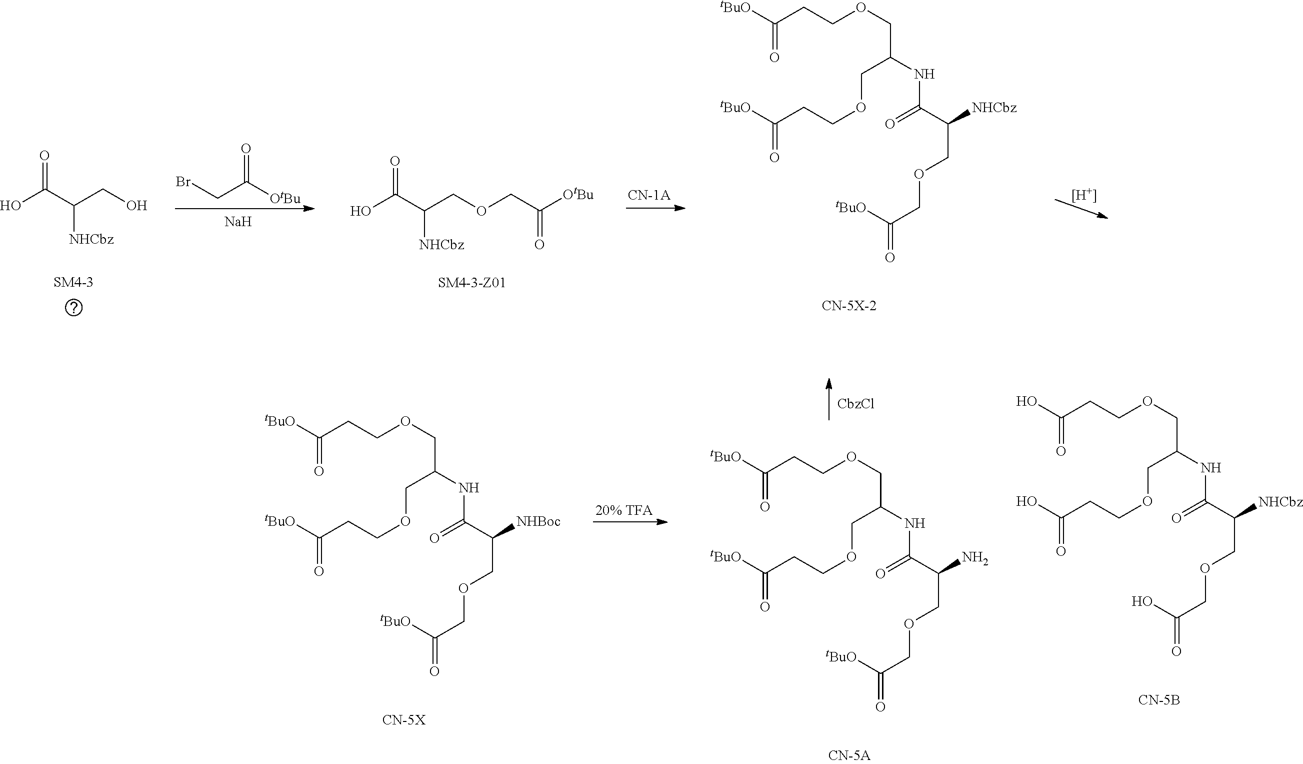

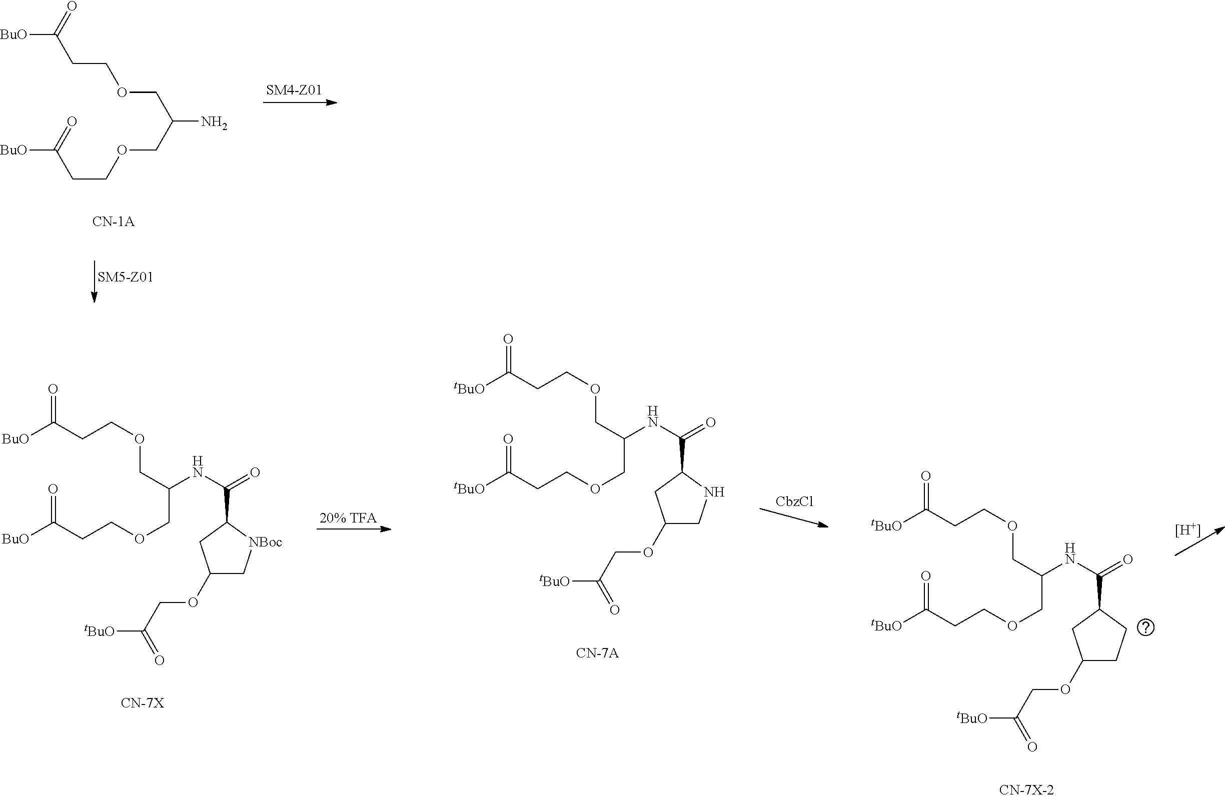

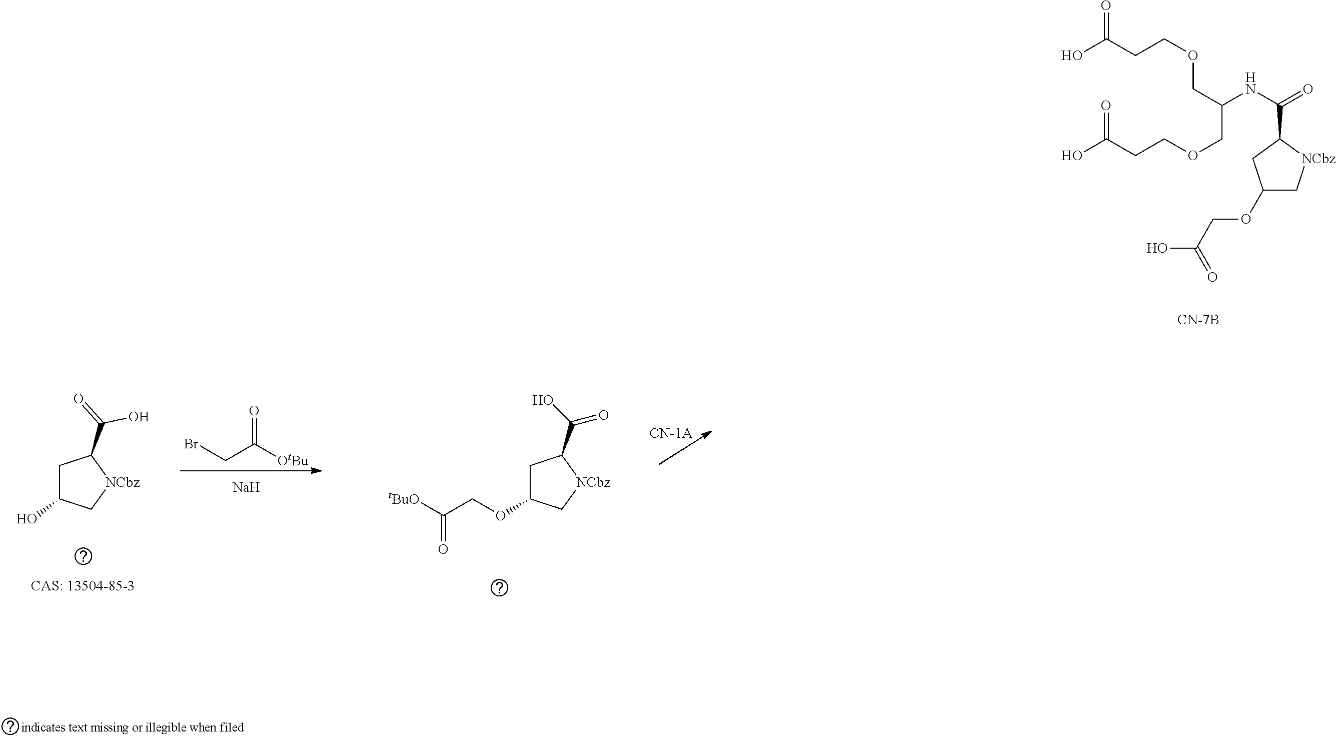

### (1) Synthesis of CN-5X (Amidation Condensation Reaction)

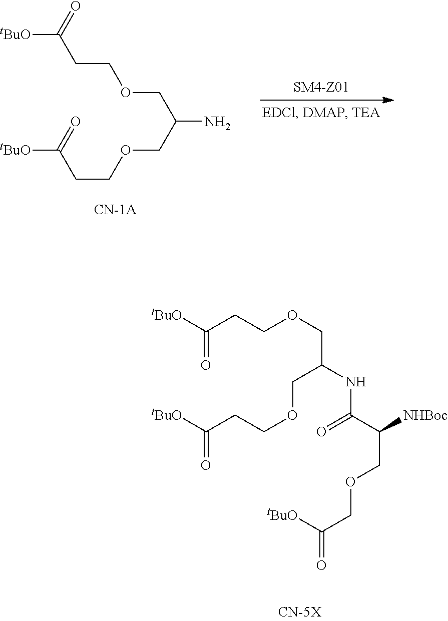 [SMILES: CC(C)(C)OC(=O)CCOCC(N)COCCC(=O)OC(C)(C)C ; CC(C)(C)OC(=O)CCOCC(COCCC(=O)OC(C)(C)C)NC(=O)[C@H](COCC(=O)OC(C)(C)C)NC(=O)OC(C)(C)C]

SM4-Z01 (3.96 g, 12.4 mmol) and 150 mL of dichloromethane were added to a flask, followed by successive addition of CN-1A (4.31 g, 12.4 mmol), EDCI (3.58 g, 18.6 mmol), triethylamine (3.76 g, 37.2 mmol) and DMAP (0.3 g, 2.4 mmol) under stirring at room temperature. The reaction was then carried out under stirring for 12 h when TLC showed that the conversion of the raw materials was complete (petroleum ether/ethyl acetate, 1/1, R_{f} of the product: 0.5). The reaction solution was washed with 100 mL of water, extracted with 150 mL of dichloromethane and washed with 26% aqueous sodium chloride solution. The obtained organic phases were combined, dried over anhydrous sodium sulfate and subjected to distillation under reduced pressure to remove solvents. The residue was purified by silica gel column chromatography (200-300 meshes, petroleum ether/ethyl acetate, 2/1) to obtain 3.77 g of a light yellow oily product with a yield of 46%.

MS (ESI): m/z [M+H]⁺, theoretical 649.4, found 649.4.

### (2) Synthesis of CN-5A (De-Tert-Butoxycarbonylation Reaction)

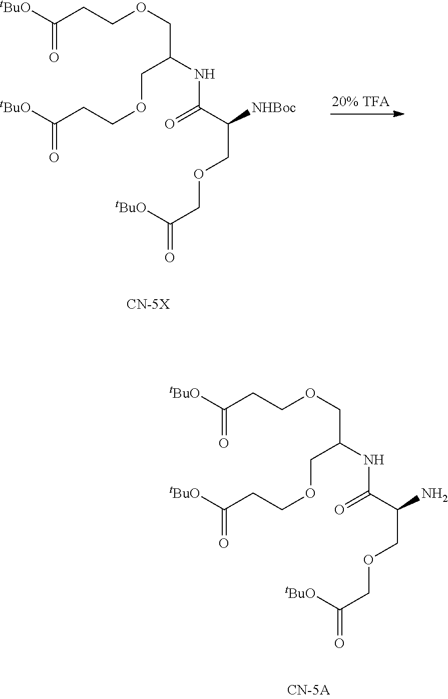 [SMILES: CC(C)(C)OC(=O)CCOCC(COCCC(=O)OC(C)(C)C)NC(=O)[C@H](COCC(=O)OC(C)(C)C)NC(=O)OC(C)(C)C ; CC(C)(C)OC(=O)CCOCC(COCCC(=O)OC(C)(C)C)NC(=O)[C@@H](N)COCC(=O)OC(C)(C)C]

CN-5X (4.16 g, 6.4 mmol) was added to a flask and dissolved with 10 mL of dichloromethane, and under stirring and at room temperature, 2 mL of trifluoroacetic acid was slowly added, and the reaction was then carried out for 20 min when TLC showed that the conversion of the raw materials was complete (adjusted to about pH7, dichloromethane/methanol, 10/1, R_{f} of the product: 0.3). The reaction solution was subjected to distillation under reduced pressure at room temperature to remove the solvent and most trifluoroacetic acid, added with 8% aqueous sodium bicarbonate solution (50 mL), stirred well, and extracted with dichloromethane (35 mL×3). The obtained organic phases were combined, dried over anhydrous sodium sulfate and subjected to distillation under reduced pressure to remove the solvent. The residue was purified by silica gel column chromatography to obtain 1.02 g of a light yellow oily product with a yield of 29%.

MS (ESI): m/z [M+H]⁺, theoretical 549.3, found 549.3.

### (3) Synthesis of CN-5B (De-Tert-Butylation Reaction)

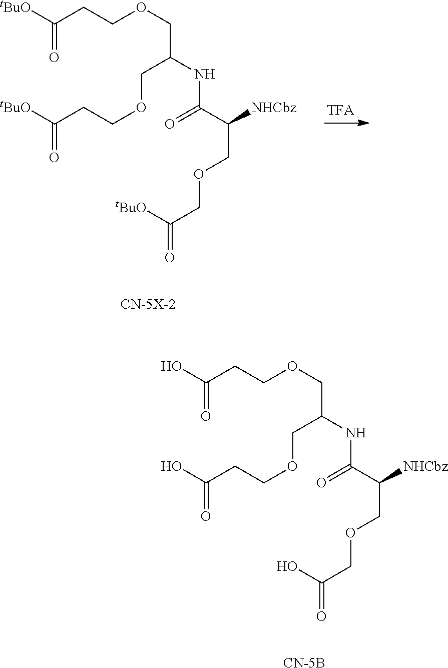 [SMILES: CC(C)(C)OC(=O)CCOCC(COCCC(=O)OC(C)(C)C)NC(=O)[C@H](COCC(=O)OC(C)(C)C)NC(=O)OCc1ccccc1 ; O=C(O)CCOCC(COCCC(=O)O)NC(=O)[C@H](COCC(=O)O)NC(=O)OCc1ccccc1 ; "CN-5B"]

CC-5B was obtained by subjecting CN-5A as a starting material to the same de-tert-butylation reaction steps for synthesis of CC-5B.

MS (ESI): m/z [M−H]⁻, theoretical 513.2, found 513.3.

### (4) Synthesis of CN-7X (Amidation Condensation Reaction)

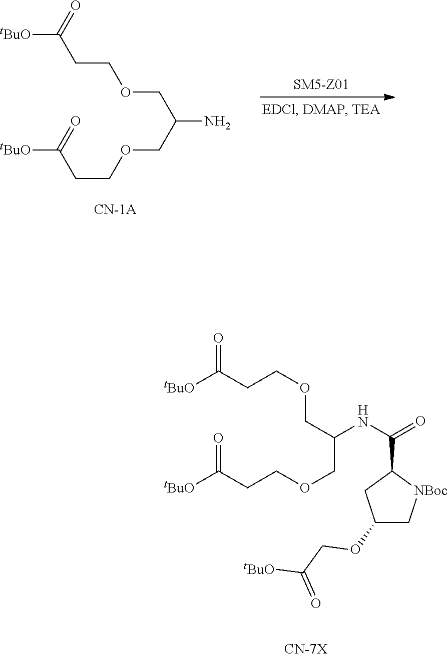 [SMILES: CC(C)(C)OC(=O)CCOCC(N)COCCC(=O)OC(C)(C)C ; CC(C)(C)OC(=O)CCOCC(COCCC(=O)OC(C)(C)C)NC(=O)[C@@H]1C[C@@H](OCC(=O)OC(C)(C)C)CN1C(=O)OC(C)(C)C]

CC-7X was obtained by subjecting SM5-Z01 and CN-1A as starting materials to the same amidation condensation reaction steps for synthesis of CC-5X.

MS (ESI): m/z [M+H]⁺, theoretical 675.4, found 675.6.

### (5) Synthesis of CN-7A (De-Tert-Butoxycarbonylation Reaction)

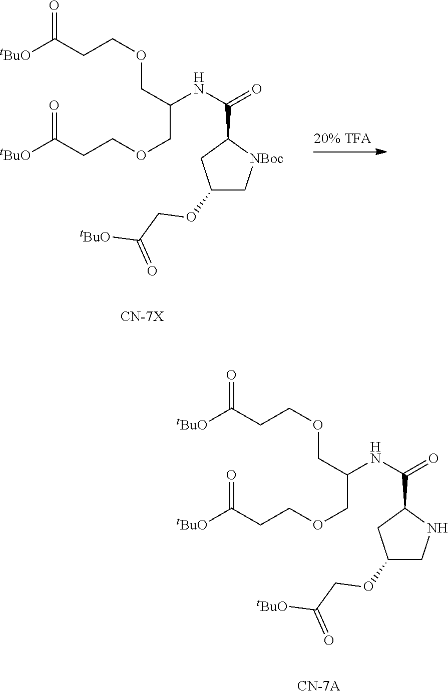 [SMILES: CC(C)(C)OC(=O)CCOCC(COCCC(=O)OC(C)(C)C)NC(=O)[C@@H]1C[C@@H](OCC(=O)OC(C)(C)C)CN1C(=O)OC(C)(C)C ; CC(C)(C)OC(=O)CCOCC(COCCC(=O)OC(C)(C)C)NC(=O)[C@@H]1C[C@@H](OCC(=O)OC(C)(C)C)CN1]

CN-7A was obtained by subjecting CN-7X as a starting material to the same de-tert-butoxycarbonylation reaction steps for synthesis of CN-5A.

MS (ESI): m/z [M+H]⁺, theoretical 575.4, found 575.5.

### (6) Synthesis of SM5-3-Z01 (Substitution Reaction)

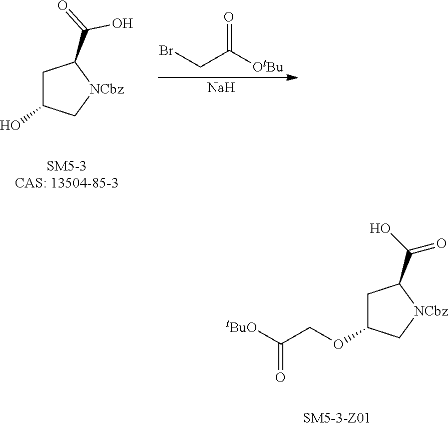

SM5-3-Z01 was obtained by subjecting SM5-3 as a starting material to the same substitution reaction steps for synthesis of SM5-Z01.

MS (ESI): m/z [M+H]⁺, theoretical 380.2, found 380.3.

### (7) Synthesis of CN-7X-2 (Amidation Condensation Reaction)

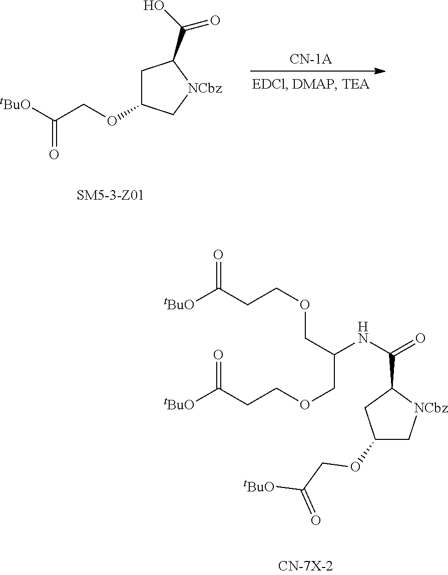 [SMILES: CC(C)(C)OC(=O)CO[C@@H]1C[C@@H](C(=O)O)N(C(=O)OCc2ccccc2)C1 ; CC(C)(C)OC(=O)CCOCC(COCCC(=O)OC(C)(C)C)NC(=O)[C@@H]1C[C@@H](OCC(=O)OC(C)(C)C)CN1C(=O)OCc1ccccc1]

CN-7X-2 was obtained by subjecting SM5-3-Z01 and CN-1A as starting materials to the same amidation condensation reaction steps for synthesis of CN-7X.

MS (ESI): m/z [M+H]⁺, theoretical 709.4, found 709.3.

### (8) Synthesis of CN-7B (De-Tert-Butylation Reaction)

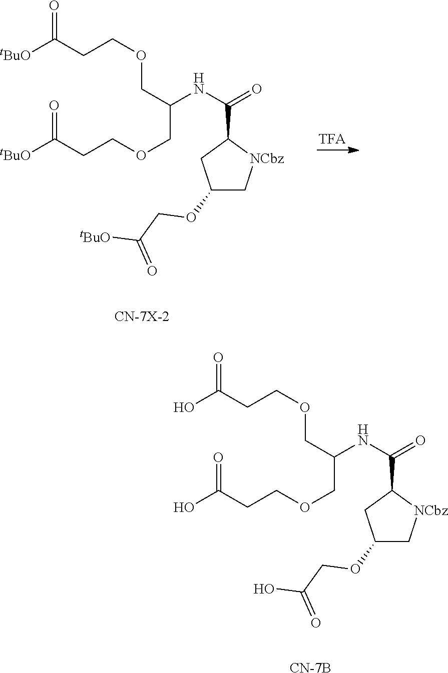 [SMILES: CC(C)(C)OC(=O)CCOCC(COCCC(=O)OC(C)(C)C)NC(=O)[C@@H]1C[C@@H](OCC(=O)OC(C)(C)C)CN1C(=O)OCc1ccccc1 ; O=C(O)CCOCC(COCCC(=O)O)NC(=O)[C@@H]1C[C@@H](OCC(=O)O)CN1C(=O)OCc1ccccc1 ; "CN-7B"]

CN-7B was obtained by subjecting CN-7X-2 as a starting material to the same de-tert-butylation reaction steps for synthesis of CN-5B.

MS (ESI): m/z [M−H]⁻, theoretical 539.2, found 539.4.

### 11. Synthesis of Intermediates CN-6A and CN-6B

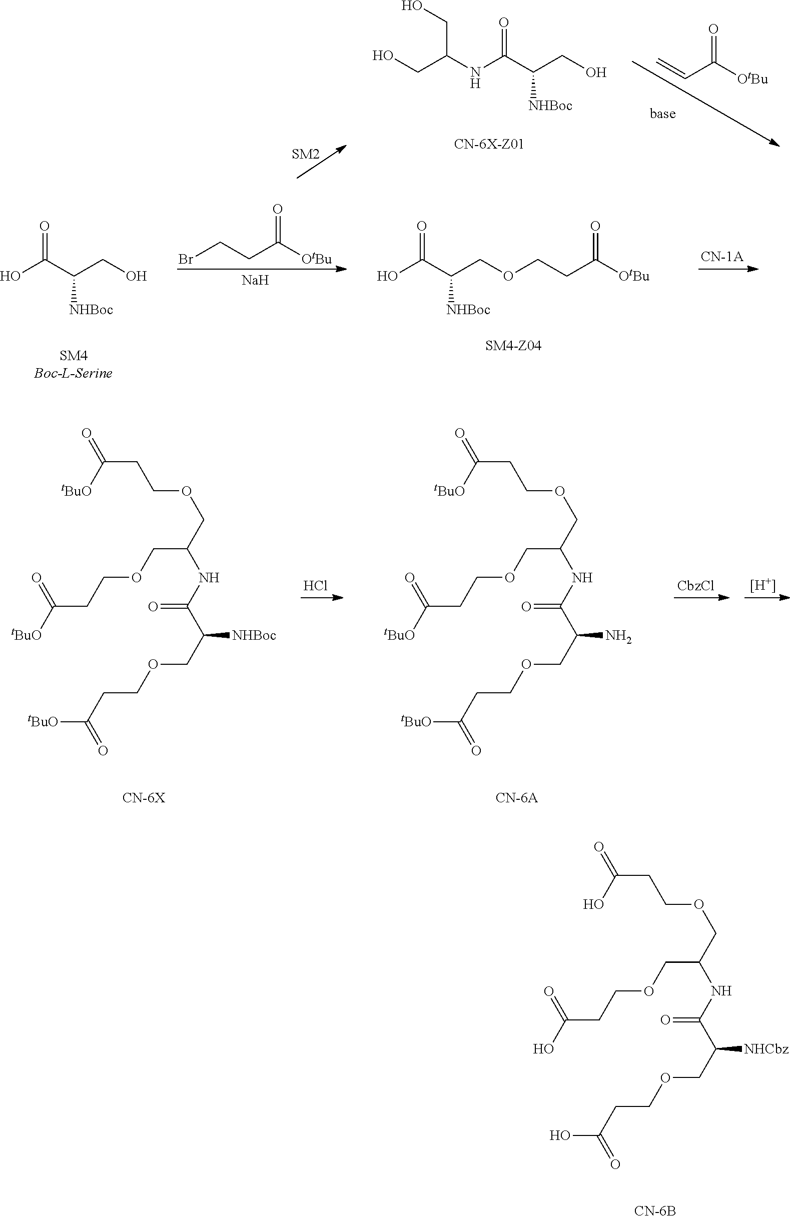

### (1) Synthesis of SM4-Z04 (Substitution Reaction)

SM4 monohydrate (7.2 g, 32.3 mmol), tetrahydrofuran (120 mL) and 20 g of anhydrous sodium sulfate were added to a flask, stirred at room temperature for 0.5 h and then filtered. The filtrate was added to a flask, cooled in an ice bath of 0-5° C., and added with sodium hydride (60%, 3.88 g, 97.0 mmol) in batches. The reaction was maintained at the temperature and carried out under stirring for 0.5 h. The reaction was then dropwise added with tert-butyl 3-bromopropionate (10.8 g, 51.7 mmol) in tetrahydrofuran (20 mL), was allowed to naturally increase to room temperature and then carried out for 16 h when TLC showed that the raw materials were completely converted (dichloromethane/methanol, 10/1, R_{f} of the product: 0.4). The reaction was quenched with 25% aqueous ammonium chloride solution (80 mL), adjusted to about pH7 with 0.5 N hydrochloric acid, and subjected to distillation under reduced pressure to remove most of tetrahydrofuran, and the residue was extracted with dichloromethane (80 mL×3). The obtained organic phases were combined, dried over anhydrous sodium sulfate, filtered and concentrated under reduced pressure. The residue was purified by silica gel column chromatography to obtain 1.94 g of a yellow oily product with a yield of 18%.

MS (ESI): m/z [M+H]⁺, theoretical 334.2, found 334.4.

### (2) Synthesis of CN-6X-Z01 (Amidation Condensation Reaction)

Under N₂ atmosphere, dichloromethane (40 mL), SM4 monohydrate (2.3 g, 25.2 mmol), SM2 (5.6 g, 25.2 mmol) and 5 g of anhydrous sodium sulfate were successively added into a flask and the reaction was carried out under stirring for 0.5 h. The reaction mixture was then successively added with EDCI (6.3 g, 32.8 mmol), HOBt (3.4 g, 25.2 mmol) and DIEA (6.5 g, 50.4 mmol) and then the reaction was carried out under stirring at room temperature for 6 h when TLC showed that the reaction was complete. The reaction solution was added with 30 mL of pure water and extracted with dichloromethane (20 mL×3). The obtained organic phases were combined, washed with 26% aqueous sodium chloride solution (40 mL), dried over anhydrous sodium sulfate, filtered and concentrated. The crude product was purified by silica gel column chromatography (200-300 meshes, dichloromethane/methanol, 30/1) to obtain 2.67 g of a white solid product with a yield of 38%.

MS (ESI): m/z [M+Na]⁺, theoretical 301.2, found 301.3.

### (3) Synthesis of CN-6X (Amidation Condensation Reaction)

Under N₂ atmosphere and at room temperature, CN-1A (1.25 g, 3.6 mmol), SM4-Z04 (1.2 g, 3.6 mmol), EDCI (0.89 g, 4.68 mmol), HOBt (0.48 g, 3.6 mmol) and DIEA (0.93 g, 7.2 mmol) were successively added to a flask containing dichloromethane (12 mL) and then the reaction was carried out under stirring at room temperature for 4 h when LCMS showed that the reaction was complete. The reaction solution was added with 12 mL of pure water and extracted with dichloromethane (10 mL×3). The obtained organic phases were combined, washed with 26% aqueous sodium chloride solution (12 mL), dried over anhydrous sodium sulfate, filtered and concentrated. The crude product was purified by silica gel column chromatography (200-300 meshes, petroleum ether/ethyl acetate, 2/1) to obtain 1.98 g of a colorless oily liquid product with a yield of 83%.

MS (ESI): m/z [M+Na]⁺, theoretical 663.4, found 663.5.

### (4) Synthesis of CN-6A (De-Tert-Butoxycarbonylation Reaction)

CN-6X (1.9 g, 2.87 mmol) was added to a flask containing 1,4-dioxane (5 mL), followed by addition of 4 M HCl/1,4-dioxane solution (5 mL) under constant stirring. The reaction was then carried out under stirring for 1 h when LCMS showed that the reaction was completed. The reaction solution was concentrated under reduced pressure and dried under vacuum at 40° C. to obtain 1.79 g of a yellow oily product (a HCl salt) with a yield of 104%.

MS (ESI): m/z [M+H]⁺, theoretical 563.4, found 563.5.

### (5) Synthesis of CN-6B (Benzyloxycarbonyl Protection and De-Tert-Butylation Reaction)

CN-6A (a HCl salt, 857 mg, 1.43 mmol) was added to a flask and dissolved with tetrahydrofuran (10 mL), and successively added with pure water (5 mL) and sodium bicarbonate (144 mg, 1.72 mmol), and under stirring, the reaction was finally dropwise added with benzyl chloroformate (268 mg, 1.57 mmol), and then carried out for 1 h when LCMS showed that the reaction was completed. The residue obtained by concentration under reduced pressure was purified by silica gel column chromatography (200-300 meshes, petroleum ether/ethyl acetate, 1/1) to obtain 903 mg of a colorless oily product, a benzyl carbamate intermediate. The obtained oily intermediate was dissolved in dichloromethane (8 mL), and under constant stirring and at room temperature, trifluoroacetic acid (6 mL) was slowly added, and the reaction was then carried out for 6 h when LCMS showed that the reaction was completed. The reaction solution was concentrated under reduced pressure to be oily. The residue was dried under vacuum at 50° C. for 1 h to obtain 702.9 mg of a white foamy product with a yield of 93% (the product contained some residual TFA and could be directly used in subsequent reaction without purification).

MS (ESI): m/z [M+H]⁺, theoretical 529.2, found 529.4.

### 12. Synthesis of Intermediates CN-8A and CN-8B

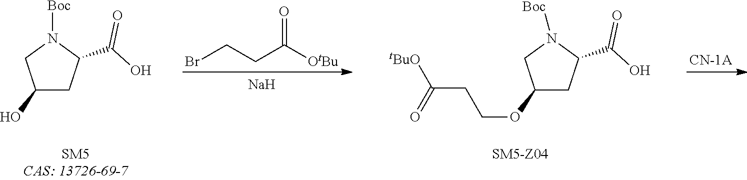

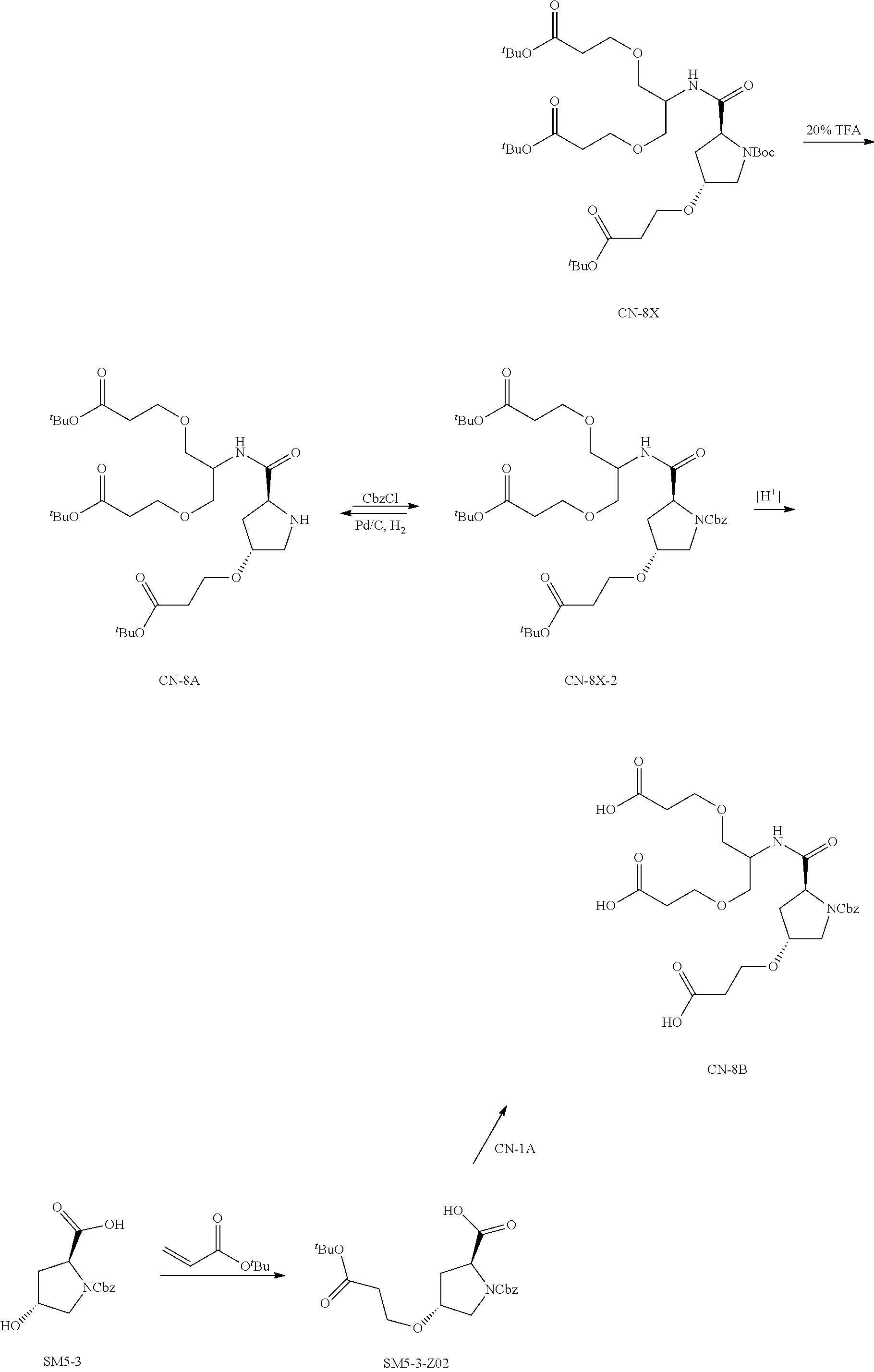

### (1) Synthesis of SM5-Z04

SM5-Z04 was obtained by subjecting SM5 and tert-butyl 3-bromopropionate as starting materials to the same substitution reaction steps for synthesis of SM5-Z01.

MS (ESI): m/z [M+H]⁺, theoretical 360.2, found 360.2.

### (2) Synthesis of CN-8X

CN-8X was obtained by subjecting SM5-Z04 and CN-1A as starting materials to the same amidation condensation reaction steps for synthesis of CN-7X.

MS (ESI): m/z [M+H]⁺, theoretical 689.4, found 689.4.

### (3) Synthesis of CN-8A

CN-8A was obtained by subjecting CN-8X as a starting material to the same de-tert-butoxycarbonylation reaction steps for synthesis of CN-5A.

MS (ESI): m/z [M+H]⁺, theoretical 589.4, found 589.5.

### (4) Synthesis of SM5-3-Z02 (Addition Reaction)

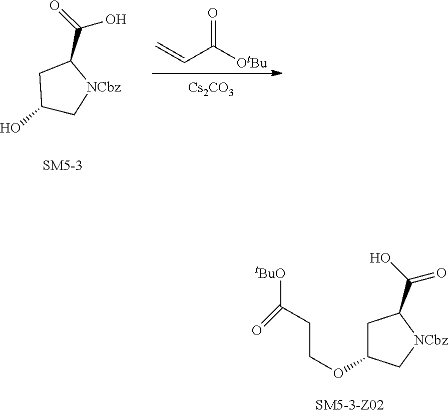

Tert-butanol (20 mL), SM5-3 (1.0 g, 3.77 mmol), tert-butyl acrylate (2.4 g, 18.9 mmol) and cesium carbonate (2.4 g, 7.5 mmol) were successively added to a flask, and under stirring, the reaction was gradually increased to 50° C. and then carried out for 20 h when TLC showed that the reaction was complete. The reaction solution was subjected to distillation under reduced pressure to remove the solvent, and the residue was added with 20 mL of dichloromethane, stirred for 0.5 h and filtered. The obtained crude product was purified by silica gel column chromatography (200-300 meshes, dichloromethane/methanol, 19/1 to 9/1) to obtain 0.58 g of a colorless transparent liquid with a yield of 39.1%.

MS (ESI): m/z [M+Na]⁺, theoretical 416.2, found 416.3.

### (5) Synthesis of CN-8X-2 (Amidation Condensation Reaction)

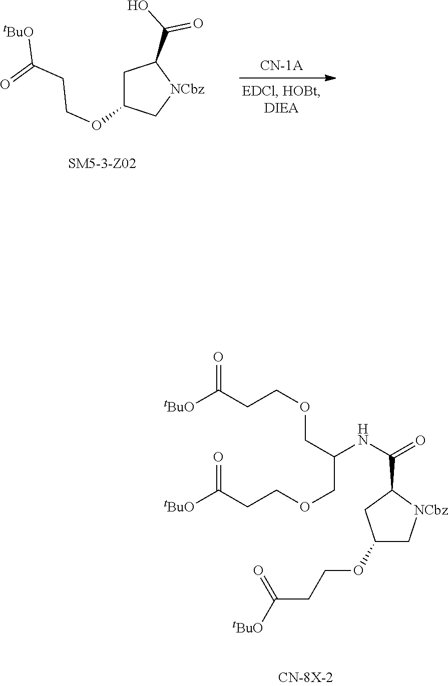 [SMILES: CC(C)(C)OC(=O)CCO[C@@H]1C[C@@H](C(=O)O)N(C(=O)OCc2ccccc2)C1 ; CC(C)(C)OC(=O)CCOCC(COCCC(=O)OC(C)(C)C)NC(=O)[C@@H]1C[C@@H](OCCC(=O)OC(C)(C)C)CN1C(=O)OCc1ccccc1]

Under N₂ atmosphere, dichloromethane (2 mL), CN-1A (90 mg, 0.25 mmol), SM5-3-Z02 (100 mg, 0.25 mmol), EDCI (50 mg, 0.25 mmol), HOBt (35 mg, 0.25 mmol) and DIEA (50 mg, 0.38 mmol) were successively added to a flask and the reaction was then carried out under stirring at room temperature for 4 h when LCMS showed that the reaction was complete. The reaction solution was added with 10 mL of pure water and extracted with dichloromethane (10 mL×3). The obtained organic phases were combined, washed with 26% aqueous sodium chloride solution (10 mL), dried over anhydrous sodium sulfate, filtered and concentrated. The crude product was purified by silica gel column chromatography (200-300 meshes, petroleum ether/ethyl acetate, 2/1) to obtain 161 mg of a yellow oily liquid with a yield of 87.8%.

MS (ESI): m/z [M+Na]⁺, theoretical 745.4, found 745.3.

### (6) Synthesis Method II of CN-8A (De-Benzyloxycarbonylation Reaction)

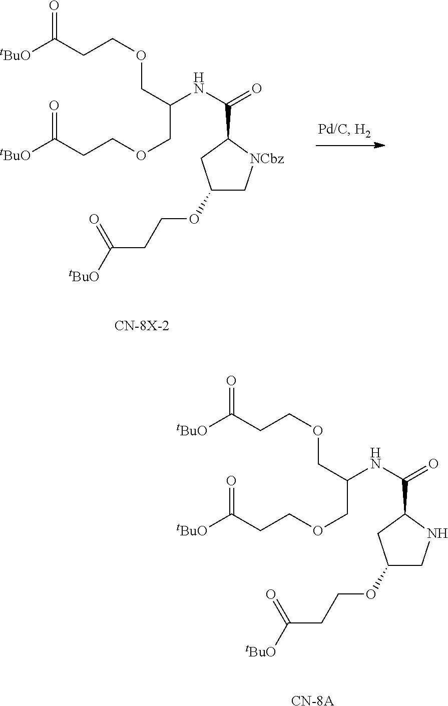 [SMILES: CC(C)(C)OC(=O)CCOCC(COCCC(=O)OC(C)(C)C)NC(=O)[C@@H]1C[C@@H](OCCC(=O)OC(C)(C)C)CN1C(=O)OCc1ccccc1 ; "CN-8X-2" ; CC(C)(C)OC(=O)CCOCC(COCCC(=O)OC(C)(C)C)NC(=O)[C@@H]1C[C@@H](OCCC(=O)OC(C)(C)C)CN1 ; "CN-8A"]

CN-8X-2 (100 mg, 0.14 mmol) was dissolved in 2 mL of tetrahydrofuran and added with Pd/C (10 mg, 10 wt %). The reaction system was replaced with hydrogen for three times and the reaction was carried out under stirring and under hydrogen atmosphere (about 10-20 kPa higher than the standard atmospheric pressure) for 12 h when LCMS showed that the reaction was complete. The obtained mixture was filtered to remove the solid and the filtrate was concentrated under reduced pressure. The obtained crude product was purified by silica gel column chromatography (200-300 meshes, dichloromethane/methanol, 9/1) to obtain 68 mg of a yellow oily liquid with a yield of 83.5%.

MS (ESI): m/z [M+H]⁺, theoretical 589.4, found 589.4.

### (7) Synthesis of CN-8B (De-Tert-Butylation Reaction)

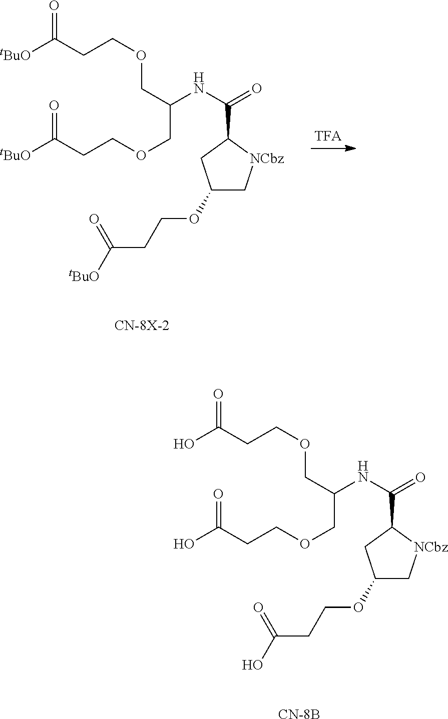 [SMILES: CC(C)(C)OC(=O)CCOCC(COCCC(=O)OC(C)(C)C)NC(=O)[C@@H]1C[C@@H](OCCC(=O)OC(C)(C)C)CN1C(=O)OCc1ccccc1 ; "CN-8X-2" ; O=C(O)CCOCC(COCCC(=O)O)NC(=O)[C@@H]1C[C@@H](OCCC(=O)O)CN1C(=O)OCc1ccccc1 ; "CN-8B"]

CN-8X-2 (60 mg, 0.083 mmol) and 2 mL of dichloromethane were added to a flask, and then added with trifluoroacetic acid (1.5 mL) under stirring at room temperature, and the reaction was then carried out under stirring or 6 h when LCMS showed that the reaction was complete. The solid was collected by filtration and purified by silica gel column chromatography (200-300 meshes, dichloromethane/methanol, 4/1) to obtain 26 mg of a product with a yield of 56.5%.

MS (ESI): m/z [M+H]⁺, theoretical 555.2, found 555.4.

### 13. Synthesis of Intermediates CN-9A, CN-9/B, CN-10 and CN-11

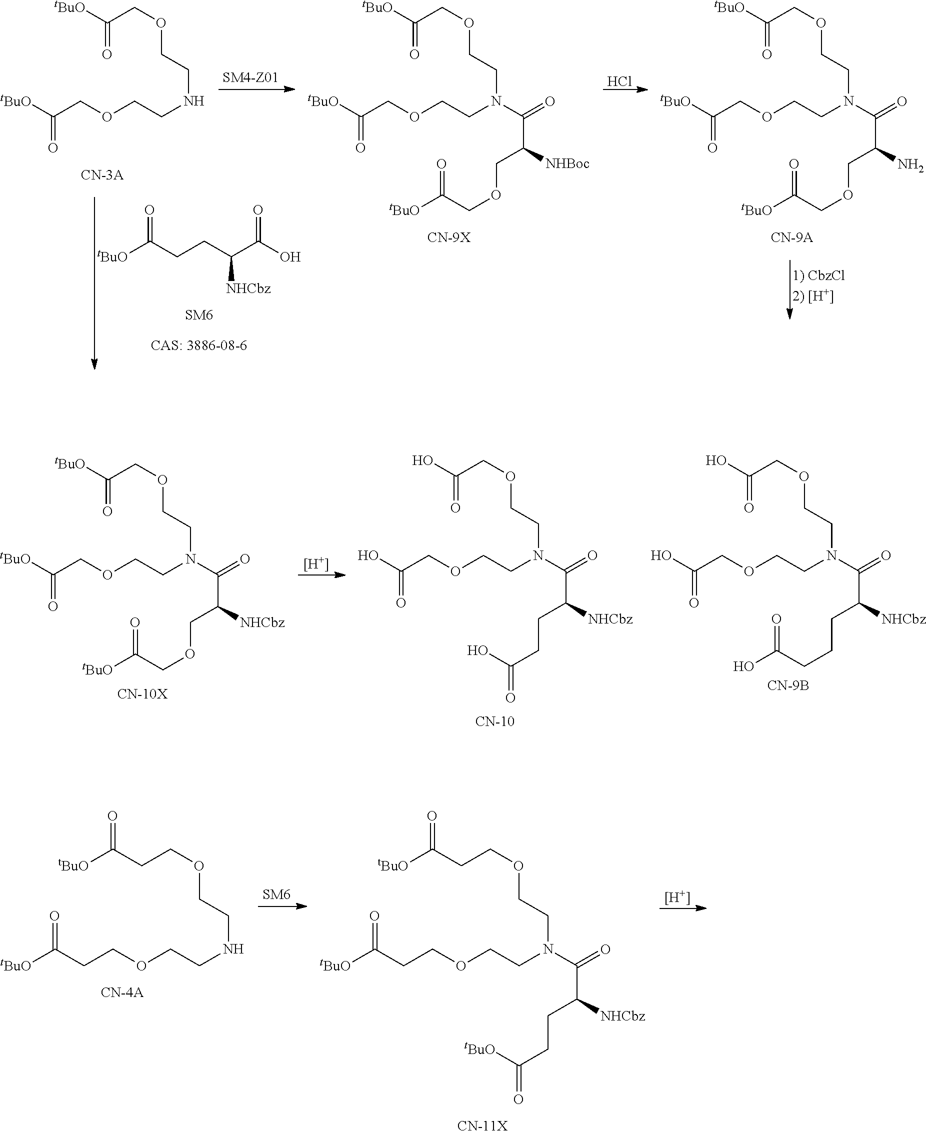

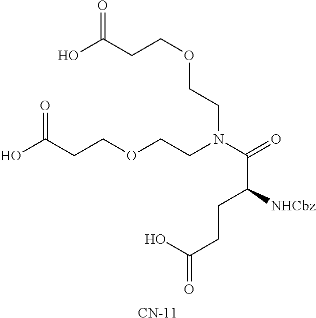 [SMILES: O=C(O)CCOCCN(CCOCCC(=O)O)C(=O)[C@H](CCC(=O)O)NC(=O)OCc1ccccc1 ; "CN-11"]

### (1) Synthesis of CN-9X (Amidation Condensation Reaction)

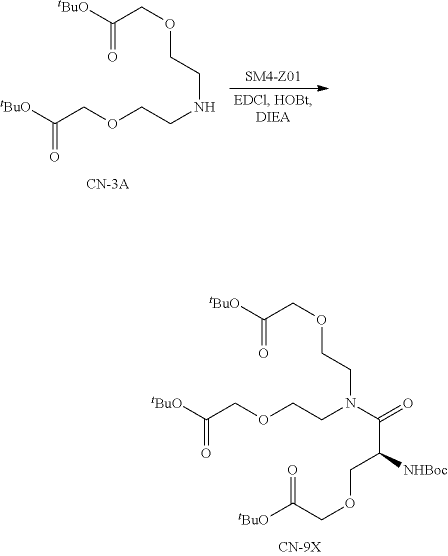 [SMILES: CC(C)(C)OC(=O)COCCNCCOCC(=O)OC(C)(C)C ; CC(C)(C)OC(=O)COCCN(CCOCC(=O)OC(C)(C)C)C(=O)[C@H](COCC(=O)OC(C)(C)C)NC(=O)OC(C)(C)C]

Under N₂ atmosphere, under stirring and at room temperature, SM4-Z01 (0.96 g, 3.0 mmol), CN-3A (1.1 g, 3.3 mmol), EDCI (0.88 g, 4.5 mmol), HOBt (0.49 g, 3.6 mmol) and DIEA (0.77 g, 6.0 mmol) were successively added to a flask containing dichloromethane (20 mL), and the reaction was then carried out for 12 h when TLC showed that the reaction was complete (dichloromethane/methanol, 10/1, R_{f} of the product: 0.8). The reaction solution was added with 50 mL of pure water and extracted with dichloromethane (50 mL×3). The obtained organic phases were combined, washed with 26% aqueous sodium chloride solution (50 mL), dried over anhydrous sodium sulfate, filtered, and subjected to distillation under reduced pressure to remove the solvent. The residue was purified by silica gel column chromatography (200-300 meshes, petroleum ether/ethyl acetate, 1/1) to obtain 1.32 g of a colorless oily product with a yield of 70%.

MS (ESI): m/z [M+H]⁺, theoretical 635.4, found 635.3.

### (2) Synthesis of CN-9A (De-Tert-Butoxycarbonylation Reaction)

CN-9X (1.07 g, 1.69 mmol) was added to a flask and dissolved with 1,4-dioxane (6 mL), and under stirring and at room temperature, 4 M HCl/1,4-dioxane solution (1 mL) was added, and then the reaction was carried out for 1 h when TLC showed that the reaction was complete (adjusted to about pH7, dichloromethane/methanol, 10/1, R_{f} of the product: 0.5). The obtained reaction solution was subjected to distillation under reduced pressure at room temperature to remove most of the solvent, added with 8% aqueous sodium bicarbonate solution (50 mL), stirred well, and extracted with dichloromethane (30 mL×3). The obtained organic phases were combined, dried over anhydrous sodium sulfate, filtered, and subjected to distillation under reduced pressure to remove the solvent. The residue was purified by silica gel column chromatography (200-300 meshes, dichloromethane/methanol, 9/1) to obtain 382 mg of a light yellow oily product with a yield of 42.3%.

MS (ESI): m/z [M+H]⁺, theoretical 535.3, found 535.5.

¹H NMR (400 MHz, DMSO-d₆) δ 3.99 (s, 2H), 3.96-3.92 (m, 3H), 3.91-3.84 (m, 1H), 3.72-3.58 (m, 3H), 3.57-3.44 (m, 4H), 3.43-3.29 (m, 4H), 1.99 (br s, 2H), 1.41 (s, 27H).

### (3) Synthesis of CN-9B (Benzyloxycarbonylation Protection and De-Tert-Butylation Reaction)

CN-9A (a HCl salt, 337 mg, 0.59 mmol) was added to a flask, dissolved with 20 mL of tetrahydrofuran, and successively added with 4 mL of pure water and sodium bicarbonate (60 mg, 0.71 mmol), and then under stirring, benzyl chloroformate (110 mg, 0.65 mmol) was slowly dropwise added, and the reaction was carried out for 1 h when TLC showed that the conversion of the raw materials was complete (petroleum ether/ethyl acetate, 1/1, R_{f} of the product: 0.4). The reaction solution was concentrated under reduced pressure, and the residue was purified by silica gel column chromatography (200-300 meshes, petroleum ether/ethyl acetate, 2/1 to 1/1) to obtain 300 mg of a colorless oily product (a benzyl carbamate intermediate) with a yield of 76%.

MS (ESI): m/z [M+H]⁺, theoretical 669.4, found 669.3.

The obtained oily intermediate was dissolved in dichloromethane (4 mL), and under constant stirring and at room temperature, trifluoroacetic acid (1.54 g, 13.5 mmol) was slowly added, and then the reaction was carried out for 0.5 h when LCMS showed that the reaction was complete. The reaction solution was concentrated under reduced pressure to be oily and dried under vacuum at 50° C. for 1 h to obtain 240 mg of a white foamy product with a yield of 107% (the product contained some residual TFA and could be directly used in subsequent reaction without purification).

MS (ESI): m/z [M+H]⁺, theoretical 501.2, found 501.1.

¹H NMR (400 MHz, DMSO-d₆) δ 7.62 (d, J=8.2 Hz, 1H), 7.41-7.25 (m, 5H), 5.15-4.90 (m, 2H), 4.71 (q, J=7.3 Hz, 1H), 4.06-3.86 (m, 6H), 3.83-3.71 (m, 1H), 3.71-3.60 (m, 3H), 3.60-3.36 (m, 6H).

### (4) Synthesis of CN-10X (Amidation Condensation Reaction)

CN-3A (666 mg, 2.0 mmol) and 16 mL of dichloromethane were added to a flask, and successively added with SM6 (741 mg, 2.2 mmol), EDCI (576 mg, 3.0 mmol), HOBt (324 mg, 2.4 mmol) and DIEA (774 mg, 6 mmol) under stirring and under N₂ atmosphere at room temperature. Then the reaction was carried out under stirring for 5 h when TLC showed that the conversion of the raw materials was complete (petroleum ether/ethyl acetate, 1/1, R_{f} of the product: 0.7). The reaction solution was added with 20 mL of pure water and extracted with dichloromethane (15 mL×3). The obtained organic phases were combined, washed with 26% aqueous sodium chloride solution, dried over anhydrous sodium sulfate, filtered, and subjected to distillation under reduced pressure to remove the solvent. The residue was purified by silica gel column chromatography (200-300 meshes, petroleum ether/ethyl acetate, 2/1) to obtain 701 mg of a colorless oily product with a yield of 53.7%.

MS (ESI): m/z [M+H]⁺, theoretical 653.4, found 653.3.

### (5) Synthesis of CN-10 (De-Tert-Butylation Reaction)

CN-10X (698 mg, 1.07 mmol) and dichloromethane (9 mL) were successively added to a flask, and under constant stirring and at room temperature, trifluoroacetic acid (2.3 g, 20.1 mmol) was slowly added, and then the reaction was carried out for 4 h when TLC showed that the reaction was complete. The reaction solution was concentrated under reduced pressure to be oily and dried under vacuum at 50° C. for 1 h to obtain 630 mg of a yellow oily product with a yield of 121% (the product contained some residual TFA and could be directly used in subsequent reaction without purification).

MS (ESI): m/z [M+H]⁺, theoretical 485.2, found 485.0.

¹H NMR (400 MHz, DMSO-d₆) δ 7.52 (d, J=8.3 Hz, 1H), 7.39-7.25 (m, 5H), 5.08-4.94 (m, 2H), 4.51-4.38 (m, 1H), 4.02 (s, 2H), 3.99 (s, 2H), 3.73-3.57 (m, 5H), 3.53 (t, J=5.8 Hz, 2H), 3.34 (dt, J=12.5, 5.8 Hz, 1H), 2.26 (t, J=7.3 Hz, 2H), 1.89-1.76 (m, 1H), 1.76-1.58 (m, 1H).

### (6) Synthesis of CN-11X

CN-11X was obtained by subjecting CN-4A and SM6 as starting materials to the same amidation condensation reaction steps for synthesis of CN-10X.

MS (ESI): m/z [M+H]⁺, theoretical 681.4, found 681.5.

### (7) Synthesis of CN-11

CN-11 was obtained by subjecting CN-11X as a starting material to the same de-tert-butylation reaction steps for synthesis of CN-10.

MS (ESI): m/z [M+H]⁺, theoretical 513.2, found 513.1.

### 14. Synthesis of Intermediates CN-12 and CN-13

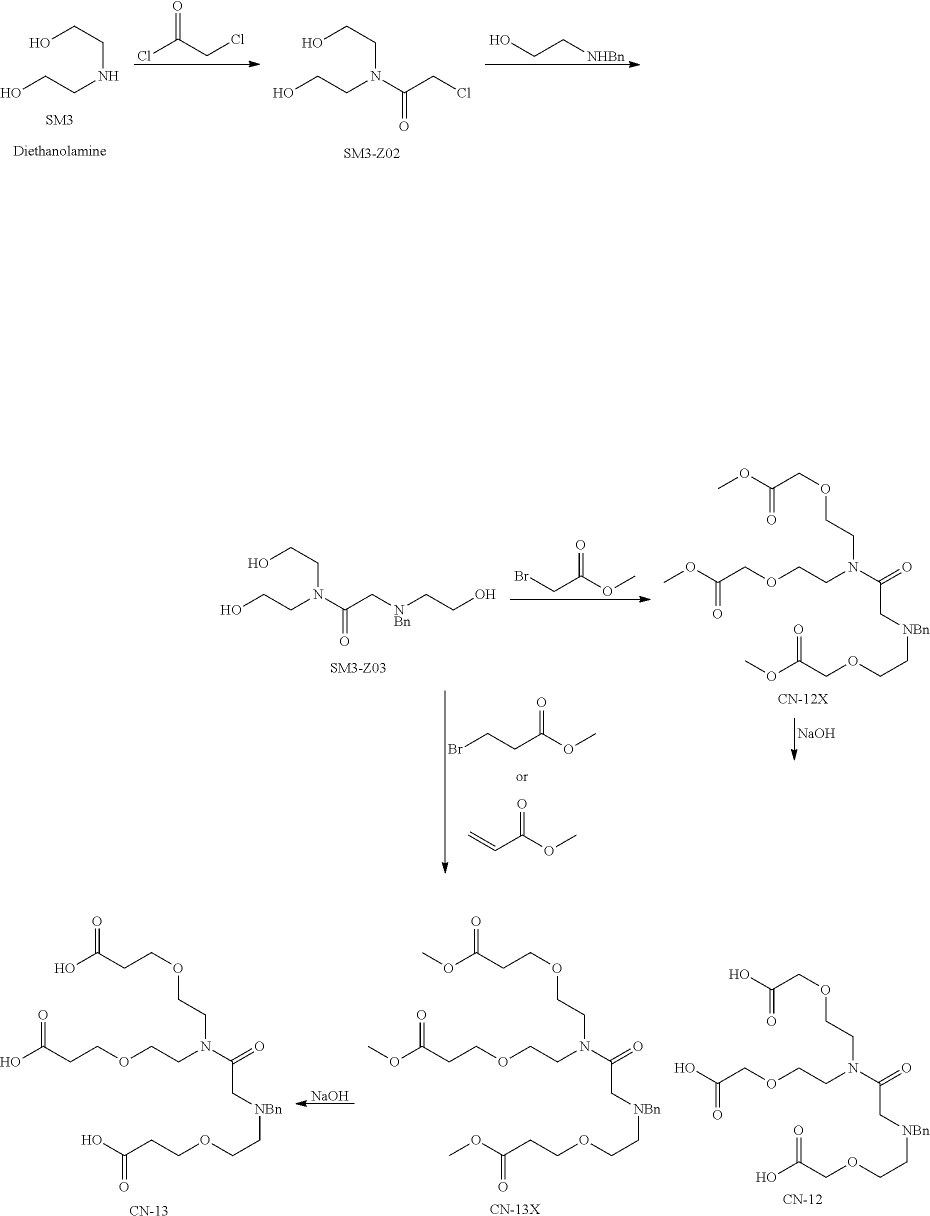

### (1) Synthesis of SM3-Z02 (Amidation Condensation Reaction)

SM3 (1.8 g, 17.1 mmol) and triethylamine (1.7 g, 17.1 mmol) were successively added to a flask containing tetrahydrofuran (12 mL), then cooled to 0-5° C. in an ice bath, and under constant stirring, chloroacetyl chloride (1.97 g, 17.4 mmol) was slowly added, and then the reaction was carried out for 1 h when LCMS showed that the reaction was complete. The reaction solution was added with pure water (20 mL), adjusted to about pH2 with 2 N hydrochloric acid, stirred at room temperature for 1 h and extracted with ethyl acetate (15 mL×3). The obtained organic phases were combined, washed with 26% aqueous sodium chloride solution, dried over anhydrous sodium sulfate, filtered, and subjected to distillation under reduced pressure to remove the solvent, and the residue was dried under vacuum to obtain 5.1 g of a light yellow oily product with a yield of 61%.

MS (ESI): m/z [M+H]⁺, theoretical 182.1, found 182.1.

### (2) Synthesis of SM3-Z03 (Substitution Reaction)

Benzylethanolamine (3.4 g, 22.5 mmol), SM3-Z02 (4 g, 22.0 mmol), potassium carbonate (3.1 g, 22.5 mmol) and sodium iodide (3.3 g, 22.0 mmol) were successively added to a flask containing acetonitrile (30 mL). The reaction was allowed to gradually increase to 50° C. and then carried out under stirring for 6 h when LCMS showed that the reaction was substantially complete. The obtained reaction solution was subjected to distillation under reduced pressure to remove the solvent, and the residue was added with ethyl acetate (30 mL), mixed well, washed with 26% aqueous sodium chloride solution (20 mL×3), dried over anhydrous sodium sulfate, filtered and concentrated under reduced pressure. The crude product was purified by silica gel column chromatography (200-300 meshes, petroleum ether/ethyl acetate, 1/1) to obtain 4.76 g of a light yellow oily product with a yield of 73%.

MS (ESI): m/z [M+H]⁺, theoretical 297.2, found 297.4.

### (3) Synthesis of CN-12X

CN-12X was obtained by subjecting SM3-Z03 and methyl bromoacetate as starting materials to similar substitution reaction steps for synthesis of CN-3X.

MS (ESI): m/z [M+H]⁺, theoretical 513.2, found 513.3.

### (4) Synthesis of CN-12 (Demethylation Reaction)

CN-12X (0.7 g, 1.36 mmol) was added to a flask containing methanol (6 mL), cooled to 0° C. in an ice water bath, and then added with 4 N aqueous sodium hydroxide solution (6 mL) under constant stirring. The reaction was allowed to naturally increase to room temperature and then carried out under stirring when for 2 h LCMS showed that the reaction was complete. The obtained reaction solution was adjusted to about pH4 with 2 N hydrochloric acid and extracted with ethyl acetate (6 mL×3) to remove the aqueous phase. The obtained organic phases were combined, dried over anhydrous sodium sulfate, filtered and concentrated under reduced pressure to obtain 0.41 g of a light yellow oily liquid product with a yield of 64%.

MS (ESI): m/z [M−H]⁻, theoretical 469.2, found 469.0.

¹H NMR (400 MHz, CD₃OD) δ 7.35-7.22 (m, 5H), 3.88-3.79 (m, 6H), 3.70 (t, J=5.9 Hz, 4H), 3.66 (s, 2H), 3.66 (t, J=5.6 Hz, 2H), 3.41 (1, J=5.8 Hz, 4H), 3.31 (s, 2H), 2.74 (t, J=5.6 Hz, 2H),

### (5) Synthesis of CN-13X

CN-13X was obtained by subjecting SM3-Z03 and methyl 3-bromopropionate as starting materials to similar substitution reaction steps for synthesis of CN-4X.

MS (ESI): m/z [M+H]⁺, theoretical 555.3, found 555.2.

### (6) Synthesis of CN-13

CN-13 was obtained by subjecting CN-13X as a starting material to similar demethylation reaction steps for synthesis of CN-12.

MS (ESI): m/z [M−H]⁻, theoretical 511.2, found 511.3.

¹H NMR (400 MHz, CD₃OD) δ 7.34-7.24 (m, 5H), 3.67 (s, 2H), 3.71-3.66 (m, 6H), 3.62-3.53 (m, 6H), 3.48-3.43 (m, 4H), 3.28 (s, 2H), 2.70 (t, J=5.8 Hz, 2H), 2.50-2.46 (m, 6H),

### 15. Synthesis of Intermediates NC-1A, NC-1B and NC-4

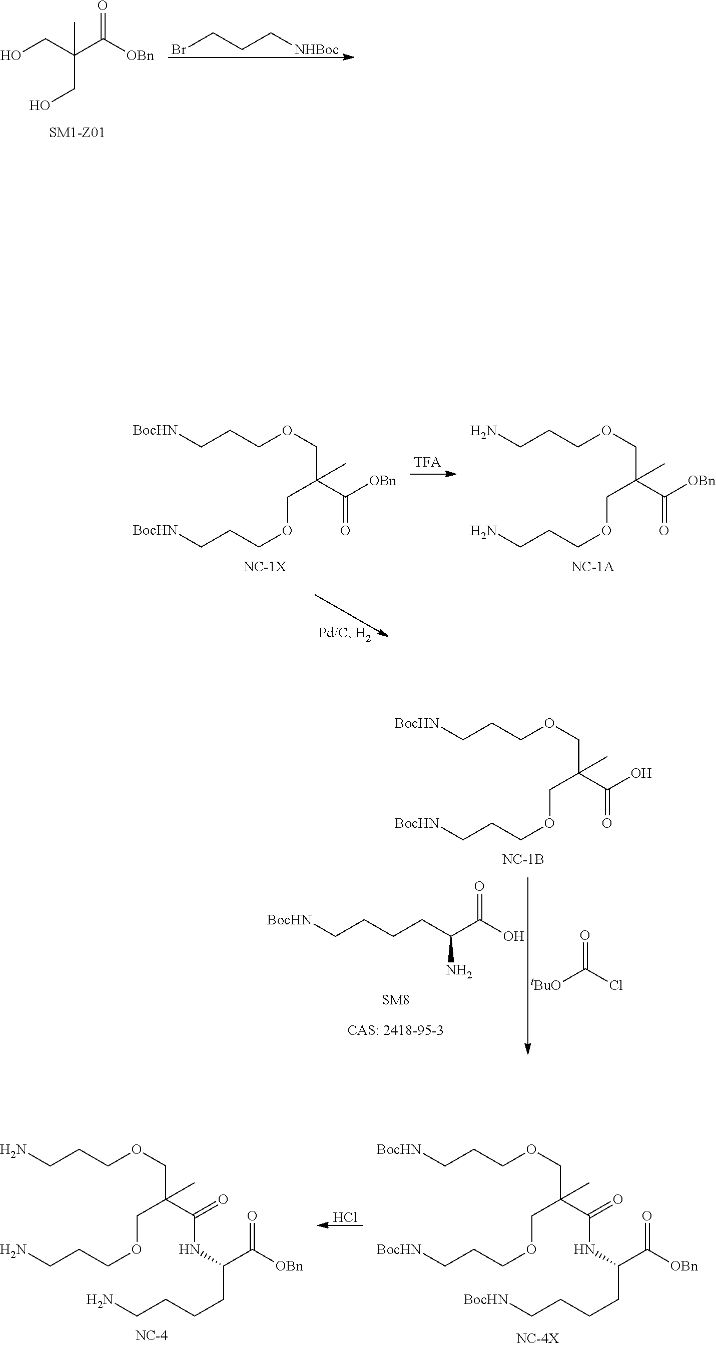

### (1) Synthesis of NC-1X (Substitution Reaction)

SM1-Z01 (2.0 g, 8.92 mmol), N-Boc-3-aminopropyl bromide (4.46 g, 18.7 mmol) and tetrabutylammonium bromide (1.44 g, 4.46 mmol) were successively added to a flask containing dichloromethane (30 mL), and then added with 37% aqueous sodium hydroxide solution (23 mL) under stirring at room temperature. The reaction was then carried out under stirring for 16 h when LCMS showed that the reaction was complete. The reaction solution was diluted with pure water (50 mL) and extracted with dichloromethane (20 mL×3). The obtained organic phases were combined, washed with 26% aqueous sodium chloride solution (40 mL), dried over anhydrous sodium sulfate, filtered and concentrated under reduced pressure. The residue was purified by silica gel column chromatography (200-300 meshes, petroleum ether/ethyl acetate, 10/1) to obtain 2.5 g of a colorless oily product with a yield of 52%.

MS (ESI): m/z [M+H]⁺, theoretical 539.3, found 539.4.

### (2) Synthesis of NC-1A (De-Tert-Butoxycarbonylation Reaction)

NC-1X (1.1 g, 2.04 mmol) was added to a flask containing dichloromethane (8 mL), and under constant stirring and at room temperature, trifluoroacetic acid (3 mL) was slowly added, and the reaction was then carried out for 2 h when LCMS showed that the reaction was completed. The reaction solution was concentrated under reduced pressure and dried under vacuum. The residue was dissolved in dichloromethane (15 mL), washed with 8% aqueous sodium bicarbonate solution (10 mL×3), dried over anhydrous sodium sulfate, filtered, and subjected to distillation under reduced pressure to remove the solvent, thereby obtaining 0.57 g of a yellow oily product with a yield of 82%.

MS (ESI): m/z [M+H]⁺, theoretical 339.2, found 339.1.

### (3) Synthesis of NC-1B

NC-1B was obtained by subjecting NC-1X as a starting material to similar debenzylation reaction steps for synthesis of CN-2B.

MS (ESI): m/z [M+H]⁺, theoretical 449.3, found 449.3.

### (4) Synthesis of NC-4X (Amidation Condensation Reaction)

Under N₂ atmosphere, NC-1B (1.0 g, 2.23 mmol) and N-methylmorpholine (0.45 g, 4.46 mmol) were successively added to a flask containing dichloromethane (12 mL), cooled to 0-5° C. in an ice bath, and slowly dropwise added with isobutyl chloroformate (0.31 g, 2.27 mmol). The reaction was then carried out under stirring for 1 h with the temperature maintained unchanged. The reaction was then added with SM8 (0.55 g, 2.23 mmol) and carried out under stirring at room temperature for 2 h when LCMS showed that the reaction was complete. The reaction solution was diluted with water (25 mL), adjusted to about pH4 with 2 N hydrochloric acid, and extracted with dichloromethane (12 mL×3). The obtained organic phases were combined, washed with 26% aqueous sodium chloride solution (20 mL), dried over anhydrous sodium sulfate, filtered and concentrated under reduced pressure. The crude product was crystallized with methyl tert-butyl ether to obtain 1.45 g of a light yellow solid product with a yield of 85%.

MS (ESI): m/z [M+H]⁺, theoretical 767.5, found 767.6.

### (5) Synthesis of NC-4

NC-4, a HCl salt, was obtained by subjecting NC-4X as a starting material to the same de-tert-butoxycarbonylation reaction steps for synthesis of CN-6A.

MS (ESI): m/z [M+H]⁺, theoretical 467.3, found 467.2.

¹H, NMR (400 MHz, DMSO-d₆) δ 7.98 (br s, 9H), 7.63 (d, J=8.5 Hz, 1H), 7.35-7.26 (m, 5H), 5.10 (s, 2H), 4.23-4.17 (m, 1H), 3.54-3.50 (m, 4H), 3.49 (dd, J=12.8, 7.9 Hz, 4H), 3.05-2.96 (m, 2H), 2.92-2.84 (m, 4H), 2.07-1.96 (m, 4H), 1.80-1.65 (m, 4H), 1.61-1.55 (m, 2H), 1.17 (s, 3H).

### 16. Synthesis of Intermediates NC-2, NC-3 and NC-5

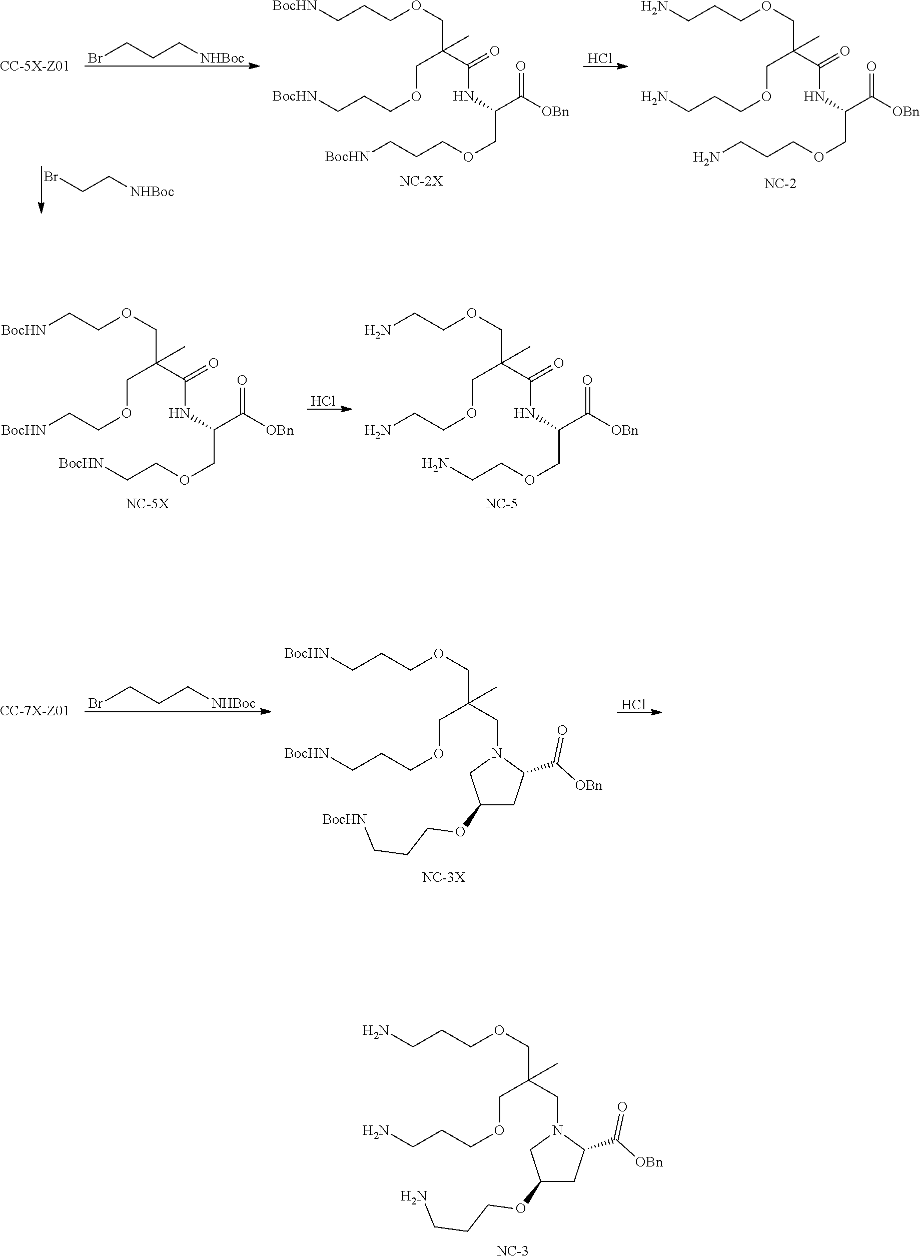

### (1) Synthesis of NC-2X

NC-2X was obtained by subjecting CC-5X-Z01 and N-Boc-3-aminopropyl bromide as starting materials to the same substitution reaction steps for synthesis of NC-1X.

MS (ESI): m/z [M+H]⁺, theoretical 783.5, found 783.6.

### (2) Synthesis of NC-2

NC-2 was obtained by subjecting NC-2X as a starting material to the same de-tert-butoxycarbonylation reaction steps for synthesis of NC-4.

MS (ESI): m/z [M+H]⁺, theoretical 483.3, found 483.4.

### (3) Synthesis of NC-5X

NC-5X was obtained by subjecting CC-5X-Z01 and N-Boc-bromoethylamine as starting materials to the same substitution reaction steps for synthesis of NC-1X.

MS (ESI): m/z [M+H]⁺, theoretical 741.4, found 741.4.

### (4) Synthesis of NC-5

NC-5 was obtained by subjecting NC-5X as a starting material to the same de-tert-butoxycarbonylation reaction steps for synthesis of NC-4.

MS (ESI): m/z [M+H]⁺, theoretical 441.3, found 441.3.

### (5) Synthesis of NC-3X

NC-3X was obtained by subjecting CC-7X-Z01 and N-Boc-3-aminopropyl bromide as starting materials to the same substitution reaction steps for synthesis of NC-1X.

MS (ESI): m/z [M+H]⁺, theoretical 809.5, found 809.6.

### (6) Synthesis of NC-3

NC-3, a HCl salt, was obtained by subjecting NC-3X as a starting material to the same de-tert-butoxycarbonylation reaction steps for synthesis of NC-4.

MS (ESI): m/z [M+H]⁺, theoretical 509.3, found 509.4.

¹H NMR (400 MHz, DMSO-d₆) δ 8.02 (br s, 9H), 7.34-7.27 (m, 5H), 5.22-5.16 (m, 2H), 4.54-4.51 (m, 1H), 4.44-4.40 (m, 1H), 3.61-3.49 (m, 10H), 2.92-2.84 (m, 7H), 2.40-2.33 (m, 1H), 2.23-2.18 (m, 1H), 2.05-1.96 (m, 7H), 1.20 (s, 3H).

### 17. Synthesis of Intermediates NN-1A, NN-1B and NN-4

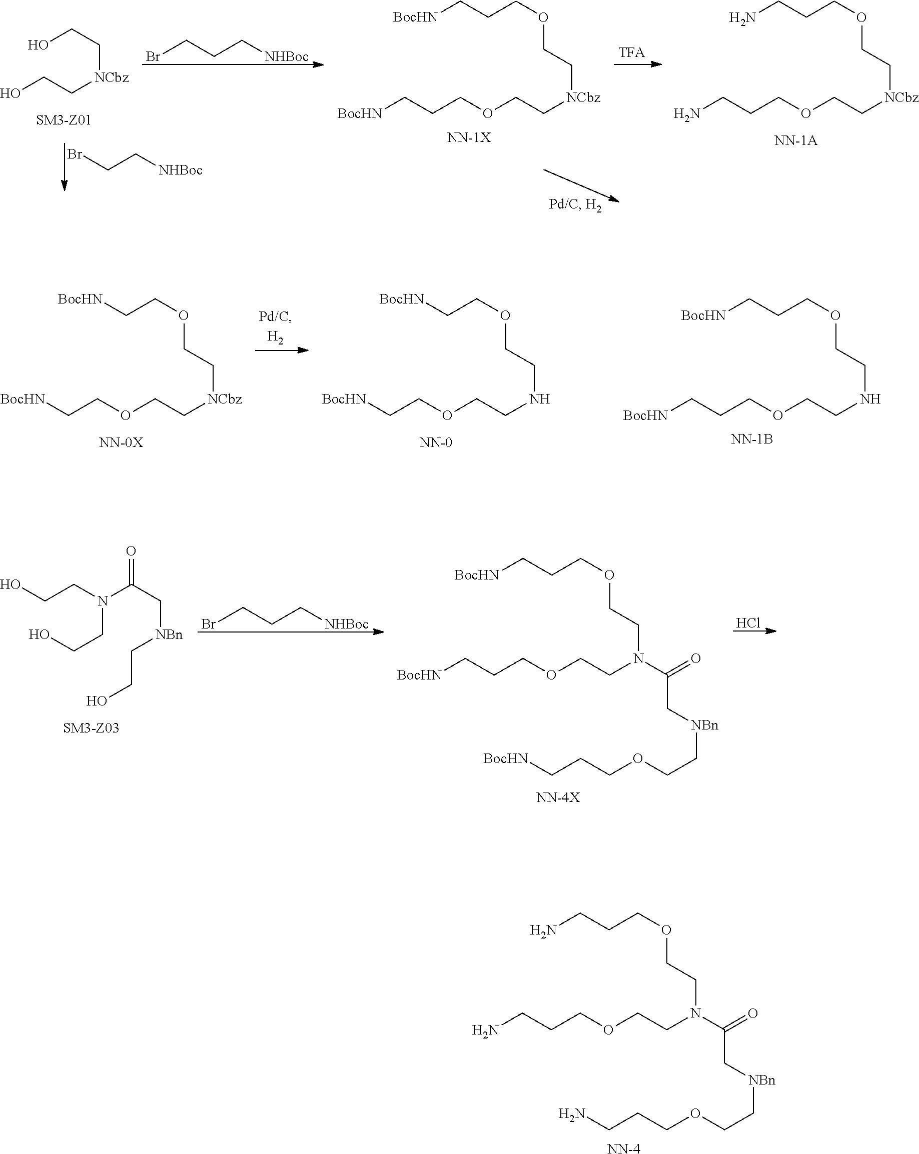

### (1) Synthesis of NN-1X

NN-1X was obtained by subjecting SM3-Z01 and N-Boc-3-aminopropyl bromide as starting materials to the same substitution reaction steps for synthesis of NC-1X.

MS (ESI): m/z [M+H]⁺, theoretical 554.3, found 554.3.

### (2) Synthesis of NN-1A

NN-1A was obtained by subjecting NN-1X as a starting material to the same de-tert-butoxycarbonylation reaction steps for synthesis of NC-1A.

MS (ESI): m/z [M+H]⁺, theoretical 354.2, found 354.2.

### (3) Synthesis of NN-1B

NN-1B was obtained by subjecting NN-1X as a starting material to the same debenzylation reaction steps for synthesis of NC-1B.

MS (ESI): m/z [M+H]⁺, theoretical 420.3, found 420.3.

### (4) Synthesis of NN-0X

NN-0X was obtained by subjecting SM3-Z01 and N-Boc-bromoethylamine as starting materials to the same substitution reaction steps for synthesis of NC-1X.

MS (ESI): m/z [M+H]⁺, theoretical 526.3, found 526.3.

### (5) Synthesis of NN-0

NN-0 was obtained by subjecting NN-0X as a starting material to the same debenzylation reaction steps for synthesis of NC-1B.

MS (ESI): m/z [M+H]⁺, theoretical 392.3, found 392.3.

### (6) Synthesis of NN-4X

NN-4X was obtained by subjecting SM3-Z03 and N-Boc-3-aminopropyl bromide as starting materials to the same substitution reaction steps for synthesis of NC-1X.

MS (ESI): m/z [M+H]⁺, theoretical 768.5, found 768.6.

### (7) Synthesis of NN-4

NN-4 was obtained by subjecting NN-4X as a starting material to the same de-tert-butoxycarbonylation reaction steps for synthesis of NC-4.

MS (ESI): m/z [M+H]⁺, theoretical 468.4, found 468.5.

### 18. Synthesis of Intermediates NN-2, NN-3 and CN-15

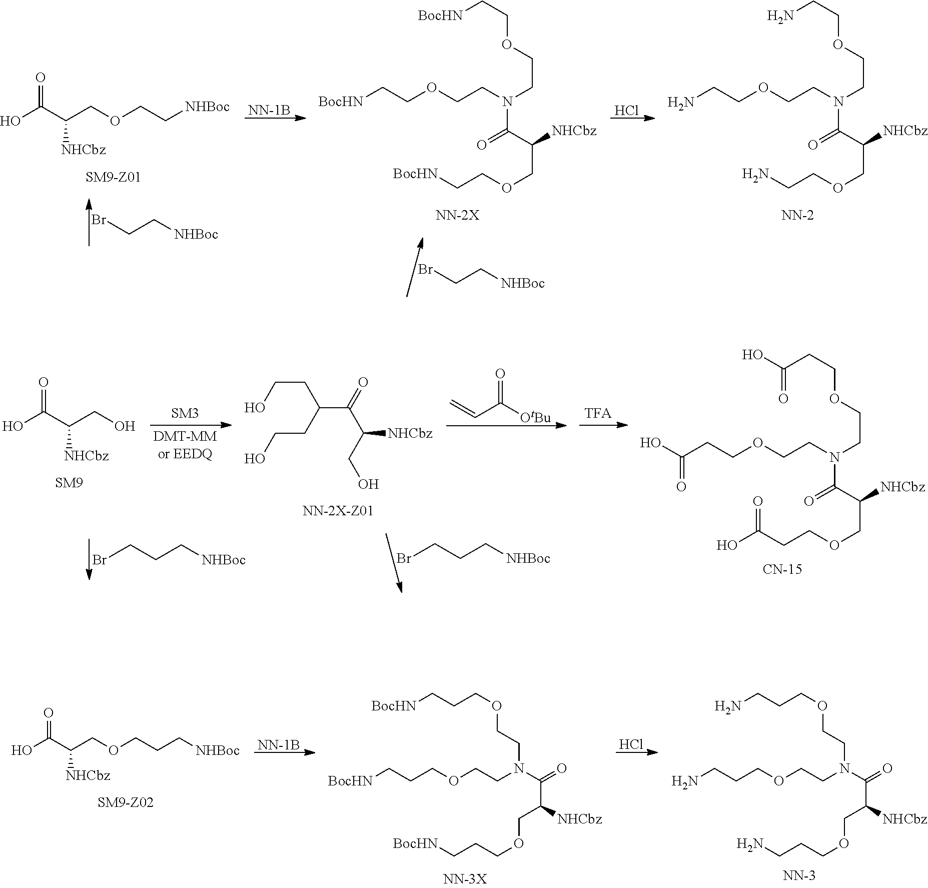

### (1) Synthesis of SM9-Z01

SM9-Z01 was obtained by subjecting SM9 and N-Boc-bromoethylamine as starting materials to the same substitution reaction steps for synthesis of SM4-Z01.

MS (ESI): m/z [M+H]⁺, theoretical 383.2, found 383.2.

### (2) Synthesis of SM9-Z02

SM9-Z02 was obtained by subjecting SM9 and N-Boc-3-aminopropyl bromide as starting materials to the same substitution reaction steps for synthesis of SM4-Z01.

MS (ESI): m/z [M+H]⁺, theoretical 397.2, found 397.3.

### (3) Synthesis of NN-2X-Z01

NN-2X-Z01 was obtained by subjecting SM9 and SM3 as starting materials to similar amidation condensation reaction steps for synthesis of CC-5X-Z01.

MS (ESI): m/z [M+H]⁺, theoretical 327.2, found 327.2.

### (4) Synthesis of NN-2X

NN-2X was obtained by subjecting SM9-Z01 and NN-1B as starting materials to similar amidation condensation reaction steps for synthesis of CN-9X.

MS (ESI): m/z [M+H]⁺, theoretical 756.4, found 756.5.

### (5) Synthesis of NN-2

NN-2, a HCl salt, was obtained by subjecting NN-2X as a starting material to similar de-tert-butoxycarbonylation reaction steps for synthesis of CN-9A.

MS (ESI): m/z [M+H]⁺, theoretical 456.3, found 456.3.

¹H NMR (400 MHz, DMSO-d₆) δ 7.96 (br s, 9H), 7.55 (d, J=7.8 Hz, 1H), 7.40-7.26 (m, 5H), 5.13-4.92 (m, 2H), 4.65 (m, 1H), 3.79-3.54 (m, 10H), 3.60-3.36 (m, 6H), 3.17-3.02 (m, 6H).

### (6) Synthesis of NN-3X

NN-3X was obtained by subjecting SM9-Z02 and NN-1B as starting materials to similar amidation condensation reaction steps for synthesis of CN-9X.

MS (ESI): m/z [M+H]⁺, theoretical 798.5, found 798.7.

### (7) Synthesis of NN-3

NN-3, a HCl salt, was obtained by subjecting NN-3X as a starting material to similar de-tert-butoxycarbonylation reaction steps for synthesis of CN-9A.

MS (ESI): m/z [M+H]⁺, theoretical 498.3, found 498.4.

### (8) Synthesis of CN-15

CN-15 was obtained by subjecting NN-2X-Z01 and tert-butyl acrylate as starting materials to similar addition reaction steps for synthesis of CN-6X from CN-6X-Z01 and to the same de-tert-butylation reaction steps for synthesis of CN-6B.

MS (ESI): m/z [M−H]⁻, theoretical 541.2, found 541.1.

### 19. Synthesis of Intermediates CL-1A, CL-1B and CL-2

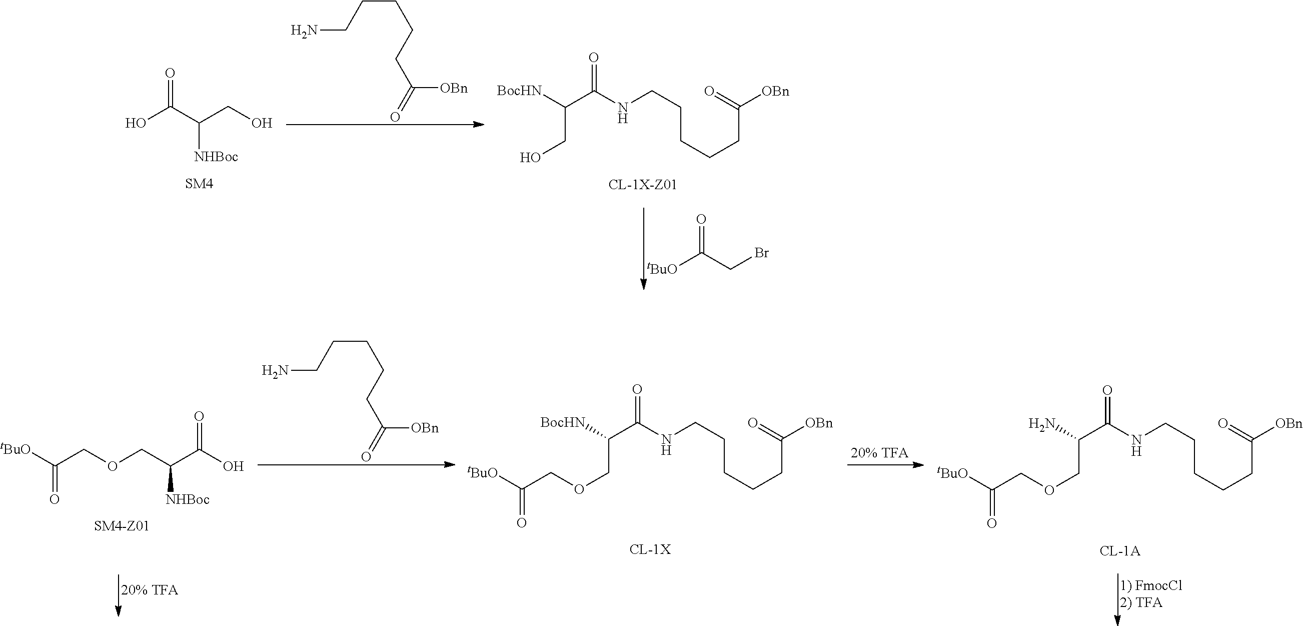

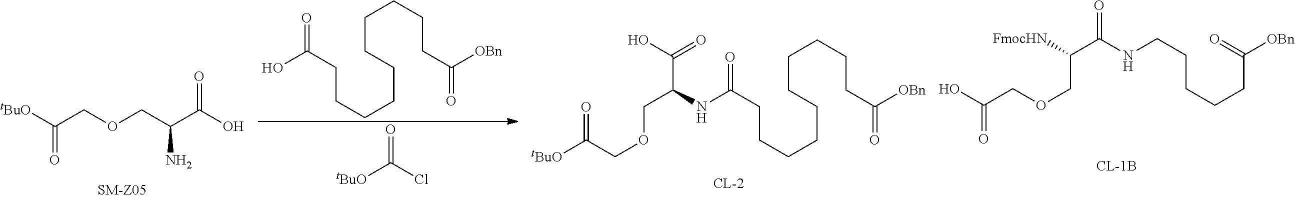

### (1) Synthesis of CL-1X-Z01 (Amidation Condensation Reaction)

Under N₂ atmosphere, SM4 (3.2 g, 15.6 mmol), benzyl 6-aminocaproate (3.45 g, 15.6 mmol), EDCI (3.87 g, 20.3 mmol), HOBt (2.1 g, 15.6 mmol) and DIEA (4.0 g, 31.2 mmol) were successively added to a flask containing dichloromethane (30 mL) and the reaction was carried out under stirring at room temperature for 6 h when LCMS showed that the reaction was complete. The reaction solution was added with pure water (60 mL) and extracted with dichloromethane (20 mL×3). The obtained organic phases were combined, washed with 26% aqueous sodium chloride solution (45 mL), dried over anhydrous sodium sulfate, filtered and concentrated under reduced pressure to obtain a crude product. The crude product was crystallized and purified with ethyl acetate and methyl tert-butyl ether to obtain 3.5 g of a white solid product with a yield of 55%.

MS (ESI): m/z [M+Na]⁺, theoretical 431.2, found 431.4.

### (2) Synthesis of CL-1X

CL-1X was obtained by subjecting SM4-Z01 and benzyl 6-aminocaproate as starting materials to the same amidation condensation reaction steps for synthesis of C1-1X-Z01.

MS (ESI): m/z [M+H]⁺, theoretical 523.3, found 523.1.

### (3) Synthesis of CL-1A

CL-1A was obtained by subjecting CN-1X as a starting material to similar de-tert-butoxycarbonylation reaction steps for synthesis of CN-5A.

MS (ESI): m/z [M+H]⁺, theoretical 423.2, found 423.2.

(4) Synthesis of CL-1B (9-Fluorenylmethoxycarbonylation Protection and de-Tert-Butylation Reaction)

CL-1A (a TFA salt, 2.6 g, 4.85 mmol) and 8% aqueous sodium bicarbonate solution (15 mL) were successively added to a flask containing 1,4-dioxane (12 mL) and cooled to 0-5° C. in an ice bath, and under constant stirring, 9-fluorenylmethoxycarbonyl chloride (1.5 g, 5.82 mmol) was slowly added, and the reaction was then carried out for 16 h when LCMS showed that the reaction was completed. The reaction solution was added with pure water (20 mL) and extracted with ethyl acetate (15 mL×3). The obtained organic phases were combined, washed with 26% aqueous sodium chloride solution (20 mL), dried over anhydrous sodium sulfate, filtered, and subjected to distillation under reduced pressure to remove the solvent. The crude product was crystallized with methyl tert-butyl ether to obtain a light yellow solid product, which was an intermediate with 9-fluorenylmethoxycarbonylated amino group. The solid was dissolved in dichloromethane (8 mL), and under constant stirring and at room temperature, trifluoroacetic acid (4 mL) was slowly added, and the reaction was then carried out for 4 h when LCMS showed that the reaction was completed. The reaction solution was concentrated under reduced pressure and dried under vacuum to obtain 2.17 g of a light yellow solid product with a yield of 76%.

MS (ESI): m/z [M+H]⁺, theoretical 589.3, found 589.4.

### (5) Synthesis of SM4-Z05

SM4-Z05 was obtained by subjecting SM4-Z01 as a starting material to similar de-tert-butoxycarbonylation reaction steps for synthesis of CL-1A.

MS (ESI): m/z [M+H]⁺, theoretical 220.1, found 220.1.

### (6) Synthesis of CL-2

CL-2 was obtained by subjecting SM4-Z05 and monobenzyl dodecanediate as starting materials to similar amidation condensation reaction steps for synthesis of BL-8-Z01.

MS (ESI): m/z [M+H]⁺, theoretical 522.3, found 522.4.

### 20. Synthesis of Intermediates CL-3A, CL-3B and CL-4

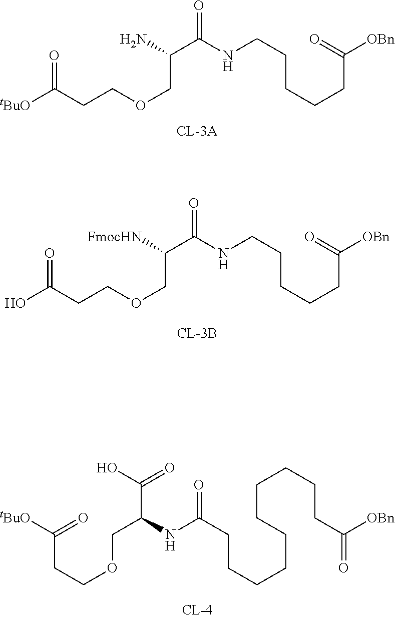

CL-3A/B was synthesized by the same steps for synthesis of CL-1A/B, except that SM4-Z01 was replaced with SM4-Z04 and tert-butyl bromoacetate was replaced with tert-butyl 3-bromopropionate;

CL-4 was synthesized by the same steps for synthesis of CL-2, except that SM4-Z01 was replaced with SM4-Z04.

### 21. Synthesis of Intermediates CL-5A, CL-5B and CL-6

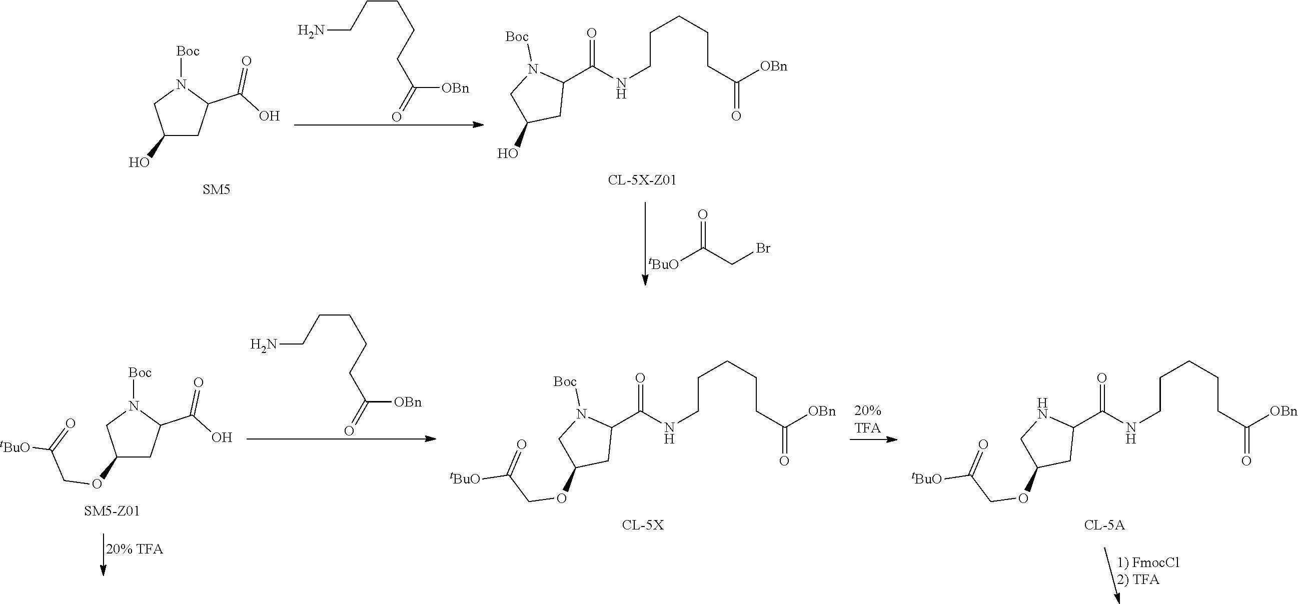

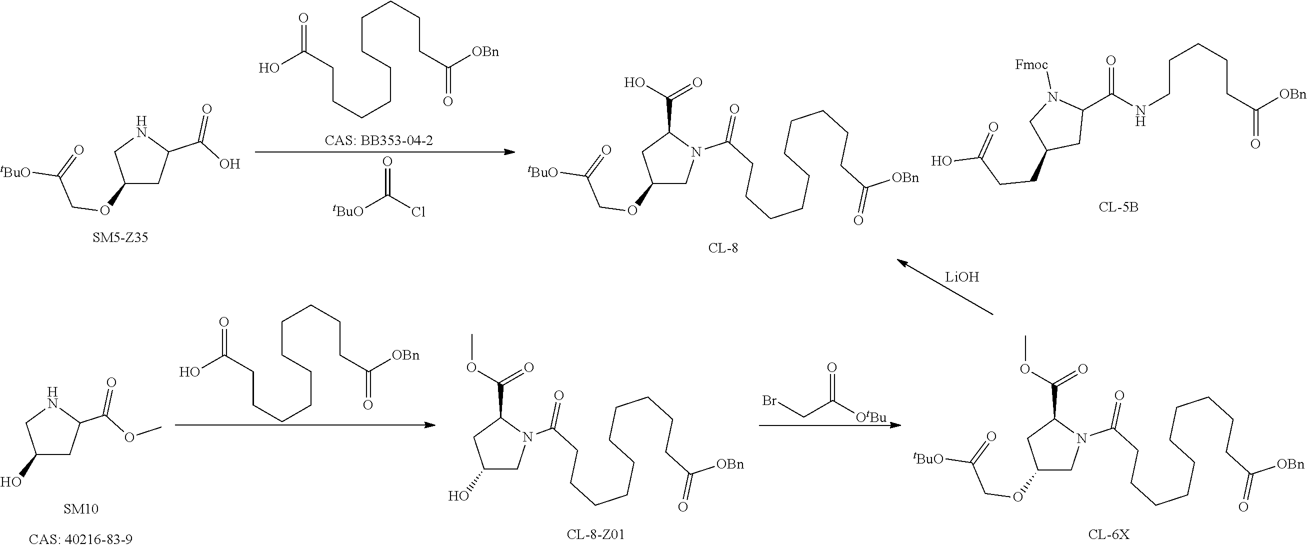

### (1) Synthesis of CL-5X-Z01

CL-5X-Z01 was obtained by subjecting SM5 and benzyl 6-aminocaproate as starting materials to the same amidation condensation reaction steps for synthesis of CL-1X-Z01.

MS (ESI): m/z [M+H]⁺, theoretical 435.2, found 435.3.

### (2) Synthesis of CL-5X

CL-5X was obtained by subjecting SM5-Z01 and benzyl 6-aminocaproate as starting materials to the same amidation condensation reaction steps for synthesis of CL-1X.

MS (ESI): m/z [M+H]⁺, theoretical 549.3, found 549.4.

### (3) Synthesis of CL-5A

CL-5A was obtained by subjecting CL-5X as a starting material to similar de-tert-butoxycarbonylation reaction steps for synthesis of CL-1A.

MS (ESI): m/z [M+H]⁺, theoretical 449.3, found 449.4.

### (4) Synthesis of CL-5B

CL-5B was obtained by subjecting CN-5A as a starting material to the same 9-fluorenylmethoxycarbonylation protection and de-tert-butylaton reaction steps for synthesis of CL-1B.

MS (ESI): m/z [M+H]⁺, theoretical 615.3, found 615.3.

### (5) Synthesis of SM5-Z05

SM5-Z05 was obtained by subjecting SM5-Z01 as a starting material to similar de-tert-butoxycarbonylation reaction steps for synthesis of SM4-Z05.

MS (ESI): m/z [M+H]⁺, theoretical 246.1, found 246.2.

### (6) Synthesis of CL-6-Z01 (Amidation Condensation Reaction)

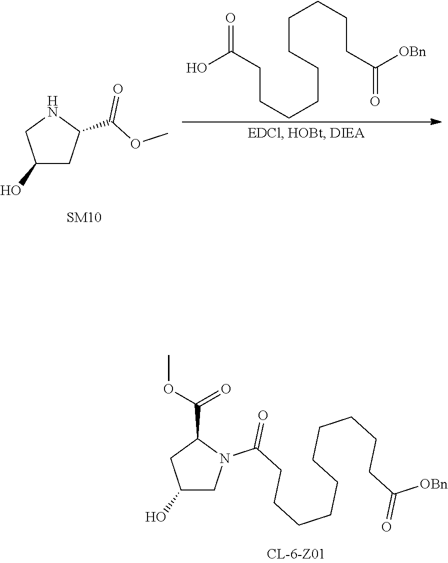

SM10 (3.62 g, 20 mmol), monobenzyl dodecanediate (6.4 g, 20 mmol), EDCI (4.22 g, 22 mmol) and HOBt (3 g, 22 mmol) were successively added to a flask containing dichloromethane (80 mL), and added slowly with DIEA (6.45 g, 50 mmol) under stirring at room temperature, and the reaction was carried out under stirring for 8 h when TLC showed that the raw materials was completely converted (dichloromethane/methanol, 10/1, R_{f} of the product: 0.4). The reaction solution was added with pure water (120 mL) and extracted with dichloromethane (50 mL×3). The obtained organic phases were combined, washed with 26% aqueous sodium chloride solution (120 mL), dried over anhydrous sodium sulfate, filtered and concentrated under reduced pressure to obtain a yellow oily liquid crude product. The crude product was purified by silica gel column chromatography (200-300 meshes, dichloromethane/methanol, 10/1 to 5/1) to obtain 6.8 g of a colorless transparent oily liquid product with a yield of 76%.

MS (ESI): m/z [M+Na]⁺, theoretical 470.2, found 470.2.

### (7) Synthesis of CL-6X (Substitution Reaction)

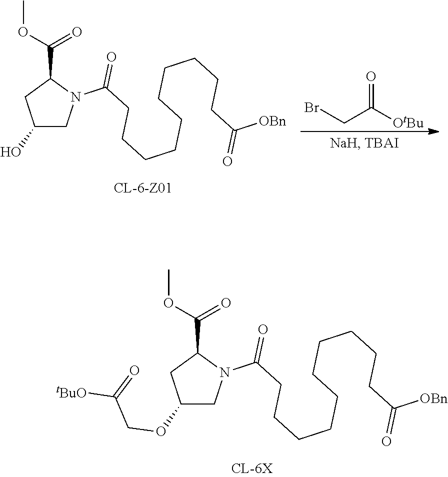 [SMILES: COC(=O)[C@@H]1C[C@@H](O)CN1C(=O)CCCCCCCCCCC(=O)OCc1ccccc1 ; CC(C)(C)OC(=O)CBr ; COC(=O)[C@@H]1C[C@@H](OCC(=O)OC(C)(C)C)CN1C(=O)CCCCCCCCCCC(=O)OCc1ccccc1]

Under N₂ atmosphere and in an ice water bath, sodium hydride (60%, 1.4 g, 36.2 mmol) was slowly added to tert-butyl bromoacetate (9.4 g, 48.3 mmol) and tetrabutylammonium iodide (0.4 g, 1.2 mmol) in tetrahydrofuran (70 mL) in batches. The reaction was allowed to increase to room temperature and then carried out under stirring for 0.5 h. The reaction was then added with CL-6-Z01 (5.4 g, 12.1 mmol) dissolved in tetrahydrofuran (50 mL) within 0.5 h, and carried out under stirring for 20 h when LCMS showed that the reaction was complete. The reaction solution was slowly added to 25% aqueous ammonium chloride solution (180 mL) cooled in an ice bath of 0-5° C. to quench the reaction, and extracted with ethyl acetate (80 mL×3). The obtained organic phases were combined, washed with 26% aqueous sodium chloride solution (100 mL), dried over anhydrous sodium sulfate, filtered and concentrated under reduced pressure to obtain 10 g of a yellow liquid crude product. The crude product was purified by silica gel column chromatography (200-300 meshes, petroleum ether/ethyl acetate, 7/3) to obtain 4.6 g of a colorless transparent oily liquid product with a yield of 67.8%.

MS (ESI): m/z [M+Na]⁺, theoretical 562.3, found 562.4.

¹H NMR (400 MHz, DMSO-d₆) δ 7.40-7.29 (m, 5H), 5.08 (s, 2H), 4.28-4.20 (m, 2H), 4.04 (s, 2H), 3.70-3.62 (m, 2H), 3.60 (s, 3H), 2.34 (t, J=7.4 Hz, 2H), 2.31-2.12 (m, 3H), 2.00-1.87 (m, 1H), 1.59-1.49 (m, 2H), 1.49-1.44 (m, 2H), 1.42 (s, 9H), 1.28-1.18 (m, 12H).

### (8) Synthesis of CL-6 (Demethylation Reaction)

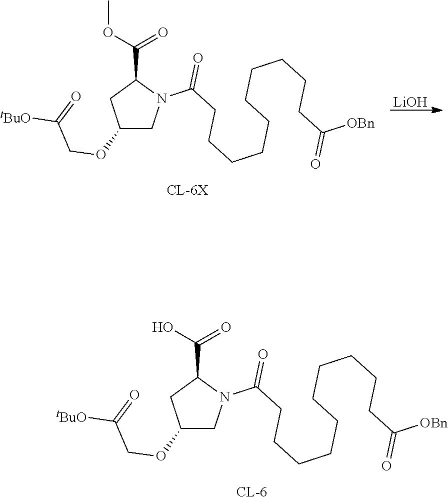 [SMILES: COC(=O)[C@@H]1C[C@@H](OCC(=O)OC(C)(C)C)CN1C(=O)CCCCCCCCCCC(=O)OCc1ccccc1 ; "LiOH" ; CC(C)(C)OC(=O)CO[C@@H]1C[C@@H](C(=O)O)N(C(=O)CCCCCCCCCCC(=O)OCc2ccccc2)C1]

CL-6X (4.6 g, 8.2 mmol) was added to a flask containing tetrahydrofuran (46 mL) and stirred until the reaction mixture became clear. The reaction was cooled to 0° C., followed by addition of 5 N aqueous lithium hydroxide solution (23 mL). The reaction was allowed to increase to room temperature and then carried out under stirring for 1 h when LCMS showed that the reaction was completed. The reaction solution was adjusted to about pH4 with 20% aqueous KHSO₄ solution and extracted with ethyl acetate (50 mL×3) to remove the aqueous phase. The obtained organic phases were combined, washed with 26% aqueous sodium chloride solution (100 mL), dried over anhydrous sodium sulfate, filtered and concentrated under reduced pressure to obtain 4.5 g of a yellow liquid crude product. The crude product was purified by silica gel column chromatography (200-300 meshes, dichloromethane/methanol, 95/5) to obtain 2.8 g of a colorless transparent oily liquid product with a yield of 62.5%.

MS (ESI): m/z [M+Na]⁺, theoretical 548.3, found 548.5.

### 22. Synthesis of Intermediates CL-7A, CL-7B and CL-8

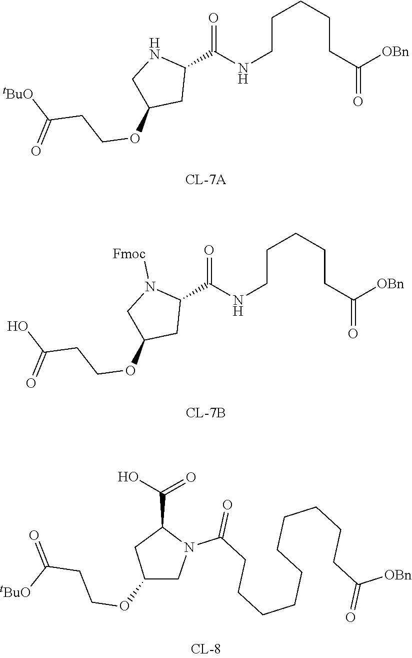

CL-7A and CL-7B were synthesized by the same steps for synthesis of CL-5A and CL-5B, respectively, except that SM5-Z01 was replaced with SM5-Z04 and tert-butyl bromoacetate was replaced with tert-butyl 3-bromopropionate.

CL-8 was synthesized by the same steps for synthesis of CL-6, except that SM5-Z01 was replaced with SM5-Z04.

### 23. Synthesis of Intermediates GN-1, GN-2 and GC-1

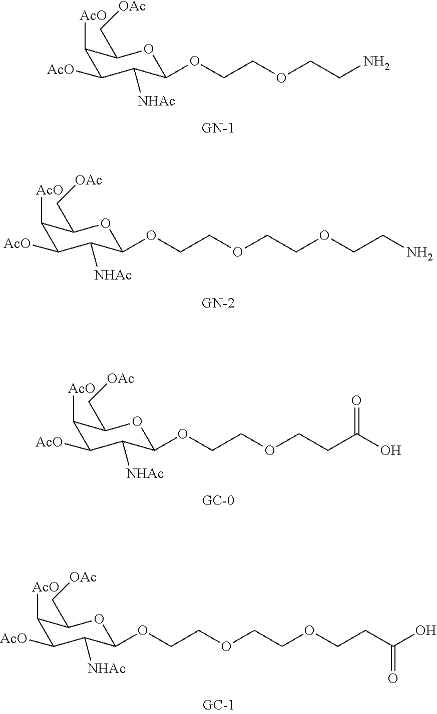 [SMILES: CC(=O)NC1C(OCCOCCN)OC(COC(C)=O)C(OC(C)=O)C1OC(C)=O ; CC(=O)NC1C(OCCOCCOCCN)OC(COC(C)=O)C(OC(C)=O)C1OC(C)=O ; CC(=O)NC1C(OCCOCCC(=O)O)OC(COC(C)=O)C(OC(C)=O)C1OC(C)=O ; CC(=O)NC1C(OCCOCCOCCC(=O)O)OC(COC(C)=O)C(OC(C)=O)C1OC(C)=O]

GN-1 and GN-2 were synthesized according to the method disclosed in patent WO2021153687A₁; GC-0 and GC-1 were synthesized according to the method disclosed in patent CN114716489A.

### 24. Synthesis of Intermediates GN-3, GN-4, GN-5 and GC-3

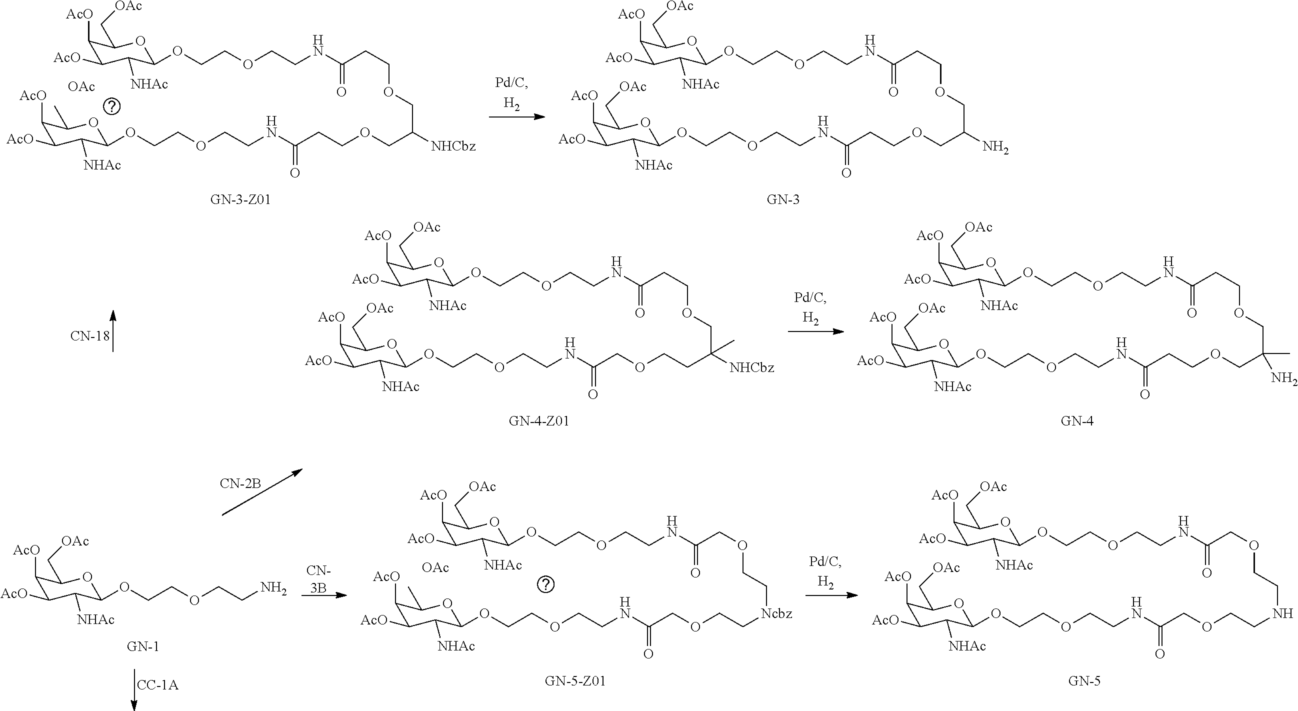

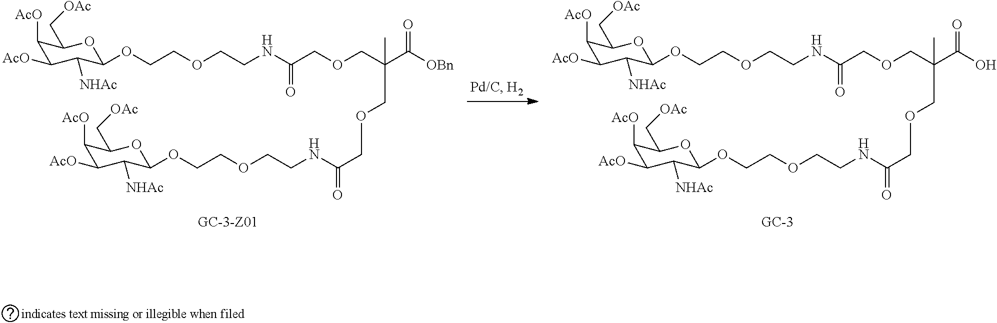 [SMILES: CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)OCc2ccccc2)OC(C)C(OC(C)=O)C1OC(C)=O ; CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)O)OC(C)C(OC(C)=O)C1OC(C)=O ; "OAc" ; "OAc"]

### (1) Synthesis of GN-3-Z01 (Amidation Condensation Reaction)

Under N₂ atmosphere, GN-1 (8.5 g, 19.6 mmol), CN-1B (3.4 g, 9.3 mmol), EDCI (4.4 g, 23.3 mmol) and HOBt (2.6 g, 19.6 mmol) were successively added to a flask containing dichloromethane (45 mL) and stirred at room temperature until the reaction mixture became clear. Then the reaction was added with DIEA (4.8 g, 37.2 mmol) and carried out under stirring for 4 h when LCMS showed that the reaction was complete. The reaction solution was added with pure water (50 mL) and extracted with dichloromethane (20 mL×4). The obtained organic phases were combined, washed with 26% aqueous sodium chloride solution (50 mL), dried over anhydrous sodium sulfate, filtered and concentrated. The obtained yellow oily product was purified by silica gel column chromatography (200-300 meshes, dichloromethane/methanol, 40/1) to obtain 7.04 g of a white solid with a yield of 63%.

MS (ESI): m/z [M+H]⁺, theoretical 1202.5, found 1202.6.

¹H NMR (400 MHz, DMSO-d₆), δ 7.91 (t, J=5.7 Hz, 2H), 7.84 (d, J=9.2 Hz, 2H), 7.40-7.28 (m, 5H), 7.15 (d, J=8.2 Hz, 1H), 5.21 (d, J=3.4 Hz, 2H), 5.04-4.94 (m, 4H), 4.54 (d, J=8.5 Hz, 2H), 4.03 (s, 6H), 3.92-3.83 (m, 2H), 3.82-3.74 (m, 2H), 3.73-3.64 (m, 1H), 3.62-3.53 (m 6H), 3.53-3.44 (m, 4H), 3.40-3.30 (m, 10H), 3.23-3.13 (m, 4H), 2.31 (t, J=6.5 Hz, 4H), 2.10 (s, 6H), 1.99 (s, 6H), 1.89 (s, 6H), 1.77 (s, 6H).

### (2) Synthesis of GN-3 (De-Benzyloxycarbonylation Reaction)

GN-3-Z01 (7.0 g, 5.8 mmol) was dissolved in tetrahydrofuran (30 mL) and added with Pd/C (0.7 g, 10 wt %). The reaction system was replaced with hydrogen for three times and then the reaction was carried out under stirring and under hydrogen atmosphere (about 10-20 kPa higher than the standard atmospheric pressure) for 3 h when LCMS showed that the reaction was complete. The reaction mixture was filtered to remove the solid and the filtrate was concentrated to obtain 5.89 g of a white solid product with a yield of 95%.

MS (ESI): m/z [M+H]⁺, theoretical 1068.5, found 1068.6.

### (3) Synthesis of GN-4-Z01 (Amidation Condensation Reaction)

Under N₂ atmosphere and at room temperature, GN-1 (9.1 g, 20.9 mmol), CN-2B (3.8 g, 9.97 mmol), EDCI (4.76 g, 24.9 mmol) and HOBt (2.8 g, 20.9 mmol) were successively added to a flask containing dichloromethane (50 mL) and stirred at room temperature until the reaction mixture became clear. The reaction was added with DIEA (5.14 g, 39.9 mmol) and then carried out under stirring for 5 h when LCMS showed that the reaction was complete. The reaction solution was added with pure water (50 mL) and extracted with dichloromethane (20 mL×4). The obtained organic phases were combined, washed with 26% aqueous sodium chloride solution (60 mL), dried over anhydrous sodium sulfate, filtered and concentrated. The obtained yellow oily product was purified by silica gel column chromatography (200-300 meshes, dichloromethane/methanol, 40/1) to obtain 8.2 g of a white solid with a yield of 68%.

MS (ESI): m/z [M+H]⁺, theoretical 1216.5, found 1216.3.

### (4) Synthesis of GN-4 (De-Benzyloxycarbonylation Reaction)

GN-4-Z01 (7.6 g, 6.25 mmol) was dissolved in tetrahydrofuran (30 mL) and added with Pd/C (0.8 g, 10 wt %). The reaction system was replaced with hydrogen for three times and then the reaction was carried out under stirring and under hydrogen atmosphere (about 10-20 kPa higher than the standard atmospheric pressure) for 3 h when LCMS showed that the reaction was complete. The reaction mixture was filtered to remove the solid and the filtrate was concentrated to obtain 6.63 g of a white solid product with a yield of 98%.

MS (ESI): m/z [M+H]⁺, theoretical 1081.5, found 1081.3.

### (5) Synthesis of GN-5-Z01 (Amidation Condensation Reaction)

Under N₂ atmosphere and at room temperature, GN-1 (6.8 g, 15.7 mmol), CN-3B (2.65 g, 7.45 mmol), EDCI (3.56 g, 18.6 mmol) and HOBt (2.1 g, 15.7 mmol) were successively added to a flask containing dichloromethane (40 mL) and stirred at room temperature until the reaction mixture became clear. The reaction was added with DIEA (3.84 g, 29.8 mmol) and then carried out under stirring for 5 h when LCMS showed that the reaction was complete. The reaction solution was added with pure water (50 mL) and extracted with dichloromethane (20 mL×4). The obtained organic phases were combined, washed with 26% aqueous sodium chloride solution (40 mL), dried over anhydrous sodium sulfate, filtered and concentrated. The obtained crude product was purified by silica gel column chromatography (200-300 meshes, dichloromethane/methanol, 40/1) to obtain 4.69 g of a white solid with a yield of 53%.

MS (ESI): m/z [M+Na]⁺, theoretical 1210.5, found 1210.7.

### (6) Synthesis of GN-5 (De-Benzyloxycarbonylation Reaction)

GN-5-Z01 (4.6 g, 3.87 mmol) was dissolved in tetrahydrofuran (30 mL) and successively added with trifluoroacetic acid (0.43 g, 3.8 mmol) and Pd/C (0.46 g, 10 wt %). The reaction system was replaced with hydrogen for three times and then the reaction was carried out under stirring and under hydrogen atmosphere (about 10-20 kPa higher than the standard atmospheric pressure) for 3 h when LCMS showed that the reaction was complete. The reaction mixture was filtered to remove the solid and the filtrate was concentrated to obtain 4.16 g of a light yellow solid product (a TFA salt) with a yield of 92%.

MS (ESI): m/z [M+H]⁺, theoretical 1054.5, found 1054.8.

### (7) Synthesis of GC-3-Z01 (Amidation Condensation Reaction)

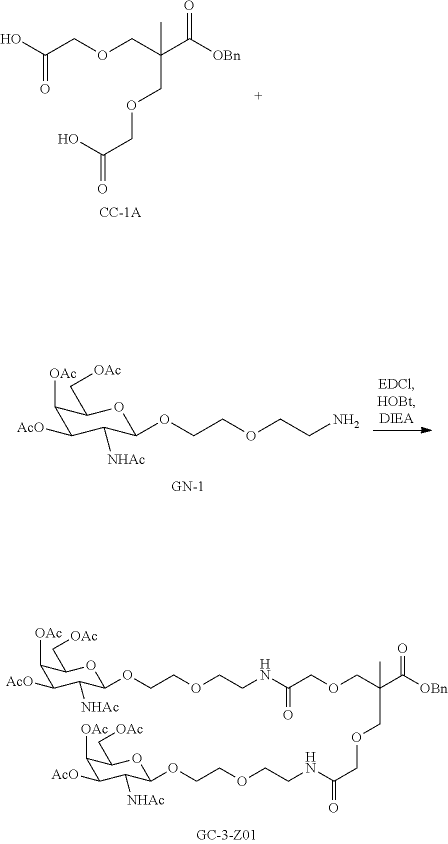 [SMILES: CC(COCC(=O)O)(COCC(=O)O)C(=O)OCc1ccccc1 ; "CC-1A" ; CC(=O)NC1C(OCCOCCN)OC(COC(C)=O)C(OC(C)=O)C1OC(C)=O ; CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)OCc2ccccc2)OC(COC(C)=O)C(OC(C)=O)C1OC(C)=O]

Under N₂ atmosphere, dichloromethane (80 mL), GN-1 (10.2 g, 23.5 mmol), CC-1A (4.0 g, 11.8 mmol), EDCI (6.8 g, 35.3 mmol) and HOBt (3.2 g, 23.5 mmol) were successively added to a flask and stirred at room temperature until the reaction mixture became clear. The reaction was added with DIEA (6.1 g, 47.0 mmol) and then carried out under stirring for 3 h when LCMS showed that the reaction was complete. The reaction solution was added with pure water (80 mL) and extracted with dichloromethane (40 mL×4). The obtained organic phases were combined, washed with 26% aqueous sodium chloride solution (80 mL), dried over anhydrous sodium sulfate, filtered and concentrated. The obtained yellow oily product was purified by silica gel column chromatography (200-300 meshes, dichloromethane/methanol, 49/1) to obtain 6.9 g of a white solid with a yield of 50%.

MS (ESI): m/z [M+Na]⁺, theoretical 1195.5, found 1195.5.

¹H NMR (500 MHz, CDCl₃) δ 7.39-7.29 (m, 5H), 7.24-7.14 (m, 2H), 6.66 (dd, J=32.7, 8.0 Hz, 2H), 5.36-5.33 (m, 2H), 5.32-5.23 (m, 2H), 5.19 (s, 2H), 4.77 (t, J=9.7 Hz, 2H), 4.17-4.07 (m, 5H), 4.04-3.98 (m, 4H), 3.95-3.88 (m, 4H), 3.76 (dd, J=8.9, 1.9 Hz, 2H), 3.73-3.69 (m, 2H), 3.67 (d, J=10.3 Hz, 2H), 3.62-3.57 (m, 4H), 3.54-3.51 (m, 4H), 3.48-3.41 (m, 2H), 3.17-3.12 (m, 6H), 2.13 (s, 6H), 2.03 (s, 6H), 1.98 (s, 6H), 1.94 (s, 6H), 1.26 (s, 3H).

### (8) Synthesis of GC-3 (Debenzylation Reaction)

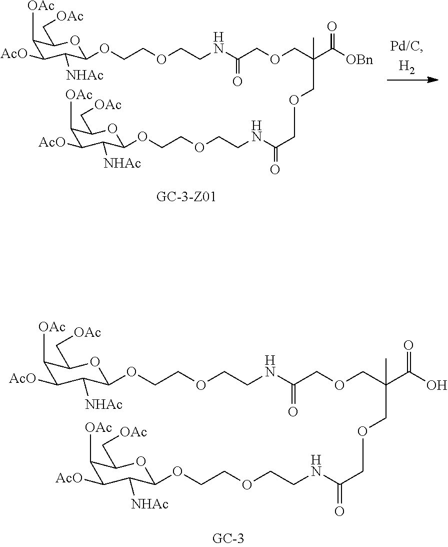 [SMILES: CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)OCc2ccccc2)OC(COC(C)=O)C(OC(C)=O)C1OC(C)=O ; CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)O)OC(COC(C)=O)C(OC(C)=O)C1OC(C)=O]

GC-3-Z01 (2.7 g, 2.3 mmol) was dissolved in tetrahydrofuran (27 mL) and added with Pd/C (0.25 g, 10 wt %). The reaction system was replaced with hydrogen for three times and the reaction was carried out under stirring and under hydrogen atmosphere (about 10-20 kPa higher than the standard atmospheric pressure) for 1.5 h when LCMS showed that the reaction was complete. The reaction mixture was filtered to remove the solid and the filtrate was concentrated to obtain 2.1 g of a white solid product with a yield of 84.3%.

MS (ESI): m/z [M+Na]⁺, theoretical 1105.4, found 1105.6.

¹H NMR (400 MHz, DMSO-d₆) δ 7.80 (dd, J=9.2, 2.9 Hz, 2H), 7.65 (t, J=5.8 Hz, 2H), 5.17 (d, J=3.4 Hz, 2H), 4.94 (dd, J=11.3, 3.3 Hz, 2H), 4.51 (d, J=8.4 Hz, 2H), 4.01-3.95 (m, 6H), 3.88-3.78 (m, 6H), 3.74 (dt, J=10.0, 5.0 Hz, 2H), 3.59-3.52 (m, 4H), 3.51-3.47 (m, 4H), 3.47-3.43 (m, 3H), 3.42-3.35 (m, 4H), 3.24-3.19 (m, 4H), 2.06 (s, 6H), 1.97-1.94 (m, 6H), 1.85 (s, 6H), 1.73 (s, 6H), 1.32 (s, 3H), 1.08 (s, 3H).

### (9) Synthesis of GC-3L, GC-4, GN-3L, GN-4L and GN-6

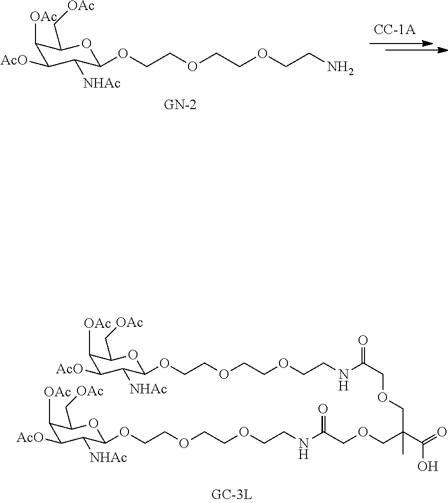 [SMILES: CC(=O)NC1C(OCCOCCOCCN)OC(COC(C)=O)C(OC(C)=O)C1OC(C)=O ; "CC-1A" ; CC(=O)NC1C(OCCOCCOCCNC(=O)COCC(C)(COCC(=O)NCCOCCOCCOC2OC(COC(C)=O)C(OC(C)=O)C(OC(C)=O)C2NC(C)=O)C(=O)O)OC(COC(C)=O)C(OC(C)=O)C1OC(C)=O]

GC-3L was synthesized by the same steps for synthesis of GC-3, except that GN-1 was replaced with GN-2.

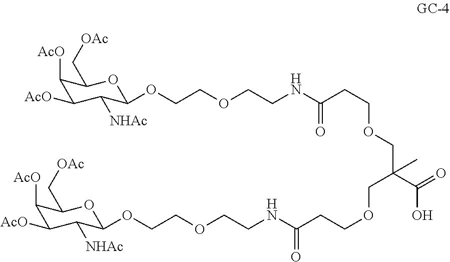 [SMILES: CC(=O)NC1C(OCCOCCNC(=O)CCOCC(C)(COCCC(=O)NCCOCCOC2OC(COC(C)=O)C(OC(C)=O)C(OC(C)=O)C2NC(C)=O)C(=O)O)OC(COC(C)=O)C(OC(C)=O)C1OC(C)=O]

GC-4 was synthesized by the same steps for synthesis of GC-3, except that CC-1A was replaced with CC-2A.

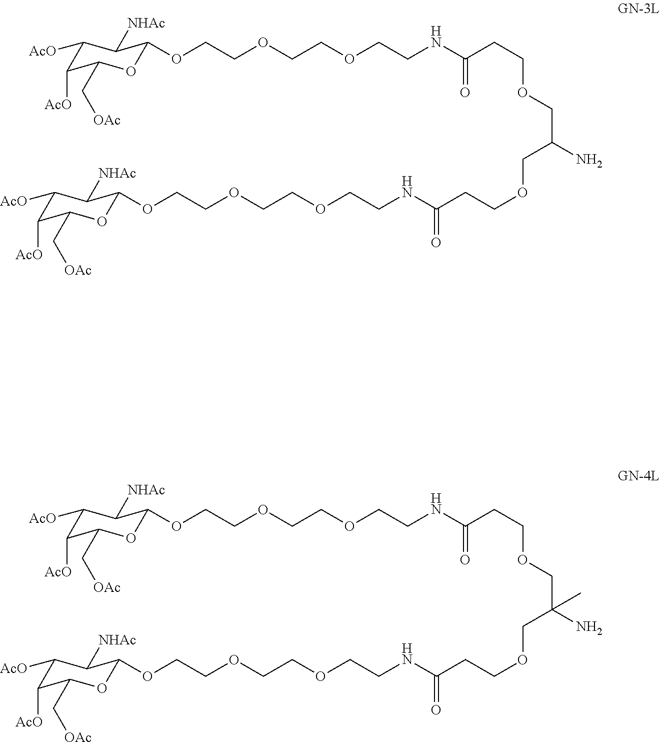 [SMILES: CC(=O)NC1C(OCCOCCOCCNC(=O)CCOCC(N)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; CC(=O)NC1C(OCCOCCOCCNC(=O)CCOCC(C)(N)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

GN-3L and GN-4L were synthesized by the same steps for synthesis of GN-3 and GN-4, respectively, except that GN-1 was replaced with GN-2.

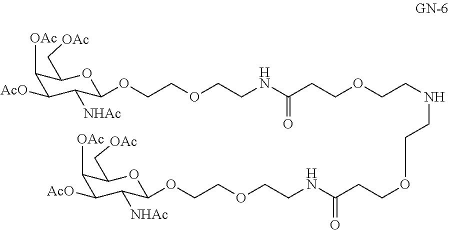 [SMILES: CC(=O)NC1C(OCCOCCNC(=O)CCOCCNCCOCCC(=O)NCCOCCOC2OC(COC(C)=O)C(OC(C)=O)C(OC(C)=O)C2NC(C)=O)OC(COC(C)=O)C(OC(C)=O)C1OC(C)=O]

GN-6 was synthesized by the same steps for synthesis of GN-3, except that CN-3B was replaced with CN-4B.

### 25. Synthesis of TC01A (a Stereoisomeric Carboxylic Acid with Peracetylated Glycosyl Group of Ligand TC01), TC01A-Pfp (an Activated Ester) and TC01A-Cy5 (a Receptor-Targeting Fluorescent Conjugate)

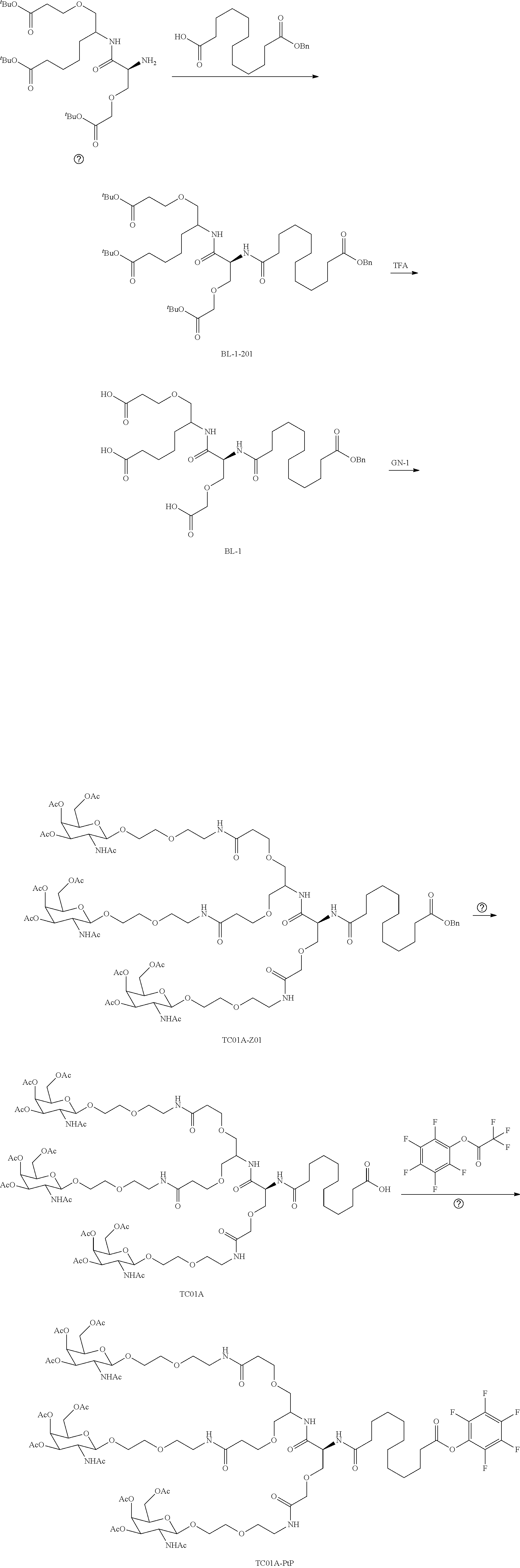

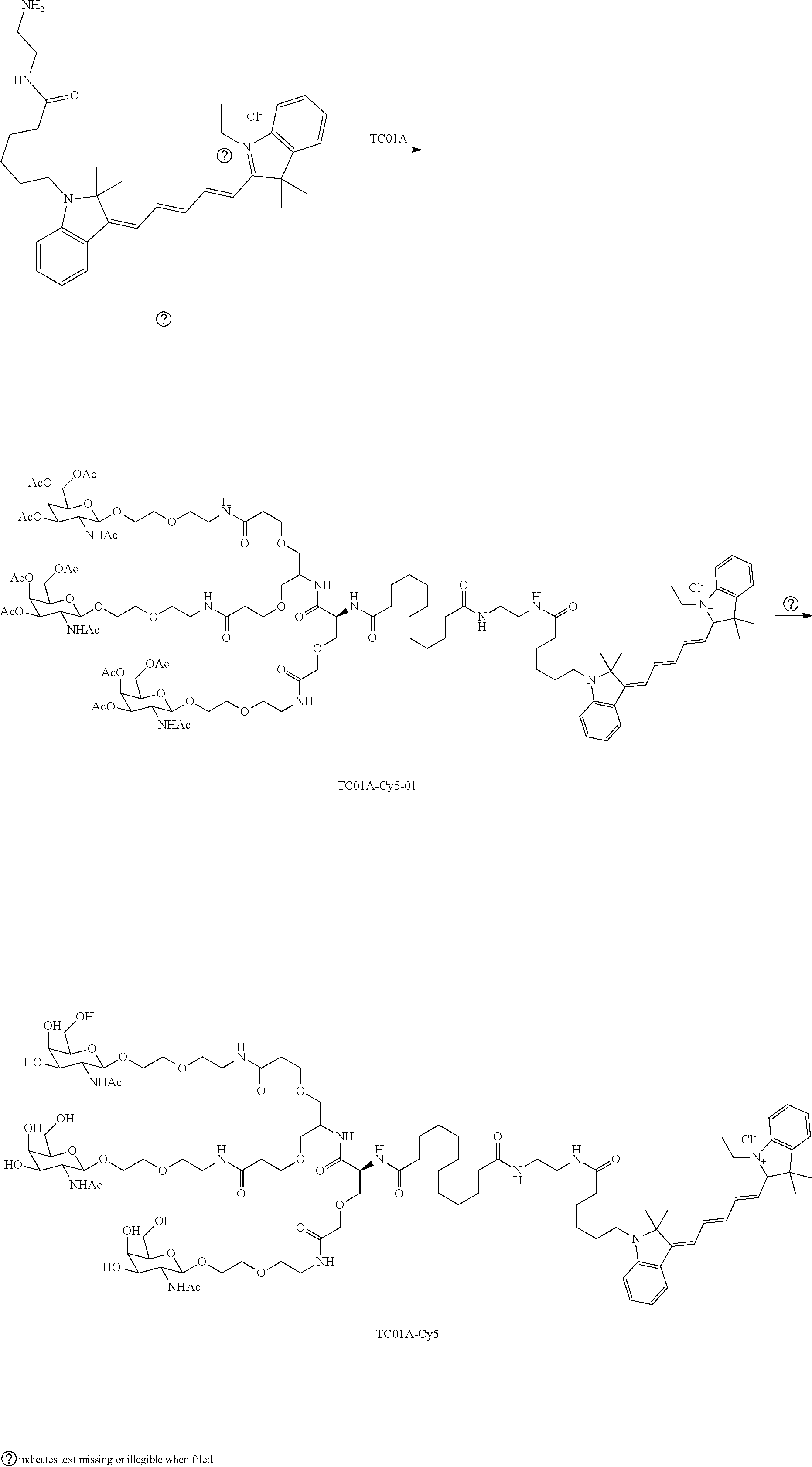

### (1) Synthesis of BL-1-Z01 (Amidation Condensation Reaction)

Under N₂ atmosphere, CN-5A (1.02 g, 1.86 mmol) was added to a flask and dissolved with 30 mL of dichloromethane, then successively added with monobenzyl dodecanediate (0.71 g, 2.2 mmol), triethylamine (0.56 g, 5.5 mmol), EDCI (0.71 g, 3.7 mmol) and DMAP (0.11 g, 0.9 mmol). The reaction was carried out under stirring at room temperature for 5 h when TLC showed that the raw materials were completely converted (petroleum ether/ethyl acetate, 1/1, R_{f} of the product: 0.5). The reaction solution was diluted with 50 mL of dichloromethane and washed with 26% aqueous sodium chloride solution (50 mL×3). The obtained organic phase was dried over anhydrous sodium sulfate, filtered and concentrated under reduced pressure. The residue was purified by silica gel column chromatography (200-300 meshes, petroleum ether/ethyl acetate, 10/1 to 1/1) to obtain 601 mg of a colorless oily product with a yield of 38%.

MS (ESI): m/z [M+H]⁺, theoretical 851.5, found 851.5.

### (2) Synthesis of BL-1 (De-Tert-Butylation Reaction)

BL-1-Z01 (596 mg, 0.7 mmol) was added to a flask, and added with 3 mL of trifluoroacetic acid. The reaction was carried out under stirring at room temperature for 12 h when TLC showed that the conversion of the raw materials was complete (dichloromethane/methanol, 10/1, R_{f} of the product: 0.1). The reaction solution was diluted with 25 mL of acetonitrile, subjected to distillation under reduced pressure to remove the solvent and dried under vacuum to obtain 525 mg of a yellow oily product with a yield of 114% (the product contained some residual TFA and could be directly used in subsequent reaction without purification).

MS (ESI): m/z [M+H]⁺, theoretical 683.3, found 683.7.

### (3) Synthesis of TC01A-Z01 (Amidation Condensation Reaction)

Under N₂ atmosphere, dichloromethane (8 mL), BL-1 (100 mg, 0.146 mmol), GN-1 (210 mg, 0.48 mmol), PyBOP (291 mg, 0.56 mmol) and triethylamine (90 mg, 0.89 mmol) were successively added to a flask, and the reaction was carried out under stirring at room temperature for 6 h when TLC showed that the conversion of the raw materials was complete (dichloromethane/methanol, 10/1, R_{f} of the product: 0.4). The reaction solution was added with pure water (15 mL) and extracted with dichloromethane (7 mL×3). The obtained organic phases were combined, dried over anhydrous sodium sulfate, filtered, and subjected to distillation under reduced pressure to remove the solvent. The residue was purified by silica gel column chromatography (200-300 meshes, dichloromethane/methanol, 100/1 to 20/1) to obtain 202 mg of a light yellow solid with a yield of 68%.

MS (ESI): m/z [1/2M+H]⁺, theoretical 966.4, found 966.4.

¹H NMR (400 MHz, DMSO-d₆) δ 8.10 (d, J=8.2 Hz, 1H), 7.97-7.87 (m, 3H), 7.84 (d, J=9.2 Hz, 3H), 7.74 (t, J=6.0 Hz, 1H), 7.46-7.20 (m, 5H), 5.21 (d, J=3.3 Hz, 3H), 5.07 (s, 2H), 4.97 (dd, J=11.2, 3.4 Hz, 3H), 4.59-4.48 (m, 4H), 4.06-4.00 (m, 10H), 3.96-3.83 (m, 6H), 3.82-3.74 (m, 3H), 3.63-3.53 (m, 10H), 3.52-3.45 (m, 6H), 3.44-3.41 (m, 2H), 3.36-3.28 (m), 4H), 3.28-3.12 (m, 6H), 2.36-2.23 (m, 6H), 2.17-2.12 (m, 2H), 2.10 (s, 9H), 1.99 (s, 10H), 1.89 (s, 9H), 1.77 (s, 9H), 1.50 (d, J=17.2 Hz, 4H), 1.26-1.20 (m, 14H).

### (4) Synthesis of TC01A (Debenzylation Reaction)

TC01A-Z01 (101 mg, 0.05 mmol) was added to a flask, dissolved with 3 mL of tetrahydrofuran, and added with Pd/C (10 mg, 10 wt %). The reaction system was replaced with hydrogen for three times and the reaction was carried out under stirring and under hydrogen atmosphere (about 10-20 kPa higher than the standard atmospheric pressure) for 12 h when LCMS showed that the reaction was complete. The reaction mixture was filtered to remove the solid and the filtrate was concentrated to obtain 78 mg of a white solid product with a yield of 81%.

MS (ESI): m/z [1/2M+H]⁺, theoretical 921.4, found 921.4.

### (5) Synthesis of TC01A-Pfp

Under N₂ atmosphere and at room temperature, TC01A (20 mg, 0.0109 mmol) and DIEA (20 μL) were successively added to a flask containing dry dichloromethane (2 mL), and under constant stirring, pentafluorophenyl trifluoroacetate (12 mg, 0.0436 mmol) was added, and then the reaction was carried out for 0.5 h when LCMS showed that the conversion of the raw materials was complete. The reaction solution was added with dichloromethane (8 mL) and washed with 8% aqueous sodium bicarbonate solution (6 mL×7). The obtained organic phase was dried over anhydrous sodium sulfate, filtered and concentrated under reduced pressure to obtain a white solid crude product. The crude product was purified by recrystallization (acetonitrile/methyl tert-butyl ether, 1/2) to obtain 14 mg of a white solid product with a yield of 64%.

MS (ESI): m/z [1/2M+H]⁺, theoretical 1004.4, found 1004.2.

### (6) Synthesis of Cy5-EtN

Cy5-EtN was synthesized by the method reported in Org. Biomol. Chem., 2012, 10, 710-715.

### (7) Synthesis of TC01A-Cy5-01

Under N₂ atmosphere, TC01A (78 mg, 0.04 mmol) was added to a flask, dissolved with 2 mL of dichloromethane, and successively added with Cy5-EtN (31 mg, 0.06 mmol), PyBOP (28 mg, 0.05 mmol) and triethylamine (12 mg, 0.12 mmol). The reaction was then carried out under stirring at room temperature for 3 h when LCMS showed that the reaction was complete. The reaction solution was subjected to distillation under reduced pressure to remove the solvent. The crude product was purified by silica gel column chromatography (200-300 meshes, dichloromethane/methanol, 100/1 to 10/1) to obtain 65 mg of a blue solid with a yield of 68%.

MS (ESI): m/z [1/3 (M+2H)]⁺, theoretical 788.4, found 788.1; m/z [1/2 (M−Cl+H)]⁺, theoretical 1182.1, found 1181.9.

### (8) Synthesis of TC01A-Cy5

TC01A-Cy5-01 (65 mg, 0.03 mmol) and methanol (1.8 mL) were added to a flask and stirred until the reaction mixture became clear, followed by addition of 25% ammonia water (0.2 mL). The reaction was increased to 40° C., and then carried out under stirring for 2.5 h when LCMS showed that the reaction was complete. The reaction solution was subjected to distillation under reduced pressure to remove the solvent. The residue was purified by prep-HPLC (C₁₈/5 μm, column temperature: 40° C.; water/acetonitrile, 95/5 to 5/95, containing 0.1% formic acid) to obtain 8.8 mg of a blue solid product with a yield of 16.1% and a purity of 99.89% (630 nm).

MS (ESI): m/z [1/2 (M−Cl+H)]⁺, theoretical 993.0, found 992.8.

### 26. Synthesis of TC07A (a Stereoisomeric Carboxylic Acid with Peracetylated Glycosyl Group of Ligand TC07), TC07A-Pfp (an Activated Ester) and TC07A-Cy5 (a Receptor-Targeting Fluorescent Conjugate)

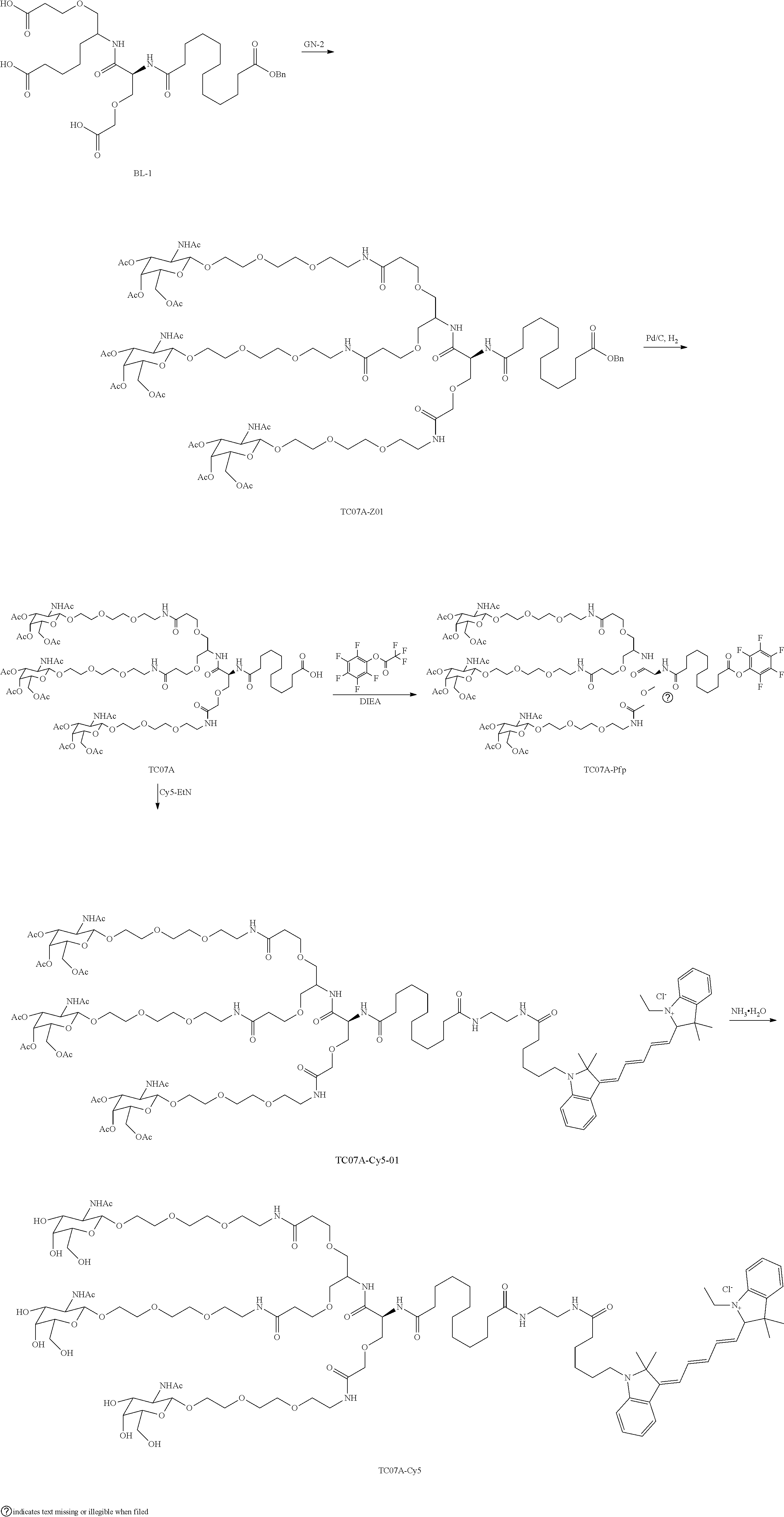

### (1) Synthesis of TC07A-Z01 (Amidation Condensation Reaction)

Under N₂ atmosphere, dichloromethane (8 mL), BL-1 (303 mg, 0.44 mmol), GN-2 (694 mg, 1.45 mmol), PyBOP (460 mg, 1.76 mmol) and triethylamine (90 mg, 2.64 mmol) were successively added to a flask and the reaction was carried out under stirring at room temperature for 6 h when TLC showed that the conversion of the raw materials was complete (dichloromethane/methanol, 10/1, R_{f} of the product: 0.4). The reaction solution was added with pure water (12 mL) and extracted with dichloromethane (8 mL×3). The obtained organic phases were combined, dried over anhydrous sodium sulfate, filtered, and subjected to distillation under reduced pressure to remove the solvent. The residue was purified by silica gel column chromatography (200-300 meshes, dichloromethane/methanol, 100/1 to 20/1) to obtain 517 mg of a light yellow solid product with a yield of 57%.

MS (ESI): m/z [1/2M+H]⁺, theoretical 1033.0, found 1032.9.

### (2) Synthesis of TC07A (Debenzylation Reaction)

TC07A-Z01 (200 mg, 0.10 mmol) was added to a flask and dissolved with 6 mL of tetrahydrofuran, followed by addition of Pd/C (20 mg, 10 wt %). The reaction system was replaced with hydrogen for three times and the reaction was carried out under stirring and under hydrogen atmosphere (about 10-20 kPa higher than the standard atmospheric pressure) for 12 h when LCMS showed that the reaction was complete. The reaction mixture was filtered to remove the solid and the filtrate was concentrated to obtain 180 mg of a white solid product with a yield of 94%.

MS (ESI): m/z [1/2M+H]⁺, theoretical 987.5, found 987.7.

¹H NMR (400 MHz, DMSO-d₆) δ 8.12 (d, J=8.1 Hz, 1H), 7.98-7.88 (m, 3H), 7.85 (d, J=9.2 Hz, 3H), 7.75 (t, J=6.0 Hz, 1H), 5.22 (d, J=3.3 Hz, 3H), 4.98 (dd, J=11.2, 3.4 Hz, 3H), 4.61-4.50 (m, 4H), 4.08-4.03 (m, 10H), 3.98-3.82 (m, 6H), 3.82-3.65 (m, 9H), 3.64-3.46 (m, 22H), 3.43-3.40 (m, 2H), 3.37-3.29 (m, 4H), 3.28-3.12 (m, 6H), 2.38-2.28 (m, 6H), 2.19-2.15 (m, 2H), 2.11 (s, 9H), 1.98 (s, 10H), 1.90 (s, 9H), 1.76 (s, 9H); 1.55 (d, J=17.2 Hz, 4H), 1.27-1.22 (m, 14H).

### (3) Synthesis of TC07A-Pfp

Under N₂ atmosphere, TC07A (40 mg, 0.0203 mmol) and DIEA (30 μL) were successively added to a flask containing dry dichloromethane (3 mL), and under constant stirring, pentafluorophenyl trifluoroacetate (17 mg, 0.061 mmol) was added, and the reaction was then carried out for 0.5 h when LCMS showed that the conversion of the raw materials was complete. The reaction solution was added with dichloromethane (10 mL) and washed with 8% aqueous sodium bicarbonate solution (6 mL×7). The obtained organic phase was dried over anhydrous sodium sulfate, filtered and concentrated under reduced pressure to obtain a white solid crude product. The crude product was purified by recrystallization (acetonitrile/methyl tert-butyl ether, 1/2) to obtain 22 mg of a white solid product with a yield of 50%.

MS (ESI): m/z [1/2M+Na]⁺, theoretical 1092.4, found 1092.5.

### (4) Synthesis of TC07A-Cy5-Z01 (Amidation Condensation Reaction)

Under N₂ atmosphere, TC07A (130 mg, 0.06 mmol) was added to a flask and dissolved with 2 mL of dichloromethane, and then successively added with Cy5-EtN (35 mg, 0.06 mmol), PyBOP (44 mg, 0.08 mmol) and triethylamine (20 mg, 0.2 mmol). The reaction was carried out under stirring at room temperature for 4 h when LCMS showed that the reaction was complete. The reaction solution was subjected to distillation under reduced pressure to remove the solvent, and the crude product was purified by silica gel column chromatography (200-300 meshes, dichloromethane/methanol, 100/1 to 10/1) to obtain 46 mg of a solid with a yield of 31%.

MS (ESI): m/z [1/3 (M−Cl+2H)]⁺, theoretical 832.4, found 832.1.

### (5) Synthesis of TC07A-Cy5

TC07A (45 mg, 0.018 mmol) and methanol (1.8 mL) were added to a flask and stirred until the reaction mixture became clear, followed by addition of 25% ammonia (0.2 mL). The reaction was increased to 40° C., and then carried out under stirring for 3 h when LCMS showed that the reaction was complete. The reaction solution was subjected to distillation under reduced pressure to remove the solvent, and the residue was purified by prep-HPLC (C₁₈/5 μm, column temperature: 40° C.; water/acetonitrile, 95/5 to 5/95, containing 0.1% formic acid), to obtain 6.3 mg of a blue solid product with a yield of 16.6% and a purity of 99.5% (630 nm).

MS (ESI): m/z [1/2(M-Cl+H)]⁺, theoretical 1059.1, found 1059.0.

### 27. Synthesis of TC02A (a Stereoisomeric Carboxylic Acid with Peracetylated Glycosyl Group of Ligand TC02)

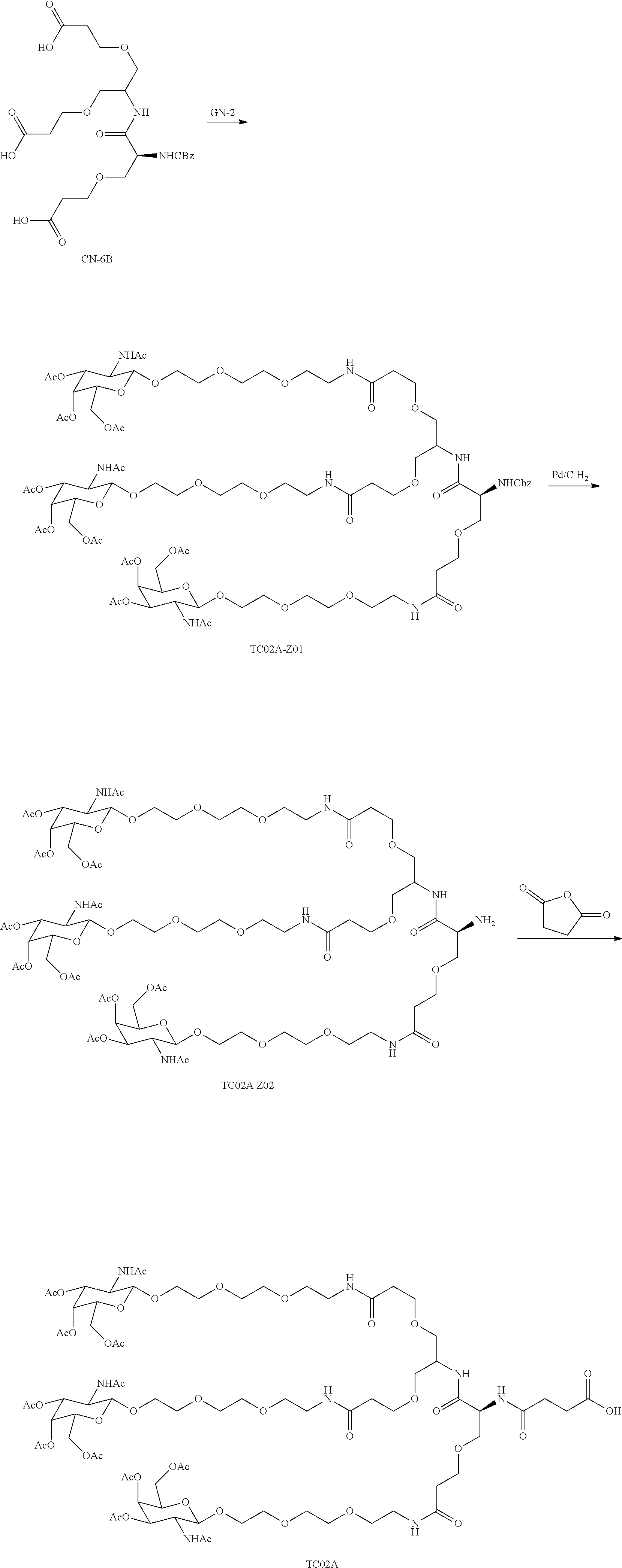

### (1) Synthesis of TC02A-Z01 (Amidation Condensation Reaction)

Under N₂ atmosphere and at room temperature, CN-6B (340 mg, 0.643 mmol), GN-2 (954 mg, 1.99 mmol), PyBOP (1.14 g, 2.19 mmol) and triethylamine (390 mg, 3.86 mmol) were successively added to a flask containing dichloromethane (14 mL) and then the reaction was carried out stirring at room temperature for 6 h when LCMS showed that the reaction was complete. The reaction solution was added with pure water (15 mL) and extracted with dichloromethane (10 mL×3). The obtained organic phases were combined, dried over anhydrous sodium sulfate, filtered, and subjected to distillation under reduced pressure to remove the solvent. The residue was purified by silica gel column chromatography (200-300 meshes, dichloromethane/methanol, 20/1) to obtain 675 mg of a light yellow solid product with a yield of 55%.

MS (ESI): m/z [1/2M+H]⁺, theoretical 955.4, found 955.5.

### (2) Synthesis of TC02A-Z02 (De-Benzyloxycarbonylation Reaction)

TC02A-Z01 (155 mg, 0.0812 mmol) was added to a flask, dissolved with 5 mL of tetrahydrofuran, and added with Pd/C (20 mg, 10 wt %). The reaction system was replaced with hydrogen for three times and the reaction was carried out under stirring and under hydrogen atmosphere (about 10-20 kPa higher than the standard atmospheric pressure) for 2 h when LCMS showed that the reaction was complete. The reaction mixture was filtered to remove the solid and the filtrate was concentrated to obtain 130 mg of a white solid product with a yield of 90%.

MS (ESI): m/z [1/2M+H]⁺, theoretical 888.4, found 888.5.

### (3) Synthesis of TC02A (Amidation Reaction)

Under N₂ atmosphere, dichloromethane (3 mL), TC02A-Z02 (65 mg, 0.0366 mmol), succinic anhydride (11 mg, 0.11 mmol) and triethylamine (15 mg, 0.15 mmol) were successively added to a flask and the reaction was carried out under stirring at room temperature for 5 h when TLC showed that the conversion of the raw materials was complete. The reaction solution was subjected to distillation under reduced pressure to remove the solvent, and the obtained crude product was purified by silica gel column chromatography (200-300 meshes, dichloromethane/methanol, 5/1) obtain 51.5 mg of a white solid product with a yield of 75%.

MS (ESI): m/z [1/2M+Na]⁺, theoretical 960.4, found 960.6.

¹H NMR (400 MHz, DMSO-d₆) δ 8.18 (d, J=8.1 Hz, 1H), 7.92-7.87 (m, 2H), 7.85 (d, J=9.2 Hz, 3H), 7.75-7.70 (m, 2H), 5.24 (d, J=3.3 Hz, 3H), 4.99 (dd, J=11.2, 3.4 Hz, 3H), 4.63-4.52 (m, 4H), 4.09-4.05 (m, 8H), 4.00-3.84 (m, 6H), 3.82-3.65 (m, 11H), 3.64-3.46 (m, 22H), 3.44-3.41 (m, 2H), 3.38-3.30 (m, 4H), 3.27-3.13 (m, 6H), 2.47-2.40 (m, 2H), 2.39-2.29 (m, 8H), 2.13 (s, 9H), 1.99 (s, 9H), 1.92 (s, 9H), 1.78 (s, 9H).

### 28. Synthesis of TC03A (a Stereoisomeric Carboxylic Acid with Peracetylated Glycosyl Group of Ligand TC03)

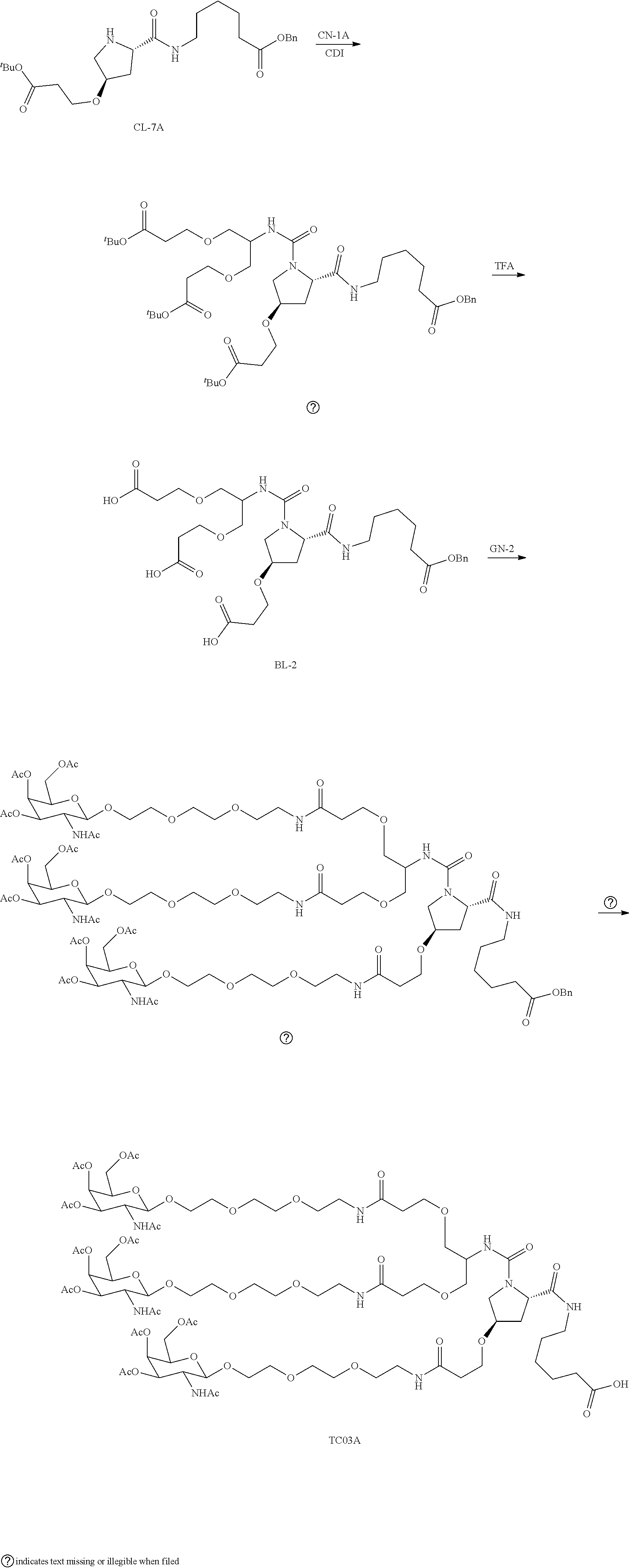

### (1) Synthesis of BL-2-Z01 (Urea Condensation Reaction)

Under N₂ atmosphere, CL-7A (0.78 g, 1.69 mmol) and triethylamine (0.34 g, 3.37 mmol) were added to a flask containing dichloromethane (8 mL), and under stirring, N,N′-carbonyldiimidazole (0.29 g, 1.77 mmol) was slowly added, and the reaction was then carried out for 1 h. The reaction was then added with CN-1A (0.61 g, 1.77 mmol) and carried out under stirring at room temperature for 8 h when LCMS showed that the reaction was complete. The reaction solution was added with pure water (10 mL) and extracted with dichloromethane (8 mL×3). The obtained organic phases were combined, washed with 26% saturated saline solution (15 mL), dried over anhydrous sodium sulfate, filtered and concentrated. The obtained crude product was crystallized with methyl tert-butyl ether to obtain 0.79 g of a light yellow solid product with a yield of 56%.

MS (ESI): m/z [M+H]⁺, theoretical 836.5, found 836.5.

### (2) Synthesis of BL-2

BL-2 was obtained by subjecting BL-2-Z01 as a starting material to the same de-tert-butylation reaction steps for synthesis of BL-1.

MS (ESI): m/z [M−H]⁻, theoretical 666.3, found 666.4.

### (3) Synthesis of TC03A-Z01

TC03A-Z01 was obtained by subjecting BL-2 and GN-2 as starting materials to the same amidation condensation reaction steps for synthesis of TC01A-Z01.

MS (ESI): m/z [M−H]⁻, theoretical 1025.0, found 1024.9.

### (4) Synthesis of TC03A (Debenzylation Reaction)

TC03A was obtained by subjecting TC03A-Z01 as a starting material to the same debenzylation reaction steps for synthesis of TC01A.

MS (ESI): m/z [1/2M+H]⁺, theoretical 979.9, found 979.9.

¹H NMR (400 MHz, DMSO-d₆) δ 7.70-7.58 (m, 4H), 7.49 (m, 3H), 6.98 (d, J=7.5 Hz, 1H), 5.20 (d, J=3.1 Hz, 3H), 4.95 (dd, J=11.0, 3.2 Hz, 3H), 4.53 (d, J=8.4 Hz, 3H), 4.46-4.28 (m, 1H), 4.17-4.10 (m, 1H), 4.07-3.98 (m, 7H), 3.92-3.84 (m, 6H), 3.82-3.72 (m, 9H), 3.70-3.53 (m, 18H), 3.53-3.44 (m, 7H), 3.43-3.37 (m, 9H), 3.31-3.13 (m, 9H), 2.50-2.42 (m, 6H), 2.38-2.25 (m, 3H), 2.17-2.10 (m, 1H), 2.06 (s, 9H), 1.98 (s, 9H), 1.87 (s, 9H), 1.80 (s, 9H), 1.60-1.47 (m, 4H), 1.41-1.31 (m, 2H).

### 29. Synthesis of TC04A (an Alcohol Compound with Peracetylated Glycosyl of Ligand TC04) and TC04A-PA (a Phosphoramidite Compound)

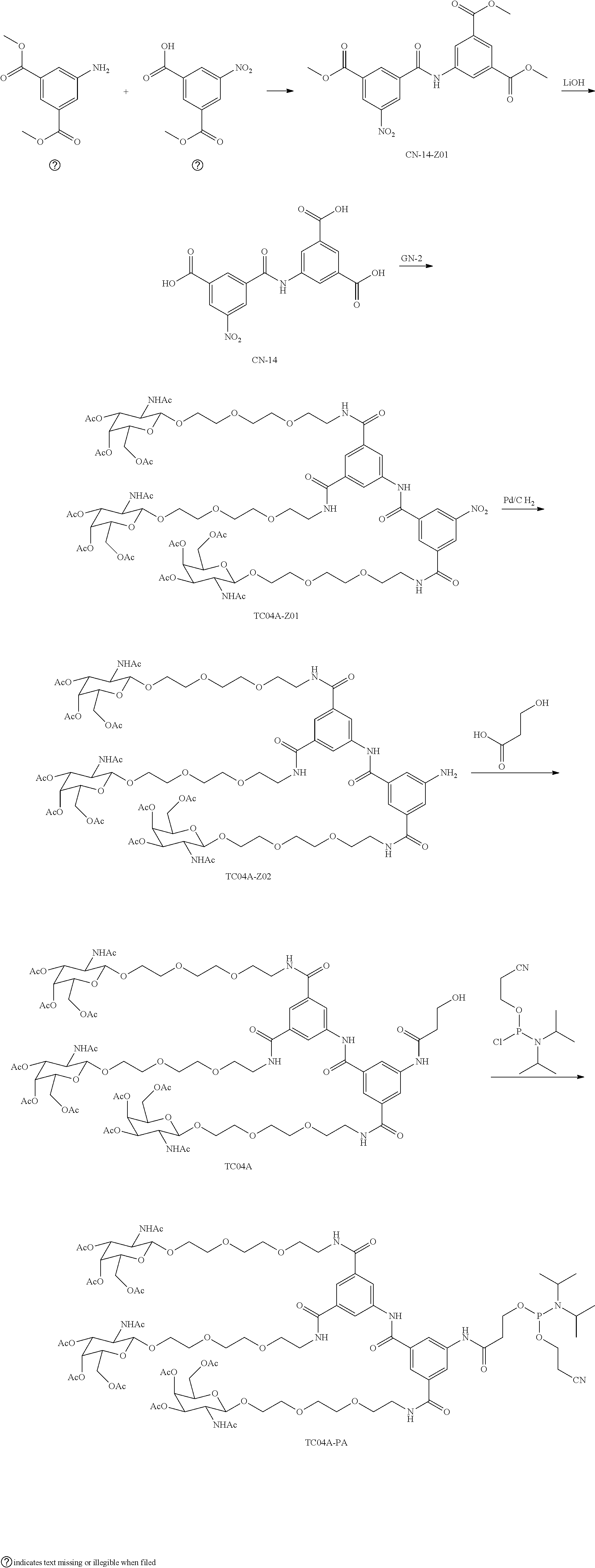

### (1) Synthesis of CN-14-Z01 (Amidation Condensation Reaction)

Under N₂ atmosphere, dichloromethane (100 mL), dimethyl 5-aminoisophthalate (2.09 g, 10.0 mmol), monomethyl 5-nitroisophthalate (3.37 g, 15.0 mmol), EDCI (2.9 g, 10 mmol), HOBt (2.0 g, 15.0 mmol) and DIEA (1.9 g, 15.0 mmol) were successively added to a flask and the reaction was carried out under stirring at room temperature for 3 h when TLC showed disappearance of the raw material dimethyl 5-aminoisophthalate (dichloromethane/anhydrous methanol, 10/1, R_{f} of the product: 0.8). The reaction solution was added with pure water (200 mL) and extracted with dichloromethane (200 mL×3). The obtained organic phases were combined, washed with 26% aqueous sodium chloride solution (300 mL), dried over anhydrous sodium sulfate, filtered, and subjected to distillation under reduced pressure to remove the solvent, thereby obtaining a yellow oily crude product. The crude product was purified by silica gel column chromatography (200-300 meshes, dichloromethane/ethyl acetate, 50/1), and the obtained solid was crystallized with ethyl acetate (30 mL) to obtain 2.77 g of a white solid with a yield of 67%.

MS (ESI): m/z [M+H]⁺, theoretical 417.1, found 417.1.

### (2) Synthesis of CN-14 (Demethylation Reaction)

Tetrahydrofuran (40 mL) and CN-14-Z01 (2.77 g, 6.65 mmol) were successively added to a flask and stirred constantly, followed by slow addition of LiOH monohydrate (1.67 g, 40 mmol) dissolved in pure water (40 mL) at room temperature. The reaction was then carried out under stirring for 1 h when LCMS showed that the reaction was complete. The reaction solution was slowly added with 10% aqueous HCl solution (100 mL) under stirring, resulting in precipitation of a small amount of white floccules, and then extracted with ethyl acetate (200 mL×2). The obtained organic phases were combined, dried over anhydrous sodium sulfate, filtered, and subjected to distillation under reduced pressure to remove the solvent, thereby obtaining 2.44 g of a white solid product with a yield of 98%.

MS (ESI): m/z [M−H]⁺, theoretical 373.0, found 373.0.

### (3) Synthesis of TC04A-Z01 (Amidation Condensation Reaction)

Under N₂ atmosphere, dichloromethane (20 mL), CN-14 (164 mg, 0.44 mmol), GN-2 (956 mg, 2 mmol), EDCI (506 mg, 2.64 mmol), triethylamine (266 mg, 2.64 mmol) and DMAP (53 mg, 0.44 mmol) were successively added to a flask and the reaction was carried out under stirring at room temperature for 16 h when LCMS showed that the reaction was complete. The reaction solution was added with pure water (100 mL) and extracted with dichloromethane (50 mL×3). The obtained organic phases were combined, dried over anhydrous sodium sulfate, filtered, and subjected to distillation under reduced pressure to remove the solvent. The crude product was purified by silica gel column chromatography (200-300 meshes, dichloromethane/methanol, 10/1) to obtain 280 mg of a white solid product with a yield of 36%.

MS (ESI): m/z [1/2M+H]⁺, theoretical 878.3, found 878.6.

### (4) Synthesis of TC04A-Z02

TC04A-Z01 (280 mg, 0.159 mmol), Pd/C (28 mg, 10 wt %), methanol (5 mL) and tetrahydrofuran (5 mL) were successively added to a flask. The reaction system was replaced with hydrogen for three times and the reaction was carried out under stirring and under hydrogen atmosphere (about 10-20 kPa higher than the standard atmospheric pressure) for 2 h when TLC showed that the conversion of the raw materials was complete. The reaction mixture was filtered to remove solid and the filtrate was concentrated to obtain 140 mg of a white solid product with a yield of 51%.

MS (ESI): m/z [1/2M+H]⁺, theoretical 863.3, found 863.5.

### (5) Synthesis of TC04A (Amidation Condensation Reaction)

TC04A-Z02 (120 mg, 0.0695 mmol) and 3-hydroxypropionic acid (11 mg, 0.083 mmol) were successively added to a flask containing methanol (40 mL), followed by addition of DMT-MM monohydrate (41 mg, 0.139 mmol) under constant stirring. The reaction was then carried out under stirring at room temperature for 6 h when LCMS showed that the reaction was complete. The reaction solution was concentrated under reduced pressure, and the obtained crude product was directly purified by silica gel column chromatography (200-300 meshes, dichloromethane/methanol, 15/1) to obtain 101 mg of a white solid product with a yield of 81%.

MS (ESI): m/z [1/2M+H]⁺, theoretical 899.4, found 899.4.

### (6) Synthesis of TC04A-PA

Under N₂ atmosphere, TC04A (90 mg, 0.05 mmol) and triethylamine (10 mg, 0.1 mmol) were successively added to a flask containing dichloromethane (3 mL), followed by addition of 2-cyanoethyl-N,N-diisopropylchlorophosphoramidite (18 mg, 0.075 mmol) in an ice bath of 0-5° C. The reaction was then carried out under stirring for 0.5 h when LCMS showed that the reaction was complete. The reaction solution was added with 8% aqueous sodium bicarbonate solution (10 mL) to quench the reaction, and extracted with dichloromethane (8 mL×3). The obtained organic phases were combined, dried over anhydrous sodium sulfate, filtered and concentrated under reduced pressure. The crude product was crystallized with ethyl acetate and methyl tert-butyl ether successively to obtain 58 mg of a white solid product with a yield of 58%.

MS (ESI): m/z [1/2M+H]⁺, theoretical 999.4, found 999.5.

### 30. Synthesis of TC05A (a Stereoisomeric Carboxylic Acid with Peracetylated Glycosyl Group of Ligand TC05)

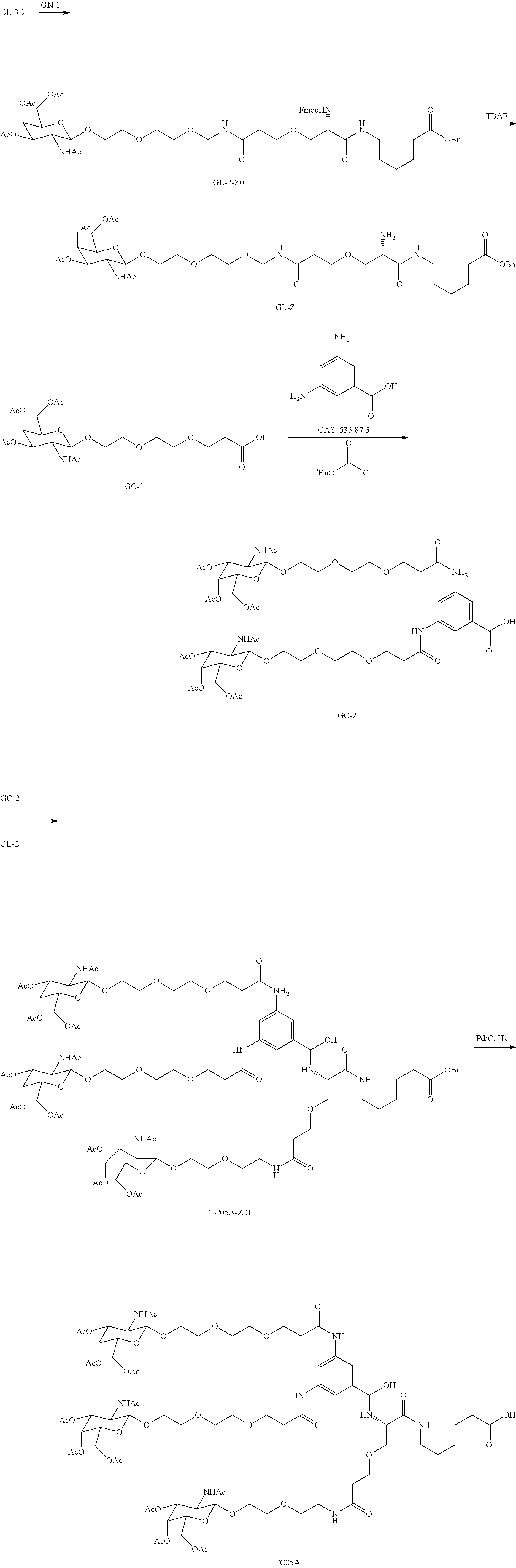

### (1) Synthesis of GL-2-Z01 (Amidation Condensation Reaction)

Under N₂ atmosphere, CL-3B (1.5 g, 2.49 mmol), GN-1 (1.08 g, 2.49 mmol), EDCI (0.62 g, 3.24 mmol), HOBt (0.34 g, 2.49 mmol) and triethylamine (0.5 g, 4.98 mmol) were successively added to a flask containing dichloromethane (16 mL) and the reaction was carried out under stirring at room temperature for 6 h when LCMS showed that the reaction was complete. The reaction solution was added with pure water (20 mL) and extracted with dichloromethane (15 mL×3). The obtained organic phases were combined, dried over anhydrous sodium sulfate, filtered, and subjected to distillation under reduced pressure to remove the solvent. The crude product was purified by silica gel column chromatography (200-300 meshes, dichloromethane/methanol, 30/1) to obtain 2.16 g of a white solid product with a yield of 85%.

MS (ESI): m/z [M+H]⁺, theoretical 1019.4, found 1019.4.

### (2) Synthesis of GL-2 (De-9-Fluorenylmethoxycarbonylation Reaction)

GL-2-Z01 (1.8 g, 1.77 mmol) was added to a flask containing N,N-dimethylformamide (10 mL), followed by addition of 1 M tetrabutylammonium fluoride in tetrahydrofuran (3.5 mL) under stirring, and then the reaction was carried out under stirring at room temperature for 6 h when LCMS showed that the reaction was complete. The reaction solution was added with pure water (40 mL) and extracted with dichloromethane (15 mL×3). The obtained organic phases were combined, washed with 26% aqueous sodium chloride solution (15 mL×3), dried over anhydrous sodium sulfate, filtered, and subjected to distillation under reduced pressure to remove the solvent. The obtained crude product was purified by silica gel column chromatography (200-300 meshes, dichloromethane/methanol, 20/1) to obtain 1.13 g of a white solid product with a yield of 80%.

MS (ESI): m/z [M+H]⁺, theoretical 797.4, found 797.6.

### (3) Synthesis of GC-2 (Amidation Condensation Reaction)

Under N₂ atmosphere, GC-1 (1.6 g, 3.15 mmol) and N-methylmorpholine (0.64 g, 6.3 mmol) were successively added to a flask containing dichloromethane (12 mL) and cooled to 0-5° C. in an ice bath, followed slow dropwise addition of isobutyl chloroformate (0.45 g, 3.3 mmol). The reaction was then carried out under stirring for 1 h with the temperature maintained unchanged. Then the reaction was added with 3,5-diaminobenzoic acid (0.24 g, 1.57 mmol) and then carried out under stirring at room temperature for 2 h when LCMS showed that the reaction was complete. The reaction solution was added with pure water (20 mL), adjusted to about pH7 with 0.5 N hydrochloric acid, and extracted with dichloromethane (15 mL×3). The obtained organic phases were combined, washed with 26% aqueous sodium chloride solution (20 mL), dried over anhydrous sodium sulfate, filtered and concentrated under reduced pressure. The crude product was crystallized with methyl tert-butyl ether to obtain 1.54 g of a light yellow solid product with a yield of 87%.

MS (ESI): m/z [M+H]⁺, theoretical 1131.4, found 1131.5.

### (4) Synthesis of TC05A-Z01

TC05A-Z01 was obtained by subjecting GC-2 and GL-2 as starting materials to the same amidation condensation reaction steps for synthesis of GL-2-Z01.

MS (ESI): m/z [1/2M+H]⁺, theoretical 944.4, found 955.6.

### (5) Synthesis of TC05A

TC05A was obtained by subjecting TC05A-Z01 as a starting material to the same debenzylation reaction steps for synthesis of TC07A.

MS (ESI): m/z [1/2M+H]⁺, theoretical 910.4, found 910.5.

### 31. Synthesis of TC06A (an Alcohol Compound with Peracetylated Glycosy Group of Ligand TC06)

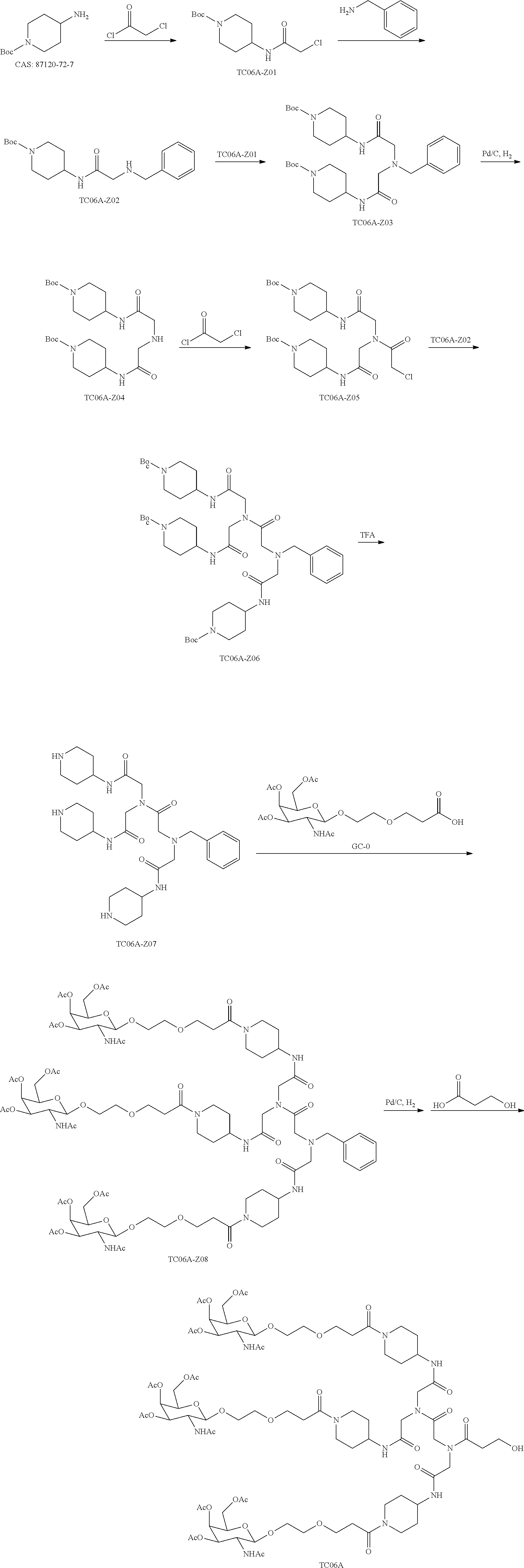

Synthesis of TC06A: (1) TC06A-Z01 was obtained by rapid amidation reaction of 1-Boc-4-amino piperidine as a starting material with chloroacetyl chloride; (2) mono-substituted product TC06A-Z02 and di-substituted product TC06A-Z03 were obtained by subjecting TC06A-Z01 to substitution reaction with benzylamine; (3) TC06A-Z04 was obtained by subjecting TC06A-Z03 to debenzylation reaction at an amine group, and TC06A-Z05 was obtained by further subjecting TC06A-Z04 to chloro-acetylation; (4) TC06A-Z06 was obtained by subjecting TC06A-Z05 to substitution with TC06A-Z02; (5) TC06A-Z07 with three secondary amino groups was obtained by subjecting TC06A-Z06 to de-tert-butylation reaction; (6) TC06A-Z08 was synthesized by subjecting TC06A-Z07 to amidation condensation with GC-0; (7) TC06A was obtained by subjecting TC06A-Z08 to debenzylation and then to amidation condensation with 3-hydroxypropionic acid.

### 32. Synthesis of TC08A (a Stereoisomeric Carboxylic Acid with Peracetylated Glycosyl Group of Ligand TC08)

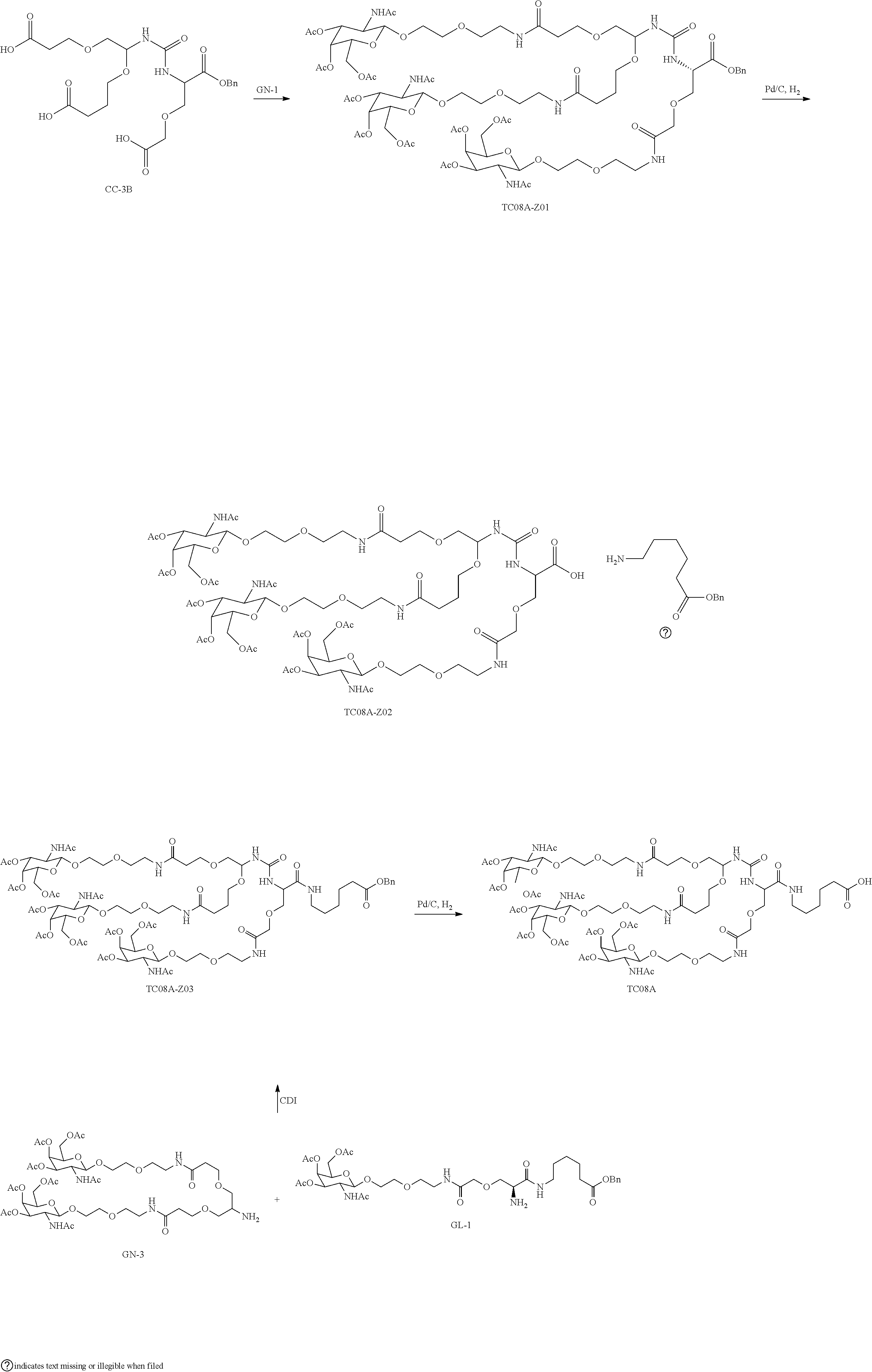

### (1) Synthesis of TC08A-Z01

TC08A-Z01 was obtained by subjecting CC-3B and GN-1 as starting materials to the same amidation condensation reaction steps for synthesis of TC07A-Z01.

MS (ESI): m/z [1/2M+H]⁺, theoretical 882.4, found 882.5.

### (2) Synthesis of TC08A-Z02

TC08A-Z02 was obtained by subjecting TC08A-Z01 as a starting material to the same debenzylation reaction steps for synthesis of TC07A.

MS (ESI): m/z [1/2M+H]⁺, theoretical 837.3, found 837.3.

### (3) Synthesis of TC08A-Z03

TC08A-Z03 was obtained by subjecting TC08A-Z02 and benzyl 6-aminocaproate as starting materials to similar amidation condensation reaction steps for synthesis of BL-1-Z01.

MS (ESI): m/z [1/2M+H]⁺, theoretical 938.9, found 939.1.

TC08A-Z03 could also be obtained by subjecting GL-1 and GN-3 as starting materials to the same urea condensation reaction steps for synthesis of BL-2-Z01.

MS (ESI): m/z [1/2M+H]⁺, theoretical 938.9, found 939.0.

### (4) Synthesis of TC08A

TC08A was obtained by subjecting TC08A-Z03 as a starting material to the same debenzylation reaction steps for synthesis of TC07A.

MS (ESI): m/z [1/2M+Na]⁺, theoretical 915.9, found 916.0.

### 33. Synthesis of TC09A (a Stereoisomeric Carboxylic Acid with Peracetyleated Glycosyl Group of Ligand TC09) and TC09A-Cy5 (a Receptor-Targeting Fluorescent Conjugate)

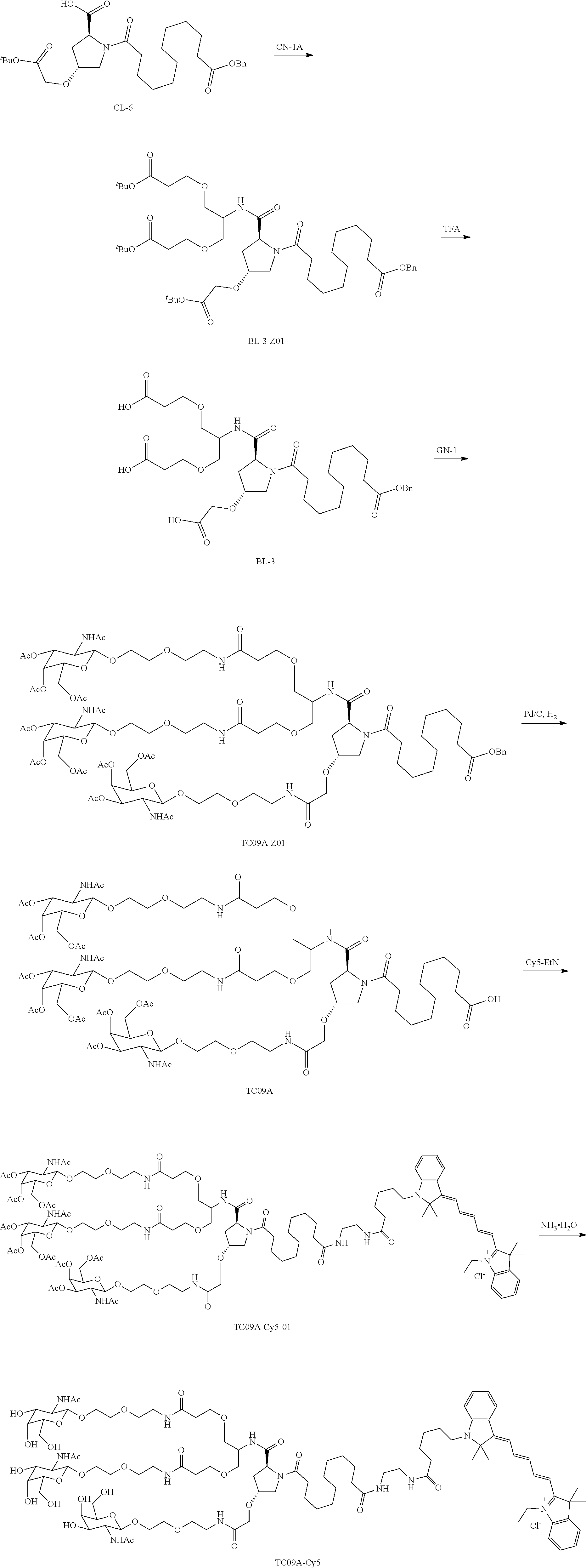

### (1) Synthesis of BL-3-Z01 (Amidation Condensation)

Under N₂ atmosphere and at room temperature, CL-6 (1.1 g, 2 mmol), CN-1A (0.7 g, 2 mmol), EDCI (0.5 g, 2.4 mmol) and HOBt (0.3 g, 2.4 mmol) were successively added to a flask containing dichloromethane (22 mL), and under constant stirring, DIEA (0.52 g, 4 mmol) was slowly added, and the reaction was then carried out for 12 h when LCMS showed that the reaction was complete. The reaction solution was added with pure water (30 mL) for quenching and dilution, and extracted with dichloromethane (30 mL×3). The obtained organic phases were combined, washed with 26% aqueous sodium chloride solution (50 mL), dried over anhydrous sodium sulfate, filtered and concentrated under reduced pressure to obtain 1.6 g of a yellow liquid crude product. The crude product was purified by silica gel column chromatography (200-300 meshes, petroleum ether/ethyl acetate, 7/3) to obtain 0.5 g of a yellow oily liquid product with a yield of 31.8%.

MS (ESI): m/z [M+Na]⁺, theoretical 877.5, found 877.4.

### (2) Synthesis of BL-3 (De-Tert-Butylation Reaction)

BL-3-Z01 (0.5 g, 0.6 mmol) was dissolved in dichloromethane (11 mL) in a flask and stirred at room temperature until the reaction mixture became clear. The reaction was then slowly added with TFA (2 g, 18.5 mmol) and then carried out under stirring for 12 h when LCMS showed that the reaction was complete. The obtained mixture was subjected to distillation under reduced pressure to remove the solvent. The obtained crude product was purified by silica gel column chromatography (200-300 meshes, petroleum ether/ethyl acetate, 1/1 to pure ethyl acetate) to obtain 0.4 g of a yellow oily liquid product with a yield of 90%.

MS (ESI): m/z [M+H]⁻, theoretical 709.4, found 709.3.

### (3) Synthesis of TC09A-Z01 (Amidation Condensation Reaction)

Under N₂ atmosphere, BL-3 (0.4 g, 0.6 mmol), GN-1 (0.8 g, 1.8 mmol), EDCI (0.4 g, 2.2 mmol) and HOBt (0.3 g, 2.2 mmol) were successively added to a flask containing dichloromethane (11 mL) and stirred at room temperature until the reaction mixture became clear, followed by slow addition of DIEA (0.5 g, 3.7 mmol). The reaction was then carried out for 12 h when LCMS showed that the reaction was completed. The reaction solution was added with pure water (20 mL) to quench the reaction and extracted with dichloromethane (20 mL×3). The obtained organic phases were combined, washed with 26% aqueous sodium chloride solution (20 mL), dried over anhydrous sodium sulfate, filtered and concentrated under reduced pressure to obtain 1.2 g of a white solid crude product. The crude product was purified by silica gel column chromatography (200-300 meshes, dichloromethane/methanol, 9/1) to obtain 0.9 g of a white solid product with a yield of 72.5%.

MS (ESI): m/z [1/2M+H]⁺, theoretical 979.5, found 979.8.

¹H NMR (400 MHz, DMSO-d₆) δ 8.00-7.90 (m, 2H), 7.85 (d, J=9.2 Hz, 3H), 7.79-7.69 (m, 2H), 7.41-7.30 (m, 5H), 5.21 (d, J=3.3 Hz, 3H), 5.07 (s, 2H), 4.97 (dd, J=11.4, 3.3 Hz, 3H), 4.54 (d, J=8.5 Hz, 3H), 4.47-4.27 (m, 1H), 4.19-4.09 (m, 1H), 4.07-3.99 (m, 9H), 3.92-3.83 (m, 6H), 3.81-3.74 (m, 3H), 3.65-3.53 (m, 10H), 3.53-3.44 (m, 7H), 3.43-3.37 (m, 9H), 3.31-3.13 (m, 9H), 2.36-2.29 (m, 6H), 2.27-2.16 (m, 2H), 2.10 (s, 9H), 1.99 (s, 9H), 1.89 (s, 9H), 1.77 (s, 9H), 1.57-1.37 (m, 4H), 1.23 (s, 12H).

### (4) Synthesis of TC09A (Debenzylation Reaction)

TC09A-Z01 (98 mg, 0.05 mmol) was dissolved in tetrahydrofuran (3 mL) and added with Pd/C (10 mg, 10 wt %). The reaction system was replaced with hydrogen for three times and the reaction was carried out under stirring and under hydrogen atmosphere (about 10-20 kPa higher than the standard atmospheric pressure) for 2 h when LCMS showed that the reaction was complete. The reaction mixture was filtered to remove the solid and then concentrated to obtain 90 mg of a white solid product with a yield of 97.5%.

MS (ESI): m/z [1/2M+H]⁺, theoretical 934.4, found 934.5.

### (5) Synthesis of TC09A-Cy5-01 (Amidation Condensation Reaction)

Under N₂ atmosphere, TC09A (90 mg, 0.048 mmol), Cy5-EtN (29 mg, 0.05 mmol), EDCI (14 mg, 0.073 mmol) and HOBt (10 mg, 0.074 mmol) were successively added to a flask containing dichloromethane (2 mL) and stirred at room temperature until the reaction mixture became clear, followed by addition of DIEA (13 mg, 0.1 mmol). The reaction was then carried out for 3 h when LCMS showed that the reaction was completed. The obtained reaction solution was subjected to distillation under reduced pressure to remove the solvent. The crude product was purified by silica gel column chromatography (200-300 meshes, dichloromethane/methanol, 97/3 to 91/9) to obtain 80 mg of a blue solid product with a yield of 67%.

MS (ESI): m/z [1/3 (M−Cl+2Na)]⁺, theoretical 811.7, found 811.6.

### (6) Synthesis of TC09A-Cy5

Under N₂ atmosphere, TC09A-Cy5-01 (80 mg, 0.03 mmol) and methanol (2.5 mL) were added to a flask and stirred at room temperature until the reaction mixture became clear, followed by addition of 25% ammonia (0.5 mL). The reaction was then carried out under stirring at room temperature for 4 h when LCMS showed that the reaction was completed. The obtained reaction solution was subjected to distillation under reduced pressure to remove the solvent and purified by prep-HPLC (C₁₈/5 μm, column temperature: 40° C.; water/acetonitrile, 95/5 to 5/95, containing 0.1% formic acid) to obtain 12.7 mg of a blue solid product with a yield of 16.6% and a purity of 99.9% (630 nm).

MS (ESI): m/z [1/2(M−Cl+Na)]⁺, theoretical 1017.1, found 1016.7.

### 34. Synthesis of TC23A (a Stereoisomeric Carboxylic Acid with Peraceylated Glycosyl Group of Ligand TC23)

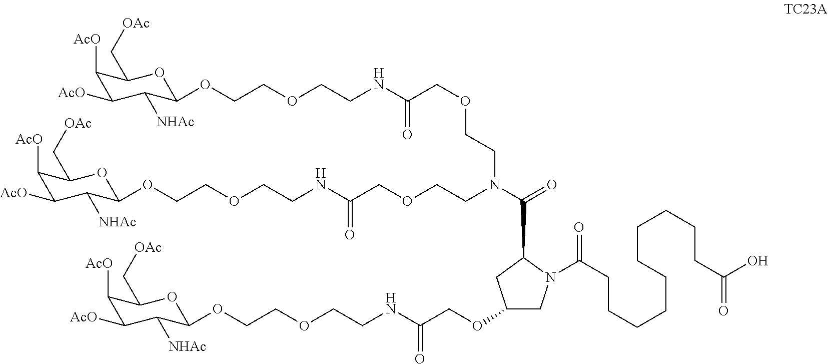 [SMILES: CC(=O)NC1C(OCCOCCNC(=O)COCCN(CCOCC(=O)NCCOCCOC2OC(COC(C)=O)C(OC(C)=O)C(OC(C)=O)C2NC(C)=O)C(=O)[C@@H]2C[C@@H](OCC(=O)NCCOCCOC3OC(COC(C)=O)C(OC(C)=O)C(OC(C)=O)C3NC(C)=O)CN2C(=O)CCCCCCCCCCC(=O)O)OC(COC(C)=O)C(OC(C)=O)C1OC(C)=O]

TC23A was synthesized by the same synthesis steps for synthesis of TC09A, except that CN-1A was replaced with CN-3A.

### 35. Synthesis of TC12A (a Stereoisomeric Carboxylic Acid with Peraceylated Glycosyl Group of Ligand TC12) and TC12A-Cy5 (a Receptor-Targeting Fluorescent Conjugate)

TC12A and the fluorescent conjugate thereof were synthesized by the same steps for synthesis of TC09A and the fluorescent conjugate thereof, except that CN-1 was replaced with GN-2.

### (1) Synthesis of TC12A-Z01

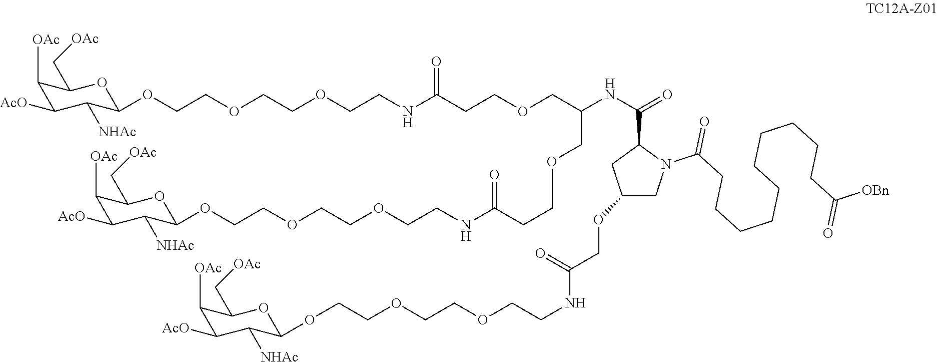 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)NC(=O)[C@@H]2C[C@@H](OCC(=O)NCCOCCOCCOC3OC(COC(C)=O)C(OC(C)=O)C(OC(C)=O)C3NC(C)=O)CN2C(=O)CCCCCCCCCCC(=O)OCc2ccccc2)OC(COC(C)=O)C(OC(C)=O)C1OC(C)=O]

BL-3 (200 mg, 0.28 mmol) and GN-2 (607 mg, 1.27 mmol) were used as starting materials for synthesis to obtain 270 mg of a white solid product with a yield of 46%.

MS (ESI): m/z [1/2M+H]⁺, theoretical 1045.5, found 1045.2.

### (2) Synthesis of TC12A

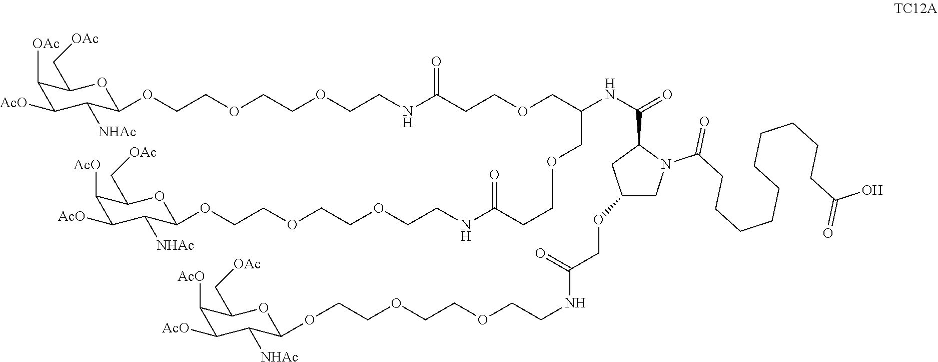 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)NC(=O)[C@@H]2C[C@@H](OCC(=O)NCCOCCOCCOC3OC(COC(C)=O)C(OC(C)=O)C(OC(C)=O)C3NC(C)=O)CN2C(=O)CCCCCCCCCCC(=O)O)OC(COC(C)=O)C(OC(C)=O)C1OC(C)=O]

TC12A-Z01 (140 mg, 0.067 mmol) was used as a starting material for synthesis to obtain 130 mg of a white solid product with a yield of 97%.

MS (ESI): m/z [1/2M+H]⁺, theoretical 1000.5, found 1000.6.

¹H NMR (400 MHz, DMSO-d₆) δ 7.98-7.88 (m, 2H), 7.83 (d, J=9.2 Hz, 3H), 7.77-7.67 (m, 2H), 5.19 (d, J=3.3 Hz, 3H), 4.95 (dd, J=11.4, 3.3 Hz, 3H), 4.52 (d, J=8.5 Hz, 3H), 4.45-4.25 (m, 1H), 4.17-4.07 (m, 1H), 4.05-3.97 (m, 9H), 3.90-3.81 (m, 6H), 3.79-3.70 (m, 8H), 3.65-3.51 (m, 16H), 3.51-3.42 (m, 8H), 3.41-3.35 (m, 9H), 3.31-3.28 (m, 2H), 3.25-3.11 (m, 7H), 2.34-2.27 (m, 6H), 2.25-2.14 (m, 2H), 2.08 (s, 9H), 1.97 (s, 9H), 1.87 (s, 9H), 1.75 (s, 9H), 1.55-1.47 (m, 2H), 1.46-1.37 (m, 2H), 1.21 (s, 12H).

### (3) Synthesis of TC12A-Cy5-01

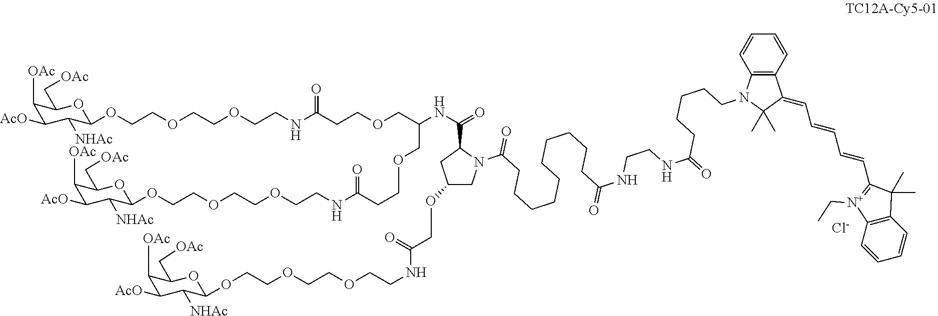 [SMILES: CC[N+]1=C(/C=C/C=C/C=C2/c3ccccc3N(CCCCCC(=O)NCCNC(=O)CCCCCCCCCCC(=O)N3C[C@H](OCC(=O)NCCOCCOCCOC4OC(COC(C)=O)C(OC(C)=O)C(OC(C)=O)C4NC(C)=O)C[C@H]3C(=O)NC(COCCC(=O)NCCOCCOCCOC3OC(COC(C)=O)C(OC(C)=O)C(OC(C)=O)C3NC(C)=O)COCCC(=O)NCCOCCOCCOC3OC(COC(C)=O)C(OC(C)=O)C(OC(C)=O)C3NC(C)=O)C2(C)C)C(C)(C)c2ccccc21 ; [Cl-]]

TC12A (70 mg, 0.035 mmol) was used as a starting material for synthesis to obtain 40 mg of a blue solid product with a yield of 45%.

MS (ESI): m/z [1/3 (M−Cl+2H)]⁺, theoretical 841.1, found 840.9.

### (4) Synthesis of TC12A-Cy5

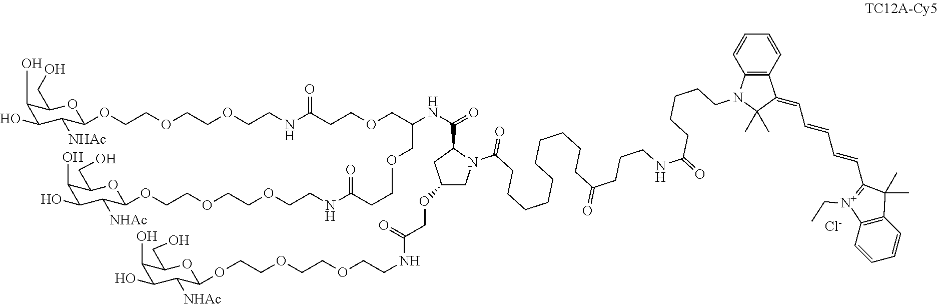 [SMILES: CC[N+]1=C(/C=C/C=C/C=C2/c3ccccc3N(CCCCCC(=O)NCCCC(=O)CCCCCCCCCCC(=O)N3C[C@H](OCC(=O)NCCOCCOCCOC4OC(CO)C(O)C(O)C4NC(C)=O)C[C@H]3C(=O)NC(COCCC(=O)NCCOCCOCCOC3OC(CO)C(O)C(O)C3NC(C)=O)COCCC(=O)NCCOCCOCCOC3OC(CO)C(O)C(O)C3NC(C)=O)C2(C)C)C(C)(C)c2ccccc21 ; [Cl-]]

TC12A-Cy5-01 (10 mg, 0.0039 mmol) was used as a starting material for synthesis and the crude product was purified by prep-HPLC to obtain 0.93 mg of a blue solid product with a yield of 10.9% and a purity of 97.1% (284 nm).

MS (ESI): m/z [1/2(M−Cl+H)]⁺, theoretical 1072.1, found 1071.7.

### 36. Synthesis of TC11A (a Stereoisomeric Carboxylic Acid with Peraceylated Glycosyl Group of Ligand TC11) and TC11A-Cy5 (a Receptor-Targeting Fluorescent Conjugate)

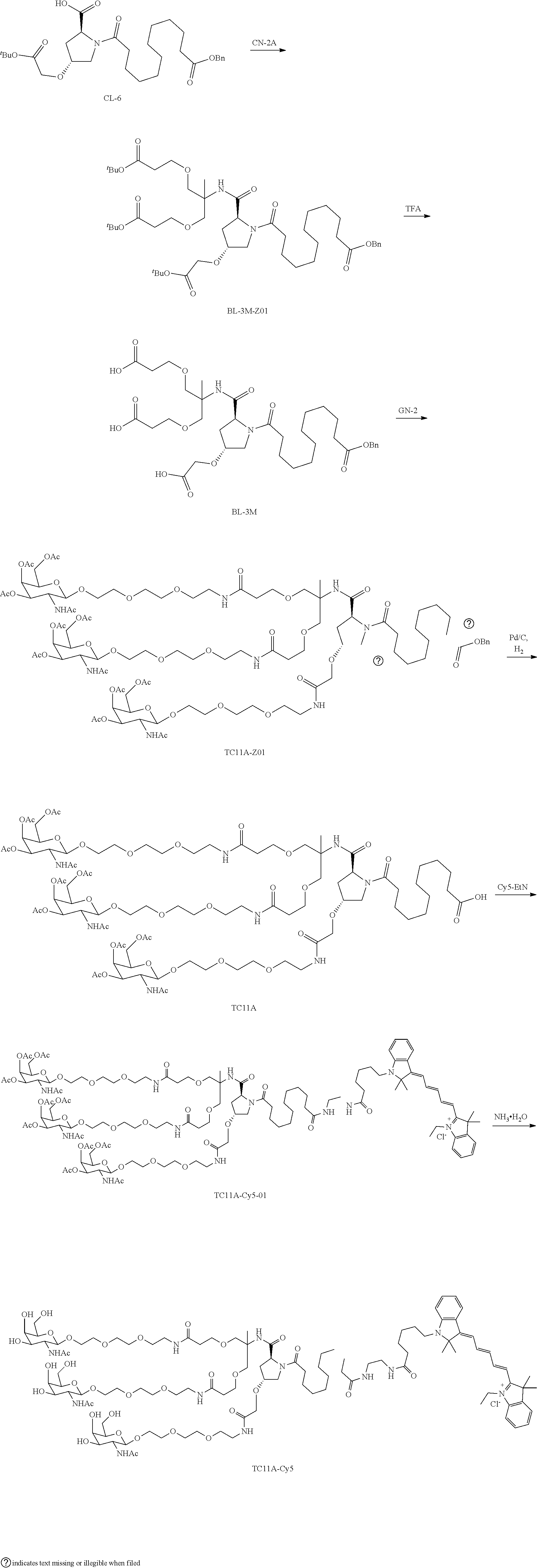

### (1) Synthesis of BL-3M-Z01 (Amidation Condensation Reaction)

Under N₂ atmosphere and at room temperature, CL-6 (670 mg, 1.2 mmol), CN-2A (442 mg, 1.2 mmol), EDCI (281 mg, 1.5 mmol) and HOBt (197 mg, 1.5 mmol) were successively added to a flask containing dichloromethane (15 mL), and under constant stirring, DIEA (314 mg, 2.4 mmol) was slowly added, and the reaction was carried out 12 h when LCMS showed that the reaction was completed. The reaction solution was added with pure water (30 mL) for quenching and dilution, and extracted with dichloromethane (30 mL×3). The obtained organic phases were combined, washed with 26% aqueous sodium chloride solution (40 mL), dried over anhydrous sodium sulfate, filtered and concentrated under reduced pressure to obtain a yellow liquid crude product. The crude product was purified by silica gel column chromatography (200-300 meshes, petroleum ether/ethyl acetate, 7/3) to obtain 430 mg of a yellow oily liquid product with a yield of 40%.

MS (ESI): m/z [M+Na]⁺, theoretical 891.6, found 891.5.

### (2) Synthesis of BL-3M (De-Tert-Butylation Reaction)

BL-3M-Z01 (430 mg, 0.5 mmol) was dissolved in dichloromethane (10 mL) in a flask and stirred at room temperature until the reaction mixture became clear, followed by slow addition of trifluoroacetic acid (1.12 g, 10 mmol). The reaction was carried out under stirring for 12 h when LCMS showed that the reaction was completed. The reaction solution was subjected to distillation under reduced pressure to remove the solvent, and the residue was dried under vacuum to obtain 370 mg of a yellow oily liquid product with a yield of 107% (the product contained some residual TFA and could be directly used in subsequent reaction without purification).

MS (ESI): m/z [M−H]⁻, theoretical 721.3, found 721.3.

### (3) Synthesis of TC11A-Z01 (Amidation Condensation Reaction)

Under N₂ atmosphere and at room temperature, BL-3M (361 mg, 0.5 mmol), GN-2 (1.07 g, 2.24 mmol), EDCI (400 mg, 2.09 mmol) and HOBt (300 mg, 2.22 mmol) were successively added to a flask containing dichloromethane (10 mL) and stirred at room temperature until the reaction mixture became clear, followed by slow addition of DIEA (387 mg, 3 mmol). The reaction was carried out for 12 h when LCMS showed that the reaction was completed. The reaction solution was quenched with pure water (20 mL) and extracted with dichloromethane (20 mL×3). The obtained organic phases were combined, washed with 26% aqueous sodium chloride solution (20 mL), dried over anhydrous sodium sulfate, filtered and concentrated under reduced pressure to obtain 1.2 g of a white solid crude product. The crude product was purified by silica gel column chromatography (200-300 meshes, dichloromethane/methanol, 9/1) to obtain 310 mg of a white solid product with a yield of 29.5%.

MS (ESI): m/z [1/3M+H]⁺, theoretical 702.0, found 702.2.

### (4) Synthesis of TC11A (Debenzylation Reaction)

TC11A-Z01 (310 mg, 0.147 mmol) was dissolved in tetrahydrofuran (6 mL) and added with Pd/C (30 mg, 10 wt %). The reaction system was replaced with hydrogen for three times and the reaction was carried out under stirring and under hydrogen atmosphere (about 10-20 kPa higher than the standard atmospheric pressure) for 2 h when LCMS showed that the reaction was complete. The mixture was filtered to remove the solid and the filtrate was concentrated to obtain 274 mg of a white solid product with a yield of 93%.

MS (ESI): m/z [1/3M+H]⁺, theoretical 672.0, found 672.1.

### (5) Synthesis of TC11A-Cy5-01 (Amidation Condensation Reaction)

Under N₂ atmosphere, TC11A (50 mg, 0.025 mmol), Cy5-EtN (14 mg, 0.025 mmol), EDCI (7 mg, 0.037 mmol) and HOBt (5 mg, 0.037 mmol) were successively added to a flask containing dichloromethane (2 mL) and stirred at room temperature until the reaction mixture became clear, followed by addition of DIEA (6.5 mg, 0.05 mmol). The reaction was carried out for 3 h when LCMS showed that the reaction was completed. The reaction solution was subjected to distillation under reduced pressure to remove the solvent, and the obtained crude product was purified by silica gel column chromatography (200-300 meshes, dichloromethane/methanol, 90/10 to 80/20) to obtain 25 mg of a blue solid product with a yield of 39%.

MS (ESI): m/z [1/3 (M−Cl+2H)]⁺, theoretical 845.8, found 845.5.

### (6) Synthesis of TC11A-Cy5

Under N₂ atmosphere, TC11A-Cy5-01 (25 mg, 0.01 mmol) and methanol (5 mL) were added to a flask and stirred until the reaction mixture became clear, followed by addition of 25% ammonia (0.5 mL). The reaction was increased to 40° C., and then carried out under stirring for 1 h when LCMS showed that the reaction was completed. The reaction solution was subjected to distillation under reduced pressure to remove the solvent, and the residue was purified by prep-HPLC (C₁₈/5 μm, column temperature: 40° C.; water/acetonitrile, 95/5 to 5/95, containing 0.1% formic acid) to obtain 8.9 mg of a blue solid product with a yield of 42% and a purity of 98.7% (285 nm).

MS (ESI): m/z [1/3 (M−Cl+2H)]⁺, theoretical 719.7, found 719.8.

### 37. Synthesis of TC10A (a Stereoisomeric Carboxylic Acid with Peracetylated Glycosyl Group of Ligand TC10) and TC10A-Cy5 (a Receptor-Targeting Fluorescent Conjugate)

TC10A and the fluorescent conjugate thereof were synthesized by the same steps for synthesis of TC11A and the fluorescent conjugate thereof, except that GN-2 was replaced with GN-1.

### (1) Synthesis of TC10A-Z01

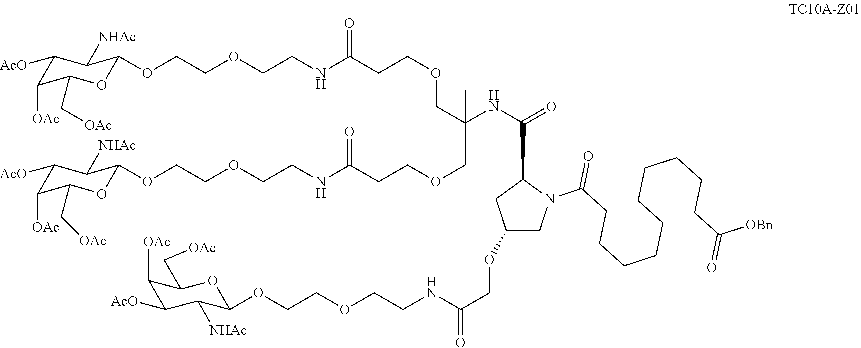 [SMILES: CC(=O)NC1C(OCCOCCNC(=O)CCOCC(C)(COCCC(=O)NCCOCCOC2OC(COC(C)=O)C(OC(C)=O)C(OC(C)=O)C2NC(C)=O)NC(=O)[C@@H]2C[C@@H](OCC(=O)NCCOCCOC3OC(COC(C)=O)C(OC(C)=O)C(OC(C)=O)C3NC(C)=O)CN2C(=O)CCCCCCCCCCC(=O)OCc2ccccc2)OC(COC(C)=O)C(OC(C)=O)C1OC(C)=O]

BL-3M (220 mg, 0.3 mmol) and GN-1 (400 mg, 0.9 mmol) were used as starting materials for synthesis to obtain 260 mg of a white solid product with a yield of 44%.

MS (ESI): m/z [1/2M+H]⁺, theoretical 986.5, found 986.1.

### (2) Synthesis of TC10A

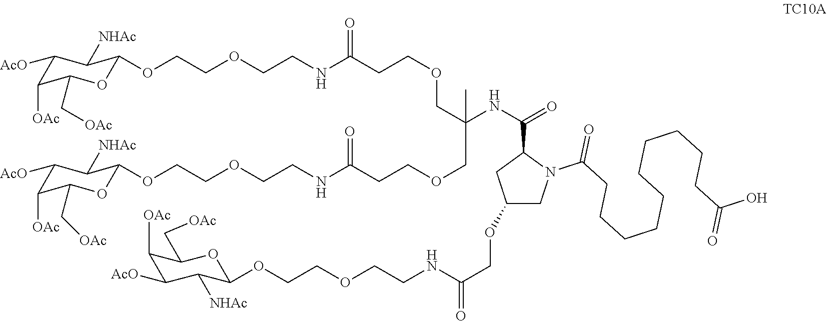 [SMILES: CC(=O)NC1C(OCCOCCNC(=O)CCOCC(C)(COCCC(=O)NCCOCCOC2OC(COC(C)=O)C(OC(C)=O)C(OC(C)=O)C2NC(C)=O)NC(=O)[C@@H]2C[C@@H](OCC(=O)NCCOCCOC3OC(COC(C)=O)C(OC(C)=O)C(OC(C)=O)C3NC(C)=O)CN2C(=O)CCCCCCCCCCC(=O)O)OC(COC(C)=O)C(OC(C)=O)C1OC(C)=O]

TC10A-Z01 (260 mg, 0.13 mmol) was used as a starting material for synthesis to obtain 220 mg of a white solid product with a yield of 88.7%.

MS (ESI): m/z [1/2M+H]⁺, theoretical 941.4, found 941.1.

¹H NMR (400 MHz, DMSO-d₆), δ 7.96 (br s, 2H), 7.83 (d, J=8.9 Hz, 3H), 7.77-7.67 (m, 2H), 5.20 (d, J=3.3 Hz, 3H), 4.96 (d, J=12.0 Hz, 3H), 4.52 (d, J=8.5 Hz, 3H), 4.45-4.24 (m, 1H), 4.05-3.97 (m, 9H), 3.91-3.81 (m, 6H), 3.80-3.72 (m, 3H), 3.64-3.51 (m, 10H), 3.50-3.43 (m, 8H), 3.42-3.36 (m, 9H), 3.30-3.21 (m, 4H), 3.20-3.11 (m, 6H), 2.35-2.27 (m, 6H), 2.25-2.14 (m, 2H), 2.08 (s, 9H), 1.98 (s, 9H), 1.87 (s, 9H), 1.76 (s, 9H), 1.55-1.47 (m, 2H), 147-1.37 (m, 2H), 1.21 (s, 12H), 1.19 (s, 3H).

### (3) Synthesis of TC10A-Cy5-01

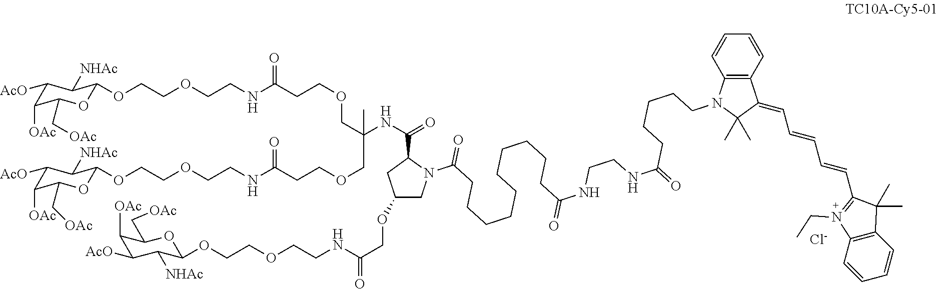

TC10A (100 mg, 0.05 mmol) was used as a starting material for synthesis to obtain 50 mg of a blue solid product with a yield of 41%.

MS (ESI): m/z [1/2(M−Cl+H)]⁺, theoretical 1202.1, found 1201.8.

### (4) Synthesis of TC10A-Cy5

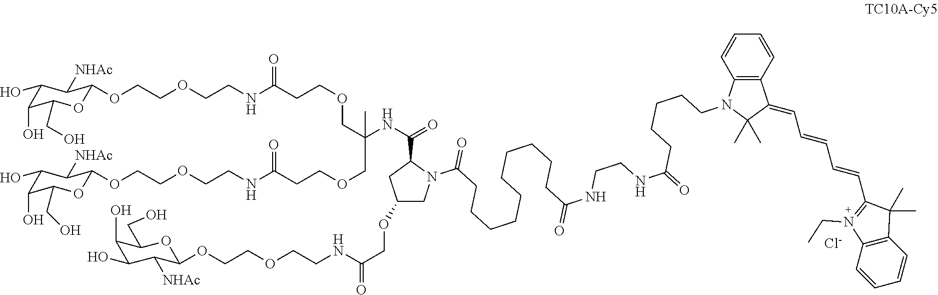

TC10A-Cy5-01 (11 mg, 0.0045 mmol) was used as a starting material for synthesis and the crude product was purified by prep-HPLC to obtain 3.17 mg of a blue solid product with a yield of 35% and a purity of 98.8% (284 nm).

MS (ESI): m/z [1/2(M−Cl+H)]⁺, theoretical 1013.1, found 1013.0.

### 38. Synthesis of TC13A (a Stereoisomeric Carboxylic Acid with Peracetylated Glycosyl Groups of Ligand TC13)

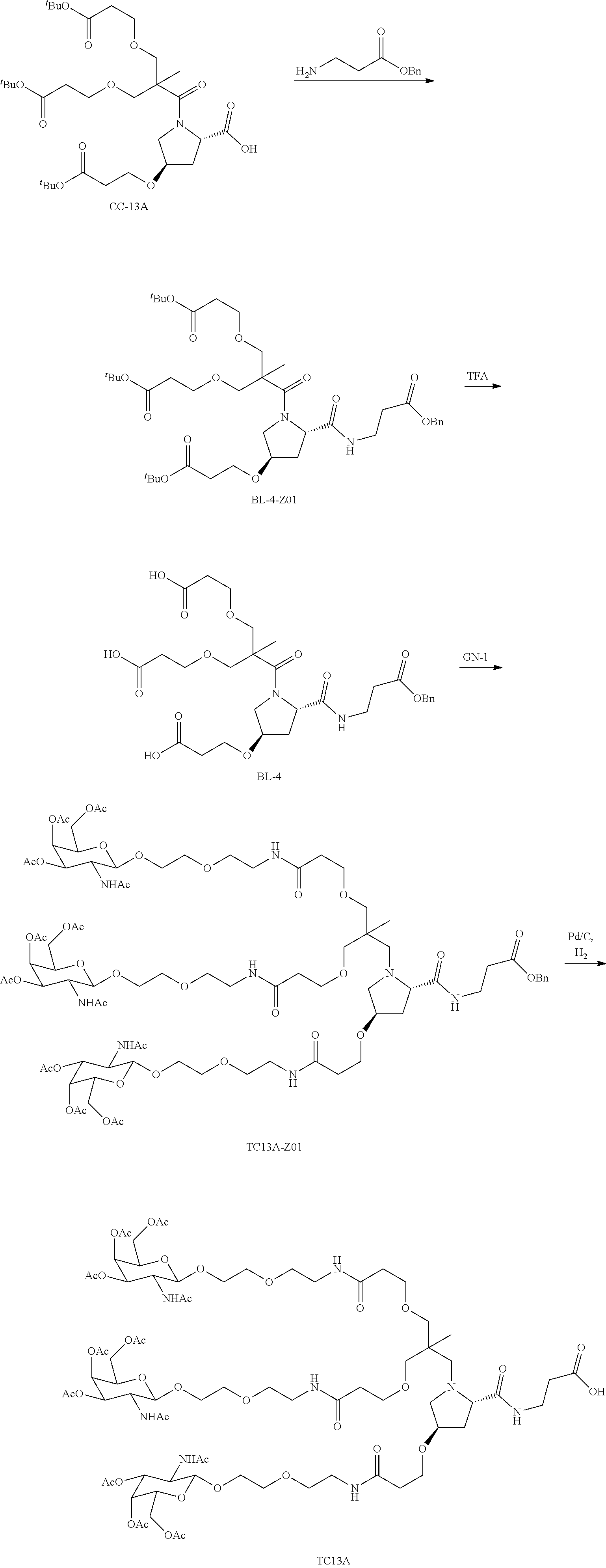

### (1) Synthesis of BL-4-Z01

L-4-Z01 was obtained by subjecting CC-13A and benzyl 3-aminopropionate as starting materials to similar amidation condensation steps for synthesis of BL-1-Z01.

MS (ESI): m/z [M+H]⁺, theoretical 793.4, found 793.7.

### (2) Synthesis of BL-4

BL-4 was obtained by subjecting BL-4-Z01 as a starting material to the same de-tert-butylation reaction steps for synthesis of BL-1.

MS (ESI): m/z [M−H]⁻, theoretical 623.3, found 623.4.

### (3) Synthesis of TC13A-Z01

TC13A-Z01 was obtained by subjecting BL-4 and GN-1 as starting materials to the same amidation condensation reaction steps for synthesis of TC01A-Z01.

MS (ESI): m/z [1/2M+H]⁺, theoretical 937.4, found 937.5.

### (4) Synthesis of TC13A

TC13A was obtained by subjecting TC13A-Z01 as a starting material to the same debenzylation reaction steps for synthesis of TC01A.

MS (ESI): m/z [1/2M+H]⁺, theoretical 892.4, found 892.4.

### 39. Synthesis of TC14A (a Stereoisomer Carboxylic Acid with Peracetylated Glycosyl Groups of Ligand TC14

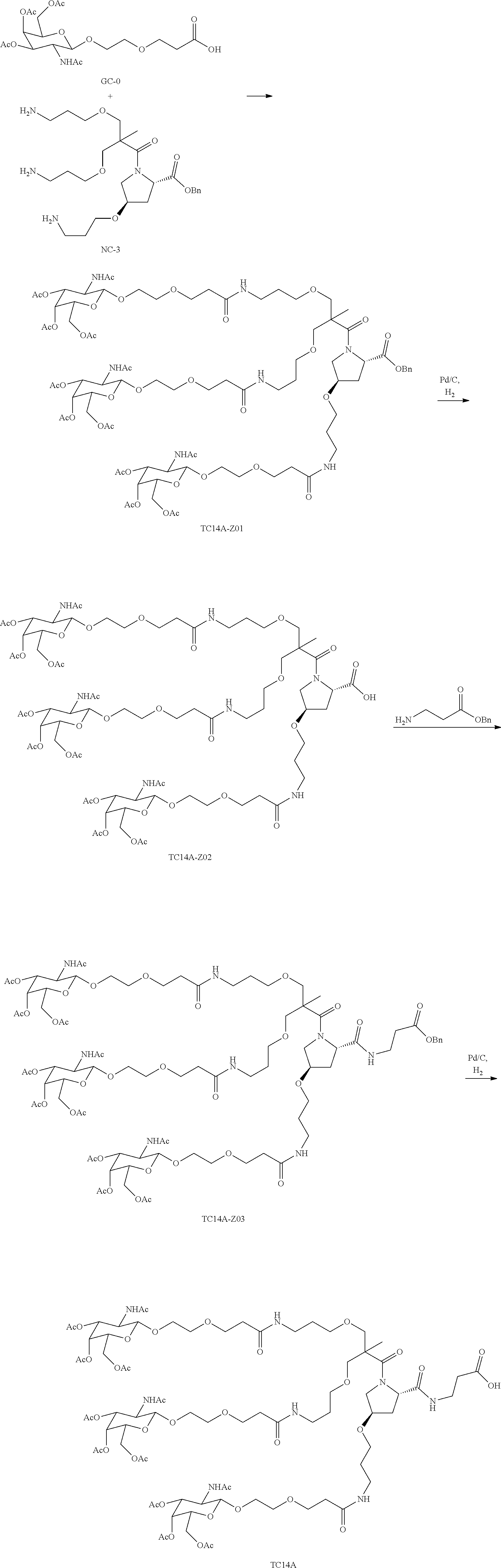

### (1) Synthesis of TC14A-Z01 (Amidation Condensation Reaction)

Under N₂ atmosphere, GC-0 (1.3 g, 2.81 mmol), EDCI (570 mg, 2.98 mmol), triethylamine (517 mg, 5.1 mmol) and HOBt (115 mg, 0.852 mmol) were successively added to a flask containing dichloromethane (16 mL), and under stirring, NC-3 (433 mg, 0.852 mmol) was added, and the reaction was carried out at room temperature for 16 h when LCMS showed that the reaction was completed. The reaction solution was added with pure water (25 mL) and extracted with dichloromethane (15 mL×3). The obtained organic phases were combined, dried over anhydrous sodium sulfate, and filtered. The reaction solution was subjected to distillation under reduced pressure, and the obtained crude product was purified by silica gel column chromatography (200-300 meshes, dichloromethane/methanol, 15/1) to obtain 1.21 g of a white solid product with a yield of 77%.

MS (ESI): m/z [1/2M+H]⁺, theoretical 922.9, found 923.1.

### (2) Synthesis of TC14A-Z02

TC14A-Z02 was obtained by subjecting TC14A-Z01 as a starting material to the same debenzylation reaction steps for synthesis of TC08A-Z02.

MS (ESI): m/z [1/2M+H]⁺, theoretical 877.9, found 877.9.

### (3) Synthesis of TC14A-Z03

TC14A-Z03 was obtained by subjecting TC14A-Z02 and benzyl 3-aminopropionate as starting materials to similar amidation condensation steps for synthesis of BL-4-Z01.

MS (ESI): m/z [M+H]⁺, theoretical 958.4, found 958.4.

### (4) Synthesis of TC14A

TC14A was obtained by subjecting TC14A-Z03 as a starting material to the same debenzylation reaction steps for synthesis of TC13A.

MS (ESI): m/z [1/2M+H]⁺, theoretical 913.4, found 913.5.

### 40. Synthesis of TC15A (a Stereoisomeric Carboxylic Acid with Peracetylated Glycosyl Groups of Ligand TC15)

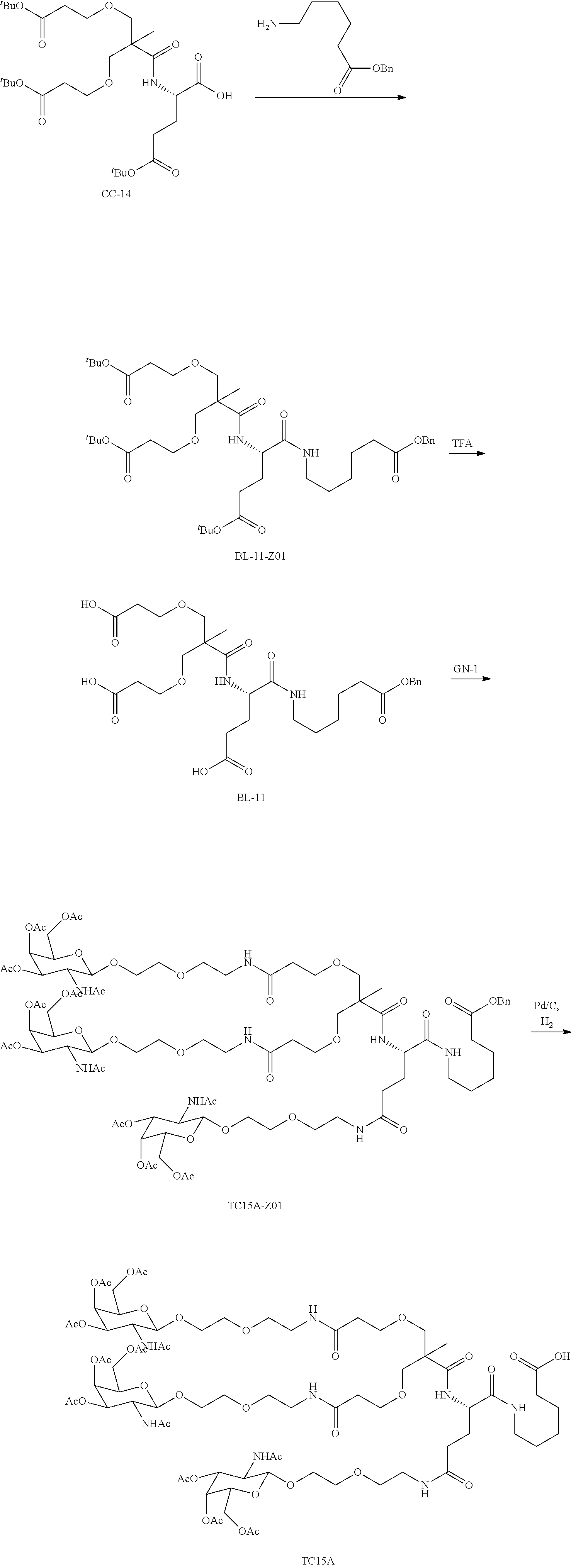

### (1) Synthesis of BL-11-Z01

BL-11-Z01 was obtained by subjecting CC-14 and benzyl 6-aminocaproate as starting materials to similar amidation condensation steps for synthesis of BL-1-Z01.

MS (ESI): m/z [M+H]⁺, theoretical 779.5, found 779.6.

### (2) Synthesis of BL-11

BL-11 was obtained by subjecting BL-11-Z01 as a starting material to the same de-tert-butylation reaction steps for synthesis of BL-1.

MS (ESI): m/z [M−H]⁻, theoretical 609.3, found 609.2.

### (3) Synthesis of TC15A-Z01

TC15A-Z01 was obtained by subjecting BL-11 and GN-1 as starting materials to the same amidation condensation reaction steps for synthesis of TC01A-Z01.

MS (ESI): m/z [1/2M+H]⁺, theoretical 930.4, found 930.6.

### (4) Synthesis of TC15A

TC15A was obtained by subjecting TC15A-Z01 as a starting material to the same debenzylation reaction steps for synthesis of TC01A.

MS (ESI): m/z [1/2M+H]⁺, theoretical 885.4, found 885.5.

### 41. Synthesis of TC16A (a Stereoisomeric Carboxylic Acid with Peracetylated Glycosyl Groups of Ligand TC16)

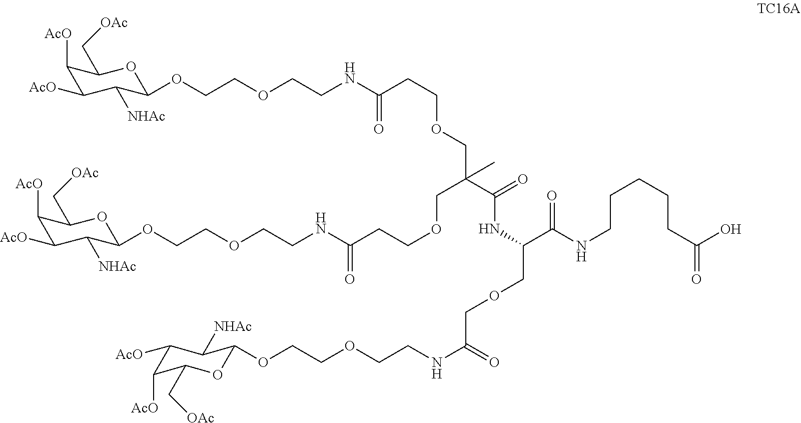 [SMILES: CC(=O)NC1C(OCCOCCNC(=O)CCOCC(C)(COCCC(=O)NCCOCCOC2OC(COC(C)=O)C(OC(C)=O)C(OC(C)=O)C2NC(C)=O)C(=O)N[C@@H](COCC(=O)NCCOCCOC2OC(COC(C)=O)C(OC(C)=O)C(OC(C)=O)C2NC(C)=O)C(=O)NCCCCCC(=O)O)OC(COC(C)=O)C(OC(C)=O)C1OC(C)=O]

TC16A was synthesized by the same steps for synthesis of TC15A, except that CC-14 was replaced with CC-6A as a starting material.

### 42. Synthesis of TC25A (a Stereoisomeric Carboxylic Acid with Peracetylated Glycosyl Groups of Ligand TC25)

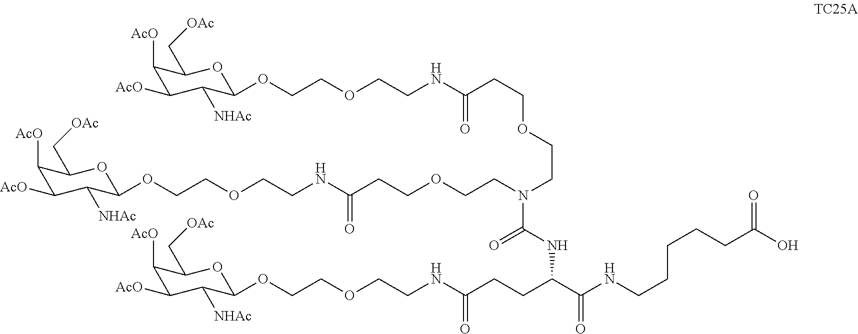 [SMILES: CC(=O)NC1C(OCCOCCNC(=O)CCOCCN(CCOCCC(=O)NCCOCCOC2OC(COC(C)=O)C(OC(C)=O)C(OC(C)=O)C2NC(C)=O)C(=O)N[C@@H](CCC(=O)NCCOCCOC2OC(COC(C)=O)C(OC(C)=O)C(OC(C)=O)C2NC(C)=O)C(=O)NCCCCCC(=O)O)OC(COC(C)=O)C(OC(C)=O)C1OC(C)=O]

TC25A was synthesized by the same steps for synthesis of TC15A, except that CC-14 was replaced with CC-12 as a starting material.

### 43. Synthesis of TC17A (a Carboxylic Acid Compound with Peracetylated Glycosyl Groups of Ligand TC17) and TC17A-Cy5 (a Receptor-Targeting Fluorescent Conjugate)

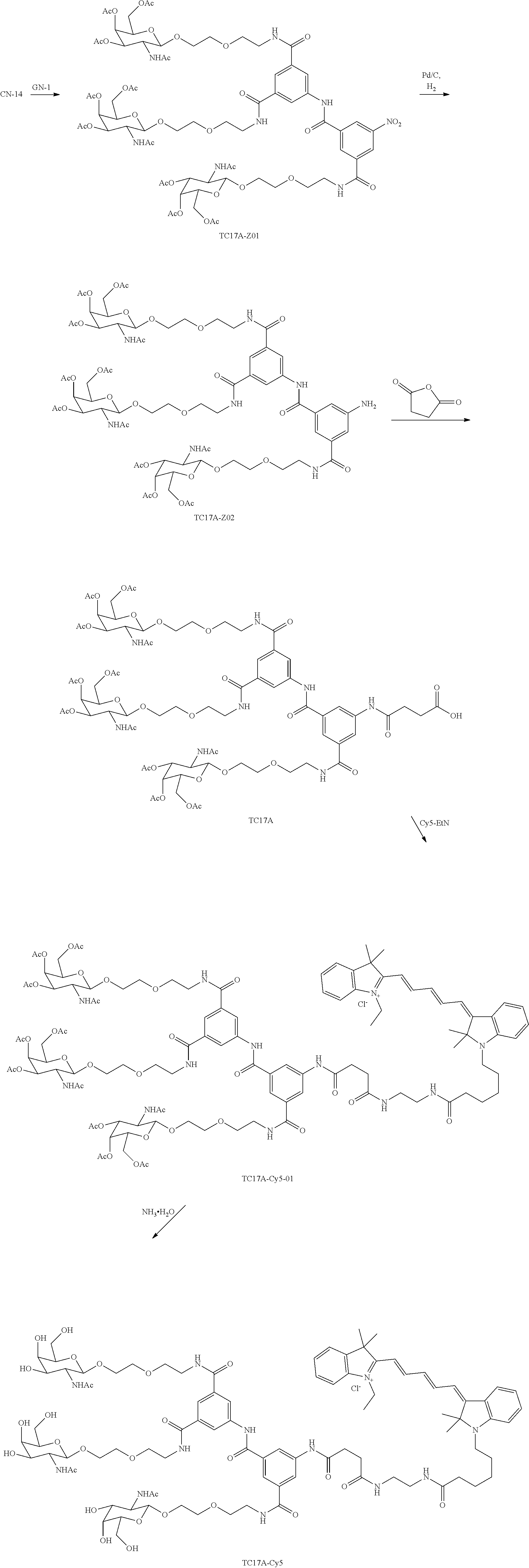

### (1) Synthesis of TC17A-Z01 (Amidation Condensation Reaction)

Under N₂ atmosphere, dichloromethane (20 mL), CN-14 (164 mg, 0.44 mmol), GN-1 (868 mg, 2.0 mmol), EDCI (506 mg, 2.64 mmol), triethylamine (266 mg, 2.64 mmol) and DMAP (53 mg, 0.44 mmol) were successively added to a flask and the reaction was carried out under stirring at room temperature for 16 h when LCMS showed that the reaction was complete. The reaction solution was added with pure water (100 mL) and extracted with dichloromethane (50 mL×3). The obtained organic phases were combined, dried over anhydrous sodium sulfate, filtered, and subjected to distillation under reduced pressure to remove the solvent. The obtained crude product was purified by silica gel column chromatography (200-300 meshes, dichloromethane/methanol, 30/1 to 10/1) to obtain 446 mg of a white solid product with a yield of 63%.

MS (ESI): m/z [1/2M+H]⁺, theoretical 812.3, found 812.3.

### (2) Synthesis of TC17A-Z02

TC17A-Z01 (300 mg, 0.185 mmol), Pd/C (30 mg, 10 wt %), methanol (1 mL) and tetrahydrofuran (10 mL) were successively added to a flask. The reaction system was replaced with hydrogen for three times and the reaction was carried out under stirring and under hydrogen atmosphere (about 10-20 kPa higher than the standard atmospheric pressure) for 2 h when TLC showed that the conversion of the raw materials was complete. The reaction mixture was filtered to remove the solid and the filtrate was concentrated under reduced pressure. The obtained crude product was purified by silica gel column chromatography (200-300 meshes, dichloromethane/methanol, 9/1) to obtain 270 mg of a yellow solid product with a yield of 92%.

MS (ESI): m/z [1/2M+H]⁺, theoretical 797.3, found 797.5.

### (3) Synthesis of TC17A (Amidation Reaction)

Under N₂ atmosphere, dichloromethane (3 mL), TC17A-Z02 (85 mg, 0.0534 mmol), succinic anhydride (16 mg, 0.16 mmol) and triethylamine (22 mg, 0.22 mmol) were successively added to a flask and the reaction was carried out under stirring at room temperature for 5 h when TLC showed that the conversion of the raw materials was complete. The reaction solution was subjected to distillation under reduced pressure to remove the solvent, and the obtained crude product was purified by silica gel column chromatography (200-300 meshes, dichloromethane/methanol, 5/1) to obtain 50 mg of a white solid product with a yield of 56%.

MS (ESI): m/z [1/2(M+H+Na)]⁺, theoretical 858.3, found 858.5.

### (4) Synthesis of TC17A-Cy5-01 (Amidation Condensation Reaction)

Under N₂ atmosphere, dichloromethane (10 mL), TC17A (20 mg, 0.0118 mmol), Cy5-EtN (6.8 mg, 0.0118 mmol), EDCI (3.4 mg, 0.0177 mmol), HOBt (1.6 mg, 0.0118 mmol) and DIEA (3.1 mg, 0.0236 mmol) were successively added to a flask. The reaction was carried out under stirring at room temperature and in the dark for 24 h when LCMS showed that the reaction was completed. The reaction solution was subjected to distillation under reduced pressure to remove the solvent, and the crude product was purified by prep-TLC (dichloromethane/methanol, 5/1) to obtain 9 mg of a blue solid product with a yield of 34%.

MS (ESI): m/z [1/3 (M−Cl+2H)]⁺, theoretical 739.0, found 738.8.

### (5) Synthesis of TC17A-Cy5

TC17A-Cy5-01 (9 mg, 0.0041 mmol) and methanol (4.5 mL) were added to a flask and stirred until the reaction mixture became clear. The reaction mixture was added with 25% ammonia (0.5 mL) and stirred at room temperature for 6.5 h when LCMS showed that the reaction was complete. The reaction solution was subjected to distillation under reduced pressure to remove the solvent, thereby obtaining 6.5 mg of a blue solid crude product. The crude product was purified by prep-HPLC (C₁₈/5 μm, column temperature: 40° C.; water/acetonitrile, 95/5 to 5/95, containing 0.1% formic acid) to obtain 1.1 mg of a blue solid product with a yield of 14.7% and a purity of 99.6% (630 nm).

MS (ESI): m/z [1/2(M−Cl+Na)]⁺, theoretical 929.4, found 929.8.

### 44. Synthesis of TC18A (a Carboxylic Acid Compound with Peracetylated Glycosyl Groups of Ligand TC18) and TC18A-Cy5 (a Receptor-Targeting Fluorescent Conjugate)

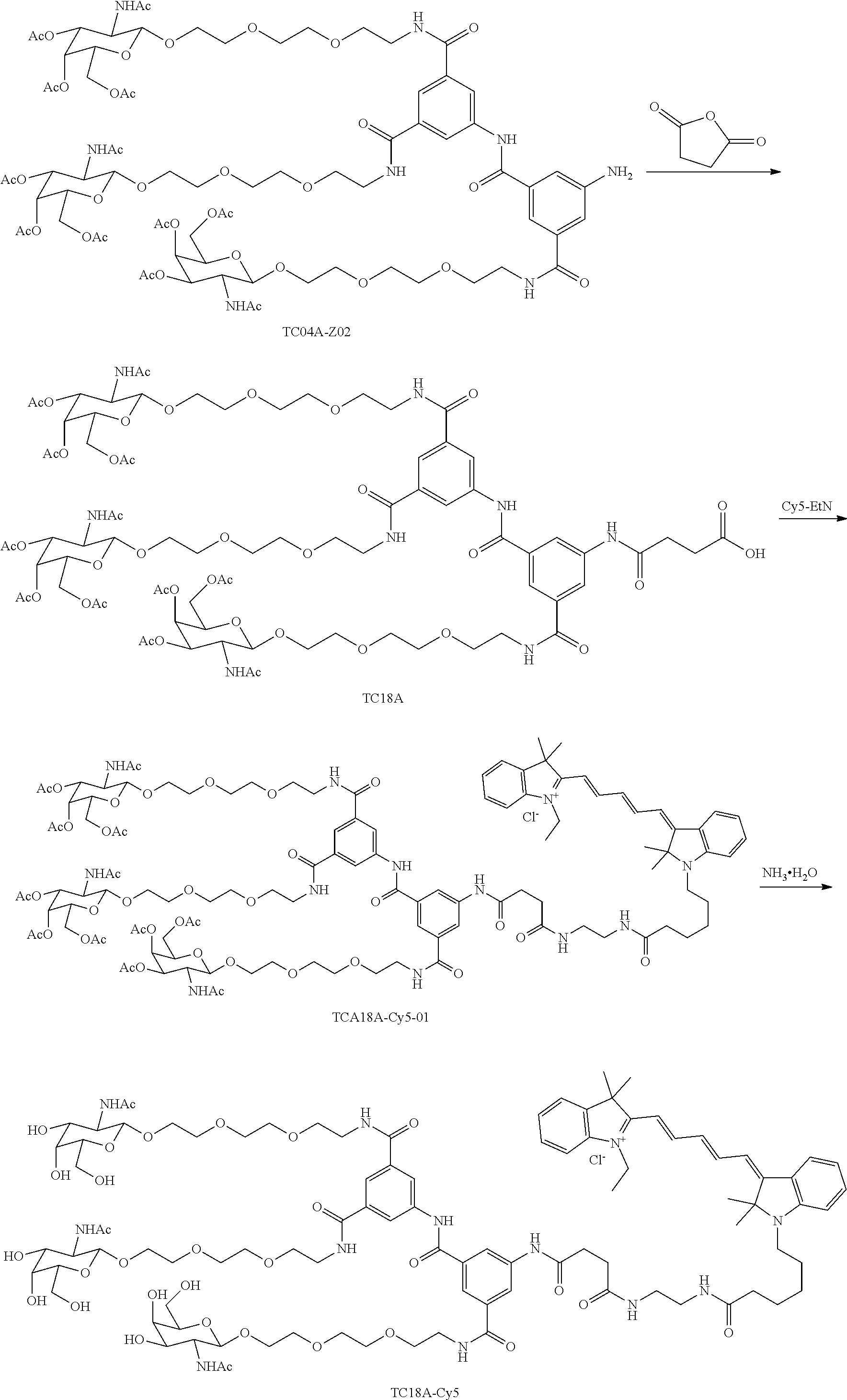

### (1) Synthesis of TC18A (Amidation Reaction)

Dichloromethane (7 mL), TC04A-Z02 (70 mg, 0.04 mmol), succinic anhydride (20 mg, 0.2 mmol) and triethylamine (20 mg, 0.2 mmol) were successively added to a flask and the reaction was carried out under stirring at 35° C. for 6 h when TLC showed that the conversion of the raw materials was complete. The reaction solution was subjected to distillation under reduced pressure to remove the solvent, and the obtained crude product was purified by silica gel column chromatography (200-300 meshes, dichloromethane/methanol, 10/1) to obtain 55 mg of a white solid product with a yield of 75%.

MS (ESI): m/z [1/2M+H]⁺, theoretical 913.4, found 913.4.

### (2) Synthesis of TC18A-Cy5-01 (Amidation Condensation Reaction)

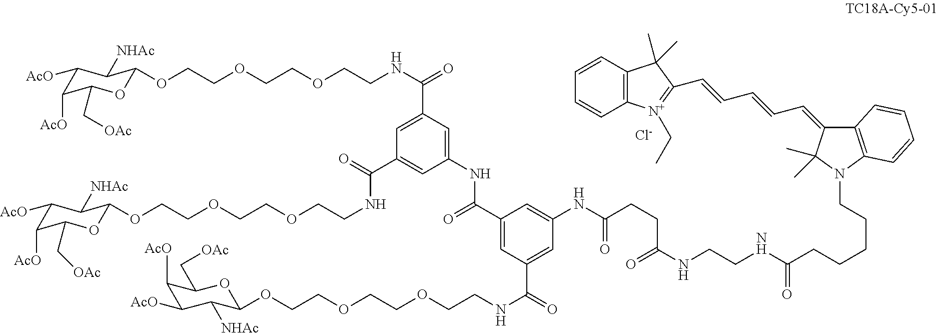 [SMILES: CC[N+]1=C(/C=C/C=C/C=C2/c3ccccc3N(CCCCCC(=O)NCCNC(=O)CCC(=O)Nc3cc(C(=O)NCCOCCOCCOC4OC(COC(C)=O)C(OC(C)=O)C(OC(C)=O)C4NC(C)=O)cc(C(=O)Nc4cc(C(=O)NCCOCCOCCOC5OC(COC(C)=O)C(OC(C)=O)C(OC(C)=O)C5NC(C)=O)cc(C(=O)NCCOCCOCCOC5OC(COC(C)=O)C(OC(C)=O)C(OC(C)=O)C5NC(C)=O)c4)c3)C2(C)C)C(C)(C)c2ccccc21 ; [Cl-]]

Under N₂ atmosphere, dichloromethane (10 mL), TC18A (55 mg, 0.03 mmol), Cy5-EtN (34.5 mg, 0.06 mmol), EDCI (11.5 mg, 0.06 mmol), HOBt (8.1 mg, 0.06 mmol) and DIEA (7.7 mg, 0.06 mmol) were successively added to a flask. The reaction was carried out under stirring at room temperature and in the dark for 27 h when LCMS showed that the reaction was completed. The reaction solution was subjected to distillation under reduced pressure to remove the solvent, and the crude product was purified by prep-TLC twice (dichloromethane/methanol, 5/1) to obtain 32 mg of a blue solid product with a yield of 45%.

MS (ESI): m/z [1/2(M−Cl+H)]⁺, theoretical 1174.0, found 1173.7.

### (3) Synthesis of TC18A-Cy5

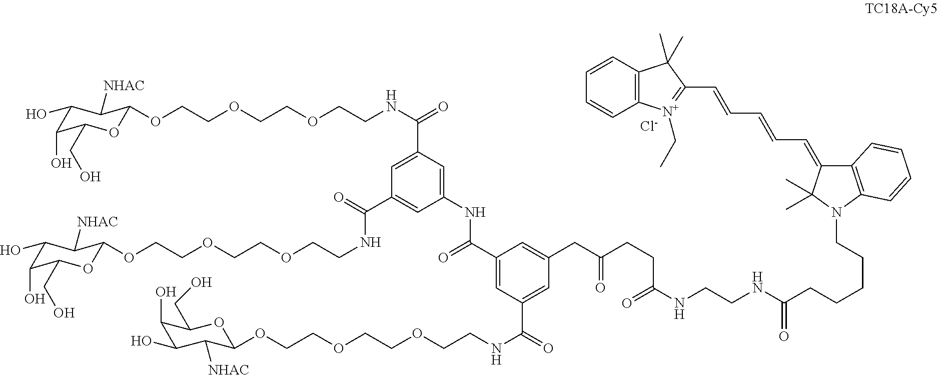

TC18A-Cy5-01 (32 mg, 0.013 mmol) and methanol (10 mL) were added to a flask and stirred until the reaction mixture became clear, followed by addition of 25% ammonia (1 mL). The reaction was carried out under stirring at room temperature for 6 h when LCMS showed that the reaction was completed. The reaction solution was subjected to distillation under reduced pressure to remove the solvent, thereby obtaining 29 mg of a blue solid crude product. The crude product was purified by prep-HPLC (C₁₈/5 μm, column temperature: 40° C.; water/acetonitrile, 95/5 to 5/95, containing 0.1% formic acid) to obtain 4.8 mg of a blue solid product with a yield of 17.8% and a purity of 99.9% (630 nm).

MS (ESI): m/z [1/2(M−Cl+Na)]⁺, theoretical 996.0, found 995.8.

### 45. Synthesis of TC19A (a Stereoisomeric Carboxylic Acid with Peracetylated Glycosyl Groups of Ligand TC19)

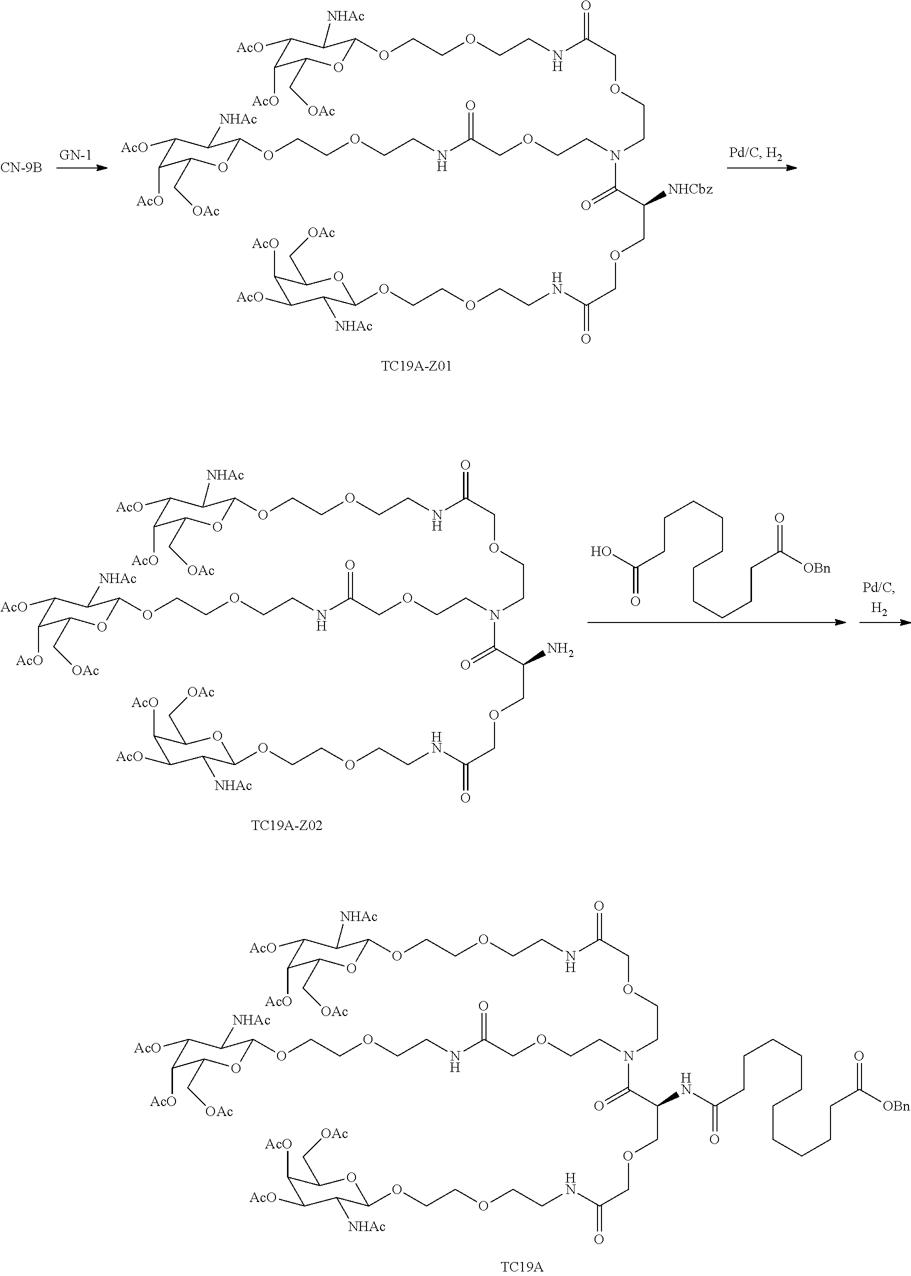

### (1) Synthesis of TC19A-Z01

TC19A-Z01 was obtained by subjecting CN-9B and GN-1 as starting materials to the same amidation condensation reaction steps for synthesis of TC02A-Z01.

MS (ESI): m/z [1/2M+H]⁺, theoretical 875.4, found 875.5.

### (2) Synthesis of TC19A-Z02

TC19A-Z02 was obtained by subjecting TC19A-Z01 as a starting material to the same de-benzyloxycarbonylation reaction steps for synthesis of TC02A-Z02.

MS (ESI): m/z [1/2M+H]⁺, theoretical 808.3, found 808.3.

### (3) Synthesis of TC19A (Amidation Condensation and Debenzylation Reaction)

Under N₂ atmosphere, TC19A-Z02 (0.78 g, 0.483 mmol), monobenzyl dodecyldiate (0.155 g, 0.483 mmol), EDCI (0.12 g, 0.63 mmol) and HOBt (0.065 g, 0.48 mmol) were successively added to a flask containing dichloromethane (12 mL), and then under constant stirring, triethylamine (0.098 g, 0.97 mmol) was added, and the reaction was carried out at room temperature for 4 h when LCMS showed that the reaction was complete. The reaction solution was diluted with dichloromethane (20 mL), washed with 26% aqueous sodium chloride solution (15 mL×2), dried over anhydrous sodium sulfate, filtered and concentrated under reduced pressure. The residue was purified by silica gel column chromatography (200-300 meshes, dichloromethane/methanol, 15/1) to obtain 0.74 g of a light yellow solid, which was a condensed intermediate. The solid was dissolved with tetrahydrofuran (6 mL) and added with Pd/C (70 mg, wt %). The reaction system was replaced with hydrogen for three times and the reaction was carried out under stirring and under hydrogen atmosphere (about 10-20 kPa higher than the standard atmospheric pressure) for 2 h when TLC showed that the conversion of the raw materials was complete. The reaction mixture was filtered to remove the solid, and the filtrate was concentrated under reduced pressure and dried under vacuum to obtain 0.53 g of a white solid product with a yield of 60%.

MS (ESI): m/z [1/2M+H]⁺, theoretical 914.4, found 914.4.

### 46. Synthesis of TC20A (a Stereoisomeric Carboxylic Acid with Peractylated Glycosyl Groups of Ligand TC20), TC26A (a Stereoisomeric Carboxylic Acid with Peractylated Glycosyl Groups of Ligand TC26) and TC24A (a Carboxylic Acid Compound with Peractylated Glycosyl Groups of Ligand TC24)

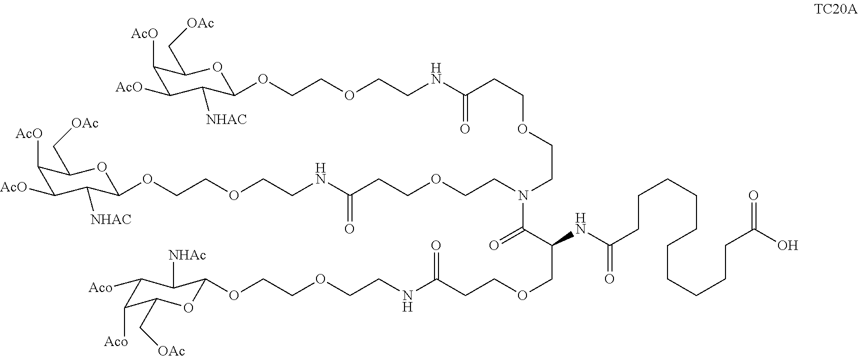

TC20A was synthesized by the same steps for synthesis of TC19A, except that CN-9B was replaced with CN-15 as a starting material.

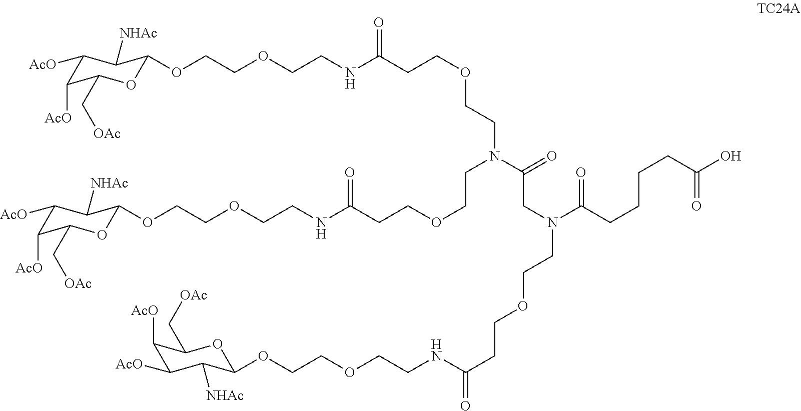 [SMILES: CC(=O)NC1C(OCCOCCNC(=O)CCOCCN(CCOCCC(=O)NCCOCCOC2OC(COC(C)=O)C(OC(C)=O)C(OC(C)=O)C2NC(C)=O)C(=O)CN(CCOCCC(=O)NCCOCCOC2OC(COC(C)=O)C(OC(C)=O)C(OC(C)=O)C2NC(C)=O)C(=O)CCCCC(=O)O)OC(COC(C)=O)C(OC(C)=O)C1OC(C)=O]

TC24A was synthesized by the same steps for synthesis of TC19A, except that CN-9B was replaced with CN-13, and monobenzyl dodecanediate was replaced with monobenzyl adipate.

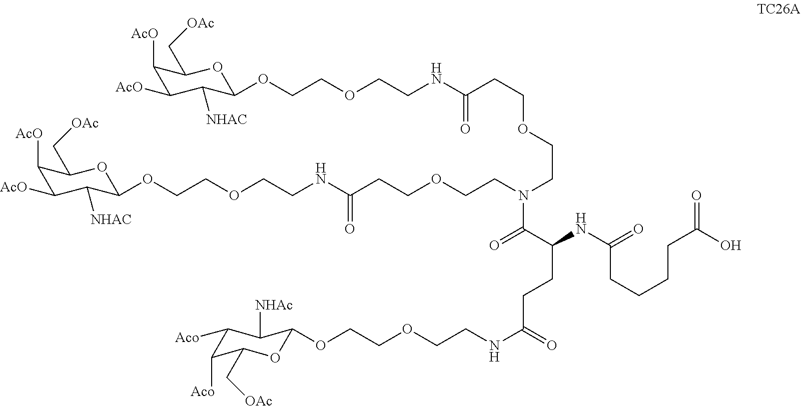

TC26A was synthesized by the same steps for synthesis of TC19A, except that CN-9B was replaced with CN-11, and monobenzyl dodecanediate with monobenzyl adipate.

### 47. Synthesis of TC21A (a Stereoisomeric Carboxylic Acid with Peractylated Glycosyl Groups of Ligand TC21)

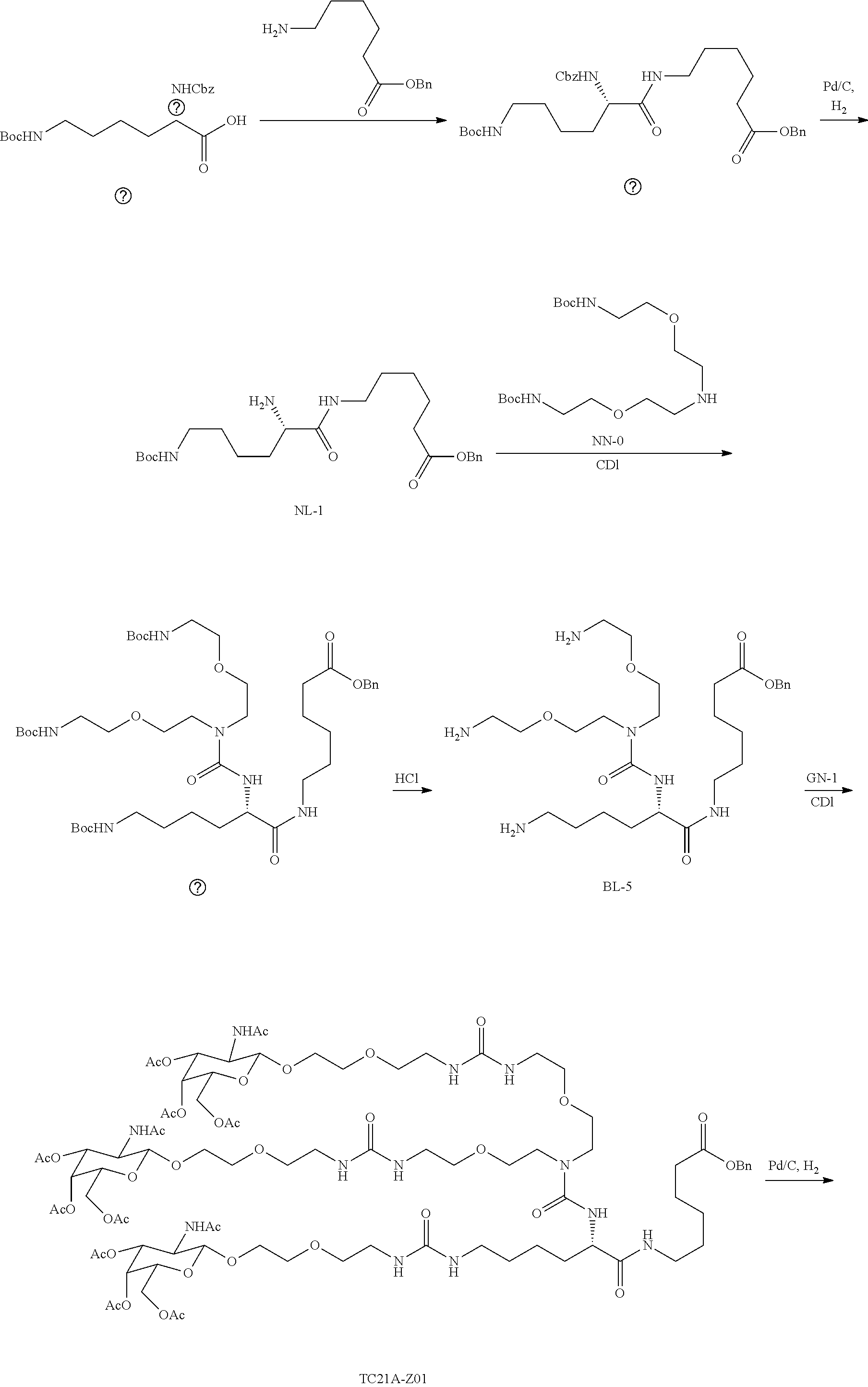

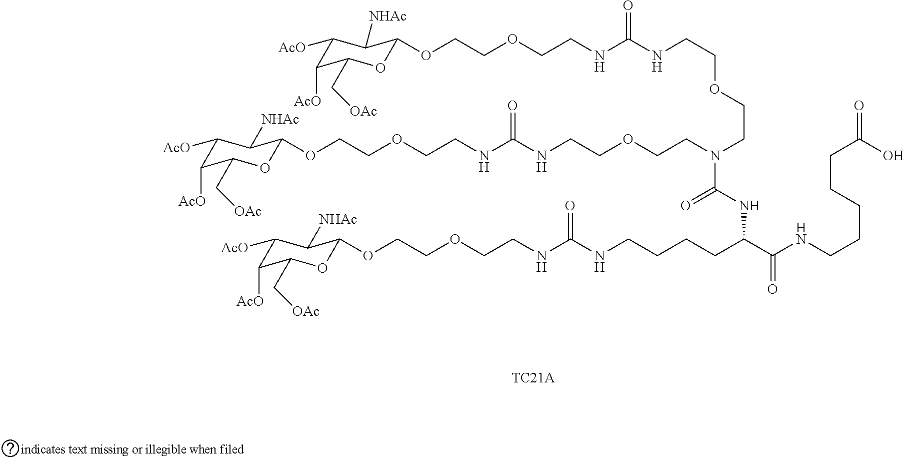

### (1) Synthesis of NL-1-Z01

NL-1-Z01 was obtained by subjecting N-α-Cbz-Nε-Boc-D-lysine and benzyl 6-aminocaproate as starting materials to similar amidation condensation reaction steps for synthesis of CL-1X.

MS (ESI): m/z [M+H]⁺, theoretical 584.3, found 584.4.

### (2) Synthesis of NL-1

NL-1 was obtained by subjecting NL-1-Z01 as a starting material to similar de-benzyloxycarbonylation reaction steps for synthesis of TC19A-Z02.

MS (ESI): m/z [M+H]⁺, theoretical 450.3, found 450.2.

### (3) Synthesis of BL-5-Z01

BL-5-Z01 was obtained by subjecting NN-0 and NL-1 as starting materials to similar urea condensation reaction steps for synthesis of CC-3X.

MS (ESI): m/z [M+H]⁺, theoretical 867.5, found 867.4.

### (4) Synthesis of BL-5

BL-5 was obtained by subjecting BL-5-Z01 as a starting material to the same de-tert-butoxycarbonylation reaction steps for synthesis of NC-4.

MS (ESI): m/z [M+H]⁺, theoretical 567.4, found 567.5.

### (5) Synthesis of TC21A-Z01 (Urea Condensation Reaction)

Under N₂ atmosphere, GN-1 (1.3 g, 2.99 mmol) and triethylamine (0.36 g, 3.59 mmol) were added to a flask containing dichloromethane (18 mL), and under stirring, N,N′-carbonyldiimidazole (0.49 g, 3.05 mmol) was slowly added, and the reaction was carried out for 1 h. The reaction was then added with BL-5 (a HCl salt, 0.67 g, 0.997 mmol) in batches, and then carried out under stirring at room temperature for 16 h when LCMS showed that the reaction was complete. The reaction solution was added with pure water (25 mL) and extracted with dichloromethane (15 mL×3). The obtained organic phases were combined, washed with 26% saturated saline solution (20 mL), dried over anhydrous sodium sulfate, filtered and concentrated. The obtained crude product was purified by silica gel column chromatography (200-300 meshes, dichloromethane/methanol, 15/1) to obtain 1.01 g of a light yellow solid product with a yield of 52%.

MS (ESI): m/z [1/2M+H]⁺, theoretical 974.4, found 974.5.

### (6) Synthesis of TC21A

TC21A was obtained by subjecting TC21A-Z01 as a starting material to the same debenzylation reaction steps for synthesis of TC03A.

MS (ESI): m/z [1/2M+H]⁺, theoretical 929.4, found 929.4.

### 48. Synthesis of TC22A (a Stereoisomeric Carboxylic Acid with Peracetylated Glycosyl Groups of Ligand TC22)

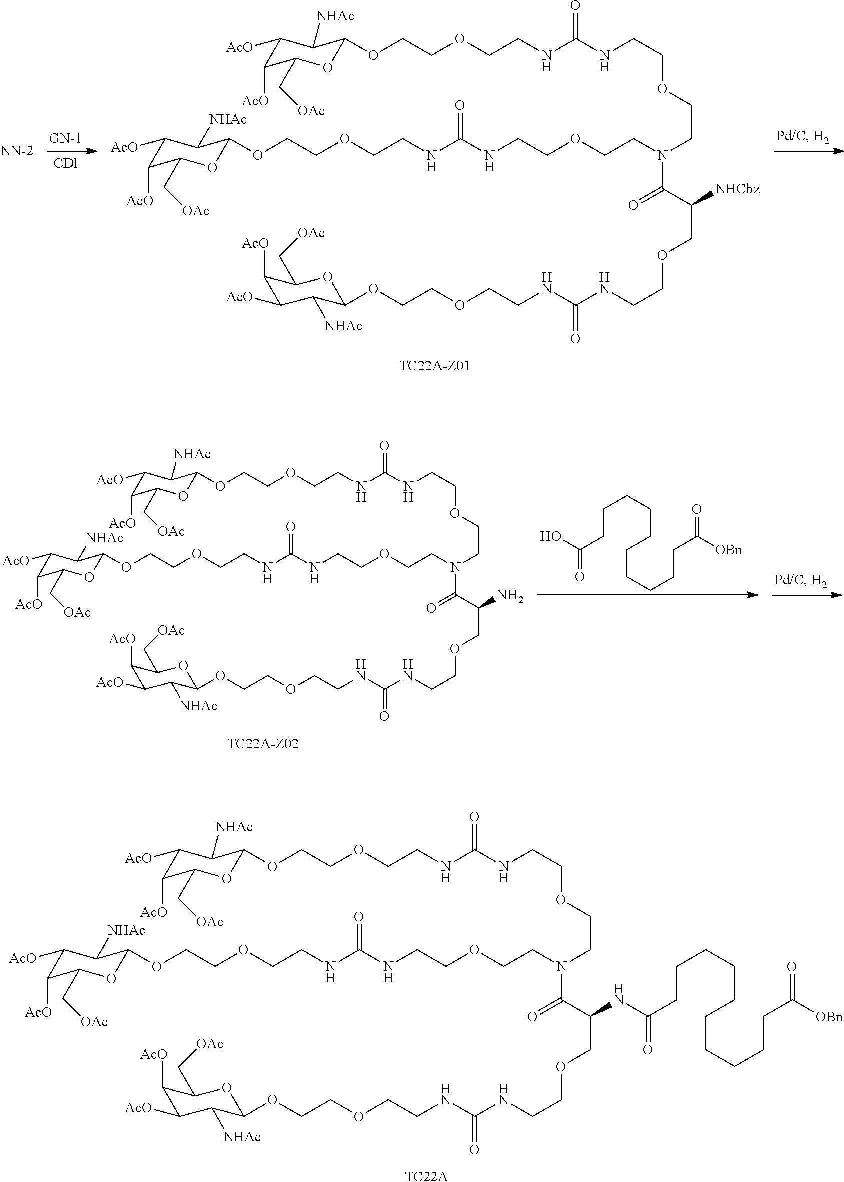

### (1) Synthesis of TC22A-Z01

TC22A-Z01 was obtained by subjecting NN-2 and GN-1 as starting materials to the same urea condensation reaction steps for synthesis of TC21A-Z01.

MS (ESI): m/z [1/2M+H]⁺, theoretical 918.9, found 918.8.

### (2) Synthesis of TC22A-Z02

TC22A-Z02 was obtained by subjecting TC22A-Z01 as a starting material to the same de-benzyloxycarbonylation reaction steps for synthesis of TC19A-Z02.

MS (ESI): m/z [1/2M+H]⁺, theoretical 851.9, found 852.0.

### (3) Synthesis of TC22A

TC22A was obtained by subjecting TC22A-Z02 and monobenzyl dodecanediate as starting materials to the same steps of amidation condensation and debenzylation reaction for synthesis of TC19A.

MS (ESI): m/z [1/2M+H]⁺, theoretical 957.9, found 957.8.

¹H NMR (400 MHz, DMSO-d₆) δ 8.08 (d, J=7.6 Hz, 1H), 7.95-7.85 (m, 3H), 7.82 (d, J=8.4 Hz, 3H), 6.04-5.88 (m, 6H), 5.19 (d, J=3.3 Hz, 3H), 4.95 (dd, J=11.2, 3.4 Hz, 3H), 4.57-4.46 (s, 4H), 4.04-3.98 (m, 10H), 3.94-3.81 (m, 6H), 3.80-3.72 (m, 2H), 3.62-3.59 (m, 4H), 3.58-3.51 (m, 16H), 3.50-3.43 (m, 2H), 3.42-3.39 (m, 2H), 3.38-3.34 (m, 4H), 3.34-3.26 (m, 4H), 3.26-3.10 (m, 6H), 2.26-2.21 (m, 6H), 2.15-2.10 (m, 2H), 2.08 (s, 9H), 1.97 (s, 10H), 1.87 (s, 9H), 1.75 (s, 9H), 1.48-1.35 (m, 4H), 1.24-1.18 (m, 12H).

### Example 2. Synthesis and Molecular Identification of Ligands (the Compound of Formula II, Activated Esters and Ligand Fluorescent Conjugates Thereof)

### 1. Synthesis of Intermediates NC-6, CN-16, CN-17 and GN-7

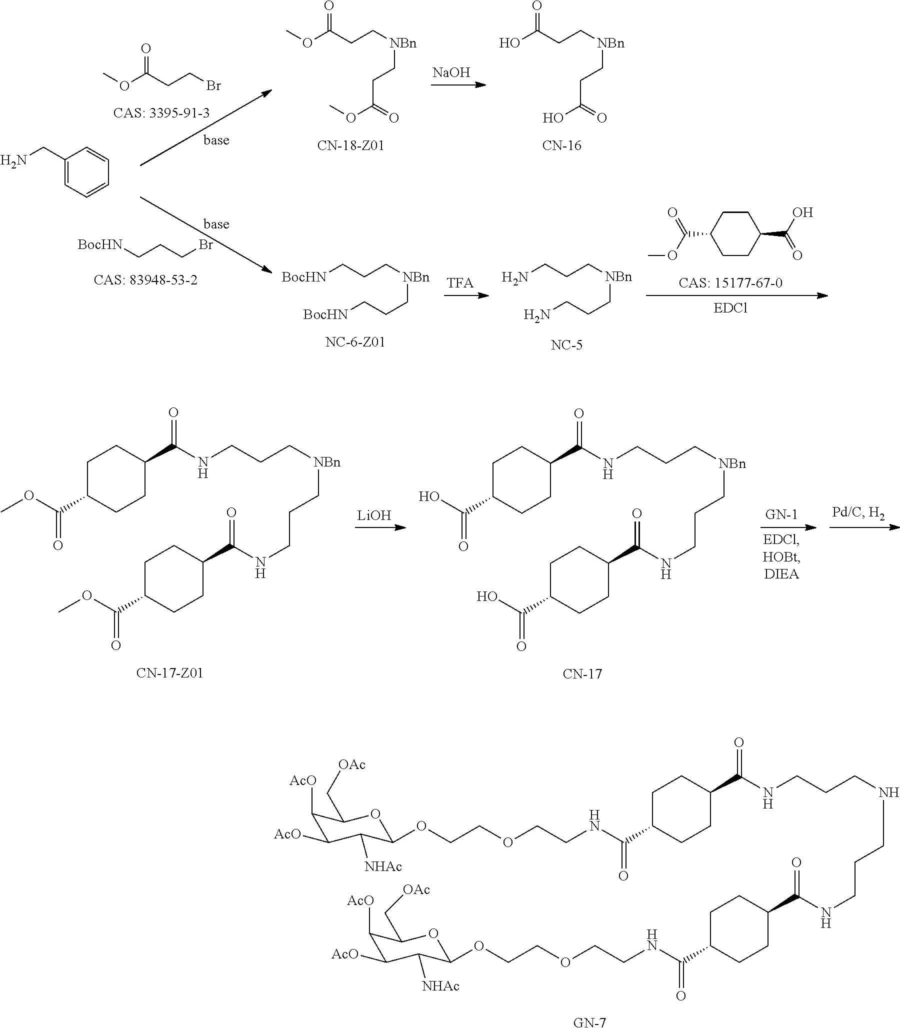

### (1) Synthesis of CN-16-Z01 (Substitution Reaction)

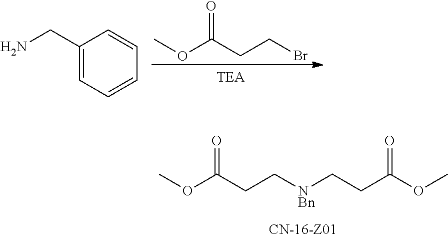

Methyl 3-bromopropionate (2.76 g, 16.5 mmol) and triethylamine (1.67 g, 16.5 mmol) were successively added to a flask containing tetrahydrofuran (10 mL), and under constant stirring and at 45° C., benzylamine (0.8 g, 7.5 mmol) was slowly dropwise added within 1 h, and the reaction was carried out for 16 h when LCMS showed that the reaction was completed. The reaction solution was diluted with pure water (20 mL) and extracted with ethyl acetate (10 mL×3). The obtained organic phases were combined, washed with 26% saturated saline solution (20 mL), dried over anhydrous sodium sulfate, filtered and concentrated under reduced pressure. The crude product was purified by silica gel column chromatography (200-300 meshes, petroleum ether/ethyl acetate, 3/2, containing 0.5% triethylamine) to obtain 1.5 g of a gray-yellow oily liquid product with a yield of 71%.

MS (ESI): m/z [M+H]⁺, theoretical 280.2, found 280.5.

### (2) Synthesis of CN-16 (Demethylation Reaction)

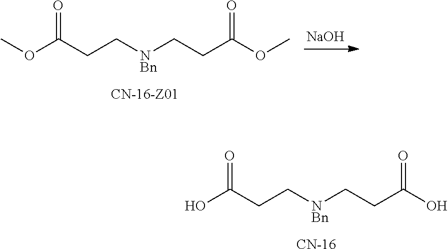

CN-16-Z01 (1.5 g, 5.4 mmol) was added to a flask containing tetrahydrofuran (6 mL) and stirred until the reaction mixture became clear, followed by addition of 4 N aqueous sodium hydroxide solution (6 mL). The reaction was carried out under stirring at 45° C. for 2 h when LCMS showed that the reaction was completed. The reaction solution was diluted with pure water (15 mL), adjusted to about pH3 with 1 N hydrochloric acid, and extracted with ethyl acetate (10 mL×4) to remove the aqueous phase. The obtained organic phases were combined, dried over anhydrous sodium sulfate, filtered and concentrated under reduced pressure to obtain 1.15 g of a light yellow solid product with a yield of 85%.

MS (ESI): m/z [M−H]⁻, theoretical 250.1, found 249.7.

### (3) Synthesis of NC-6-Z01 (Substitution Reaction)

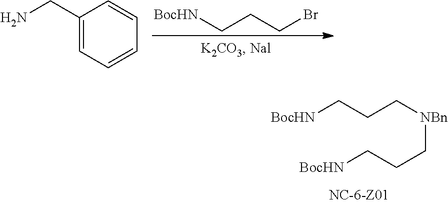 [SMILES: NCc1ccccc1 ; CC(C)(C)OC(=O)NCCCBr ; CC(C)(C)OC(=O)NCCCN(CCCNC(=O)OC(C)(C)C)Cc1ccccc1]

Benzylamine (1.1 g, 10.2 mmol), N-Boc-3-aminopropyl bromide (5 g, 21.0 mmol), potassium carbonate (2.9 g, 21.0 mmol) and sodium iodide (0.77 g, 5.1 mmol) were successively added to a flask containing acetonitrile (20 mL). Then the reaction was gradually increased to 70° C., and then carried out under stirring for 12 h when LCMS showed that the reaction was substantially completed. The reaction solution was subjected to distillation under reduced pressure to remove the solvent, and the residue was added with pure water (30 mL), mixed well, and extracted with ethyl acetate (15 mL×3). The obtained organic phases were combined, washed with 26% aqueous sodium chloride solution (20 mL), dried over anhydrous sodium sulfate, filtered and concentrated under reduced pressure. The crude product was purified by silica gel column chromatography (200-300 meshes, petroleum ether/ethyl acetate, 3/2, containing 0.5% triethylamine) to obtain 3.3 g of a brown-yellow viscous liquid product with a yield of 77%.

MS (ESI): m/z [M+H]⁺, theoretical 422.3, found 422.3.

### (4) Synthesis of NC-6 (De-Tert-Butoxycarbonylation Reaction)

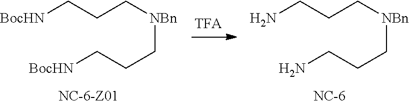

NC-6-Z01 (5.9 g, 14.0 mmol) was added to a flask containing dichloromethane (5 mL), followed by slow dropwise addition of trifluoroacetic acid (5 mL) under stirring at room temperature. The reaction was carried out under stirring for 2 h when LCMS showed that the reaction was completed. The reaction solution was subjected to distillation under reduced pressure to remove the solvent and dried under vacuum to obtain 6.2 g of a brown-yellow oily product (a TFA salt) with a yield of 98%.

MS (ESI): m/z [M+H]⁺, theoretical 222.2, found 222.1.

### (5) Synthesis of CN-17-Z01 (Amidation Condensation Reaction)

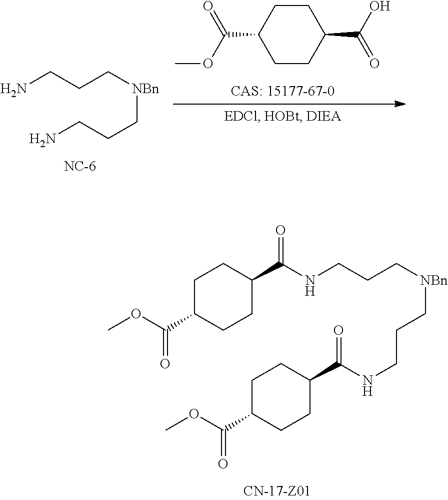

Under N₂ atmosphere, under stirring and at room temperature, NC-6 (4.85 g, 10.8 mmol), trans-1,4-cyclohexanedicarboxylic acid monomethyl ester (4.2 g, 22.8 mmol), EDCI (5.4 g, 28.1 mmol) and HOBt (3.1 g, 22.8 mmol) were successively added to a flask containing dichloromethane (25 mL), and added slowly with DIEA (4.9 g, 37.8 mmol), and the reaction was carried out for 6 h when LCMS showed that the reaction was completed. The reaction solution was added with pure water (40 mL) and extracted with dichloromethane (300 mL×3). The obtained organic phases were combined, washed with 26% aqueous sodium chloride solution (30 mL), dried over anhydrous sodium sulfate, filtered and concentrated under reduced pressure to obtain a white solid crude product. The crude product was crystallized and purified with methyl tert-butyl ether to obtain 5.0 g of a white solid product with a yield of 83%.

MS (ESI): m/z [M+Na]⁺, theoretical 558.4, found 558.5.

### (6) Synthesis of CN-17 (Demethylation Reaction)

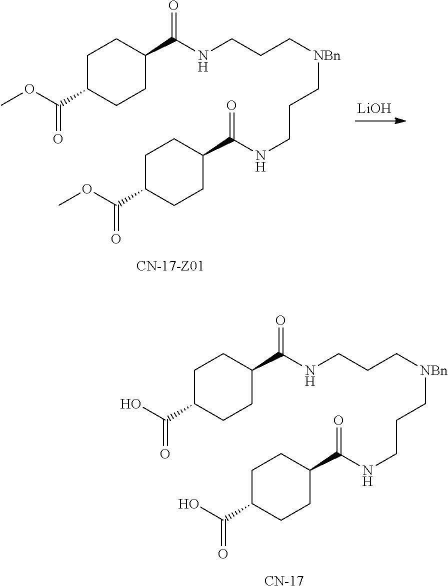

CN-17-Z01 (3.5 g, 6.3 mmol) was added to a flask containing methanol (5 mL) and stirred until the reaction mixture became clear, followed by addition of 3 N aqueous lithium hydroxide solution (6 mL). The reaction was carried out under stirring at 45° C. for 2 h when LCMS showed that the reaction was completed. The reaction solution was diluted with pure water (12 mL), adjusted to about pH2 with 1 N hydrochloric acid, and extracted with ethyl acetate (8 mL×4) to remove the aqueous phase. The obtained organic phases were combined, dried over anhydrous sodium sulfate, filtered and concentrated under reduced pressure. The crude product was crystallized with ethyl acetate to obtain 2.6 g of a light yellow solid product with a yield of 79%.

MS (ESI): m/z [M−H]⁻, theoretical 528.3, found 528.4.

### (7) Synthesis of GN-7 (Amidation Condensation and Debenzylation Reaction)

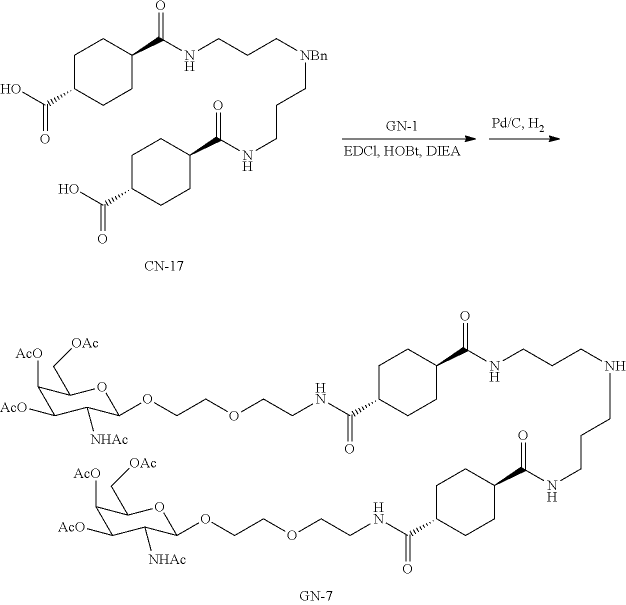

Under N₂ atmosphere, GN-1 (2.1 g, 4.76 mmol), CN-17 (1.2 g, 2.27 mmol), EDCI (0.91 g, 4.76 mmol) and HOBt (0.43 g, 3.2 mmol) were successively added to a flask containing dichloromethane (25 mL) and stirred at room temperature until the reaction mixture became clear. DIEA (0.82 g, 6.3 mmol) was added, and the reaction was carried out under stirring for 6 h when LCMS showed that the reaction was completed. The reaction solution was added with pure water (20 mL) and extracted with dichloromethane (10 mL×3). The obtained organic phases were combined, washed with 26% aqueous sodium chloride solution (20 mL), dried over anhydrous sodium sulfate, filtered and concentrated under reduced pressure. The crude product was purified by silica gel column chromatography (200-300 meshes, dichloromethane/methanol, 40/1) to obtain 2.0 g of a white solid, which was a condensed intermediate. The obtained solid was dissolved in tetrahydrofuran (12 mL), followed by successive addition of trifluoroacetic acid (0.17 g, 1.5 mmol) and Pd/C (0.4 g, 10 wt %). The reaction system was replaced with hydrogen for three times and the reaction was carried out under stirring and under hydrogen atmosphere (about 10-20 kPa higher than the standard atmospheric pressure) for 2 h when LCMS showed that the reaction was complete. The reaction mixture was filtered to remove the solid, and the filtrate was concentrated under pressure and dried under vacuum to obtain 1.89 g of a white solid product with a yield of 65.4%.

MS (ESI): m/z [M+H]⁺, theoretical 1272.6, found 1272.9.

### 2. Synthesis of Intermediates BL-6, BL-7, BL-8 and BL-9

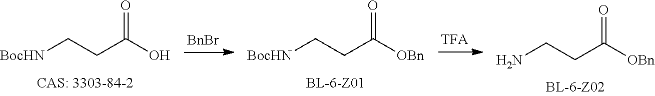

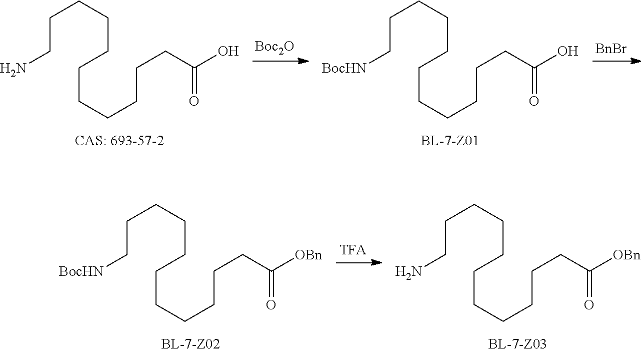 [SMILES: "Boc2O" ; NCCCCCCCCCCCC(=O)O ; CC(C)(C)OC(=O)NCCCCCCCCCCCC(=O)O ; "BnBr" ; CC(C)(C)OC(=O)NCCCCCCCCCCCC(=O)OCc1ccccc1 ; NCCCCCCCCCCCC(=O)OCc1ccccc1]

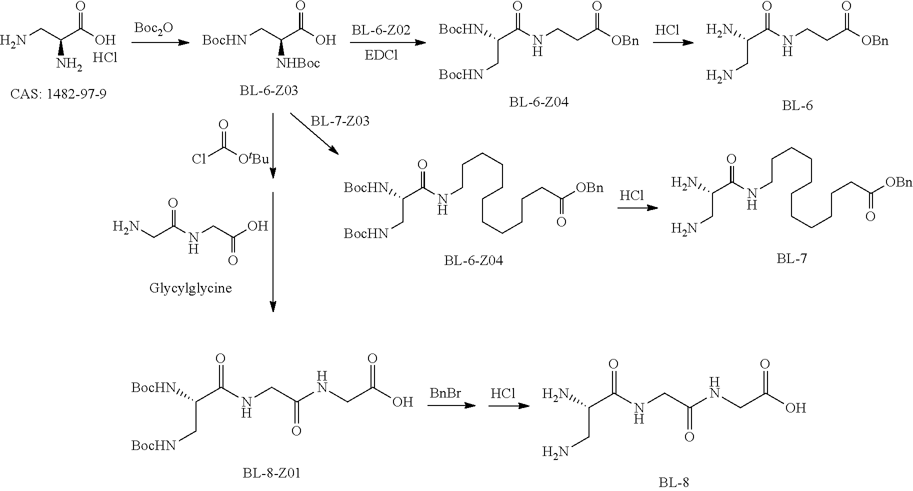

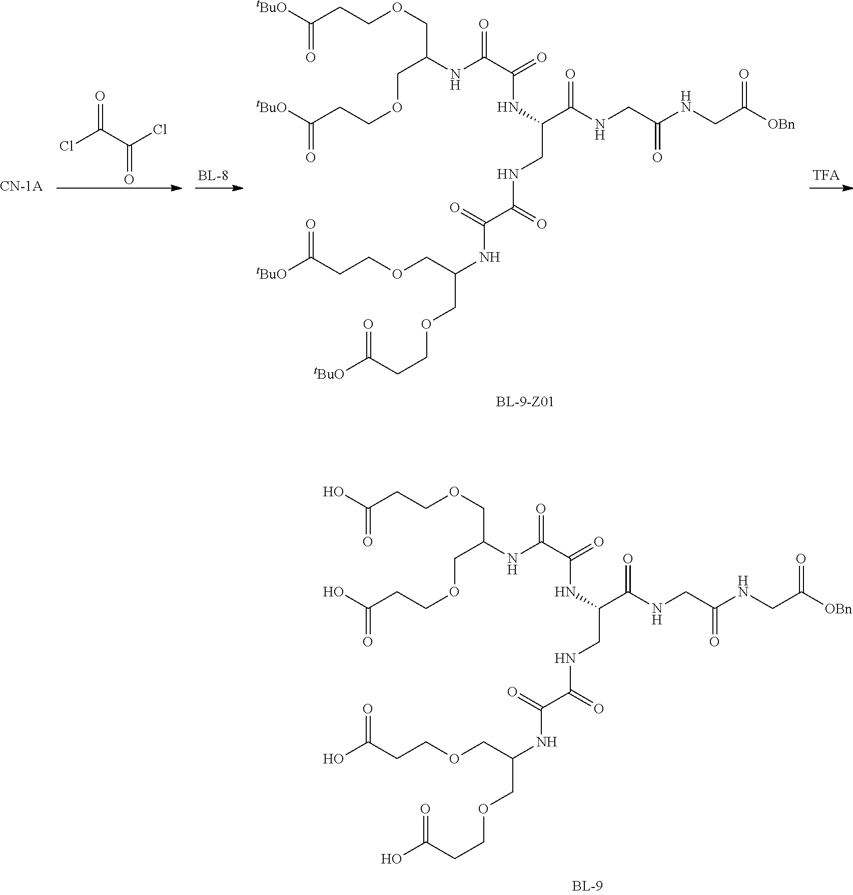 [SMILES: O=C(Cl)C(=O)Cl ; CC(C)(C)OC(=O)CCOCC(COCCC(=O)OC(C)(C)C)NC(=O)C(=O)NC[C@H](NC(=O)C(=O)NC(COCCC(=O)OC(C)(C)C)COCCC(=O)OC(C)(C)C)C(=O)NCC(=O)NCC(=O)OCc1ccccc1 ; O=C(O)CCOCC(COCCC(=O)O)NC(=O)C(=O)NC[C@H](NC(=O)C(=O)NC(COCCC(=O)O)COCCC(=O)O)C(=O)NCC(=O)NCC(=O)OCc1ccccc1]

### (1) Synthesis of BL-6-Z01 (Benzylation Protection Reaction)

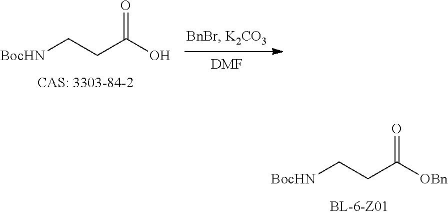

Boc-β-alanine (9.99 g, 52.8 mmol) and potassium carbonate (7.7 g, 55.5 mmol) were added to N,N-dimethylformamide (100 mL), followed by slow dropwise addition of benzyl bromide (9.5 g, 55.5 mmol) under stirring at room temperature in 20 min. The reaction was carried out under stirring for 20 h when TLC showed disappearance of raw materials (petroleum ether/ethyl acetate, 4/1, R_{f} of the product: 0.3). The reaction solution was added with pure water (300 mL) and extracted with ethyl acetate (200 mL×3). The obtained organic phases were combined, washed with 26% aqueous sodium chloride solution (300 mL), dried over anhydrous sodium sulfate, filtered and concentrated under reduced pressure to obtain a colorless liquid crude product. The crude product was purified by silica gel column chromatography (200-300 meshes, petroleum ether/ethyl acetate, 10/1 to 4/1) to obtain 12.6 g of a colorless transparent liquid with a yield of 85%.

MS (ESI): m/z [M+Na]⁺, theoretical 302.2, found 302.2.

### (2) Synthesis of BL-6-Z02 (De-Tert-Butoxycarbonylation Reaction)

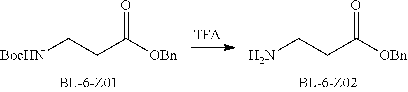 [SMILES: CC(C)(C)OC(=O)NCCC(=O)OCc1ccccc1 ; NCCC(=O)OCc1ccccc1]

BL-6-Z01 (12.6 g, 45.1 mmol) was added to dichloromethane (125 mL) and stirred at room temperature until the reaction mixture became clear, followed by slow dropwise addition of trifluoroacetic acid (25.7 g, 225 mmol). The reaction was carried out under stirring for 2 h when LCMS showed that the reaction was completed. The reaction solution was subjected to distillation under reduced pressure to remove the solvent, thereby obtaining a colorless oily crude product. The crude product was purified by silica gel column chromatography (petroleum ether/ethyl acetate, 1/1 to pure ethyl acetate) to obtain 6.8 g of a white solid (a TFA salt) with a yield of 51%.

MS (ESI): m/z [M−H]⁻, theoretical 178.1, found 178.1.

### (3) Synthesis of BL-6-Z03 (Tert-Butoxycarbonylation Protection Reaction)

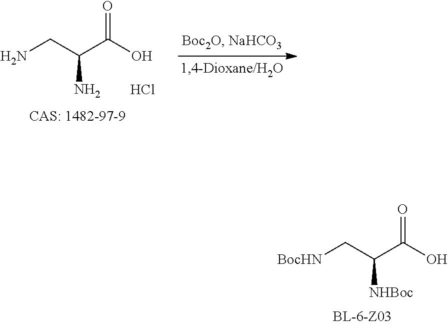 [SMILES: NC[C@H](N)C(=O)O ; Cl ; CC(C)(C)OC(=O)NC[C@H](NC(=O)OC(C)(C)C)C(=O)O]

1,4-Dioxane (100 mL) and 3-amino-L-alanine hydrochloride (10 g, 71.1 mmol) were added to a flask and stirred until the reaction mixture became clear, then added with NaHCO₃ (29.5 g, 355 mmol) and pure water (100 mL), and finally added with Boc₂O (17.8 g, 85.3 mmol) in batches. The reaction was carried out under stirring for 20 h when LCMS showed that the reaction was completed. The reaction solution was added with pure water (100 mL), adjusted to about pH4 with 20% aqueous KHSO₄ solution, and extracted with dichloromethane (200 mL×3) to remove the aqueous phase. The obtained organic phases were combined, washed with 26% aqueous sodium chloride solution (100 mL), dried over anhydrous sodium sulfate, filtered and concentrated under reduced pressure to obtain 12.6 g of a colorless liquid with a yield of 58.3%.

MS (ESI): m/z [M+Na]⁺, theoretical 327.2, found 327.2.

### (4) Synthesis of BL-6-Z04 (Amidation Condensation Reaction)

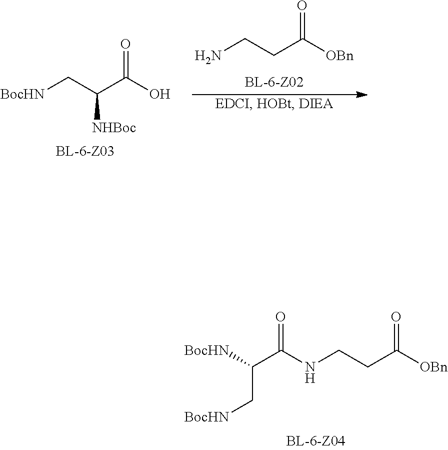 [SMILES: CC(C)(C)OC(=O)NC[C@H](NC(=O)OC(C)(C)C)C(=O)O ; NCCC(=O)OCc1ccccc1 ; "EDCI, HOBt, DIEA" ; CC(C)(C)OC(=O)NC[C@H](NC(=O)OC(C)(C)C)C(=O)NCCC(=O)OCc1ccccc1]

Under N₂ atmosphere, BL-6-Z03 (4.0 g, 13.2 mmol), BL-6-Z02 (TFA salt, 3.87 g, 13.2 mmol), EDCI (2.8 g, 14.5 mmol), HOBt (2.0 g, 14.5 mmol) and DIEA (3.4 g, 26.4 mmol) were successively added to a flask containing dichloromethane (40 mL) and stirred at room temperature for 5 h when LCMS showed that the reaction was complete. The reaction solution was added with pure water (50 mL) and extracted with dichloromethane (20 mL×3). The obtained organic phases were combined, washed with 26% aqueous sodium chloride solution (50 mL), dried over anhydrous sodium sulfate, filtered and concentrated under reduced pressure to obtain a white solid crude product. The crude product was purified by silica gel column chromatography (200-300 meshes, petroleum ether/ethyl acetate, 10/1) to obtain 3.1 g of a white solid with a yield of 51%.

MS (ESI): m/z [M+Na]⁺, theoretical 488.3, found 488.3.

### (5) Synthesis of BL-6 (De-Tert-Butoxycarbonylation Reaction)

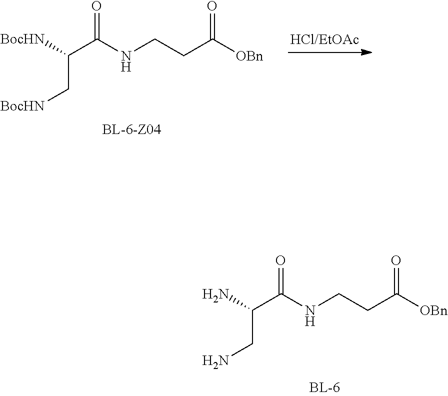 [SMILES: CC(C)(C)OC(=O)NC[C@H](NC(=O)OC(C)(C)C)C(=O)NCCC(=O)OCc1ccccc1 ; NC[C@H](N)C(=O)NCCC(=O)OCc1ccccc1]

BL-6-Z04 (1.02 g, 2.2 mmol) was dissolved in 2 M HCl/ethyl acetate solution (15 mL) and the reaction was carried out under stirring at room temperature for 2 h when LCMS showed that the reaction was complete. The solid was collected by filtration and dried under vacuum to obtain 0.5 g of a white solid (a HCl salt) with a yield of 67.4%.

MS (ESI): m/z [M+H]⁺, theoretical 266.1, found 266.1.

### (6) Synthesis of BL-7-Z02 (Benzylation Protection Reaction)

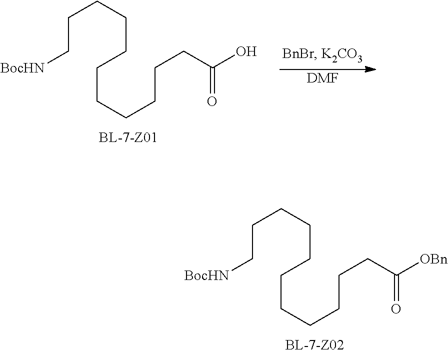

BL-7-Z01 (CAS 18934-81-1, 20 g, 63.5 mmol) and potassium carbonate (9.2 g, 66.6 mmol) were added to a flask containing N,N-dimethylformamide (120 mL), and under stirring and at room temperature, benzyl bromide (11.2 g, 66.6 mmol) was slowly dropwise added within 20 min, and then the reaction was carried out for 20 h when TLC showed that the conversion of the raw materials was complete (petroleum ether/ethyl acetate, 4/1, R_{f} of the product: 0.3). The reaction solution was added with pure water (150 mL) and extracted with ethyl acetate (50 mL×3). The obtained organic phases were combined, washed with 26% aqueous sodium chloride solution (80 mL), dried over anhydrous sodium sulfate, filtered and concentrated under reduced pressure to obtain a colorless liquid crude product. The crude product was purified by silica gel column chromatography (200-300 meshes, petroleum ether/ethyl acetate, 10/1 to 7/1) to obtain 23 g of a white solid product with a yield of 89%.

MS (ESI): m/z [M+H]⁺, theoretical 406.3, found 406.3.

### (7) Synthesis of BL-7-Z03 (De-Tert-Butoxycarbonylation Reaction)

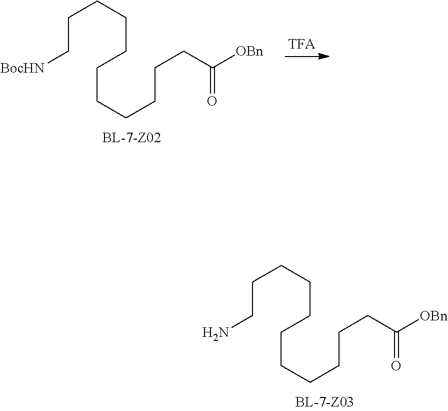 [SMILES: CC(C)(C)OC(=O)NCCCCCCCCCCCC(=O)OCc1ccccc1 ; NCCCCCCCCCCCC(=O)OCc1ccccc1]

BL-7-Z02 (23 g, 56.8 mmol) was added to a flask containing dichloromethane (100 mL) and stirred at room temperature until the reaction mixture became clear, followed by slow dropwise addition of trifluoroacetic acid (32.3 g, 284 mmol). Then the reaction was carried out for 2 h when LCMS showed that the reaction was complete. The reaction solution was subjected to distillation under reduced pressure to remove the solvent, thereby obtaining a light yellow oily crude product. The crude product was crystallized (ethyl acetate/petroleum ether, 1/10) to obtain 22.4 g of a white solid product (a TFA salt) with a yield of 94%.

MS (ESI): m/z [M+H]⁺, theoretical 306.2, found 306.2.

### (8) Synthesis of BL-7-Z04 (Amidation Condensation Reaction)

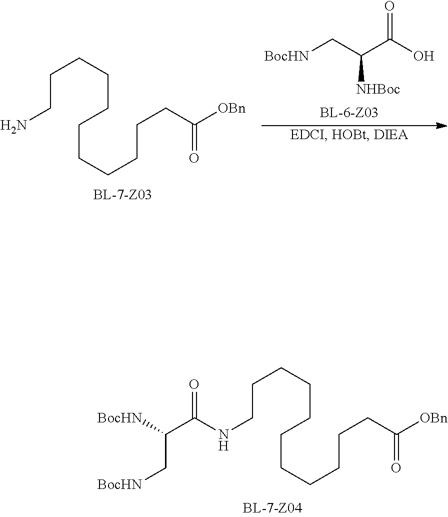 [SMILES: NCCCCCCCCCCCC(=O)OCc1ccccc1 ; "EDCI, HOBt, DIEA" ; CC(C)(C)OC(=O)NC[C@H](NC(=O)OC(C)(C)C)C(=O)O ; CC(C)(C)OC(=O)NC[C@H](NC(=O)OC(C)(C)C)C(=O)NCCCCCCCCCCCC(=O)OCc1ccccc1]

Under N₂ atmosphere, BL-6-Z03 (2.0 g, 6.6 mmol), BL-7-Z03 (a TFA salt, 2.77 g, 6.6 mmol), EDCI (1.38 g, 7.2 mmol), HOBt (973 mg, 7.2 mmol) and DIEA (1.7 g, 13.2 mmol) were successively added to a flask containing dichloromethane (20 mL) and the reaction was carried out under stirring at room temperature for 3 h when LCMS showed that the reaction was complete. The reaction solution was added with pure water (50 mL) and extracted with dichloromethane (15 mL×3). The obtained organic phases were combined, washed with 26% aqueous sodium chloride solution (40 mL), dried over anhydrous sodium sulfate, filtered and concentrated under reduced pressure to obtain a white solid crude product. The crude product was purified by silica gel column chromatography (200-300 meshes, petroleum ether/ethyl acetate, 10/1) to obtain 2.1 g of a white solid, which was recrystallized (dichloromethane/petroleum ether, 1/10) to obtain 2.46 g of a white solid product with a yield of 63%.

MS (ESI): m/z [M+H]⁺, theoretical 592.4, found 592.4.

### (9) Synthesis of BL-7 (De-Tert-Butoxycarbonylation Reaction)

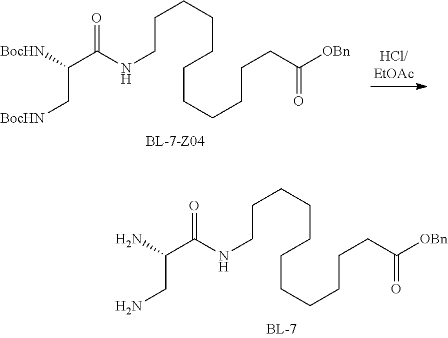 [SMILES: CC(C)(C)OC(=O)NC[C@H](NC(=O)OC(C)(C)C)C(=O)NCCCCCCCCCCCC(=O)OCc1ccccc1 ; NC[C@H](N)C(=O)NCCCCCCCCCCCC(=O)OCc1ccccc1]

BL-7-Z04 (2.4 g, 4.06 mmol) was dissolved in 2 M HCl/ethyl acetate solution (30 mL) and the reaction was carried out under stirring at room temperature for 2 h when LCMS showed that the reaction was complete. The solid was collected by filtration and dried under vacuum to obtain 1.58 g of a white solid (a HCl salt) with a yield of 84%.

MS (ESI): m/z [M+H]⁺, theoretical 392.3, found 392.3.

¹H NMR (400 MHz, DMSO-d₆) δ 8.90 (t, J=5.4 Hz, 1H), 8.70 (br s, 6H), 7.40-7.28 (m, 5H), 5.07 (s, 2H), 4.23 (t, J=5.6 Hz, 1H), 3.25 (d, J=5.6 Hz, 2H), 3.20-2.99 (m, 2H), 2.33 (t, J=7.3 Hz, 2H), 1.56-1.40 (m, 4H), 1.34-1.18 (m, 14H).

### (10) Synthesis of BL-8-Z01 (Amidation Condensation Reaction)

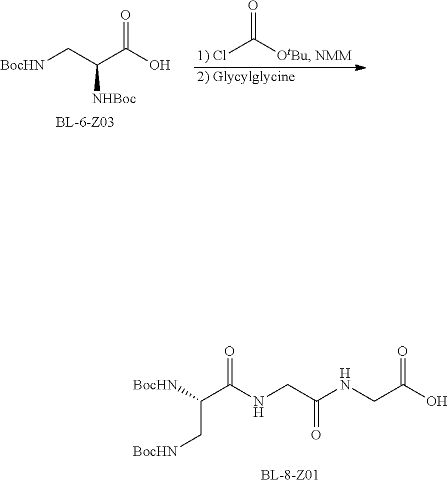

BL-6-Z03 (2.0 g, 6.57 mmol) and N-methylmorpholine (1.3 g, 13.1 mmol) were successively added to a flask containing tetrahydrofuran (12 mL), followed by slow dropwise addition of isobutyl chloroformate (0.9 g, 6.57 mmol) under N₂ atmosphere and in an ice water bath of 0-5° C. The reaction was carried out under stirring for 1 h with the temperature maintained unchanged, followed by addition of glycylglycine (CAS 556-50-3, 2.0 g, 6.57 mmol) in batches. The reaction was allowed to naturally increase to the room temperature and then carried out for 4 h when LCMS showed that the reaction was completed. The reaction solution was adjusted to about pH7 with 0.5 N hydrochloric acid and extracted with ethyl acetate (15 mL×3). The obtained organic phases were combined, washed with 26% aqueous sodium chloride solution (25 mL), dried over anhydrous sodium sulfate, filtered and concentrated under reduced pressure to obtain 2.8 g of a light yellow solid product, which could be used directly in subsequent reaction without purification.

MS (ESI): m/z [M+H]⁺, theoretical 419.2, found 419.2.

### (11) Synthesis of BL-8 (De-Tert-Butoxycarbonylation Reaction)

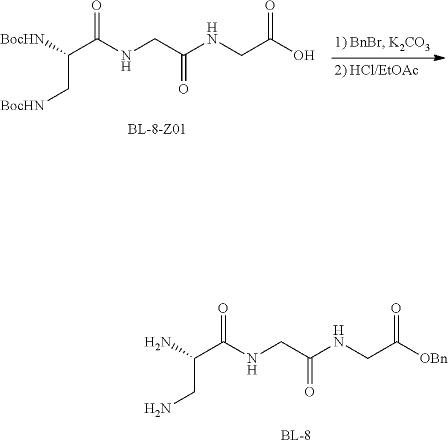 [SMILES: CC(C)(C)OC(=O)NC[C@H](NC(=O)OC(C)(C)C)C(=O)NCC(=O)NCC(=O)O ; NC[C@H](N)C(=O)NCC(=O)NCC(=O)OCc1ccccc1]

BL-8-Z01 (2.1 g, 5.0 mmol) and potassium carbonate (0.9 g, 6.52 mmol) were successively added to a flask containing acetonitrile (14 mL) and stirred at room temperature for 0.5 h. The reaction was then dropwise added with benzyl bromide (1.03 g, 6.0 mmol) within 15 min and the reaction was carried out under stirring for 16 h when TLC showed that the conversion of the raw materials was complete (adjusted to about pH7, petroleum ether/ethyl acetate, 2/1, R_{f} of the product: 0.5). The reaction solution was subjected to distillation under reduced pressure to remove the solvent, and the residue was added with pure water (20 mL), mixed well, and extracted with ethyl acetate (15 mL×3). The obtained organic phases were combined, washed with 26% aqueous sodium chloride solution (25 mL), dried over anhydrous sodium sulfate, filtered and concentrated under reduced pressure to obtain a light yellow solid product. The product was purified by silica gel column chromatography (200-300 meshes, petroleum ether/ethyl acetate, 2/1) to obtain 2.2 g of a light yellow solid, which was a carboxybenzyl ester intermediate. The obtained solid was dissolved in 2 M HCl/ethyl acetate solution (9 mL) and the reaction was carried out under stirring for 2 h when LCMS showed that the reaction was complete. The reaction solution was subjected to distillation under reduced pressure to remove the solvent, and the crude product was crystallized and purified with methyl tert-butyl ether to obtain 1.43 g of a light yellow solid product (a HCl salt) with a yield of 75%.

MS (ESI): m/z [M+H]⁺, theoretical 309.2, found 309.1.

### (12) Synthesis of BL-9-Z01 (Continuous Amidation Condensation Reaction)

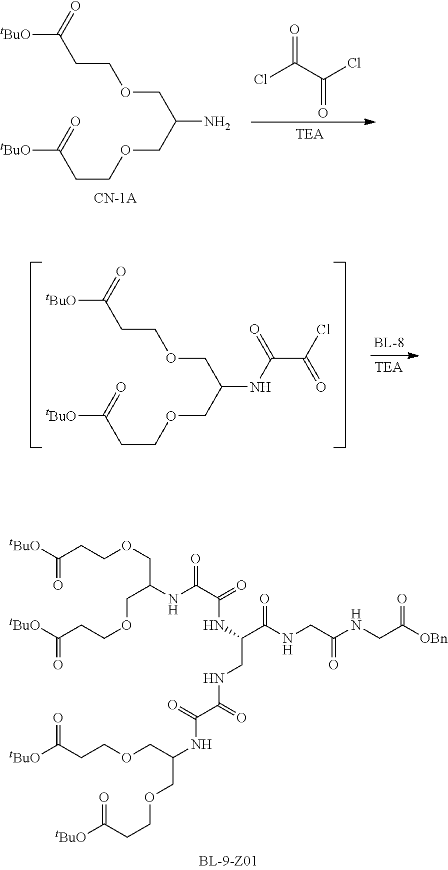

Oxalyl chloride (0.58 g, 4.53 mmol) was added to a flask containing dichloromethane (4 mL), and under stirring, under N₂ atmosphere and in an ice bath of 0-5° C., the reaction was dropwise added with CN-1A (1.5 g, 4.32 mmol) and triethylamine (0.44 g, 4.32 mL) homogenously dissolved in dichloromethane (8 mL) within 0.5 h, allowed to naturally increase to room temperature and then carried out under stirring for 1 h. The reaction was then slowly added with BL-8 (a HCl salt, 0.82 g, 2.16 mmol) and triethylamine (0.44 g, 4.32 mL) dissolved in dichloromethane (6 mL) and was carried out under stirring for 1 h when LCMS showed that the reaction was complete. The reaction solution was added with pure water (20 mL) and extracted with dichloromethane (10 mL×2). The obtained organic phases were combined, washed with 26% aqueous sodium chloride solution (25 mL), dried over anhydrous sodium sulfate, filtered and concentrated under reduced pressure. The crude product was purified by silica gel column chromatography (200-300 meshes, petroleum ether/ethyl acetate, 1/1) to obtain 1.49 g of a light yellow solid product with a yield of 62%.

MS (ESI): m/z [M+Na]⁺, theoretical 1133.6, found 1133.4.

### (13) Synthesis of BL-9 (De-Tert-Butylation Reaction)

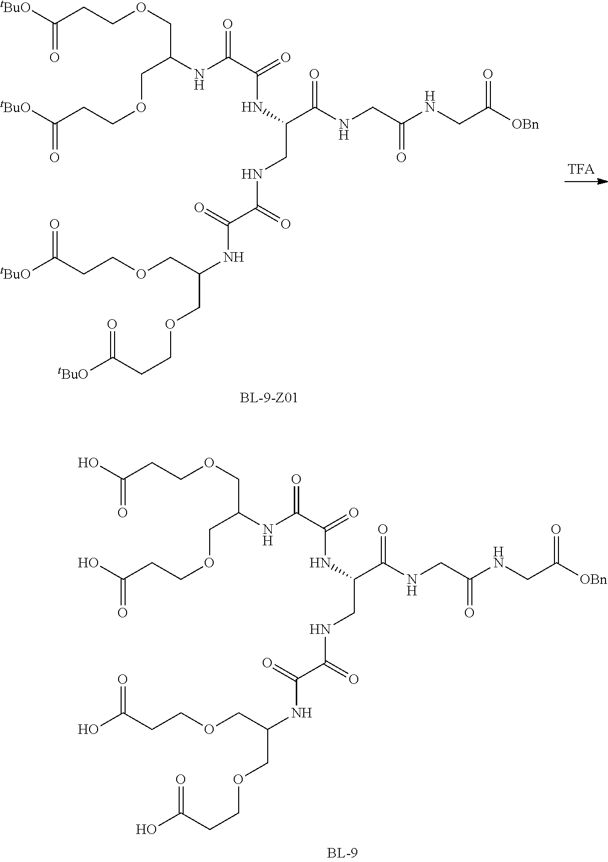 [SMILES: CC(C)(C)OC(=O)CCOCC(COCCC(=O)OC(C)(C)C)NC(=O)C(=O)NC[C@H](NC(=O)C(=O)NC(COCCC(=O)OC(C)(C)C)COCCC(=O)OC(C)(C)C)C(=O)NCC(=O)NCC(=O)OCc1ccccc1 ; O=C(O)CCOCC(COCCC(=O)O)NC(=O)C(=O)NC[C@H](NC(=O)C(=O)NC(COCCC(=O)O)COCCC(=O)O)C(=O)NCC(=O)NCC(=O)OCc1ccccc1]

BL-9-Z01 (1.49 g, 1.34 mmol) was added to a flask containing dichloromethane (8 mL) and stirred at room temperature until the reaction mixture became clear. The reaction was then slowly added with trifluoroacetic acid (8 mL) and carried out under stirring for 2 h when LCMS showed that the reaction was complete. The reaction solution was subjected to distillation under reduced pressure to remove the solvent, and the residue was dissolved with acetonitrile/water (15 mL, 1/4) and freeze-dried to obtain 1.34 g of a light yellow oily liquid product with a yield of 113% (some residual TFA remained).

MS (ESI): m/z [M−H]⁻, theoretical 885.3, found 885.1.

### 3. Synthesis of Intermediate GN-8

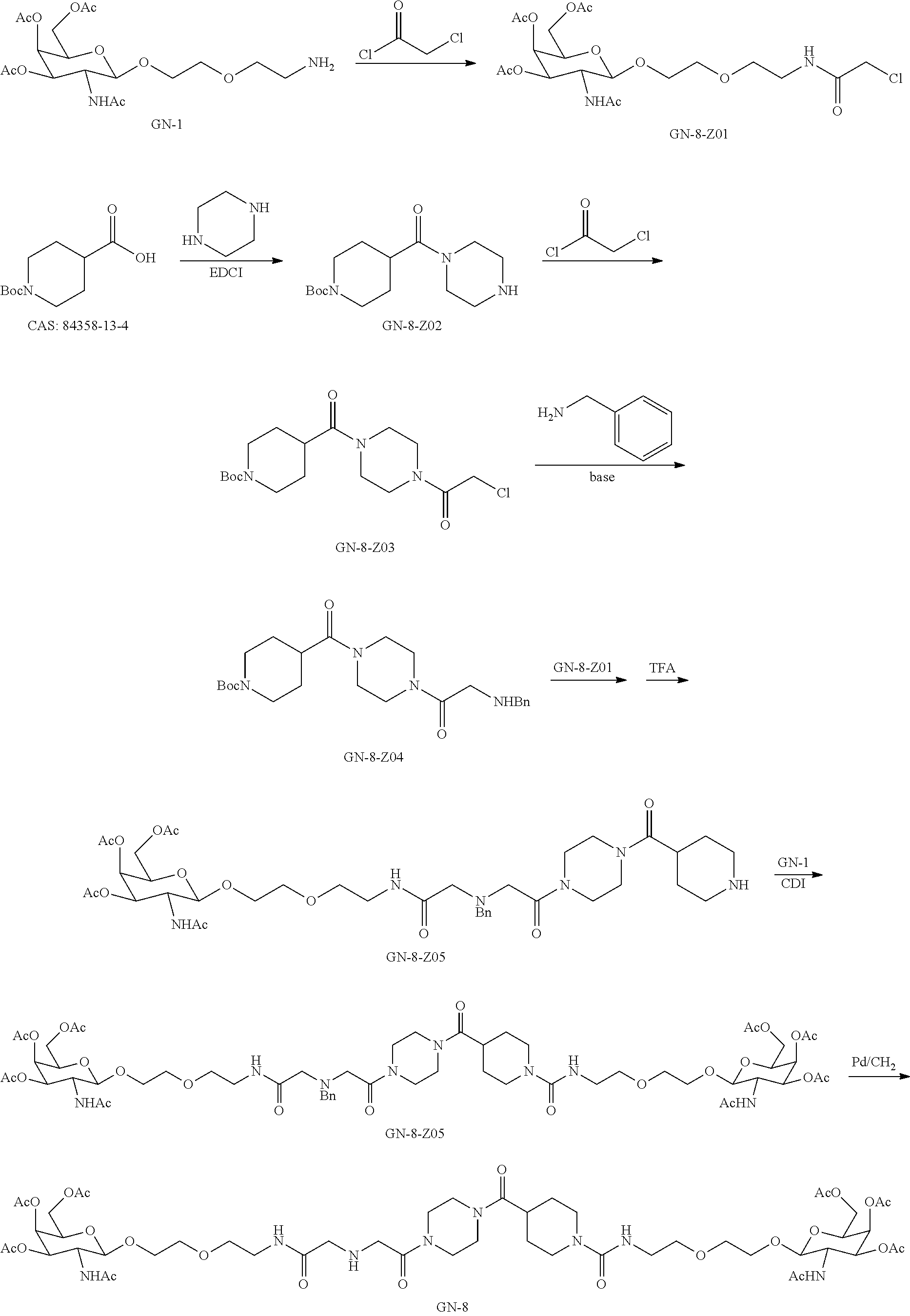

Synthesis of GN-8: (1) GN-8-Z01 was obtained by subjecting GN-1 to reaction with chloroacetyl chloride; (2) GN-8-Z02 was obtained by subjecting N-Boc-piperidine-4-formic acid (CAS: 84358-13-4) to amidation condensation reaction with piperazine; (3) GN-8-Z03 was obtained by subjecting GN-8-Z02 to reaction with chloroacetyl chloride, and GN-8-Z04 was obtained by subjecting GN-8-Z03 to substitution reaction with benzylamine; (4) GN-8-Z05 was obtained by subjecting GN-8-Z04 to substitution reaction with GN-8-Z01 and then subjecting the resultant product to de-tert-butoxycarbonylation reaction; (5) GN-8-Z06 was obtained by subjecting GN-8-Z05 to urea condensation reaction with GN-1; (6) GN-8 was obtained by subjecting GN-8-Z06 to debenzylation reaction.

### 4. Synthesis of TEC01A (a Stereoisomeric Amine with Peracetylated Glycosyl Groups of Ligand TEC01A)

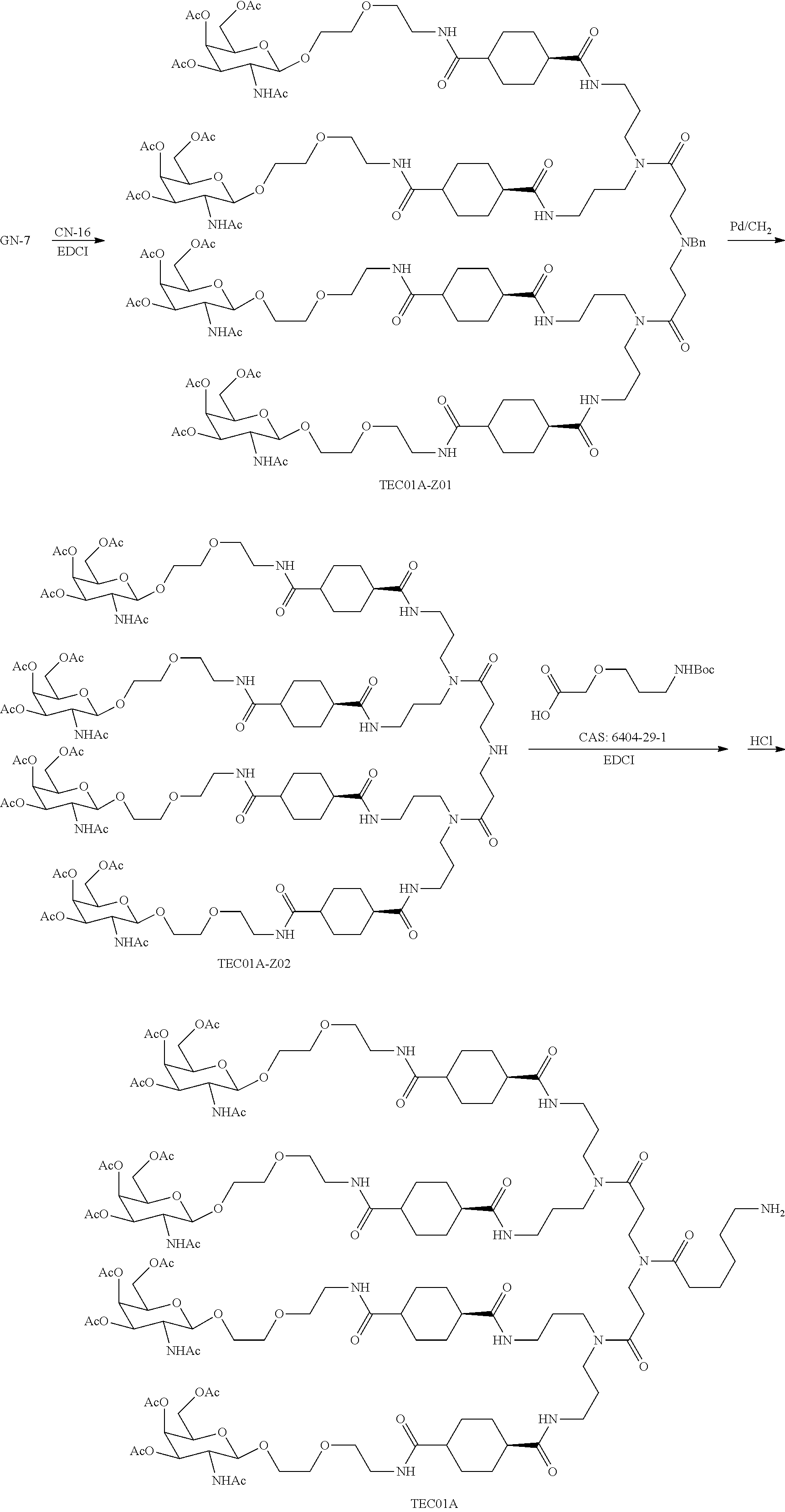

### (1) Synthesis of TEC01A-Z01 (Amidation Condensation Reaction)

Under N₂ atmosphere, GN-7 (1.85 g, 1.45 mmol), CN-16 (0.17 g, 0.69 mmol), EDCI (0.32 g, 1.66 mmol) and HOBt (0.19 g, 1.38 mmol) were successively added to a flask containing dichloromethane (10 mL) and stirred at room temperature until the reaction mixture became clear. The reaction was then added with DIEA (0.36 g, 2.76 mmol) and carried out under stirring for 6 h when LCMS showed that the reaction was complete. The reaction solution was added with pure water (15 mL) and extracted with dichloromethane (10 mL×3). The obtained organic phases were combined, washed with 26% aqueous sodium chloride solution (15 mL), dried over anhydrous sodium sulfate, filtered and concentrated under reduced pressure to obtain a solid crude product. The crude product was purified by silica gel column chromatography (200-300 meshes, dichloromethane/methanol, 19/1) to obtain 1.35 g of a white solid with a yield of 71%.

MS (ESI): m/z [1/2M+H]⁺, theoretical 1380.7, found 1380.6.

### (2) Synthesis of TEC01A-Z02 (Debenzylation Reaction)

TEC01A-Z01 (0.7 g, 0.25 mmol) was dissolved in tetrahydrofuran (6 mL), and successively added with trifluoroacetic acid (0.029 g, 0.25 mmol) and Pd/C (0.07 g, 10 wt %). The reaction system was replaced with hydrogen for three times and the reaction was carried out under stirring and under hydrogen atmosphere (about 10-20 kPa higher than the standard atmospheric pressure) for 1.5 h when LCMS showed that the reaction was complete. The reaction mixture was filtered to remove the solid, concentrated under reduced pressure and dried under vacuum to obtain 0.6 g of a light yellow solid product with a yield of 90.5%.

MS (ESI): m/z [1/2M+Na]⁺, theoretical 1357.6, found 1357.4.

### (3) Synthesis of TEC01A (Amidation Condensation and De-Tert-Butoxycarbonylation Reaction)

Under N₂ atmosphere, TEC01A-Z02 (0.5 g, 0.187 mmol), N-Boc-6-aminocaproic acid (CAS 6404-29-1, 0.046 g, 0.2 mmol), EDCI (0.046 g, 0.24 mmol), HOBt (0.025 g, 0.187 mmol) and DIEA (0.048 g, 0.37 mmol) were successively added to a flask containing dichloromethane (6 mL) and the reaction was carried out under stirring at room temperature for 3 h when LCMS showed that the reaction was complete. The reaction solution was added with pure water (18 mL) and extracted with dichloromethane (12 mL×3). The obtained organic phases were combined, washed with 26% aqueous sodium chloride solution (20 mL), dried over anhydrous sodium sulfate, filtered and concentrated under reduced pressure to obtain a solid crude product. The crude product was purified by silica gel column chromatography (200-300 meshes, dichloromethane/methanol, 20/1) to obtain 0.43 g of a white solid, which was a condensed intermediate. The obtained solid was dissolved in ethyl acetate (2 mL), slowly added with 2 M HCl/ethyl acetate solution (1 mL) under stirring, and then the reaction was carried out under stirring for 1 h when LCMS showed that the reaction was complete. The reaction solution was subjected to distillation under reduced pressure to remove the solvent, and the crude product was crystallized and purified with methyl tert-butyl ether to obtain 0.43 g of a light yellow solid product with a yield of 81%, which is a HCl salt.

MS (ESI): m/z [M+H]⁺, theoretical 1392.2, found 1392.4.

### 5. Synthesis of TEC02A (a Stereoisomeric Carboxylic Acid with Peracetylated Glycosyl Groups of Ligand TEC02), TEC02A-Pfp (an Activated Ester) and TEC02A-Cy5 (a Receptor-Targeting Fluorescent Conjugate)

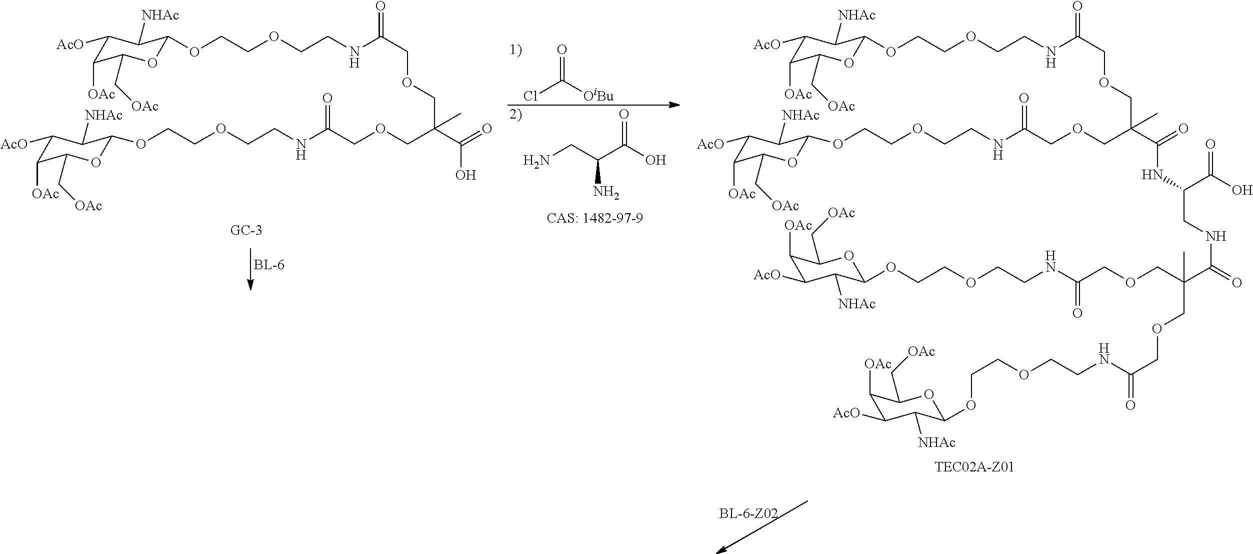

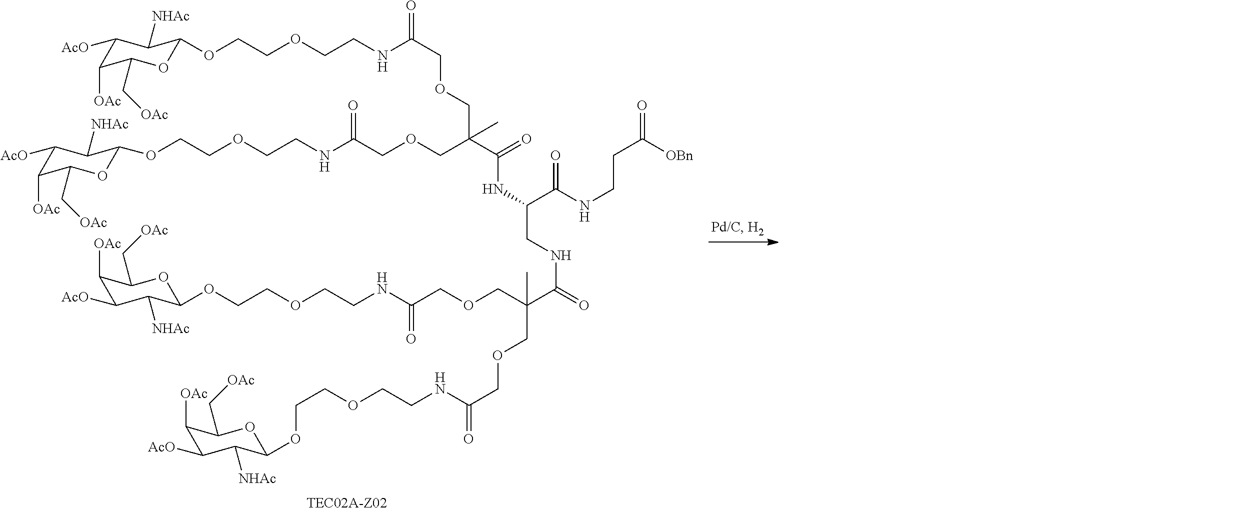 [SMILES: CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)NC[C@H](NC(=O)C(C)(COCC(=O)NCCOCCOC2OC(COC(C)=O)C(OC(C)=O)C(OC(C)=O)C2NC(C)=O)COCC(=O)NCCOCCOC2OC(COC(C)=O)C(OC(C)=O)C(OC(C)=O)C2NC(C)=O)C(=O)NCCC(=O)OCc2ccccc2)OC(COC(C)=O)C(OC(C)=O)C1OC(C)=O]

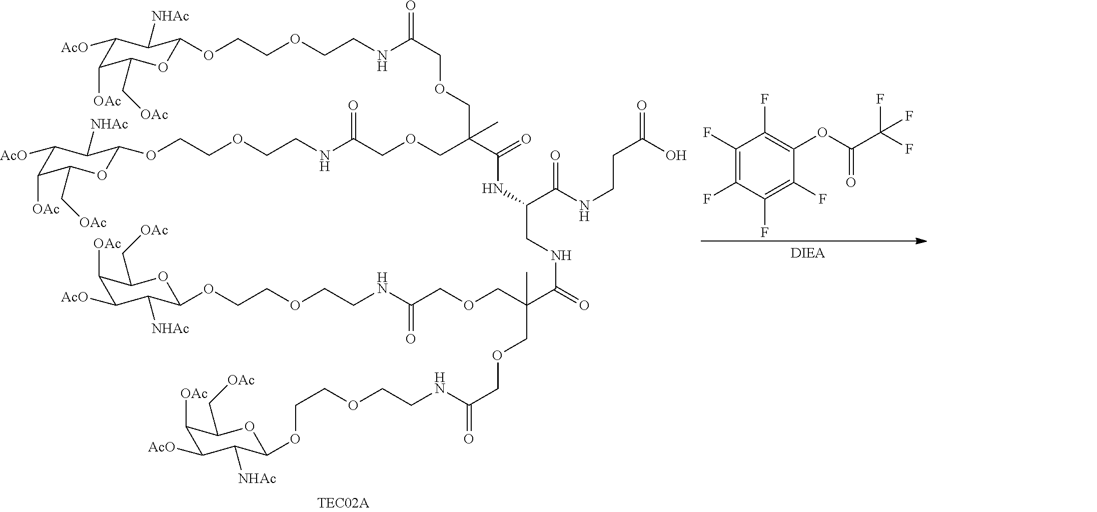

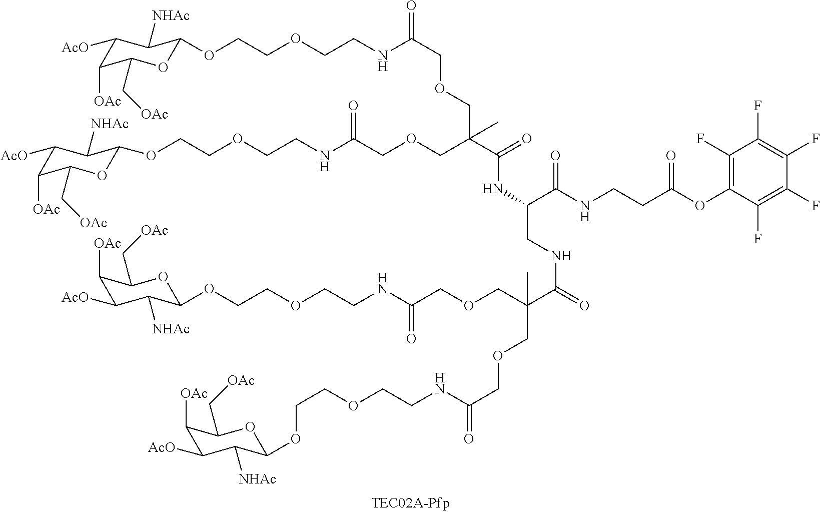 [SMILES: CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)NC[C@H](NC(=O)C(C)(COCC(=O)NCCOCCOC2OC(COC(C)=O)C(OC(C)=O)C(OC(C)=O)C2NC(C)=O)COCC(=O)NCCOCCOC2OC(COC(C)=O)C(OC(C)=O)C(OC(C)=O)C2NC(C)=O)C(=O)NCCC(=O)Oc2c(F)c(F)c(F)c(F)c2F)OC(COC(C)=O)C(OC(C)=O)C1OC(C)=O]

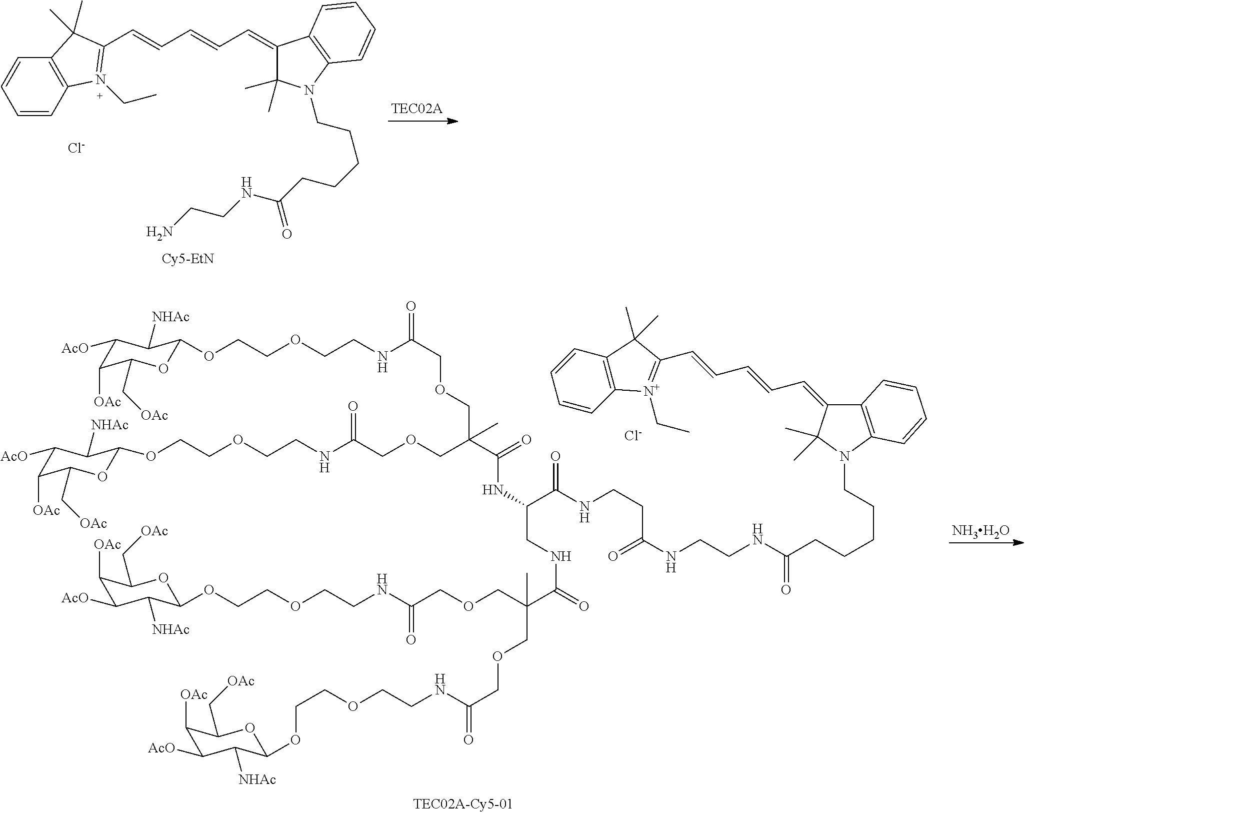

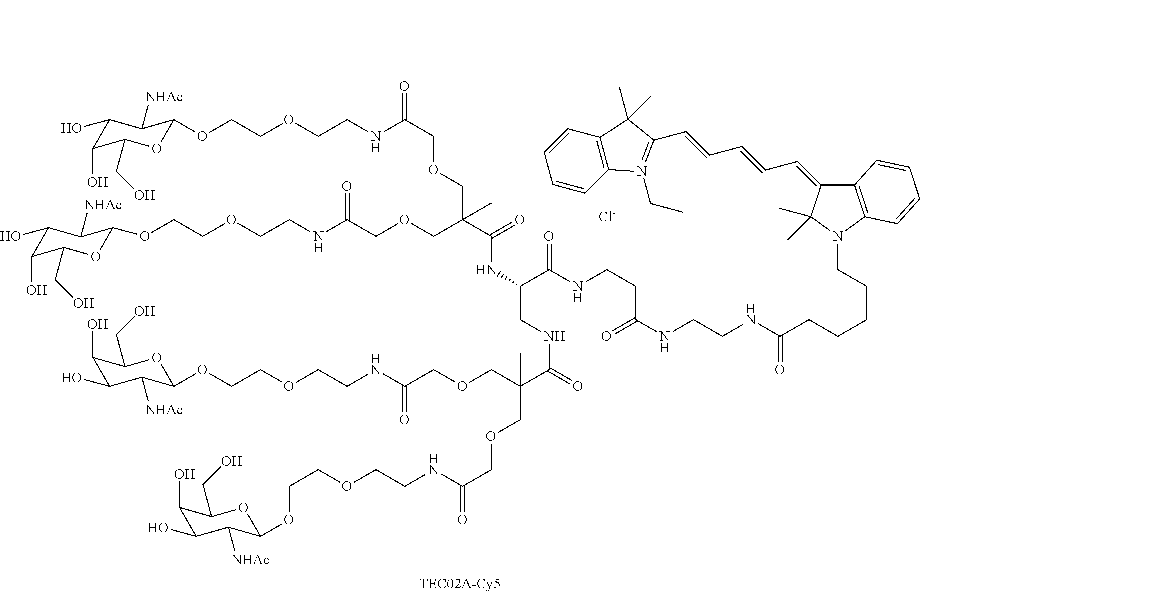 [SMILES: CC[N+]1=C(/C=C/C=C/C=C2/c3ccccc3N(CCCCCC(=O)NCCNC(=O)CCNC(=O)[C@H](CNC(=O)C(C)(COCC(=O)NCCOCCOC3OC(CO)C(O)C(O)C3NC(C)=O)COCC(=O)NCCOCCOC3OC(CO)C(O)C(O)C3NC(C)=O)NC(=O)C(C)(COCC(=O)NCCOCCOC3OC(CO)C(O)C(O)C3NC(C)=O)COCC(=O)NCCOCCOC3OC(CO)C(O)C(O)C3NC(C)=O)C2(C)C)C(C)(C)c2ccccc21 ; [Cl-]]

### (1) Synthesis of TEC02A-Z01 (Amidation Condensation Reaction)

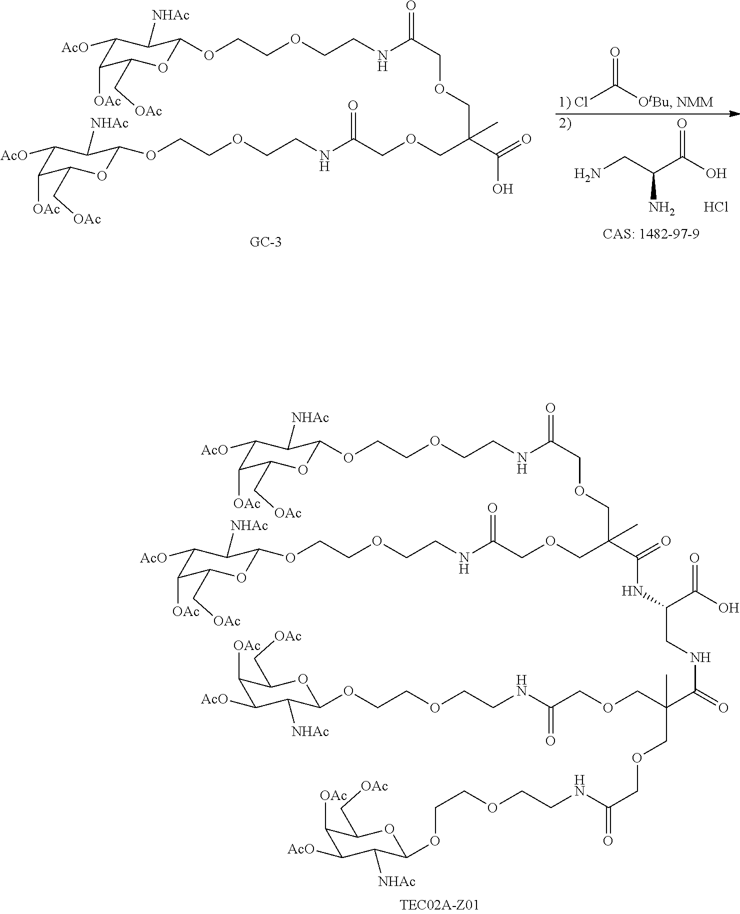

GC-3 (1.2 g, 1.1 mmol) and N-methylmorpholine (0.22 g, 2.2 mmol) were successively added to a flask containing tetrahydrofuran (8 mL), followed by slow dropwise addition of isobutyl chloroformate (0.157 g, 1.15 mmol) under N₂ atmosphere and in an ice bath of 0-5° C. The reaction was carried out under stirring for 1 h with the temperature maintained unchanged, followed by gradual addition of 3-amino-L-alanine hydrochloride (0.077 g, 0.55 mmol). The reaction was allowed to naturally increase to room temperature, and then carried out for 2 h when LCMS showed that the reaction was complete. The reaction solution was diluted with pure water (20 mL), adjusted to about pH4 with 20% aqueous KHSO₄ solution, and extracted with ethyl acetate (11 mL×3). The obtained organic phases were combined, washed with 26% aqueous sodium chloride solution (20 mL), dried over anhydrous sodium sulfate, filtered and concentrated under reduced pressure to obtain 1.07 g of a light yellow solid product with a yield of 87%.

MS (ESI): m/z [1/2M+H]⁺, theoretical 1118.0, found 1117.9.

### (2) Synthesis of TEC02A-Z02 (Amidation Condensation Reaction)

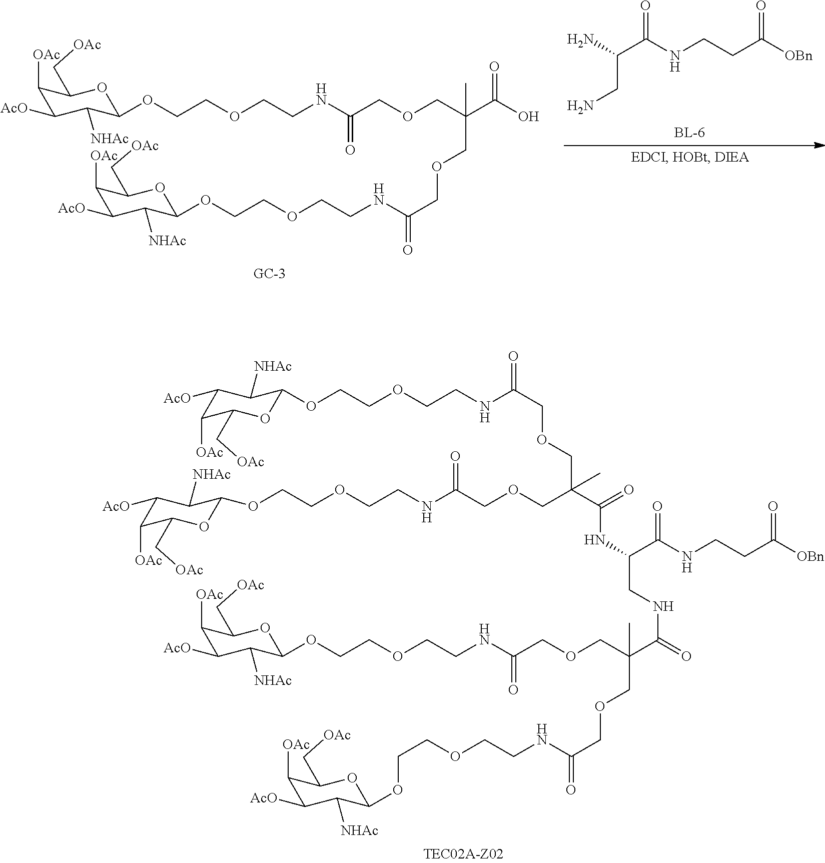 [SMILES: CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)O)OC(COC(C)=O)C(OC(C)=O)C1OC(C)=O ; NC[C@H](N)C(=O)NCCC(=O)OCc1ccccc1 ; CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)NC[C@H](NC(=O)C(C)(COCC(=O)NCCOCCOC2OC(COC(C)=O)C(OC(C)=O)C(OC(C)=O)C2NC(C)=O)COCC(=O)NCCOCCOC2OC(COC(C)=O)C(OC(C)=O)C(OC(C)=O)C2NC(C)=O)C(=O)NCCC(=O)OCc2ccccc2)OC(COC(C)=O)C(OC(C)=O)C1OC(C)=O]

Under N₂ atmosphere, GC-3 (1.8 g, 1.6 mmol), BL-6 (a HCl salt, 0.27 g, 0.8 mmol), EDCI (0.5 g, 2.4 mmol) and HOBt (0.2 g, 1.6 mmol) were successively added to a flask containing dichloromethane (20 mL) and stirred at room temperature until the reaction mixture became clear. The reaction was then added with DIEA (0.4 g, 3.2 mmol) and carried out under stirring for 6 h when LCMS showed that the reaction was complete. The reaction solution was added with pure water (20 mL) and extracted with dichloromethane (10 mL×3). The obtained organic phases were combined, washed with 26% aqueous sodium chloride solution (10 mL), dried over anhydrous sodium sulfate, filtered and concentrated under reduced pressure to obtain 3.8 g of a white solid crude product. The crude product was purified by silica gel column chromatography (200-300 meshes, dichloromethane/methanol, 49/1 to 19/1) to obtain 1.5 g white solid with a yield of 78.3%.

MS (ESI): m/z [1/2M+H]⁺, theoretical 1198.5, found 1198.1.

¹H NMR (400 MHz, CDCl₃) δ 8.80-8.01 (m, 2H), 7.79-7.45 (m, 5H), 7.40-7.27 (m, 6H), 7.05-6.40 (m, 3H), 5.38-5.30 (m, 4H), 5.29-5.19 (m, 3H), 5.11-5.05 (m, 3H), 4.78-4.72 (m, 3H), 4.60-4.49 (m, 1H), 4.20-4.06 (m, 9H), 4.06-3.95 (m, 11H), 3.96-3.86 (m, 9H), 3.76-3.68 (m, 6H), 3.66-3.29 (m, 38H), 2.59-2.54 (m, 2H), 2.13 (s, 12H), 2.02 (s, 12H), 1.96 (s, 12H), 1.94 (s, 12H), 1.20 (br s, 3H), 1.15 (br s, 3H).

¹H NMR (400 MHz, DMSO-d₆) δ 8.16 (br s, 1H), 7.99 (br s, 1H), 7.95-7.90 (m), 1H), 7.90-7.79 (m, 8H), 7.42-7.27 (m, 5H), 5.21 (d, J=3.4 Hz, 4H), 5.07 (s, 2H), 4.98 (dd, J=11.2, 3.4 Hz, 4H), 4.55 (d, J=8.5 Hz, 4H), 4.29 (q, J=6.7 Hz, 1H), 4.05-3.98 (m, 12H), 3.93-3.84 (m, 12H), 3.81-3.73 (m, 4H), 3.61-3.46 (m, 22H), 3.44-3.39 (m, 10H), 3.29-3.22 (m, 10H), 2.55-2.51 (m, 2H), 2.10 (s, 12H), 1.99 (s, 12H), 1.89 (s, 12H), 1.77 (s, 12H), 1.12 (s, 3H), 1.08 (s, 3H).

TEC02A-Z02 was synthesized by subjecting TEC02A-Z01 and BL-6-Z02 as starting materials to similar amidation condensation reaction steps for synthesis of BL-6-Z04.

### (3) Synthesis of TEC02A (Debenzylation Reaction)

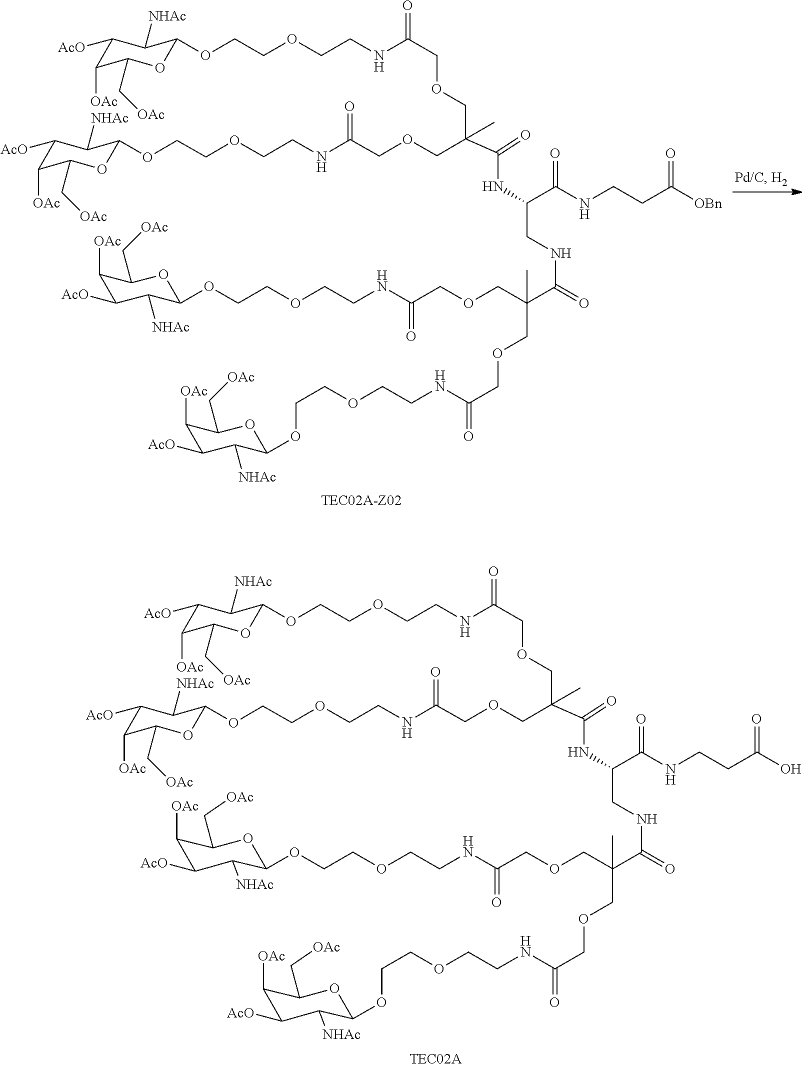

TEC02A-Z02 (1.45 g, 0.61 mmol) was dissolved in tetrahydrofuran (30 mL) and added with 10% Pd/C (0.15 g, 10 wt %). The reaction system was replaced with hydrogen for three times and the reaction was carried out under stirring and under hydrogen atmosphere (about 10-20 kPa higher than the standard atmospheric pressure) for 1.5 h when LCMS showed that the reaction was complete. The reaction mixture was filtered to remove the solid, concentrated under reduced pressure and dried under vacuum to obtain 1.28 g of a white solid product with a yield of 92%.

MS (ESI): m/z [1/2M+Na]⁺, theoretical 1175.5, found 1175.4.

### (4) Synthesis of TEC02A-Pfp

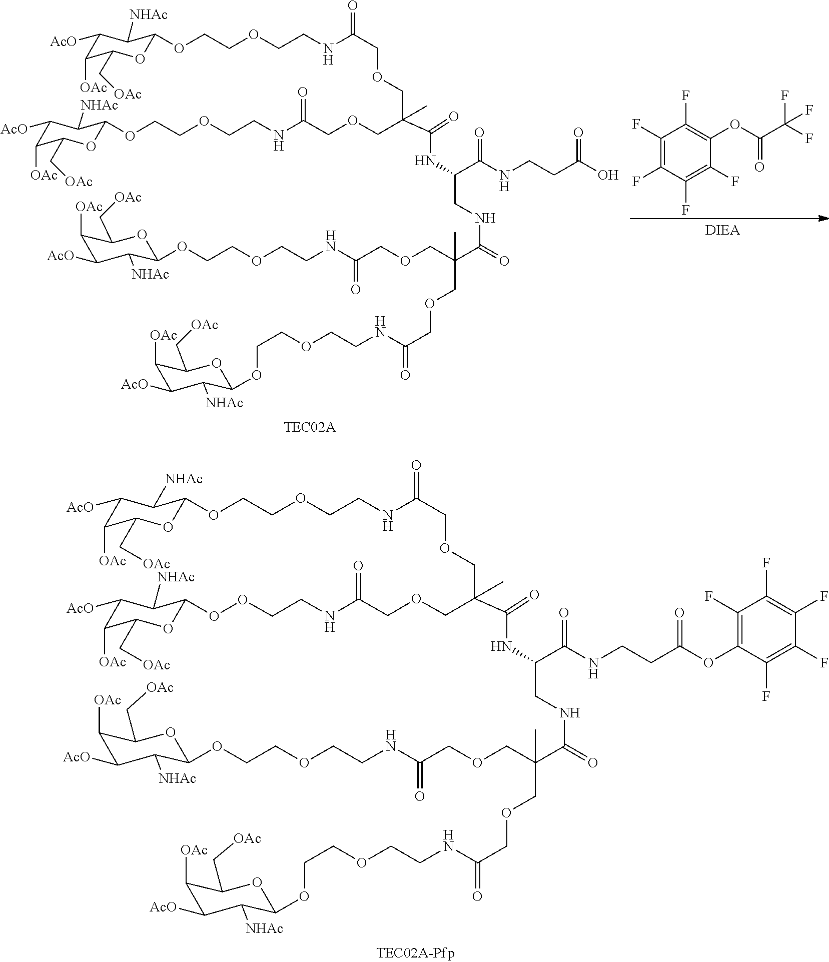

Under N₂ atmosphere and at room temperature, TEC02A (1.2 g, 0.52 mmol) and DIEA (0.3 g, 2.0 mmol) were successively added to a flask containing dichloromethane (12 mL) and stirred until the reaction mixture became clear. The reaction was dropwise added with pentafluorophenyl trifluoroacetate (0.3 g, 1.0 mmol) and carried out under stirring at room temperature for 0.5 h when LCMS showed that the conversion of the raw materials was completed. The reaction solution was washed with 8% aqueous sodium bicarbonate solution (12 mL×7) and extracted with dichloromethane (12 mL×3). The obtained organic phases were combined, washed with 26% aqueous sodium chloride solution (12 mL), dried over anhydrous sodium sulfate, filtered and concentrated under reduced pressure to obtain a white solid crude product. The crude product was recrystallized (acetonitrile/methyl tert-butyl ether, 1/2) to obtain 1.1 g of a white solid product with a yield of 85.5%.

MS (ESI): m/z [1/3M+H]⁺, theoretical 824.6, found 824.3.

### (5) Synthesis of TEC02A-Cy5-01 (Amidation Condensation Reaction)

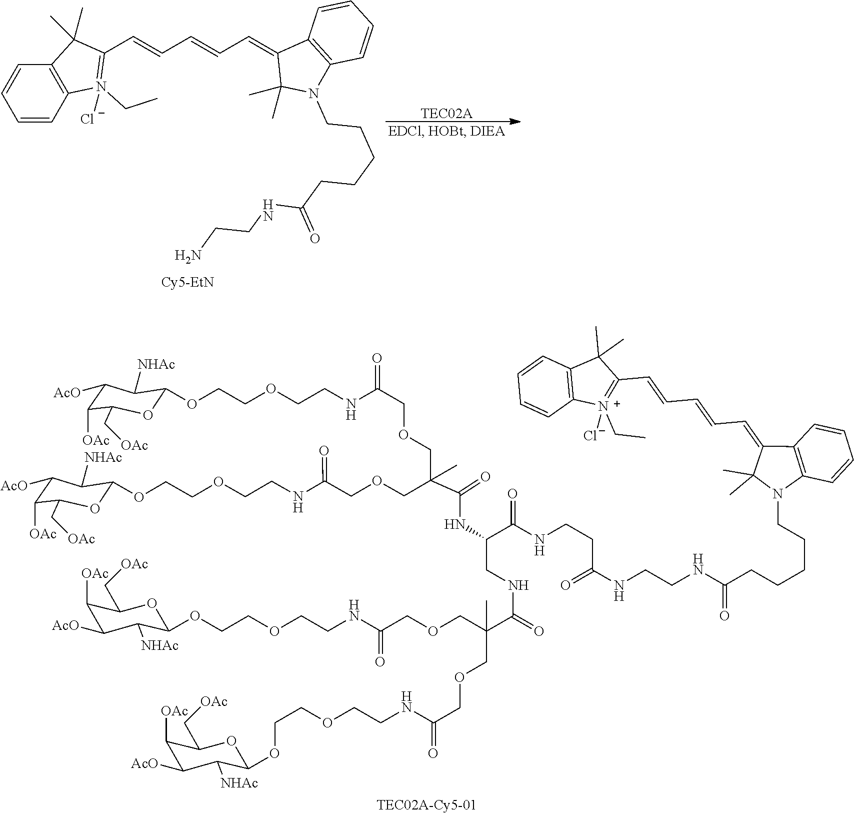

Under N₂ atmosphere and at room temperature, TEC02A (40 mg, 0.017 mmol), Cy5-EtN (10 mg, 0.02 mmol), EDCI (4.8 mg, 0.03 mmol) and HOBt (2.4 mg, 0.02 mmol) were successively added to a flask containing dichloromethane (2 mL) and stirred at room temperature until the reaction mixture became clear. The reaction was then added with DIEA (7.8 mg, 0.06 mmol) and carried out under stirring for 20 h when LCMS showed that the reaction was complete. The reaction solution was subjected to distillation under reduced pressure to remove the solvent, and the crude product was purified by silica gel column chromatography (200-300 meshes, dichloromethane/methanol, 9/1 to 4/1) to obtain 19.9 mg of a blue solid product with a yield of 40.8%.

MS (ESI): m/z [1/3 (M+2Na)]⁺, theoretical 957.4, found 957.1.

### (6) Synthesis of TEC02A-Cy5

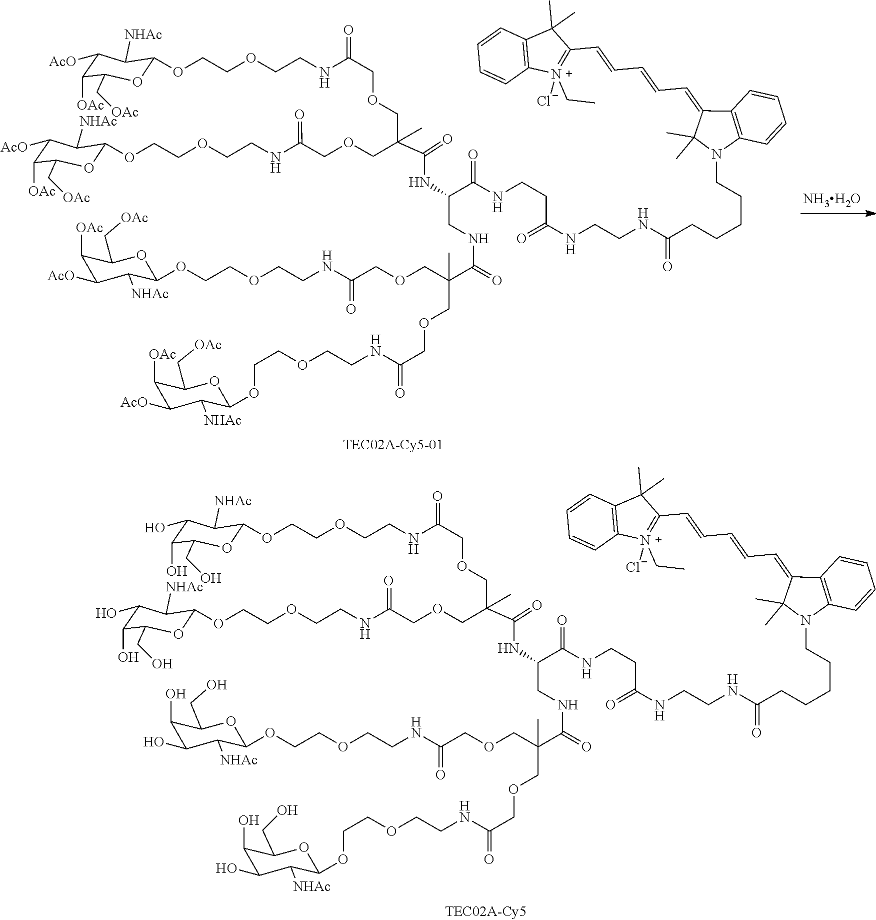

Under N₂ atmosphere and at room temperature, TEC02A-Cy5-01 (10 mg, 0.0035 mmol) was added to a flask containing methanol (0.9 mL) and stirred until the reaction mixture became clear. The reaction was then added with 25% ammonia (0.1 mL), increased to 40° C. and carried out under stirring for 1 h when TLC showed that conversion of most of the raw materials was completed. The reaction solution was subjected to distillation under reduced pressure to remove the solvent, and the crude product was purified by prep-HPLC (C₁₈/5 μm, column temperature: 40° C.; water/acetonitrile, 95/5 to 5/95, containing 0.1% formic acid) to obtain 1.28 mg of a blue solid product with a yield of 15.5% and a purity of 98.4% (284 nm).

MS (ESI): m/z [1/2(M+Na)]⁺, theoretical 1172.6, found 1172.3.

### 6. Synthesis of TEC14A (a Stereoisomeric Carboxylic Acid with Peracetylated Glycosyl Groups of Ligand TEC14) and TEC14A-Cy5 (a Receptor-Targeting Fluorescent Conjugate)

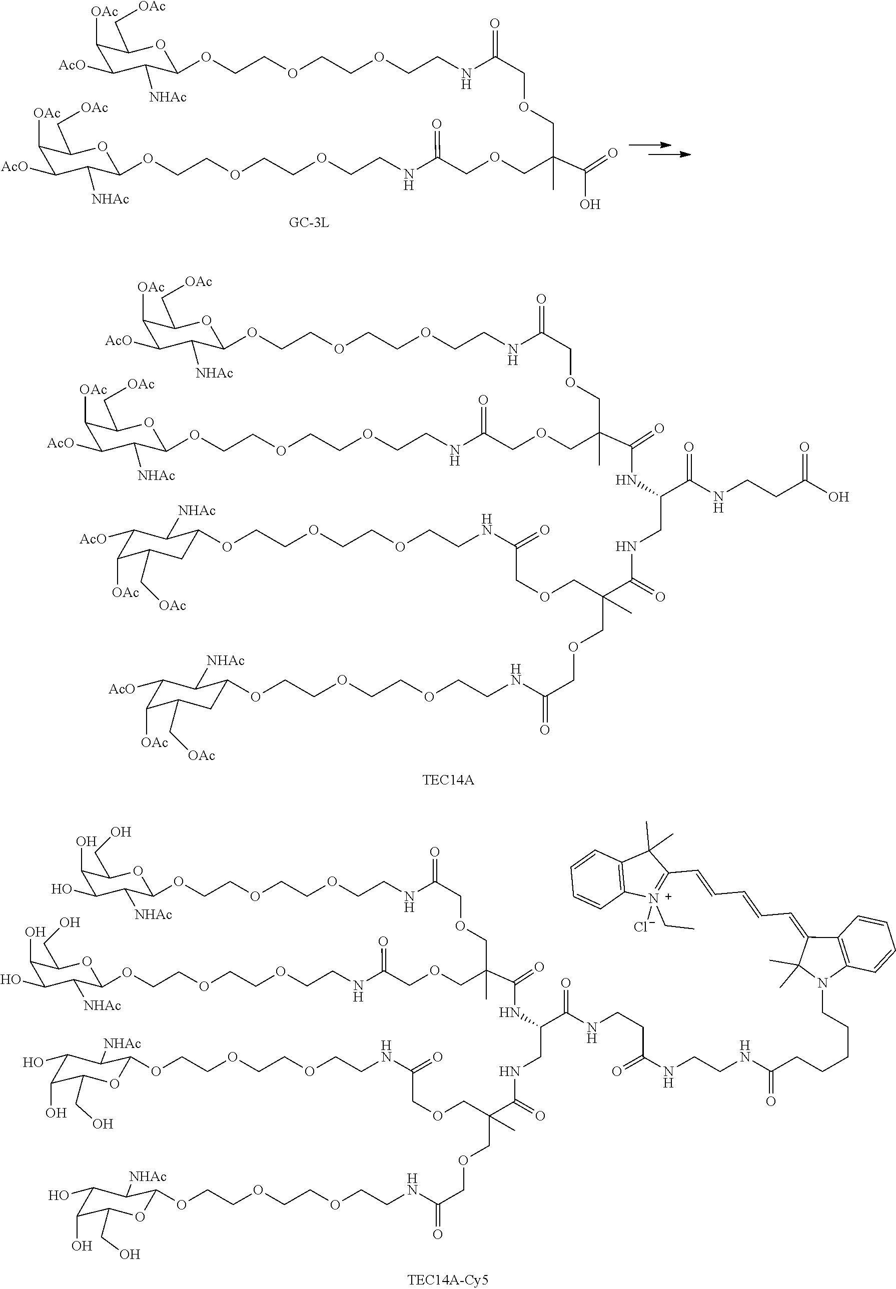

TEC14A and TEC14A-Cy5 were synthesized by the same steps for synthesis of TEC02A and TEC02A-Cy5, except that GC-3 was replaced with GC-3L as a starting material. TEC14A, MS (ESI): m/z [1/2M+H]⁺, theoretical 1241.5, found 1241.4.

TEC14A-Cy5, MS (ESI): m/z [1/3 (M+2Na)]⁺, theoretical 848.1, found 848.3.

### 7. Synthesis of TEC06A (a Stereoisomeric Carboxylic Acid with Peracetyalted Glycosyl Groups of Ligand TEC06), TEC06A-Pfp (an Activated Ester) and TEC02A-Cy5 (a Receptor-Targeting Fluorescent Conjugate)

TEC06A, TEC06A-Pfp and TEC06A-Cy5 were synthesized by the same steps for synthesis of TEC02A, TEC02A-Pfp and TEC02A-Cy5, respectively, except that BL-6 was replaced with BL-7 and BL-6-Z02 was replaced with with BL-7-Z03.

### (1) Synthesis of TEC06A-Z01 (Amidation Condensation Reaction)

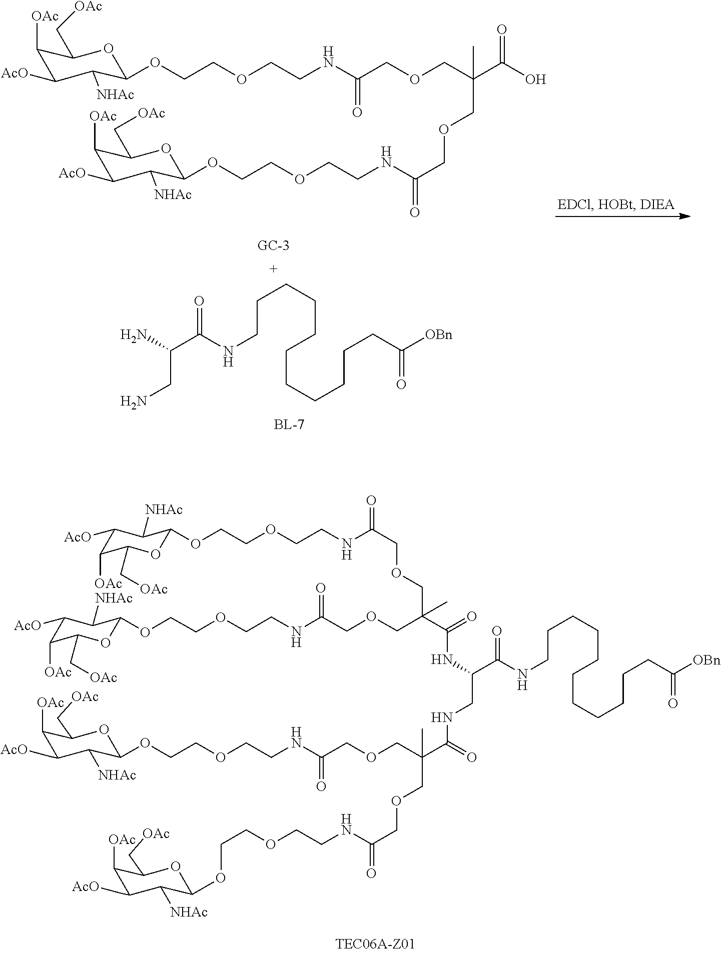 [SMILES: CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)O)OC(COC(C)=O)C(OC(C)=O)C1OC(C)=O ; NC[C@H](N)C(=O)NCCCCCCCCCCCC(=O)OCc1ccccc1 ; CC(=O)NC1C(OCCOCCNC(=O)COCC(C)(COCC(=O)NCCOCCOC2OC(COC(C)=O)C(OC(C)=O)C(COC(C)=O)C2NC(C)=O)C(=O)NC[C@H](NC(=O)C(C)(COCC(=O)NCCOCCOC2OC(COC(C)=O)C(OC(C)=O)C(OC(C)=O)C2NC(C)=O)COCC(=O)NCCOCCOC2OC(COC(C)=O)C(OC(C)=O)C(OC(C)=O)C2NC(C)=O)C(=O)NCCCCCCCCCCCC(=O)OCc2ccccc2)OC(COC(C)=O)C(OC(C)=O)C1COC(C)=O]

Under N₂ atmosphere and at room temperature, GC-3 (650 mg, 0.6 mmol), BL-7 (a HCl salt, 127 mg, 0.27 mmol), EDCI (157 mg, 0.82 mmol) and HOBt (74 mg, 0.55 mmol) were successively added to dichloromethane (10 mL) and stirred at room temperature until the reaction mixture became clear. The reaction was then added with DIEA (211 mg, 1.6 mmol) and carried out under stirring for 3 h when LCMS showed that the reaction was complete. The reaction solution was added with pure water (20 mL) to quench the reaction, and extracted with dichloromethane (10 mL×3), and the obtained organic phases were combined, washed with 26% aqueous sodium chloride solution (10 mL), dried over anhydrous sodium sulfate, filtered and concentrated under reduced pressure to obtain a white solid crude product. The crude product was purified by silica gel column chromatography (200-300 meshes, dichloromethane/methanol, 50/1 to 10/1) to obtain 310 mg of a white solid product with a yield of 45%.

MS (ESI): m/z [1/3M+H]⁺, theoretical 841.4, found 841.1; m/z [1/2M+H]⁺, theoretical 1261.6, found 1261.5.

¹H NMR (400 MHz, DMSO-d₆) δ 8.15-8.09 (m, 1H), 7.97 (d, J=7.1 Hz, 1H), 7.91-7.81 (m, 8H), 7.73 (t, J=5.7 Hz, 1H), 7.40-7.29 (m, 5H), 5.21 (d, J=3.4 Hz, 4H), 5.07 (s, 2H), 4.98 (dd, J=11.2, 3.4 Hz, 4H), 4.55 (d, J=8.5 Hz, 4H), 4.30 (q, J=6.9 Hz, 1H), 4.06-3.99 (m, 12H), 3.93-3.83 (m, 12H), 3.82-3.74 (m, 4H), 3.64-3.55 (m, 8H), 3.54-3.48 (m, 12H), 3.45-3.40 (m, 8H), 3.31-3.22 (m, 8H), 3.17-3.08 (m, 1H), 3.07-2.93 (m, 2H), 2.33 (t, J=7.4 Hz, 2H), 2.10 (s, 12H), 1.99 (s, 12H), 1.89 (s, 12H), 1.77 (s, 12H), 1.56-1.48 (m, 2H), 1.40-1.32 (m, 2H), 1.30-1.24 (m, 10H), 1.22-1.18 (m, 10H), 1.11 (s, 3H), 1.07 (s, 3H).

### (2) Synthesis of TEC06A (Debenzylation Reaction)

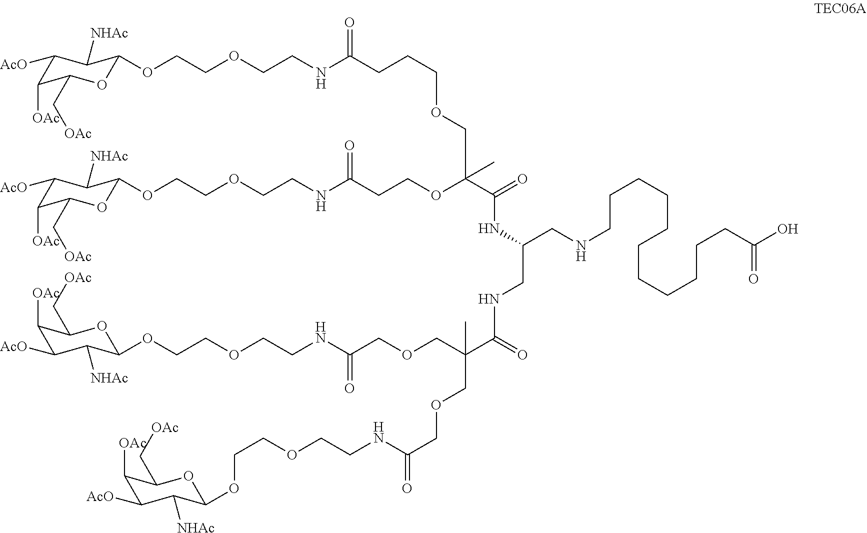 [SMILES: CC(=O)NC1C(OCCOCCNC(=O)CCCOCC(C)(OCCC(=O)NCCOCCOC2OC(COC(C)=O)C(OC(C)=O)C(OC(C)=O)C2NC(C)=O)C(=O)N[C@H](CNCCCCCCCCCCCC(=O)O)CNC(=O)C(C)(COCC(=O)NCCOCCOC2OC(COC(C)=O)C(OC(C)=O)C(OC(C)=O)C2NC(C)=O)COCC(=O)NCCOCCOC2OC(COC(C)=O)C(OC(C)=O)C(OC(C)=O)C2NC(C)=O)OC(COC(C)=O)C(OC(C)=O)C1OC(C)=O]

TEC06A-Z01 (300 mg, 0.119 mmol) was dissolved in tetrahydrofuran (30 mL) and added with Pd/C (30 mg, 10 wt %). The reaction system was replaced with hydrogen for three times and the reaction was carried out under stirring and under hydrogen atmosphere (about 10-20 kPa higher than the standard atmospheric pressure) for 1.5 h when LCMS showed that the reaction was complete. The reaction mixture was filtered to remove the solid and then concentrated under reduced pressure to obtain 263 mg of a white solid product with a yield of 91%.

MS (ESI): m/z [1/3M+H]⁺, theoretical 811.4, found 811.2; m/z [1/2M+H]⁺, theoretical 1216.5, found 1216.5.

### (3) Synthesis of TEC06A-Pfp

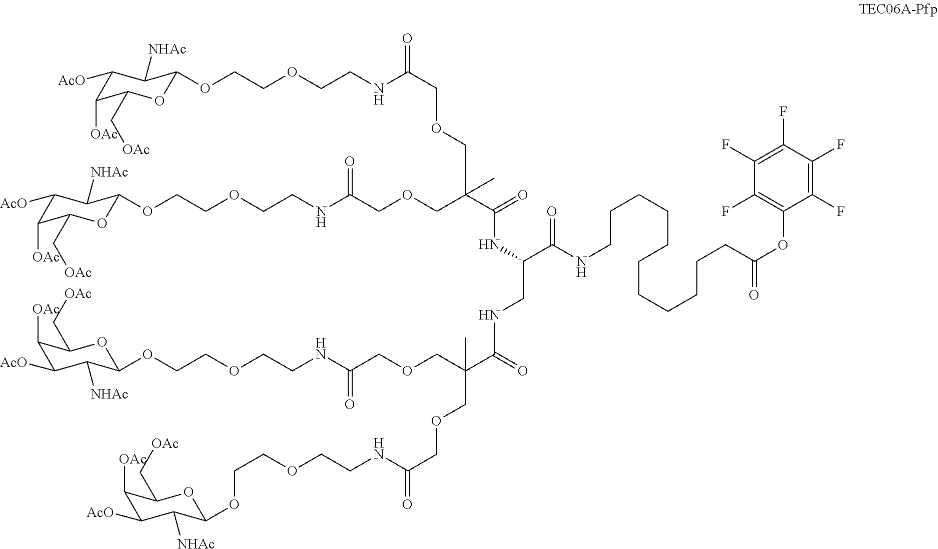 [SMILES: CC(=O)NC1C(OCCOCCNC(=O)COCC(C)(COCC(=O)NCCOCCOC2OC(COC(C)=O)C(OC(C)=O)C(OC(C)=O)C2NC(C)=O)C(=O)NC[C@H](NC(=O)C(C)(COCC(=O)NCCOCCOC2OC(COC(C)=O)C(OC(C)=O)C(OC(C)=O)C2NC(C)=O)COCC(=O)NCCOCCOC2OC(COC(C)=O)C(OC(C)=O)C(OC(C)=O)C2NC(C)=O)C(=O)NCCCCCCCCCCCC(=O)Oc2c(F)c(F)c(F)c(F)c2F)OC(COC(C)=O)C(OC(C)=O)C1OC(C)=O]

Under N₂ atmosphere and at room temperature, TEC06A (140 mg, 0.058 mmol) and DIEA (30 mg, 0.23 mmol) were successively added to a flask containing dichloromethane (12 mL) and stirred until the reaction mixture became clear. The reaction was dropwise added with pentafluorophenyl trifluoroacetate (32 mg, 0.11 mmol) and carried out under stirring for 4 h when LCMS showed that the conversion of the raw materials was complete. The reaction solution was washed with 8% aqueous sodium bicarbonate solution (10 mL×7) and extracted with dichloromethane (12 mL×3), and the obtained organic phases were combined, washed with 26% aqueous sodium chloride solution (15 mL), dried over anhydrous sodium sulfate, filtered and concentrated under reduced pressure to obtain a white solid crude product. The crude product was crystallized and purified (acetonitrile/methyl tert-butyl ether, 1/2) to obtain 90 mg of a white solid product with a yield of 60%.

MS (ESI): m/z [1/3M+H]⁺, theoretical 866.7, found 866.3.

¹H NMR (400 MHz, DMSO-d₆) δ 8.16-8.09 (m, 1H), 8.00-7.93 (m, 1H), 7.93-7.81 (m, 8H), 7.77-7.70 (m, 1H), 5.21 (d, J=3.3 Hz, 4H), 4.97 (dd, J=11.2, 3.4 Hz, 4H), 4.54 (d, J=8.4 Hz, 4H), 4.29 (q, J=6.9 Hz, 1H), 4.02 (s, 12H), 3.93-3.83 (m, 12H), 3.82-3.73 (m, 4H), 3.65-3.47 (m, 22H), 3.45-3.40 (m, 8H), 3.30-3.21 (m, 8H), 3.13 (q, J=7.5 Hz, 2H), 3.06-2.93 (m, 2H), 2.77 (t, J=7.2 Hz, 1H), 2.10 (s, 12H), 1.99 (s, 12H), 1.89 (s, 12H), 1.77 (s, 12H), 1.70-1.60 (m, 1H), 1.40-1.31 (m, 3H), 1.27-1.23 (m, 16H), 1.11 (s, 3H), 1.07 (s, 3H).

### (4) Synthesis of TEC06A-Cy5-01 (Amidation Condensation Reaction)

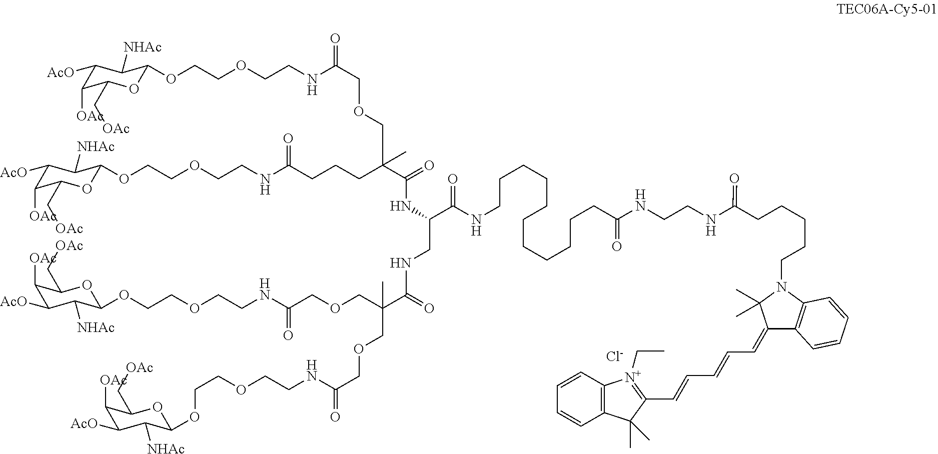 [SMILES: CC[N+]1=C(/C=C/C=C/C=C2/c3ccccc3N(CCCCCC(=O)NCCNC(=O)CCCCCCCCCCCNC(=O)[C@H](CNC(=O)C(C)(COCC(=O)NCCOCCOC3OC(COC(C)=O)C(OC(C)=O)C(OC(C)=O)C3NC(C)=O)COCC(=O)NCCOCCOC3OC(COC(C)=O)C(OC(C)=O)C(OC(C)=O)C3NC(C)=O)NC(=O)C(C)(CCCC(=O)NCCOCCOC3OC(COC(C)=O)C(OC(C)=O)C(OC(C)=O)C3NC(C)=O)COCC(=O)NCCOCCOC3OC(COC(C)=O)C(OC(C)=O)C(OC(C)=O)C3NC(C)=O)C2(C)C)C(C)(C)c2ccccc21 ; [Cl-]]

Under N₂ atmosphere and at room temperature, TEC06A-Z01 (20 mg, 0.0082 mmol), Cy5-EtN (4.7 mg, 0.01 mmol), EDCI (2.4 mg, 0.015 mmol) and HOBt (1.7 mg, 0.015 mmol) were successively added to a flask containing dichloromethane (2 mL) and stirred at room temperature until the reaction mixture became clear. The reaction was then added with DIEA (3.9 mg, 0.03 mmol) and carried out under stirring for 20 h when LCMS showed that there were no residual raw materials. The reaction solution was subjected to distillation under reduced pressure to remove the solvent, and the crude product was purified by silica gel column chromatography (200-300 meshes, dichloromethane/methanol, 9/1 to 4/1) to obtain 20 mg of a blue solid product with a yield of 81%.

MS (ESI): m/z [1/3 (M−Cl+2Na)]⁺, theoretical 999.5, found 999.3.

### (5) Synthesis of TEC06A-Cy5

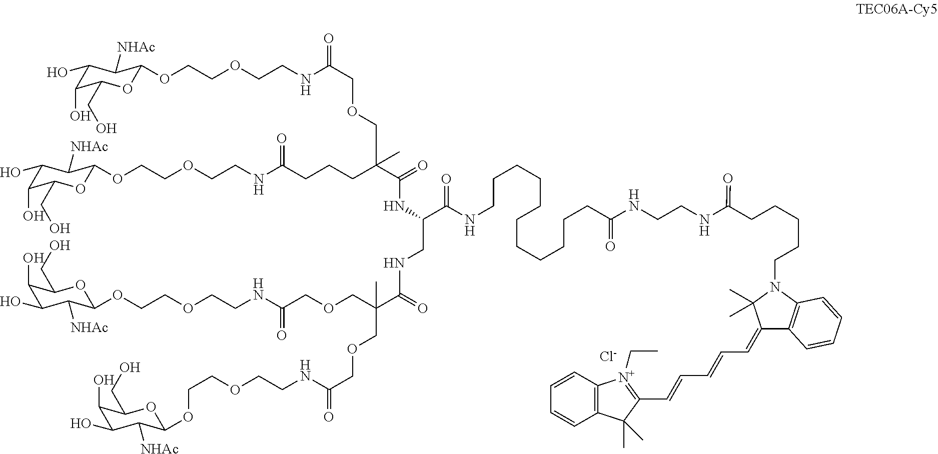 [SMILES: CC[N+]1=C(/C=C/C=C/C=C2/c3ccccc3N(CCCCCC(=O)NCCNC(=O)CCCCCCCCCCCNC(=O)[C@H](CNC(=O)C(C)(COCC(=O)NCCOCCOC3OC(CO)C(O)C(O)C3NC(C)=O)COCC(=O)NCCOCCOC3OC(CO)C(O)C(O)C3NC(C)=O)NC(=O)C(C)(CCCC(=O)NCCOCCOC3OC(CO)C(O)C(O)C3NC(C)=O)COCC(=O)NCCOCCOC3OC(CO)C(O)C(O)C3NC(C)=O)C2(C)C)C(C)(C)c2ccccc21 ; [Cl-]]

Under N₂ atmosphere and at room temperature, TEC06A-Cy5-01 (20 mg, 0.0067 mmol) was added to a flask containing methanol (3 mL) and stirred until the reaction mixture became clear. The reaction was added with 25% ammonia (0.5 mL), increased to 40° C. and carried out under stirring for 1 h when LCMS showed that there were no residual raw materials. The reaction solution was subjected to distillation under reduced pressure to remove the solvent, and the crude product was purified by prep-HPLC (C₁₈/5 μm, column temperature: 40° C.; water/acetonitrile, 95/5 to 5/95, containing 0.1% formic acid) to obtain 4.1 mg of a blue solid product with a yield of 24% and a purity of 99.9% (630 nm).

MS (ESI): m/z [1/3 (M−Cl+2Na)]⁺, theoretical 831.4, found 831.0.

### 8. Synthesis of TEC03A (a Stereoisomeric Carboxylic Acid with Peracetylated Glycosyl Groups of Ligand TEC03) and TEC03A-OSu (an Activated Ester)

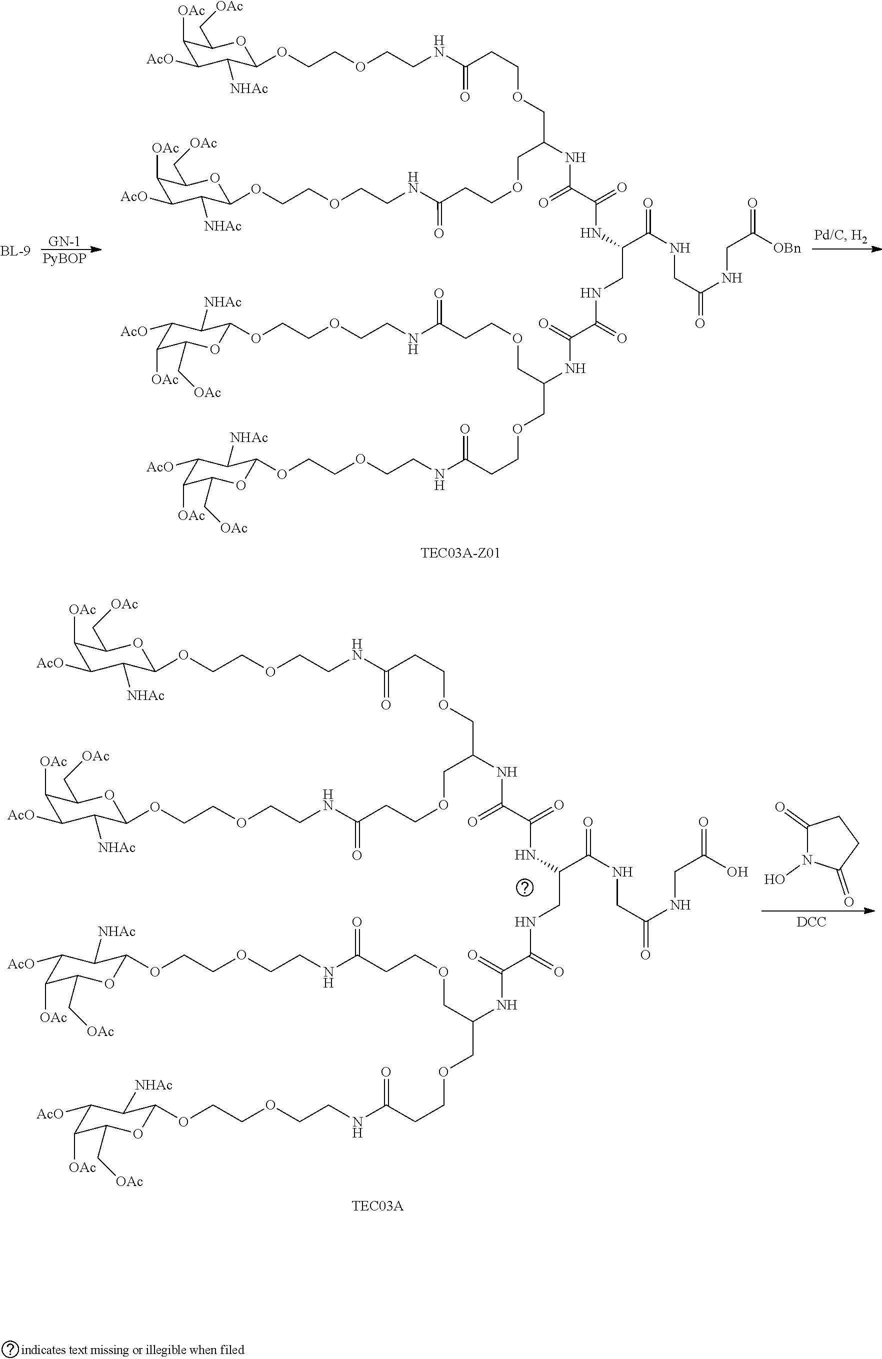

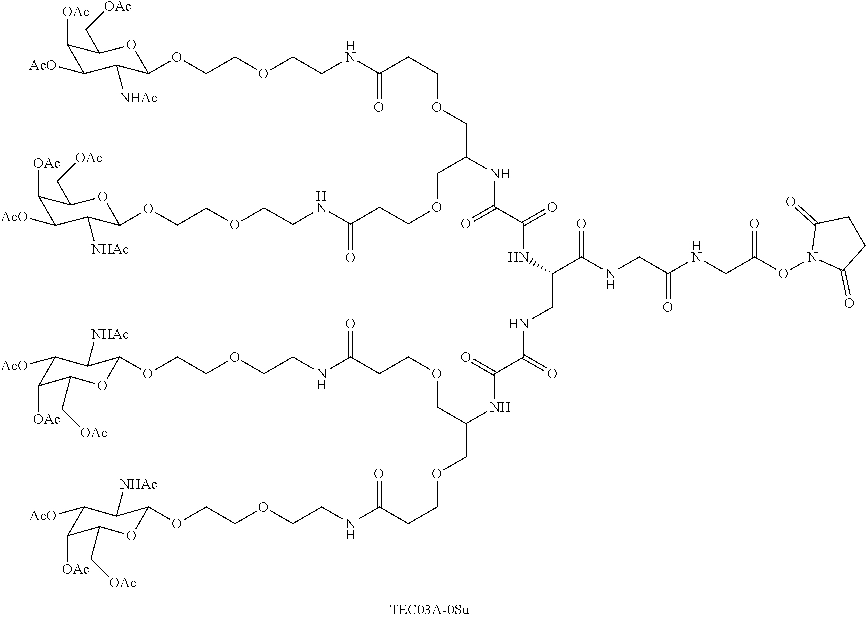 [SMILES: CC(=O)NC1C(OCCOCCNC(=O)CCOCC(COCCC(=O)NCCOCCOC2OC(COC(C)=O)C(OC(C)=O)C(OC(C)=O)C2NC(C)=O)NC(=O)C(=O)NC[C@H](NC(=O)C(=O)NC(COCCC(=O)NCCOCCOC2OC(COC(C)=O)C(OC(C)=O)C(OC(C)=O)C2NC(C)=O)COCCC(=O)NCCOCCOC2OC(COC(C)=O)C(OC(C)=O)C(OC(C)=O)C2NC(C)=O)C(=O)NCC(=O)NCC(=O)ON2C(=O)CCC2=O)OC(COC(C)=O)C(OC(C)=O)C1OC(C)=O]

### (1) Synthesis of TEC03A-Z01 (Amidation Condensation Reaction)

Under N₂ atmosphere, BL-9 (350 mg, 0.395 mmol), GN-1 (720 mg, 1.66 mmol), PyBOP (925 mg, 1.78 mmol) and triethylamine (279 mg, 2.76 mmol) were successively added to a flask containing dichloromethane (10 mL) and then the reaction was carried out under stirring at room temperature for 6 h when LCMS showed that the reaction was complete. The reaction solution was added with pure water (15 mL) and extracted with dichloromethane (10 mL×3). The obtained organic phase was dried over anhydrous sodium sulfate, filtered, and subjected to distillation under reduced pressure to remove the solvent. The residue was purified by silica gel column chromatography (200-300 meshes, dichloromethane/methanol, 20/1) to obtain 383 mg of a light yellow solid with a yield of 37%.

MS (ESI): m/z [1/3M+H]⁺, theoretical 851.3, found 851.4.

### (2) Synthesis of TEC03A (Debenzylation Reaction)

TEC03A-Z01 (370 mg, 0.145 mmol) was dissolved in tetrahydrofuran (8 mL) and added with Pd/C (35 mg, 10 wt %). The reaction system was replaced with hydrogen for three times and the reaction was carried out under stirring and under hydrogen atmosphere (about 10-20 kPa higher than the standard atmospheric pressure) for 2 h when LCMS showed that the reaction was complete. The reaction mixture was filtered to remove the solid and the filtrate was concentrated under reduced pressure to obtain 321 mg of a white solid product with a yield of 90%.

MS (ESI): m/z [1/2M+H]⁺, theoretical 1232.0, found 1231.9.

¹H NMR (400 MHz, DMSO-d₆) δ 8.92 (t, J=6.5 Hz, 1H), 8.54 (d, J=7.6 Hz, 1H), 8.17-8.09 (m, 3H), 8.05 (s, 1H), 7.62 (d, J=7.0 Hz, 4H), 7.49 (t, J=5.1 Hz, 4H), 5.21 (d, J=3.4 Hz, 4H), 5.02-4.95 (m, 8H), 4.68-4.60 (m, 1H), 4.25-4.12 (m, 8H), 4.12-4.02 (m, 4H), 3.97-3.89 (m, 4H), 3.89-3.70 (m, 14H), 3.66-3.57 (m, 17H), 3.57-3.46 (m, 17H), 3.43-3.35 (m, 2H), 3.35-3.29 (m, 8H), 2.32 (t, J=6.3 Hz, 8H), 2.10 (m, 12H), 1.98 (m, 12H), 1.88 (s, 12H), 1.78 (s, 12H).

### (3) Synthesis of TEC03A-OSu

Under N₂ atmosphere and at room temperature, TEC03A (80 mg, 0.0325 mmol) and N-hydroxysuccinimide (6 mg, 0.0488 mmol) were successively added to a flask containing dichloromethane (3 mL), and under constant stirring, N,N′-dicyclohexylcarbodiimide (14 mg, 0.065 mmol) was added, and the reaction was carried out for 2 h when LCMS showed that the conversion of the raw materials was complete. The crude product obtained by concentration under reduced pressure was crystallized and purified with ethyl acetate and methyl tert-butyl ether to obtain 60.7 mg of a white solid product with a yield of 73%.

MS (ESI): m/z [1/3M+H]⁺, theoretical 854.0, found 854.1.

### 9. Synthesis of TEC04A (a Stereoisomeric Alcohol with Peracetylated Glycosyl Groups of Ligand TEC04) and TEC04A-PA (a Phosphoramidite Compound)

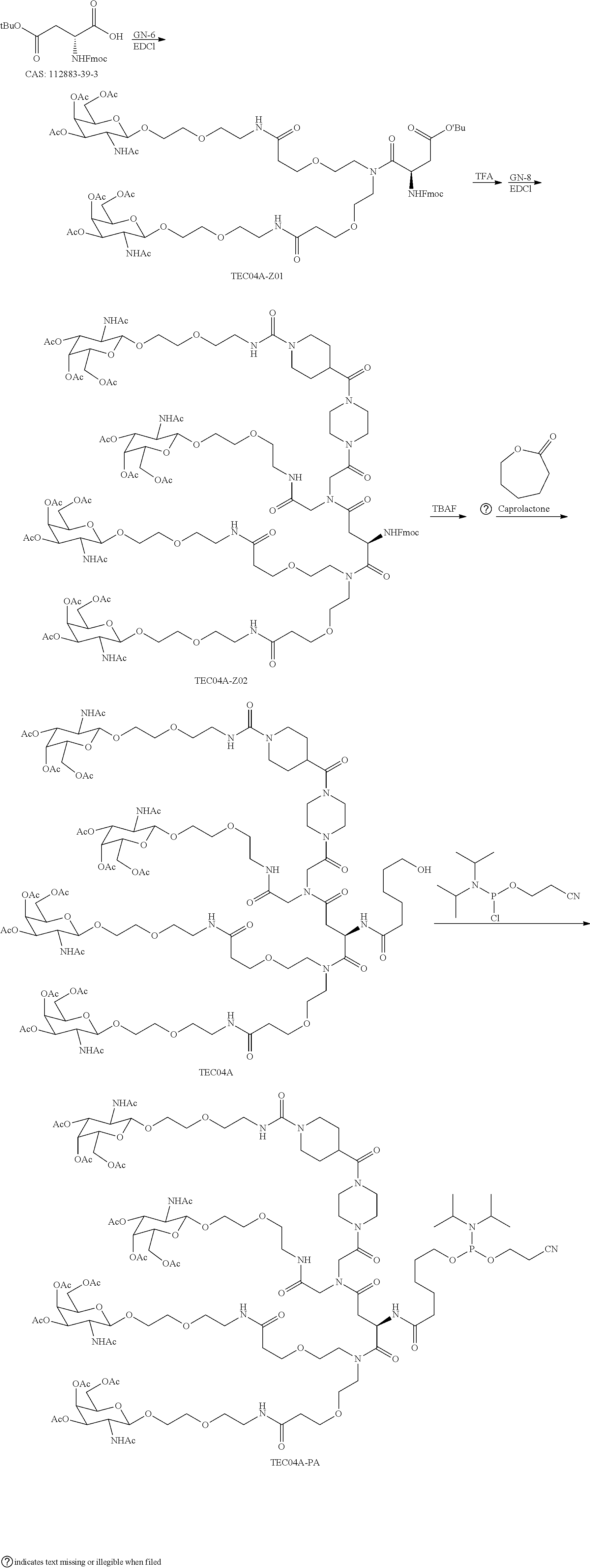

Synthesis of TEC04A: (1) TEC04A-Z01 was obtained by subjecting N-Fmoc-D-aspartic acid-4-tert-butyl ester as a start material to amidation condensation reaction with GN-6; (2) TEC04A-Z02 was obtained by subjecting TEC04A-Z01 to a de-tert-butylation reaction and then to amidation condensation reaction with GN-8; (3) TEC04A was obtained by subjected TEC04A-Z02 to de-9-fluorenylmethoxycarbonylation reaction and further to reaction with ε-caprolactone. TEC04A-PA was synthesized by subjecting TEC04A to reaction with a phosphoramidite reagent.

### 10. Synthesis of TEC05A (a Stereoisomeric Carboxylic Acid with Peracetylated Glycosyl Groups of Ligand TEC05)

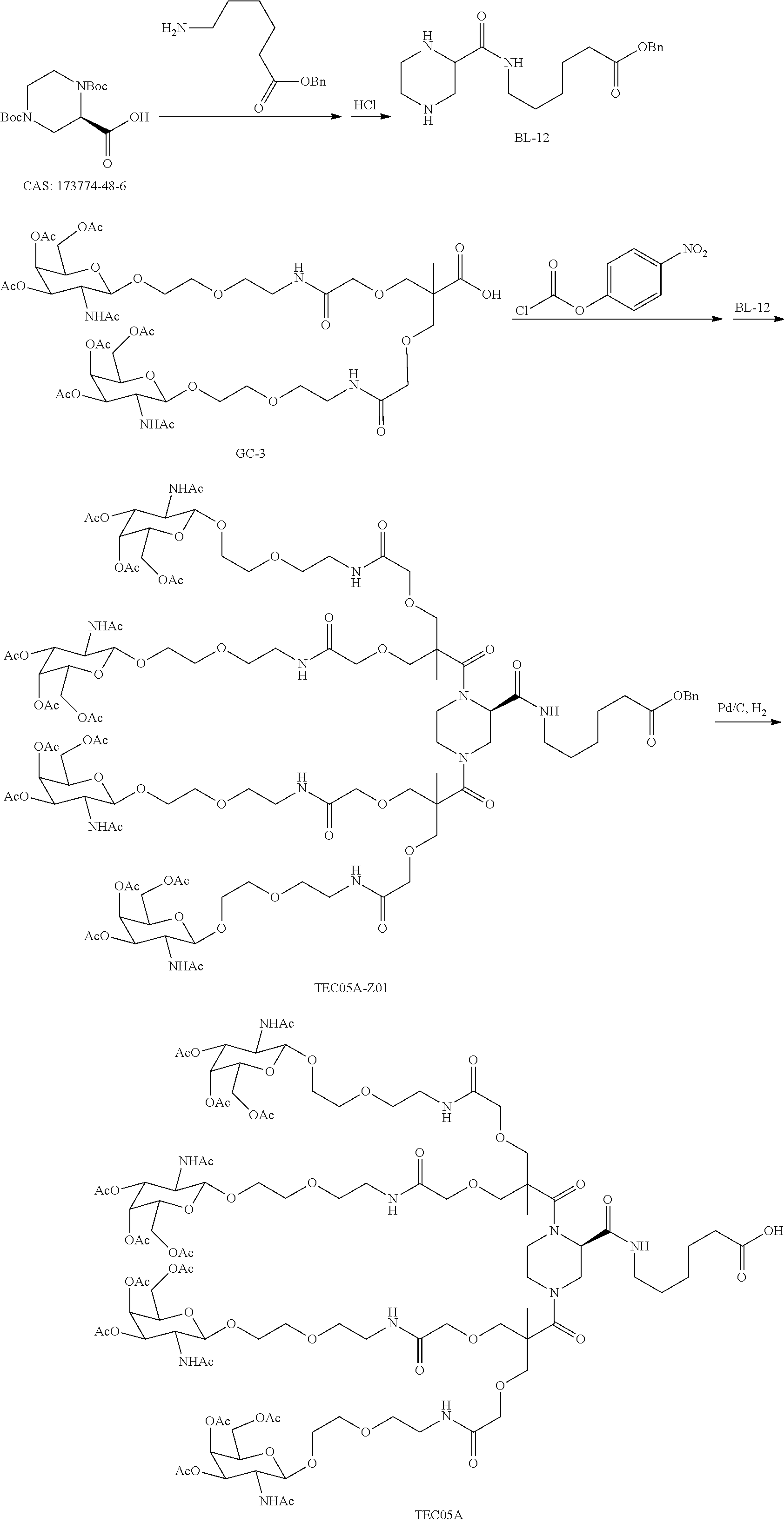

### (1) Synthesis of BL-12 (Amidation Condensation and De-Tert-Butoxycarbonylation Reaction)

Under N₂ atmosphere, under stirring and at room temperature, benzyl 6-aminocaproate (1.48 g, 6.67 mmol), (R)-1,4-bis-Boc-piperazine-2-formic acid (2.1 g, 6.36 mmol), EDCI (1.82 g, 9.54 mmol) and HOBt (0.859 g, 6.36 mmol) were successively added to a flask containing dichloromethane (16 mL), and then added with DIEA (1.64 g, 12.7 mmol), and the reaction was carried out for 4 h when LCMS showed that the reaction was completed. The reaction solution was added with pure water (20 mL) and extracted with dichloromethane (15 mL×3). The obtained organic phases were combined, washed with 26% aqueous sodium chloride solution (45 mL), dried over anhydrous sodium sulfate, filtered and concentrated under reduced pressure to obtain a crude product. The crude product was purified by silica gel column chromatography (200-300 meshes, petroleum ether/ethyl acetate, 8/1) obtain 2.91 g of a light yellow solid, which was a condensed intermediate with two Boc groups. The obtained solid was added to a flask containing 1,4-dioxane (4 mL), and then added with 4 M HCl/1,4-dioxane solution (8 mL), and the reaction was carried out under stirring for 1 h when LCMS showed that the reaction was complete. The reaction solution was subjected to distillation under reduced pressure to remove the solvent, and the crude product was crystallized and purified with ethyl acetate to obtain 2.1 g of a light yellow solid product (a HCl salt) with a yield of 80%.

MS (ESI): m/z [M+H]⁺, theoretical 334.2, found 334.2.

¹H NMR (400 MHz, DMSO-d₆) δ 9.45 (s, 3H), 8.76 (s, 1H), 7.41-7.28 (m, 3H), 5.07 (s, 1H), 4.06 (d, J=11.0 Hz, 1H), 3.68-3.53 (m, 1H), 3.22-2.90 (m, 4H), 2.34 (t, J=7.4 Hz, 2H), 1.58-1.47 (m, 2H), 1.46-1.36 (m, 2H), 1.32-1.20 (m, 2H).

### (2) Synthesis of TEC05A-Z01 (Amidation Condensation Reaction)

Under N₂ atmosphere, GC-3 (1.5 g, 1.38 mmol) and triethylamine (0.21 g, 2.1 mmol) were successively added to a flask containing tetrahydrofuran (10 mL), and in an ice water bath of 0-5° C., 4-nitrophenyl chloroformate (0.31 g, 1.52 mmol) was added slowly, and the reaction was carried out under stirring for 1 h, and then added with pure water (20 mL) and extracted with dichloromethane (15 mL×3). The obtained organic phases were combined, washed with 26% aqueous sodium chloride solution (25 mL), dried over anhydrous sodium sulfate, filtered and concentrated under reduced pressure to obtain 4.1 g of a light yellow solid crude product, which was a carboxylic acid active ester intermediate. The intermediate was dissolved in pyridine/dichloromethane (5/10 mL), and added with BL-12 (a HCl salt, 0.25 g, 0.627 mmol) under stirring. The reaction was then carried out at room temperature for 16 h when LCMS showed that the reaction was complete. The reaction solution was concentrated under reduced pressure, and the residue was dissolved with ethyl acetate (20 mL) and washed with 26% aqueous sodium chloride solution (15 mL×2). The obtained organic phase was dried over anhydrous sodium sulfate, filtered, and concentrated under reduced pressure, and the residue was purified by silica gel column chromatography (200-300 meshes, dichloromethane/methanol, 15/1) to obtain 525 mg of a light yellow solid product with a yield of 34%.

MS (ESI): m/z [1/3M+H]⁺, theoretical 822.0, found 822.2.

### (3) Synthesis of TEC05A (Debenzylation Reaction)

TEC05A-Z01 (202 mg, 0.082 mmol) was dissolved in tetrahydrofuran (5 mL) and added with Pd/C (20 mg, 10 wt %). The reaction system was replaced with hydrogen three times and the reaction was carried out under stirring and under hydrogen atmosphere (about 10-20 kPa higher than the standard atmospheric pressure) for 1 h when LCMS showed that the reaction was complete. The reaction mixture was filtered to remove the solid, then concentrated under reduced pressure and dried under vacuum to obtain 173 mg of a white solid product with a yield of 89%.

MS (ESI): m/z [1/2M+H]⁺, theoretical 1187.5, found 1187.3.

### 11. Synthesis of TEC07A (a Carboxylic Acid Compound with Peracetylated Glycosyl Groups of Ligand TEC07)

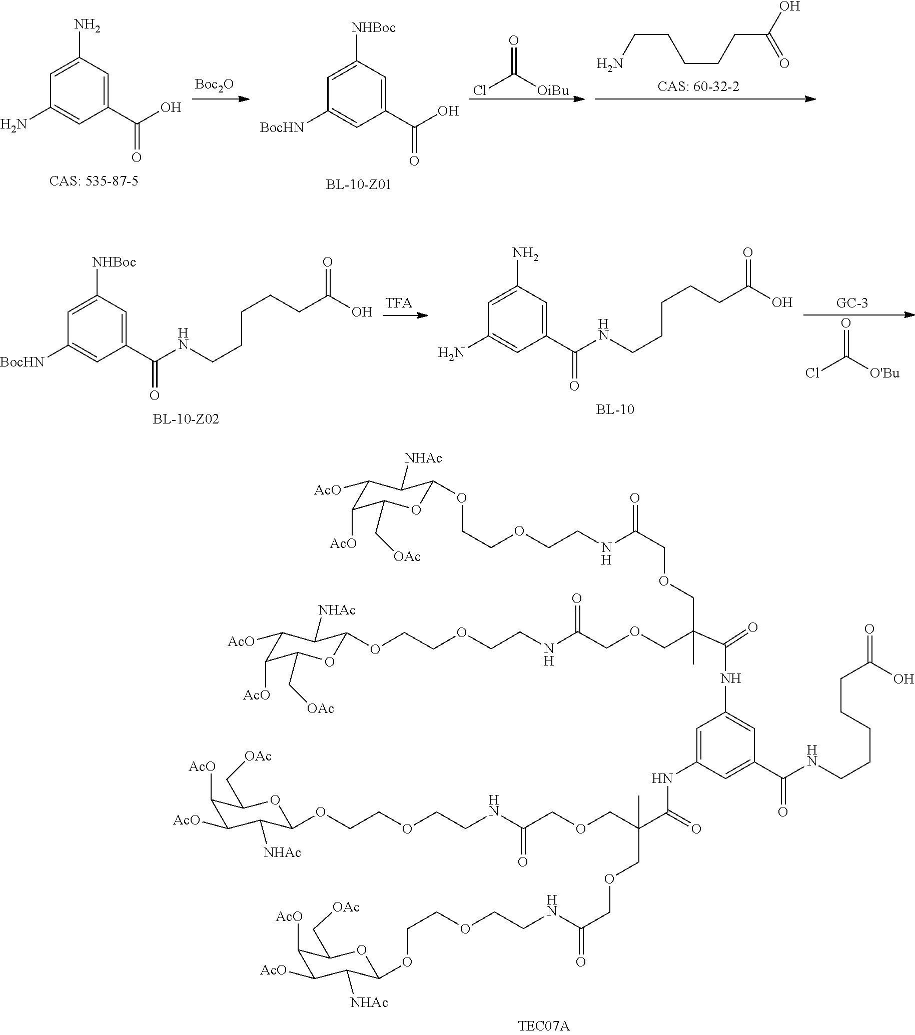

### (1) Synthesis of BL-10-Z02

BL-10-Z02 was obtained by subjecting BL-10-Z01 and 6-aminocaproic acid as starting materials to similar amidation condensation reaction steps for synthesis of BL-8-Z01.

MS (ESI): m/z [M+H]⁺, theoretical 466.3, found 466.3.

### (2) Synthesis of BL-10

BL-10 was obtained by subjecting BL-10-Z02 as a starting material to similar de-tert-butoxycarbonylation reaction steps for synthesis of BL-8.

MS (ESI): m/z [M+H]⁺, theoretical 266.1, found 266.1.

### (3) Synthesis of TEC07A

TEC07A was obtained by subjecting BL-10 and GC-3 as starting materials to similar amidation condensation reaction steps for synthesis of TEC02A-Z01.

MS (ESI): m/z [1/2M+H]⁺, theoretical 1198.5, found 1198.7.

¹H NMR (400 MHz, DMSO-d₆) δ 9.01 (br s, 2H), 8.28 (t, J=5.0 Hz, 1H), 8.11 (t, J=2.2 Hz, 1H), 7.95-7.91 (m, 2H), 7.84 (dd, J=9.2, 2.9 Hz, 4H), 7.69 (t, J=5.8 Hz, 4H), 5.21 (d, J=3.4 Hz, 4H), 4.98 (dd, J=11.3, 3.3 Hz, 4H), 4.55 (d, J=8.4 Hz, 4H), 4.05-3.99 (m, 13H), 3.91-3.84 (m, 12H), 3.82-3.74 (m, 4H), 3.73-3.65 (m, 8H), 3.61-3.47 (m, 14H), 3.44-3.41 (m, 8H), 3.29-3.24 (m, 8H), 3.21-3.16 (m, 2H), 2.23 (d, J=8.9 Hz, 2H), 2.10 (s, 12H), 1.99 (s, 12H), 1.89 (s, 12H), 1.77 (s, 12H), 1.61-1.49 (m, 4H), 1.40-1.32 (m, 2H), 1.12 (s, 6H).

### 12. Synthesis of TEC08A (a Stereoisomeric Alcohol with Peracetylated Glycosyl Groups of Ligand TEC08) and TEC12A (a Stereoisomeric Carboxylic Acid with Peracetylated Glycosyl Groups of Ligand TEC12)

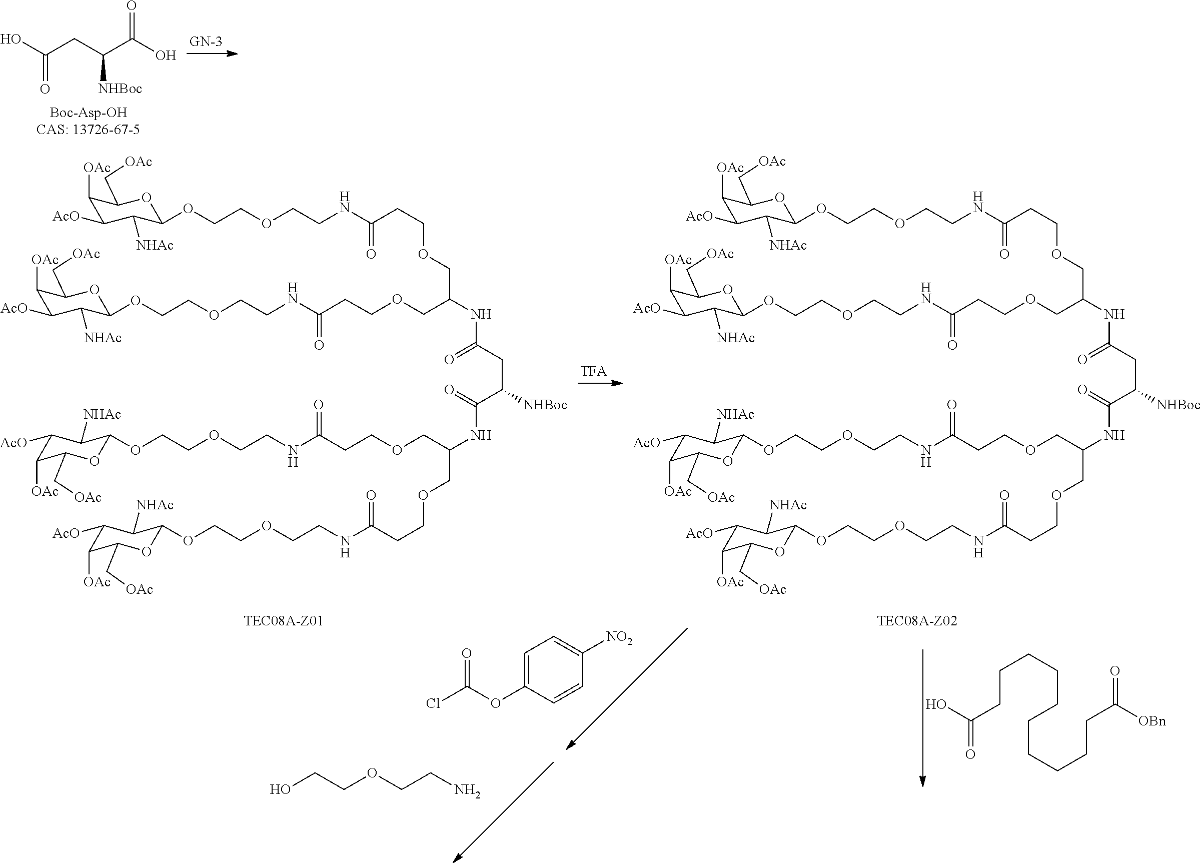

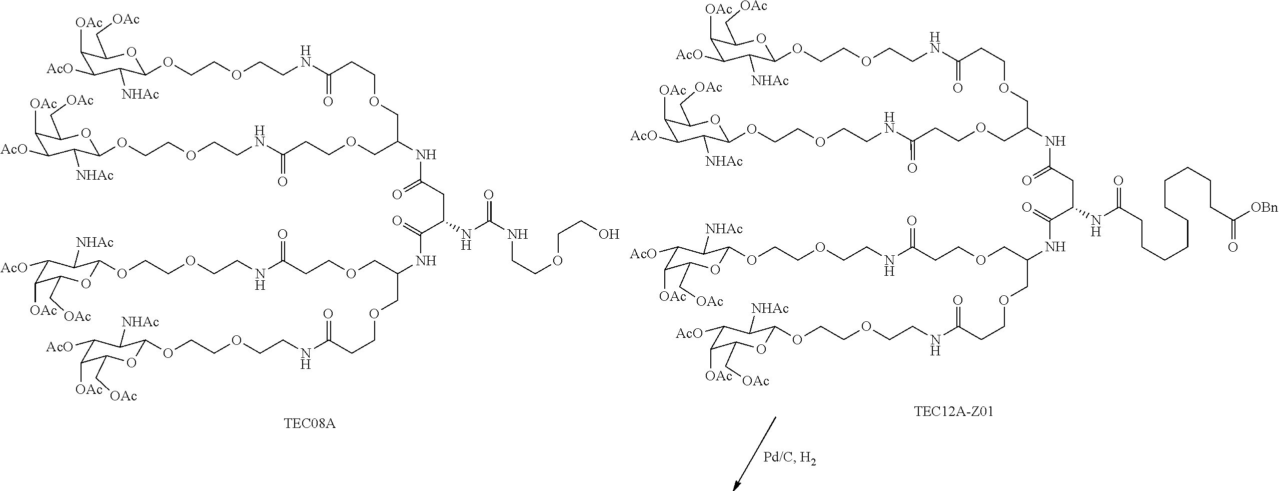

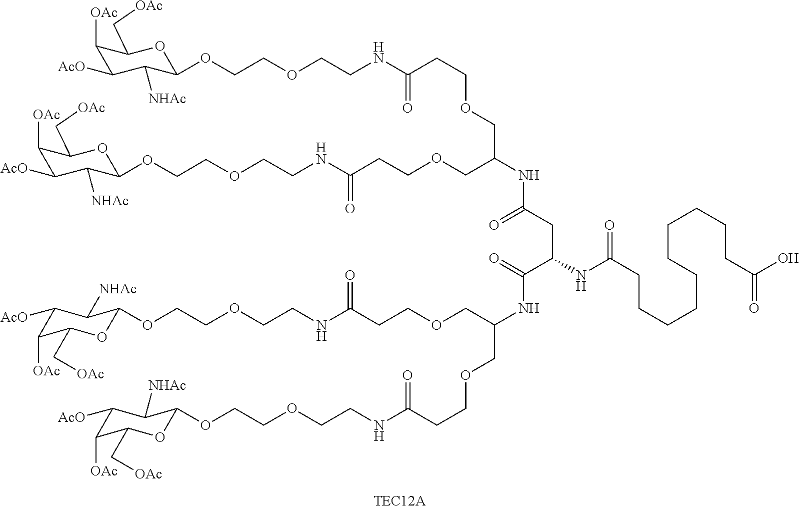 [SMILES: CC(=O)NC1C(OCCOCCNC(=O)CCOCC(COCCC(=O)NCCOCCOC2OC(COC(C)=O)C(OC(C)=O)C(OC(C)=O)C2NC(C)=O)NC(=O)C[C@H](NC(=O)CCCCCCCCCCC(=O)O)C(=O)NC(COCCC(=O)NCCOCCOC2OC(COC(C)=O)C(OC(C)=O)C(OC(C)=O)C2NC(C)=O)COCCC(=O)NCCOCCOC2OC(COC(C)=O)C(OC(C)=O)C(OC(C)=O)C2NC(C)=O)OC(COC(C)=O)C(OC(C)=O)C1OC(C)=O]

### (1) Synthesis of TEC08A-Z01 (Amidation Condensation Reaction)

Under N₂ atmosphere, GN-3 (5.54 g, 5.2 mmol) and Boc-L-aspartic acid (CAS: 13726-67-5, 0.55 g, 2.36 mmol), EDCI (1.13 g, 5.9 mmol) and HOBt (0.57 g, 4.25 mmol) were successively added to a flask containing dichloromethane (30 mL) and the reaction was carried out under stirring at room temperature until the reaction mixture became clear. The reaction was then added with DIEA (1.22 g, 9.44 mmol) and then carried out under stirring for 8 h when LCMS showed the reaction was complete. The reaction solution was added with pure water (30 mL) and extracted with dichloromethane (20 mL×3). The obtained organic phases were combined, washed with 26% aqueous sodium chloride solution (30 mL), dried over anhydrous sodium sulfate, filtered and concentrated under reduced pressure. The crude product was purified by silica gel column chromatography (200-300 meshes, dichloromethane/methanol, 25/1) to obtain 3.37 g of a white solid with a yield of 64%.

MS (ESI): m/z [1/2M+H]⁺, theoretical 1167.5, found 1167.2.

### (2) Synthesis of TEC08A-Z02 (De-Tert-Butoxycarbonylation Reaction)

TEC08A-Z01 (3.1 g, 1.33 mmol) was added to a flask and dissolved with dichloromethane (10 mL), and under stirring at room temperature, trifluoroacetic acid (2 mL) was added slowly, and the reaction was carried out for 0.5 h when LCMS showed that the reaction was completed. The reaction solution was subjected to distillation under reduced pressure to remove the solvent and dried under vacuum to obtain 3.1 g of a light yellow solid product (TFA salt) with a yield of 99%.

MS (ESI): m/z [1/2M+H]⁺, theoretical 1117.5, found 1117.6.

### (3) Synthesis of TEC08A (Urea Condensation Reaction)

Under N₂ atmosphere, TEC08A-Z02 (a TFA salt, 0.37 g, 0.158 mmol) and triethylamine (0.032 g, 0.32 mmol) were successively added to a flask containing dichloromethane (8 mL), followed by slow addition of 4-nitrophenyl chloroformate (0.042 g, 0.21 mmol). The reaction was carried out for 1 h, then added with pure water (8 mL) and extracted with dichloromethane (8 mL×3). The obtained organic phases were combined, washed with cooled 0.5 N hydrochloric acid (5 mL) and 26% aqueous sodium chloride solution (8 mL), dried over anhydrous sodium sulfate, filtered and concentrated under reduced pressure to obtain a light yellow solid crude product, which is a carbamate intermediate. The intermediate was dissolved in N,N-dimethylformamide (6 mL), and under stirring, diglycolamine (0.02 g, 0.19 mmol) was added, and the reaction was carried out at room temperature for 6 h when LCMS showed that the reaction was complete. The reaction solution was added with pure water (15 mL) and extracted with dichloromethane (10 mL×3). The obtained organic phases were combined, washed with 26% aqueous sodium chloride solution (15 mL×2), dried over anhydrous sodium sulfate, filtered and concentrated under reduced pressure. The crude product was crystallized and purified with methyl tert-butyl ether to obtain 0.11 g of an off-white solid product with a yield of 54%.

MS (ESI): m/z [1/2M+H]⁺, theoretical 1183.0, found 1183.2.

¹H NMR (400 MHz, DMSO-d₆) δ 7.89 (t, J=5.7 Hz, 4H), 7.86 (d, J=8.2 Hz, 1H), 7.82 (d, J=9.2 Hz, 4H), 7.53 (d, J=7.8 Hz, 1H), 7.20 (d, J=7.1 Hz, 1H), 7.10 (t, J=4.6 Hz, 1H), 5.20 (d, J=3.4 Hz, 4H), 4.99-4.93 (m, 4H), 4.62-4.54 (m, 1H), 4.53 (d, J=8.5 Hz, 4H), 4.36-4.30 (m, 1H), 4.01 (s, 12H), 3.91-3.82 (m, 4H), 3.80-3.73 (m, 4H), 3.72-3.62 (m, 2H), 3.61-3.51 (m, 15H), 3.52-3.41 (m, 10H), 3.39-3.34 (m, 8H), 3.34-3.29 (m, 12H), 3.21-3.12 (m, 8H), 3.11-3.06 (m, 2H), 2.56 (d, J=6.9 Hz, 2H), 2.29 (t, J=6.5 Hz, 8H), 2.11-2.07 (m, 12H), 2.00-1.96 (m, 12H), 1.90-1.86 (m, 12H), 1.76 (s, 12H).

### (4) Synthesis of TEC12A-Z01 (Amidation Condensation Reaction)

TEC08A-Z02 (a TFA salt, 0.52 g, 0.22 mmol), monobenzyl dodecanediate (0.077 g, 0.24 mmol), EDCI (0.059 g, 0.31 mmol) and HOBt (0.03 g, 0.22 mmol) were successively added to a flask containing dichloromethane (8 mL), and under stirring and at room temperature, DIEA (0.067 g, 0.66 mmol) was added, and the reaction was carried out under stirring for 8 h when LCMS showed that the reaction was completed. The reaction solution was added with pure water (10 mL) and extracted with dichloromethane (10 mL×3). The obtained organic phases were combined, washed with 26% aqueous sodium chloride solution (15 mL), dried over anhydrous sodium sulfate, filtered and concentrated under reduced pressure. The crude product was purified by silica gel column chromatography (200-300 meshes, dichloromethane/methanol, 10/1) to obtain 0.435 g of a white solid product with a yield of 78%.

MS (ESI): m/z [1/2M+Na]⁺, theoretical 1290.6, found 1290.9.

### (5) Synthesis of TEC12A (Debenzylation Reaction)

TEC12A-Z01 (305 mg, 0.12 mmol) was dissolved in tetrahydrofuran (8 mL) and added with Pd/C (30 mg, 10 wt %). The reaction system was replaced with hydrogen for three times and the reaction was carried out under stirring and under hydrogen atmosphere (about 10-20 kPa higher than the standard atmospheric pressure) for 2 h when LCMS showed that the reaction was complete. The reaction mixture was filtered to remove the solid, concentrated under reduced pressure and dried under vacuum to obtain 270 mg of a white solid product with a yield of 92%.

MS (ESI): m/z [1/2M+H]⁺, theoretical 1223.5, found 1223.6.

¹H NMR (400 MHz, DMSO-d₆) δ 8.54 (d, J=7.9 Hz, 1H), 7.87 (t, J=5.6 Hz, 4H), 7.80 (d, J=8.6 Hz, 4H), 7.58-7.51 (m, 2H), 5.18 (d, J=3.5 Hz, 4H), 4.97-4.91 (m, 4H), 4.51 (d, J=8.4 Hz, 4H), 4.50-4.41 (m, 1H), 3.99 (s, 12H), 3.89-3.80 (m, 4H), 3.78-3.71 (m, 4H), 3.70-3.60 (m, 2H), 3.58-3.54 (m, 4H), 3.54-3.50 (m, 8H), 3.48-3.40 (m, 7H), 3.37-3.32 (m, 8H), 3.32-3.27 (m, 12H), 3.19-3.10 (m, 8H), 2.58 (dd, J=7.2, 1.8 Hz, 2H), 2.27 (t, J=6.6 Hz, 8H), 2.23 (t, J=8.3 Hz, 2H), 2.20-2.14 (m, 2H), 2.09-2.05 (m, 12H), 1.98-1.94 (m, 12H), 1.88-1.84 (m, 12H), 1.74 (s, 12H), 1.59-1.47 (m, 4H), 1.29-1.19 (m, 12H).

### 13. Synthesis of TEC09A (an Alcohol Compound with Peracetylated Glycosyl Groups of Ligand TEC09) and TEC13A (a Carboxylic Acid Compound with Peracetylated Glycosyl Groups of Ligand TEC13)

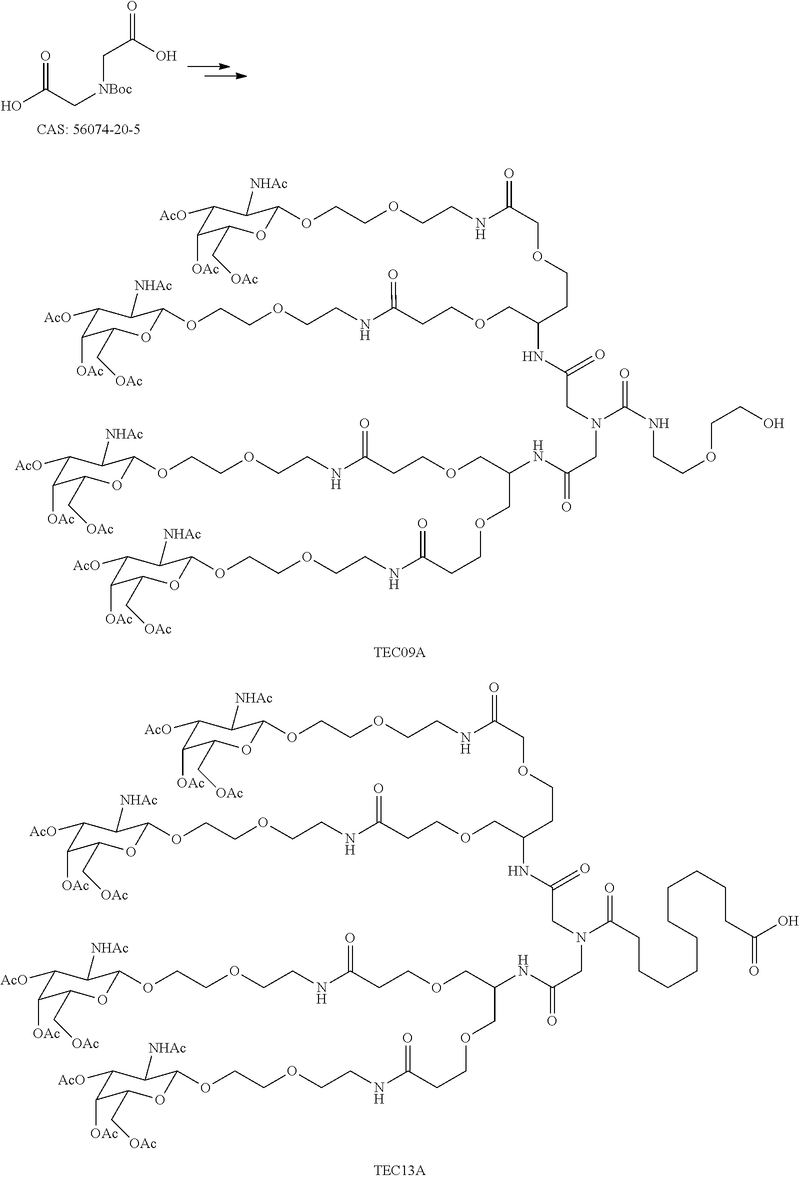

TEC09A and TEC13A were synthesized by the same steps for synthesis of TEC08A and TEC12A, except that Boc-L-aspartic acid (CAS: 13726-67-5) was replaced with N-Boc-iminodiacetic acid (CAS: 56074-20-5).

TEC09A, MS (ESI): m/z [1/2M+Na]⁺, theoretical 1205.0, found 1205.3.

TEC13A, MS (ESI): m/z [1/2M+H]⁺, theoretical 1223.5, found 1223.5.

### 14. Synthesis of TEC10A (a Carboxylic Acid Compound with Peracetylated Glycosyl Groups of Ligand TEC10)

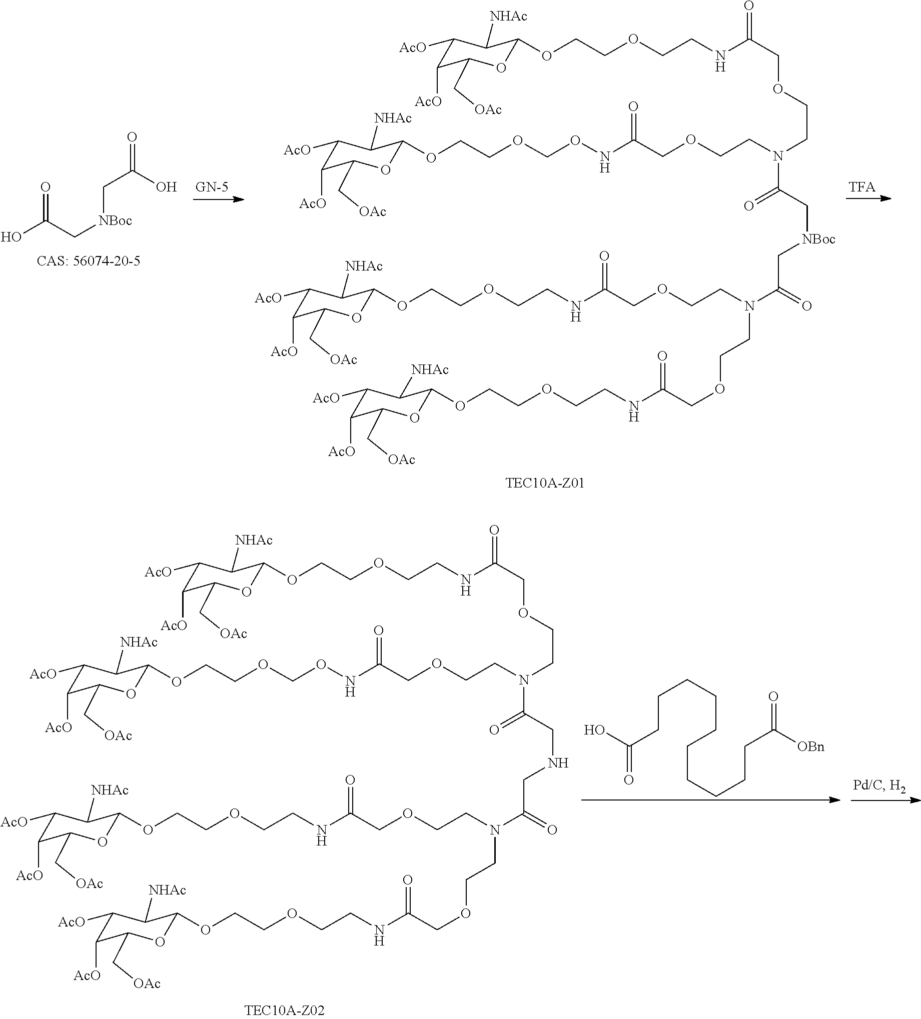

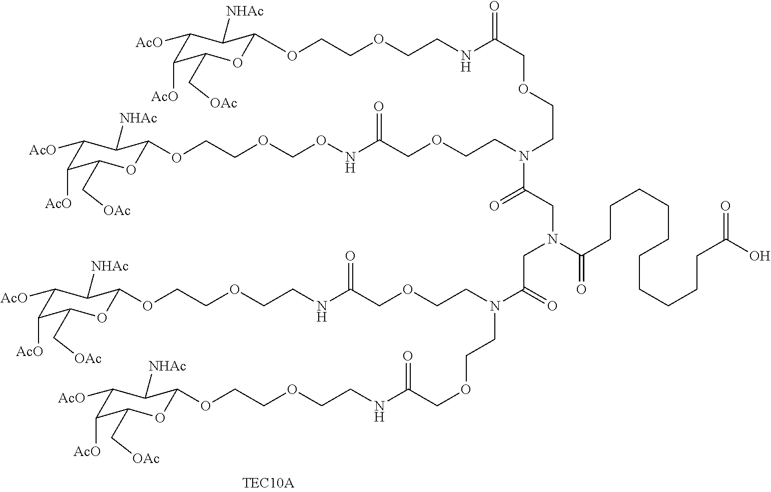 [SMILES: CC(=O)NC1C(OCCOCCNC(=O)COCCN(CCOCC(=O)NCCOCCOC2OC(COC(C)=O)C(OC(C)=O)C(OC(C)=O)C2NC(C)=O)C(=O)CN(CC(=O)N(CCOCC(=O)NCCOCCOC2OC(COC(C)=O)C(OC(C)=O)C(OC(C)=O)C2NC(C)=O)CCOCC(=O)NOCOCCOC2OC(COC(C)=O)C(OC(C)=O)C(OC(C)=O)C2NC(C)=O)C(=O)CCCCCCCCCCC(=O)O)OC(COC(C)=O)C(OC(C)=O)C1OC(C)=O]

TEC10A was synthesized by the same steps for synthesis of TEC13, except that GN-3 was replaced with GN-5.

MS (ESI): m/z [1/2M+H]⁺, theoretical 1209.5, found 1209.8.

¹H NMR (400 MHz, DMSO-d₆) δ 7.88 (t, J=5.7 Hz, 4H), 7.81 (d, J=9.2 Hz, 4H), 5.18 (d, J=3.4 Hz, 4H), 4.97-4.91 (m, 4H), 4.51 (d, J=8.5 Hz, 4H), 4.03 (s, 4H), 4.00 (s, 12H), 3.99-3.94 (m, 4H), 3.90-3.78 (m, 8H), 3.79-3.71 (m, 4H), 3.71-3.54 (m, 14H), 3.54-3.50 (m, 6H), 3.39-3.29 (m, 9H), 3.27-3.21 (m, 12H), 3.20-3.10 (m, 8H), 2.32-2.03 (m, 4H), 2.07 (s, 12H), 1.96 (s, 12H), 1.86 (s, 12H), 1.74 (s, 12H), 1.60-1.43 (m, 4H), 1.27-1.17 (m, 12H).

### 15. Synthesis of TEC11A (an Alcohol Compound with Peracetylated Glycosyl Groups of Ligand TEC11)

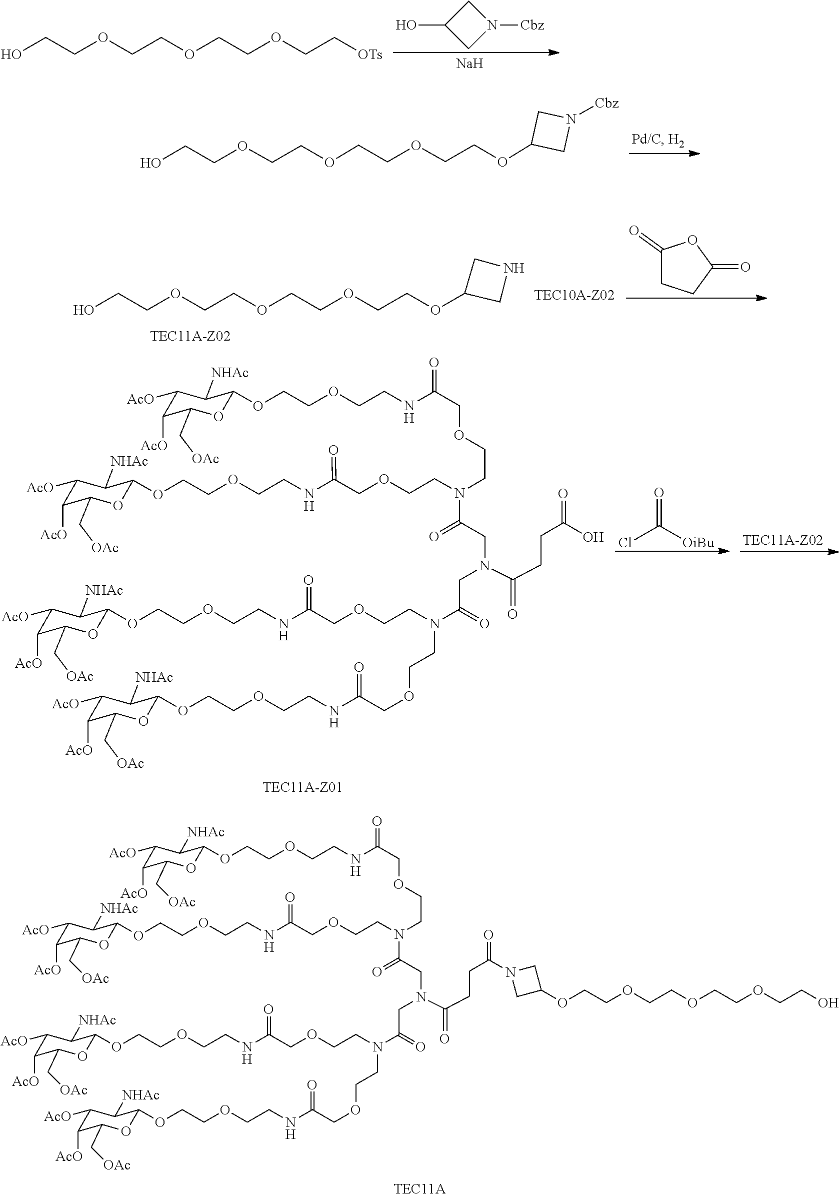

### (1) Synthesis of TEC11A-Z01 (Amidation Condensation Reaction)

Under N₂ atmosphere, dichloromethane (5 mL), TC10A-Z02 (112 mg, 0.0508 mmol) and triethylamine (15 mg, 0.152 mmol) were successively added to a flask, and under constant stirring, succinic anhydride (10 mg, 0.1 mmol) was added, and then the reaction was carried out for 1 h when LCMS showed that the conversion of the raw materials was complete. The reaction solution was subjected to distillation under reduced pressure to remove the solvent, and the residue was dissolved in dichloromethane (12 mL), washed with 8% aqueous sodium bicarbonate solution (5 mL×2), dried over anhydrous sodium sulfate, filtered, concentrated under reduced pressure, and crystallized and purified with methyl tert-butyl ether to obtain 72.6 mg of a white solid product with a yield of 62%.

MS (ESI): m/z [1/2M+H]⁺, theoretical 1153.5, found 1153.3.

### (2) Synthesis of TEC11A-Z02 (De-Benzyloxycarbonylation Reaction)

Under N₂ atmosphere, 1-Cbz-3-hydroxyazetidine (0.9 g, 4.34 mmol) was added to a flask containing tetrahydrofuran (10 mL), added with sodium hydride (60%, 165 mg, 4.12 mmol) in batches and stirred at room temperature for 0.5 h. The obtained mixture was slowly added to tetraethylene glycol monotosylate (1.43 g, 4.12 mmol) in tetrahydrofuran (12 mL) and the reaction was then carried out under stirring for 8 h when LCMS showed that the reaction was complete. The reaction was added with 25% aqueous ammonium chloride solution (30 mL) and extracted with ethyl acetate (15 mL×3). The obtained organic phases were combined, washed with 26% aqueous sodium chloride solution (25 mL), dried over anhydrous sodium sulfate and concentrated to obtain a crude product. The crude product was purified by silica gel column chromatography (200-300 meshes, petroleum ether/ethyl acetate, 4/1) to obtain a colorless transparent liquid. The obtained compound was dissolved in tetrahydrofuran (6 mL) and added with Pd/C (40 mg, 10 wt %). The reaction system was replaced with hydrogen for three times and the reaction was carried out under stirring and under hydrogen atmosphere (about 10-20 kPa higher than the standard atmospheric pressure) for 2 h when LCMS showed that the reaction was complete. The mixture was filtered to remove the solid, concentrated under reduced pressure and dried under vacuum to obtain 257 mg of a white solid product with a yield of 25%.

MS (ESI): m/z [M+H]⁺, theoretical 250.2, found 250.2.

### (3) Synthesis of TEC11A (Amidation Condensation Reaction)

Under N₂ atmosphere, TEC11A-Z01 (62 mg, 0.027 mmol) and N-methylmorpholine (8 mg, 0.081 mmol) were successively added to a flask containing dichloromethane (7 mL), cooled in an ice bath to below 5° C., and added with isobutyl chloroformate (7 mg, 0.054 mmol), and the reaction was carried out under stirring for 1 h. The reaction solution was then concentrated under reduced pressure and dried under vacuum to obtain a light yellow solid. The solid was added with dichloromethane (7 mL) again, and under stirring, TC11A-Z02 (10 mg, 0.04 mmol) was added, and the reaction was carried out for 2 h when LCMS showed that the reaction was completed. The reaction solution was concentrated under reduced pressure, and the residue was crystallized and purified with ethyl acetate and methyl tert-butyl ether to obtain 36 mg of a light yellow solid product with a yield of 52%.

MS (ESI): m/z [1/3M+H]⁺, theoretical 846.4, found 846.5.

¹H NMR (400 MHz, DMSO-d₆) δ 7.92 (t, J=5.5 Hz, 4H), 7.84 (d, J=8.9 Hz, 4H), 5.20 (d, J=3.6 Hz, 4H), 4.98-4.93 (m, 4H), 4.52 (d, J=8.7 Hz, 4H), 4.10-4.05 (m, 1H), 4.02 (s, 4H), 3.99 (s, 12H), 3.96-3.92 (m, 4H), 3.91-3.79 (m, 10H), 3.77-3.72 (m, 6H), 3.70-3.55 (m, 14H), 3.54-3.49 (m, 10H), 3.47-3.28 (m, 21H), 3.28-3.19 (m, 12H), 3.20-3.09 (m, 8H), 2.72-2.63 (m, 2H), 2.52-2.48 (m, 2H), 2.09 (s, 12H), 1.97 (s, 12H), 1.88 (s, 12H), 1.75 (s, 12H).

### Example 3. Ligand-Nucleic Acid Conjugation

Generally, a ligand compound has saccharide hydroxyl groups all protected by acetyl (Ac) groups and can be linked to a nucleic acid by chemical synthesis. The nucleic acid can be prepared by biological methods such as biological extraction, in vitro transcription, etc., and chemical solid phase synthesis technology. For the solid phase synthesis of nucleic acid, see the phosphoramidite coupling technique reported in J. Org. Chem., 2012, 77, 4566-4577 and Curr. Protoc. Nucleic Acid Chem., 2020, 81, e107.

The phosphoramidite-modified ligand compound can also be linked to a nucleic acid by the above solid phase synthesis. A carboxylic acid- and activated ester-modified ligand compound can be linked via an amide bond to an amino-modified nucleic acid by amidation condensation reaction (Bioorg. Med. Chem. Lett., 2004, 14, 801-804; ChemBioChem, 2019, 20, 1599-1605). The ligand of the present disclosure can also be modified with a maleimide group, and then subjected to Michael addition reaction with a sulfydryl-modified nucleic acid to form a carbon-sulfur bond (C—S) (Bioconjugate Chem. 2012, 23, 300-307; Chem. Commun., 2013, 49, 309-311). Alternatively, the ligand is modified with a pyridyldithiol group, and then linked to a sulfydryl-modified nucleic acid to form a disulfide bond (S—S) (Curr. Protoc. Nucleic Acid Chem., 2006, 4.28.1-4.28.21).

### Example 4. Biological Activity Test of the Ligand (on Primary Liver Cells)

The binding rate of the ligand of the present disclosure to the ASGPR receptor of liver cancer cells was tested, with the ligand with hepatocellular ASGPR-targeting property, loaded in Inclisiran, a small interfering nucleic acid drug marketed by Alnylam, as a positive control (PC).

The ligand-receptor dissociation constant (K_{d}, which is the drug concentration required for half of the maximum effect, i.e. 50% of the receptors are occupied) was used as a measure of ligand-receptor binding capacity; and the lower the K_{d} value, the higher the binding capacity.

### Test Compounds:

Test compounds: PC-Cy5 (positive control), TC01A-Cy5, TC07A-Cy5, TC09A-Cy5, TC10A-Cy5, TC12A-Cy5, TC17A-Cy5, TC18A-Cy5, TEC02A-Cy5, TEC06A-Cy5, TEC14A-Cy5

### Experimental Protocol:

C57 mouse primary liver cells were isolated, and the cells were in a good state, had viability of 92%, and were at a density of 2×10⁵ cells/400 μL.

The test compounds were dissolved with PBS and then diluted with complete medium for primary liver cells to various concentration gradients.

The cells was added with 100 μL of the test compound solution prepared according to the set concentration gradients (the test compounds was first prepared with PBS into a stock solution, which was then diluted to the set concentration gradients with complete medium) and incubated at 37° C. in the dark for 30 min.

After incubation, the cells were washed once with 500 μL of PBS.

The cells were added with typsin to remove fluorescence on the cell surface.

The cells were washed again with 500 μL of PBS and detected on machine.

Experimental results are shown in the following table:

| Test compounds | K_{d} (nM) |
|---|---|
| PC-Cy5 | 0.4081 |
| TC01A-Cy5 | 0.9078 |
| TC07A-Cy5 | 0.7636 |
| TC09A-Cy5 | 0.8306 |
| TC10A-Cy5 | 0.8669 |
| TC12A-Cy5 | 0.6960 |
| TC17A-Cy5 | 1.264 |
| TC18A-Cy5 | 0.6312 |
| TEC02A-Cy5 | 0.1982 |
| TEC06A-Cy5 | 0.4962 |
| TEC14A-Cy5 | 0.3332 |

The experimental results show that the ligand compounds of the present disclosure had a hepatocellular ASGPR receptor-binding capacity that reached the same order of magnitude as that of the positive control, indicating that they have good liver-targeting properties.

### Example 5. Biological Activity Test of the Ligands (on HepG2 Cell Line)

The binding rate of the ligand of the present disclosure to the ASGPR receptor of liver cancer cells was tested, with the ligand with hepatocellular ASGPR-targeting property, loaded in Inclisiran, a small interfering nucleic acid drug marketed by Alnylam, as a positive control (PC).

The ligand-receptor dissociation constant (K_{d}, which is the drug concentration required for half of the maximum effect, i.e. 50% of the receptors are occupied) was used as a measure of ligand-receptor binding capacity, and the lower the K_{d} value, the higher the binding capacity.

### Test Compounds:

### Positive Control (PC-Cy5)

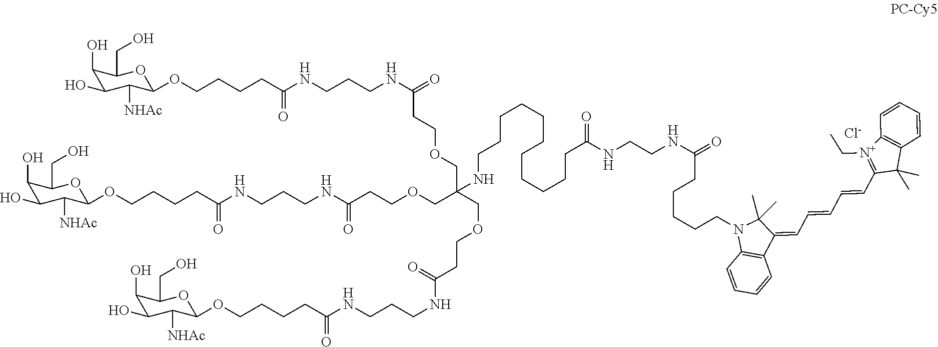 [SMILES: CC[N+]1=C(/C=C/C=C/C=C2/c3ccccc3N(CCCCCC(=O)NCCNC(=O)CCCCCCCCCCCNC(COCCC(=O)NCCCNC(=O)CCCCOC3OC(CO)C(O)C(O)C3NC(C)=O)(COCCC(=O)NCCCNC(=O)CCCCOC3OC(CO)C(O)C(O)C3NC(C)=O)COCCC(=O)NCCCNC(=O)CCCCOC3OC(CO)C(O)C(O)C3NC(C)=O)C2(C)C)C(C)(C)c2ccccc21 ; [Cl-]]

### TEC02A-Cy5

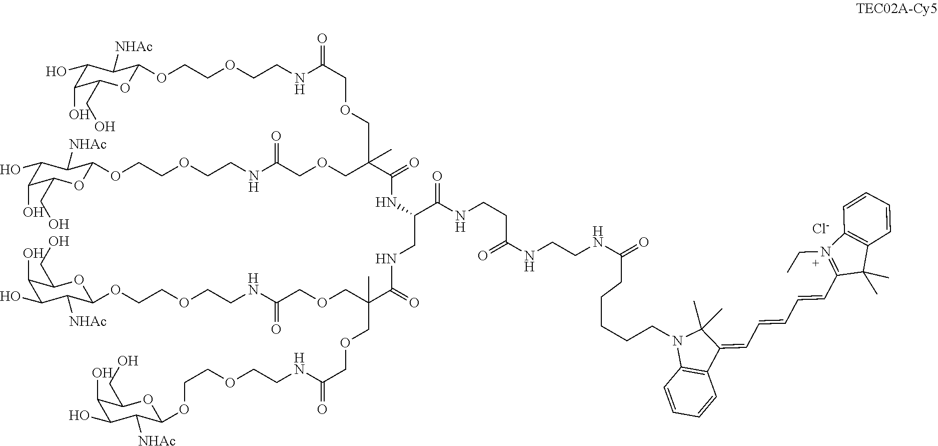

### Experimental Protocol:

1. HepG2 cells grown to confluence of greater than 80% and in a good state were digested, centrifuged and counted.
2. The test compounds were diluted with PBS to various concentration gradients.
3. The cells were washed once with PBS, added with 100 μL of the prepared compounds, and incubated at room temperature in the dark for 30 min.
4. After incubation, the cells were washed twice with 1 mL of PBS and detected on machine.

### Experimental Results:

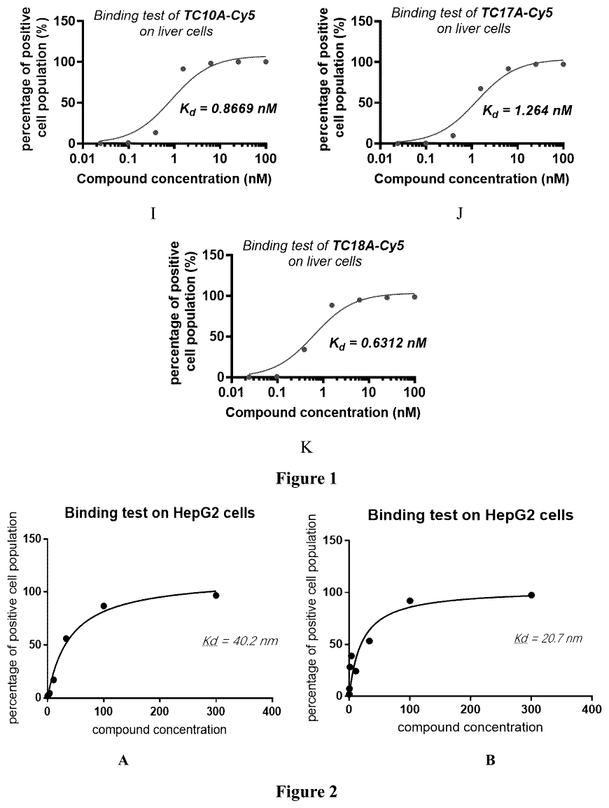

The binding curve of the positive control (PC-Cy5) to the receptor is shown as FIG. 2A, and the binding curve of TEC02A-Cy5 to the receptor is shown as FIG. 2B.

The dissociation constants are shown in the following table:

| Test compounds | K_{d} (nM) |
|---|---|
| PC-Cy5 | 40.2 |
| TEC02A-Cy5 | 20.7 |

The experimental results show that the ligand of the present disclosure (TEC02A) has superior binding capacity to the hepatocellular ASGPR receptor to the positive control, and indicating that it has a good targeting property.

Finally, it should be noted that the above examples are only used to assist those skilled in the art to understand the essence of the present disclosure, and are not intended to limit the protection scope of the present disclosure.

1. A ligand having a structure of Formula II: 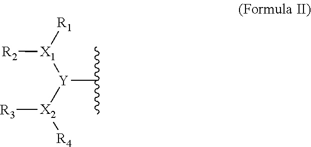
wherein,
R₁, R₂, R₃ and R₄ are each a chain structure with a terminal glycosyl group, and
X, X₁, X₂ and Y are each a backbone structure in a triple-branched form for linking other chemical groups
X₁ has a structure of 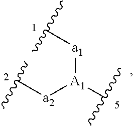
X₂ has a structure of 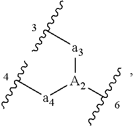
Y has a structure of 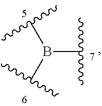 [SMILES: *B(*)*]
X₁ is linked to Y at residue position 5, X₂ is linked to Y at residue position 6, and A₁, A₂ and B are each a backbone structure having a ring, chain or branch and in a triple-branched form for linking other chemical groups;
a₁, a₂, a₃ and a₄ may be same or different or absent;
where a₁, a₂, a₃ and a₄ are present, a₁ to a₄ are selected from O, S and NQ₁; wherein,
Q₁ is selected from H, methyl, ethyl, n-propyl, isopropyl, formyl, acetyl, n-propionyl, isopropionyl and 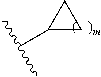
(m is an integer from 1 to 4).

2. The ligand of claim 1, wherein, in the ligand having a structure of Formula I,
R₁ has a structure of 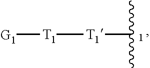
R₂ has a structure of 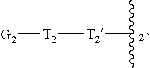
R₃ has a structure of 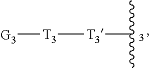
and
R₄ has a structure of 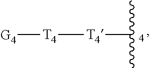
wherein,
T₁ to T₄ and T₁′ to T₄′ are each a structure comprising at least one chain, cyclic or branched chemical moiety, wherein T₁′ to T₄′ may be present or absent;
G₁ to G₄ are each a glycosyl group with or without a protective group on hydroxyl;
wherein G₁ to G₄ are each independently selected from lactosyl, galactosyl, 2-aminogalactosyl, 2-formamidogalactosyl, 2-acetamidogalactosyl and 2-propionamidogalactosyl group, preferably, 2-acetamidogalactosyl group; 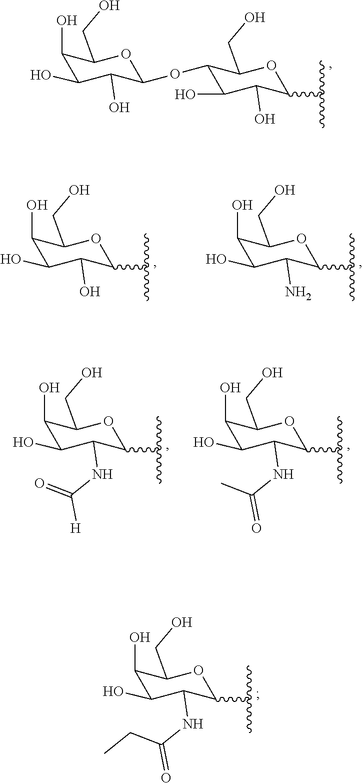
G₁ to G₄ are linked to T₁ to T₄ via an O-glycosidic bond, an S-glycosidic bond or an N-glycosidic bond.

3.-9. (canceled)

10. The ligand of claim 1, wherein, in the ligand having a structure of Formula II, Y has a structure of 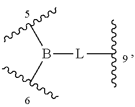
L is linked to B at residue position 7 by directly forming a bond with each other or by a linker (K), and K is selected from 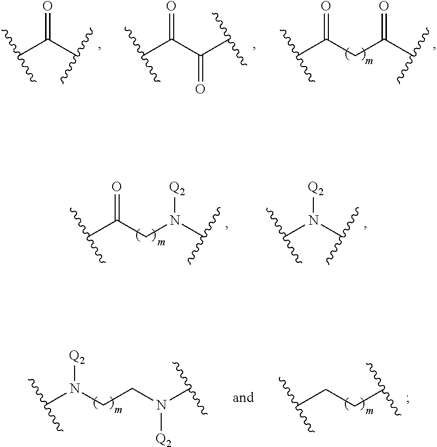
Q₂ is selected from H, methyl, ethyl, n-propyl, isopropyl or 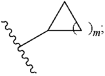
m is an integer from 1 to 4;
L is selected from: M₁; 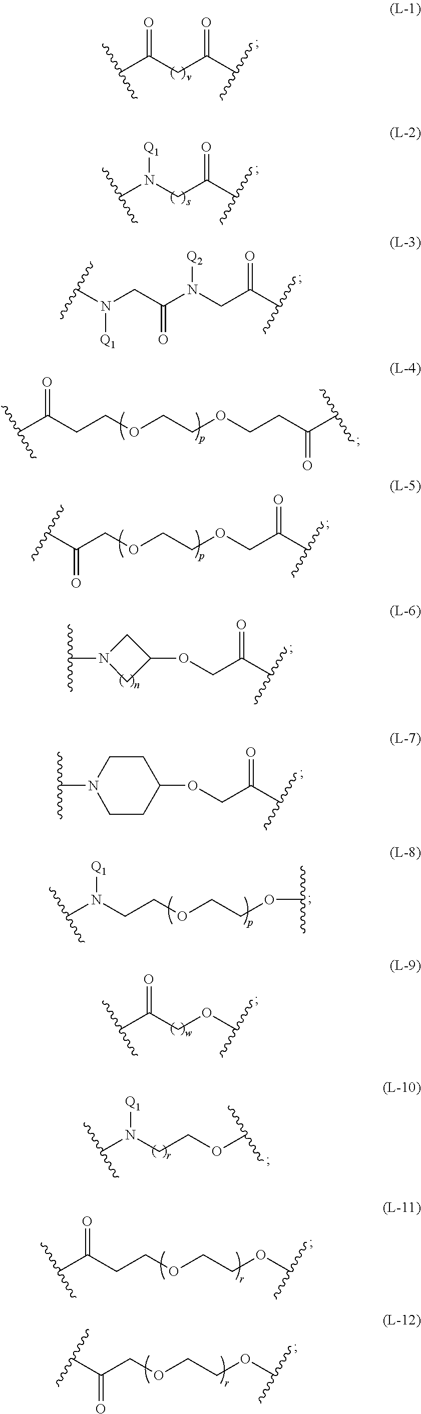 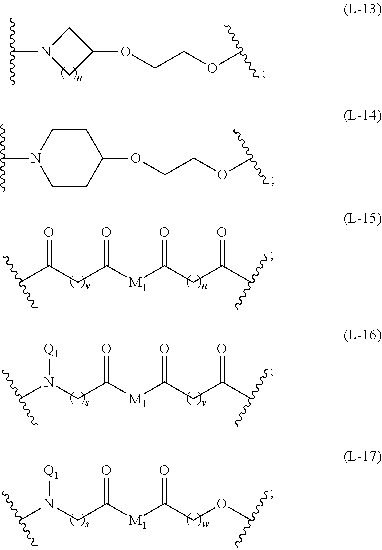
wherein,
M₁ is selected from: 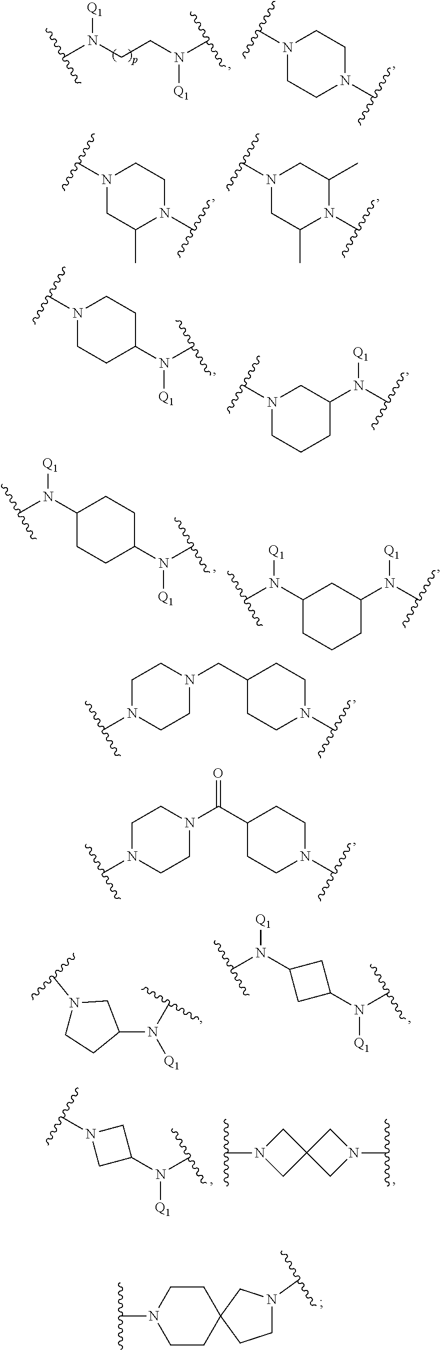
in the above formulas, e and f are each an integer from 1 to 2; n is an integer from 1 to 3; m is an integer from 1 to 4; j is an integer from 1 to 5; p and q are each an integer from 1 to 6; r is an integer from 1 to 5; s and t are each an integer from 1 to 17; v and u are each an integer from 1 to 18; w is an integer from 1 to 19.

11. The ligand of claim 10, wherein, in the ligand having a structure of Formula II,
where a₁, a₂, a₃ and a₄ are all present, A₁ and A₂ are each independently selected from: 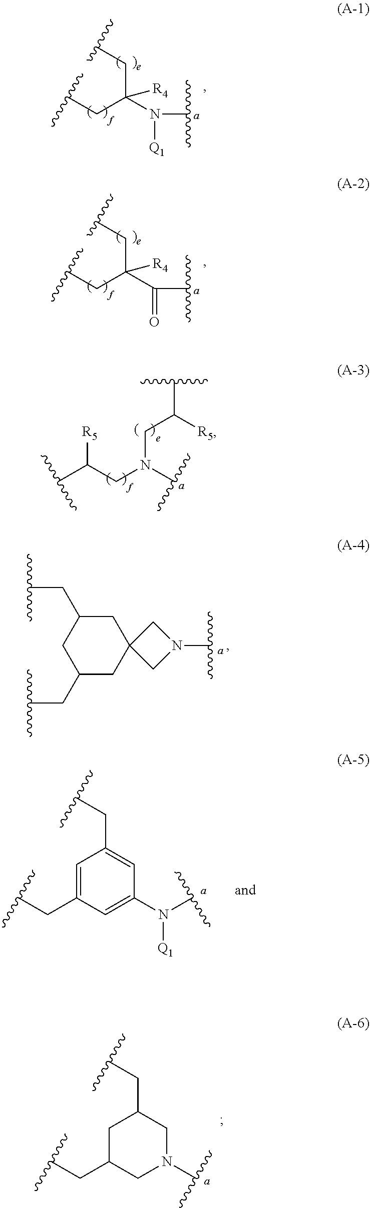
wherein,
R₄ is H, methyl or ethyl;
R₅ is H or methyl;
where a₁-a₄ are all absent, A₁ and A₂ are each independently selected from: 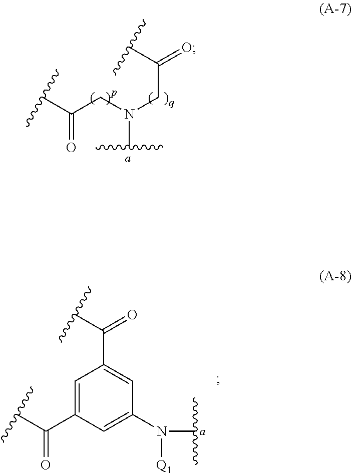
and
a nitrogen atom (A-9);
where a₁ and a₂ are present and a₃ and a₄ are absent, A₁ is selected from (A-1) to (A-6), and A₂ is selected from (A-7) to (A-9);
where a₃ and a₄ are present and a₁ and a₂ are absent, A₁ is selected from (A-7) to (A-9), and A₂ is selected from (A-1) to (A-6);
residue position a corresponds to residue position 5 of X₁ and residue position 6 of X₂.

12. The ligand of claim 1, wherein, in the ligand having a structure of Formula II,
B is selected from: 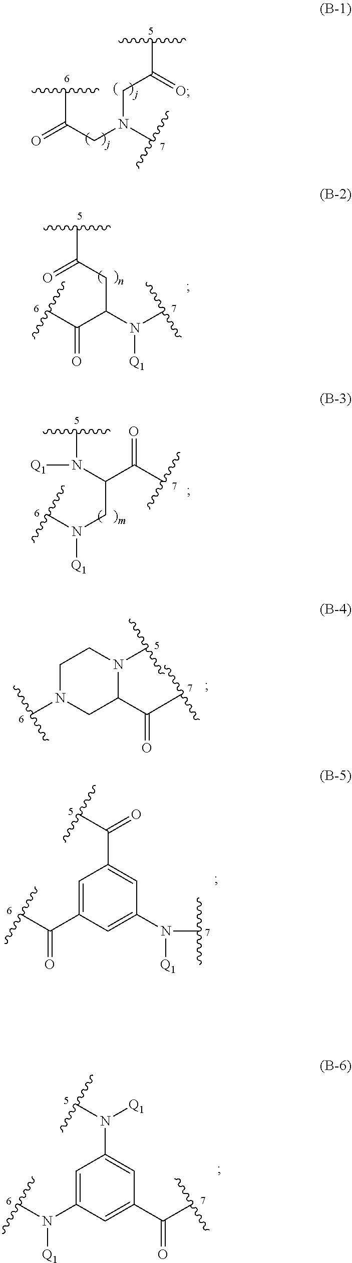
A₁ is linked to B at residue position 5 and A₂ is linked to B at residue position 6, by directly forming a bond with each other or by linker K;
in the above formulas, e and f are each an integer from 1 to 2; n is an integer from 1 to 3; m is an integer from 1 to 4; j is an integer from 1 to 5; p and q are each an integer from 1 to 6; r is an integer from 1 to 5; s and t are each an integer from 1 to 17; v and u are each an integer from 1 to 18; w is an integer from 1 to 19.

13. The ligand of claim 2, wherein, in the ligand having a structure of Formula II,
T₁′ to T₄′ are linked to a₁ to a₄, or A₁ or A₂ at residue positions 1-4, by directly forming a bond with each other or by linker K; T₁′ to T₄′ are independently selected from: 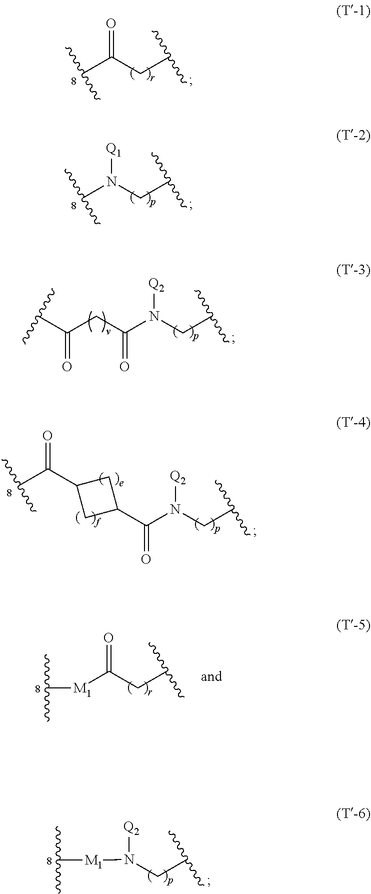
M₂ is selected from: 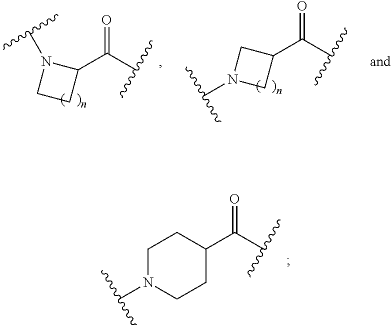
in the above formulas, e and f are each an integer from 1 to 2; n is an integer from 1 to 3; m is an integer from 1 to 4; j is an integer from 1 to 5; p and q are each an integer from 1 to 6; r is an integer from 1 to 5; s and t are each an integer from 1 to 17; v and u are each an integer from 1 to 18; w is an integer from 1 to 19.

14. The ligand of claim 2, wherein, the ligand has any one of the following structures:

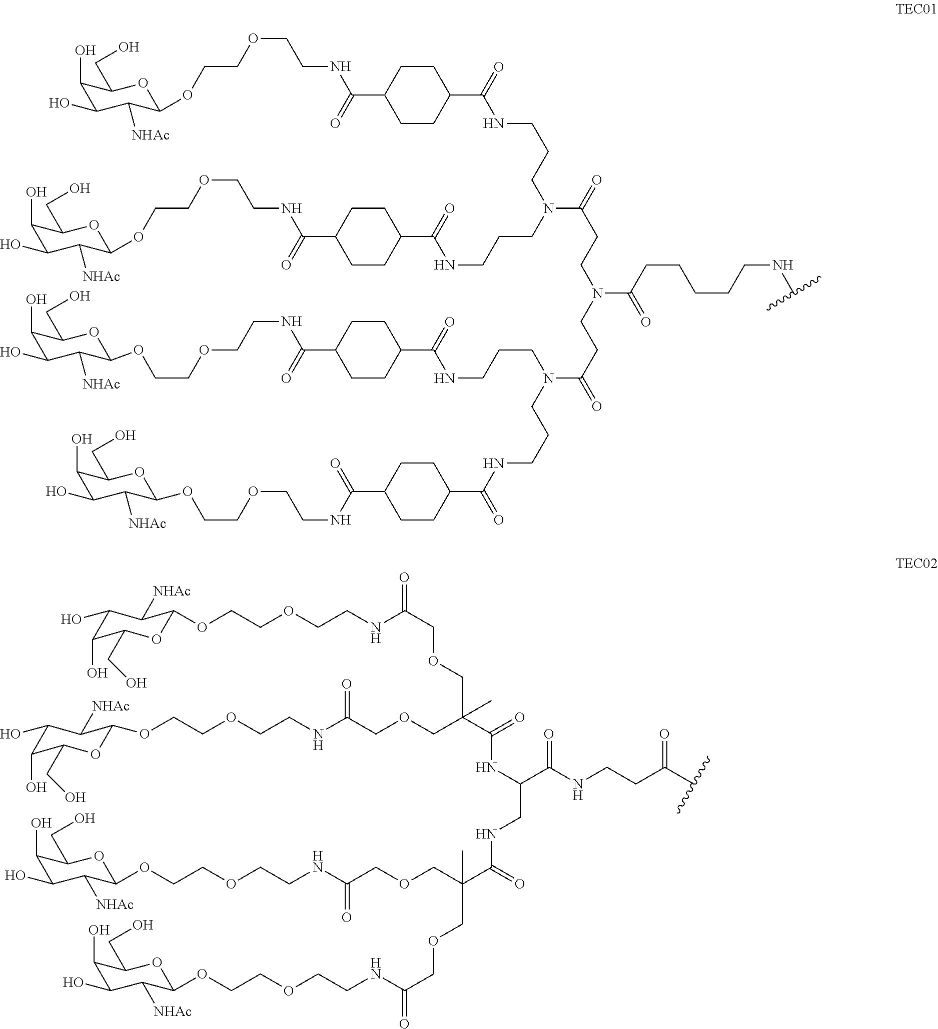 [SMILES: *NCCCCCC(=O)N(CCC(=O)N(CCCNC(=O)C1CCC(C(=O)NCCOCCOC2OC(CO)C(O)C(O)C2NC(C)=O)CC1)CCCNC(=O)C1CCC(C(=O)NCCOCCOC2OC(CO)C(O)C(O)C2NC(C)=O)CC1)CCC(=O)N(CCCNC(=O)C1CCC(C(=O)NCCOCCOC2OC(CO)C(O)C(O)C2NC(C)=O)CC1)CCCNC(=O)C1CCC(C(=O)NCCOCCOC2OC(CO)C(O)C(O)C2NC(C)=O)CC1 ; *C(=O)CCNC(=O)C(CNC(=O)C(C)(COCC(=O)NCCOCCOC1OC(CO)C(O)C(O)C1NC(C)=O)COCC(=O)NCCOCCOC1OC(CO)C(O)C(O)C1NC(C)=O)NC(=O)C(C)(COCC(=O)NCCOCCOC1OC(CO)C(O)C(O)C1NC(C)=O)COCC(=O)NCCOCCOC1OC(CO)C(O)C(O)C1NC(C)=O]

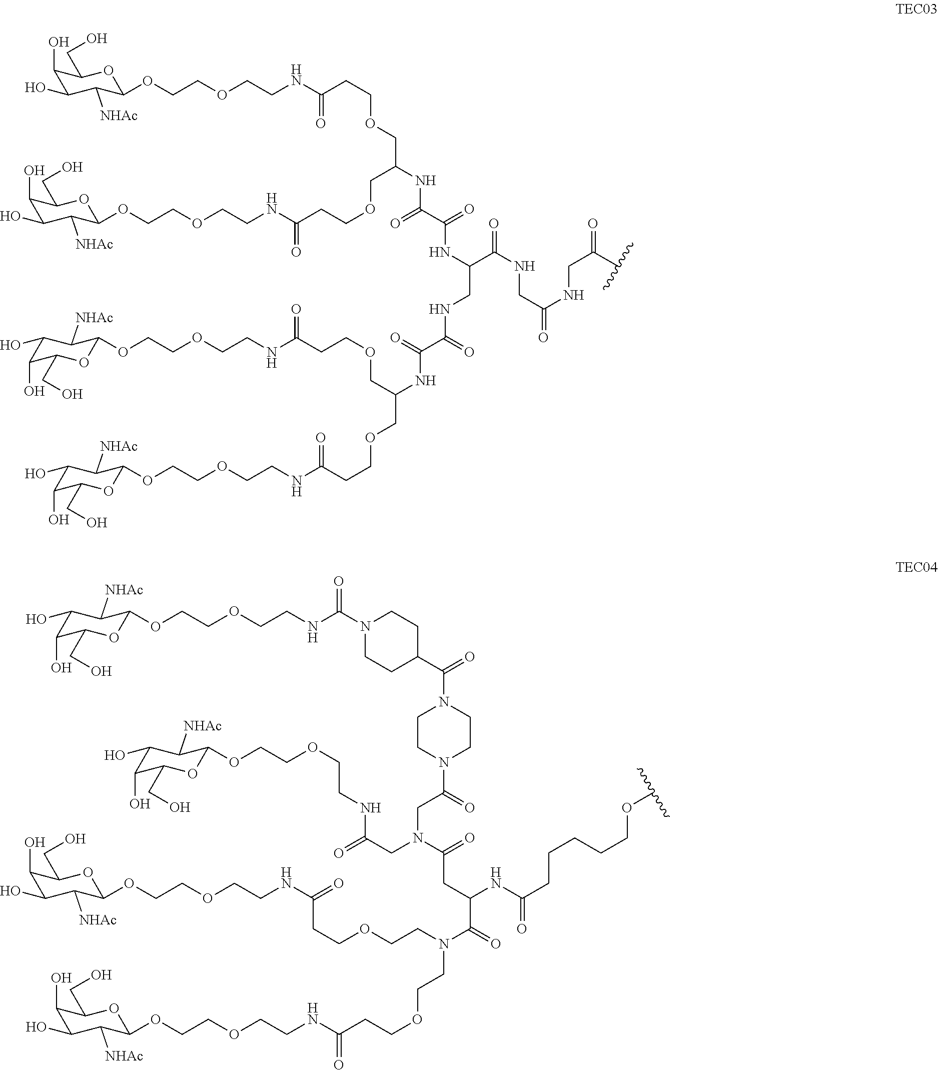 [SMILES: *C(=O)CNC(=O)CNC(=O)C(CNC(=O)C(=O)NC(COCCC(=O)NCCOCCOC1OC(CO)C(O)C(O)C1NC(C)=O)COCCC(=O)NCCOCCOC1OC(CO)C(O)C(O)C1NC(C)=O)NC(=O)C(=O)NC(COCCC(=O)NCCOCCOC1OC(CO)C(O)C(O)C1NC(C)=O)COCCC(=O)NCCOCCOC1OC(CO)C(O)C(O)C1NC(C)=O ; *OCCCCCC(=O)NC(CC(=O)N(CC(=O)NCCOCCOC1OC(CO)C(O)C(O)C1NC(C)=O)CC(=O)N1CCN(C(=O)C2CCN(C(=O)NCCOCCOC3OC(CO)C(O)C(O)C3NC(C)=O)CC2)CC1)C(=O)N(CCOCCC(=O)NCCOCCOC1OC(CO)C(O)C(O)C1NC(C)=O)CCOCCC(=O)NCCOCCOC1OC(CO)C(O)C(O)C1NC(C)=O]

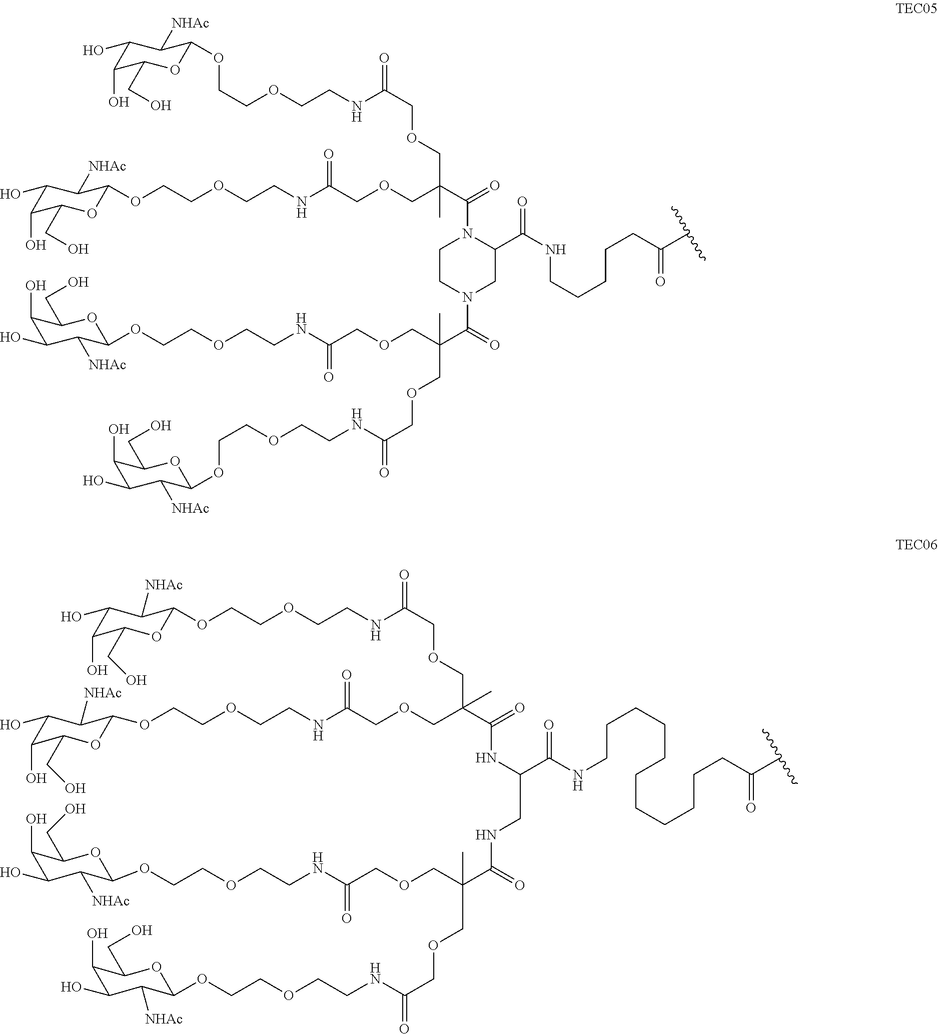 [SMILES: *C(=O)CCCCCNC(=O)C1CN(C(=O)C(C)(COCC(=O)NCCOCCOC2OC(CO)C(O)C(O)C2NC(C)=O)COCC(=O)NCCOCCOC2OC(CO)C(O)C(O)C2NC(C)=O)CCN1C(=O)C(C)(COCC(=O)NCCOCCOC1OC(CO)C(O)C(O)C1NC(C)=O)COCC(=O)NCCOCCOC1OC(CO)C(O)C(O)C1NC(C)=O ; *C(=O)CCCCCCCCCCCNC(=O)C(CNC(=O)C(C)(COCC(=O)NCCOCCOC1OC(CO)C(O)C(O)C1NC(C)=O)COCC(=O)NCCOCCOC1OC(CO)C(O)C(O)C1NC(C)=O)NC(=O)C(C)(COCC(=O)NCCOCCOC1OC(CO)C(O)C(O)C1NC(C)=O)COCC(=O)NCCOCCOC1OC(CO)C(O)C(O)C1NC(C)=O]

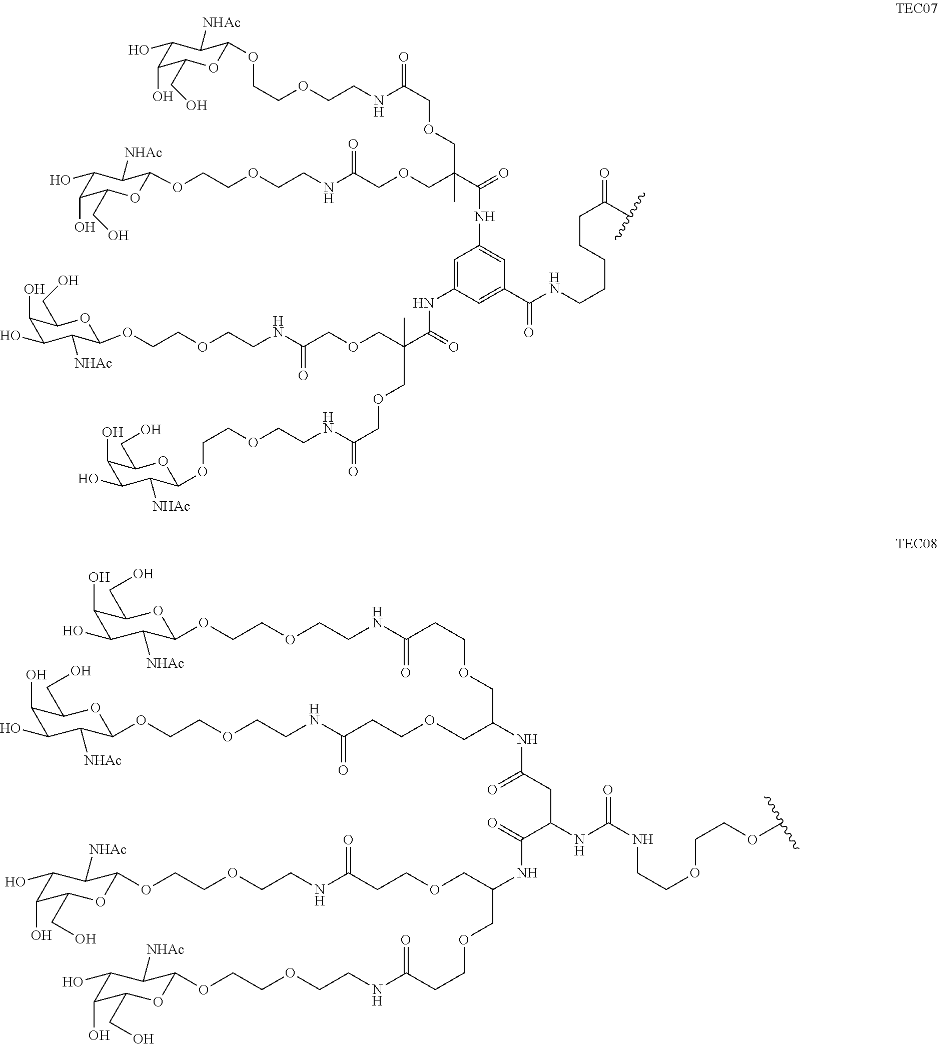 [SMILES: *C(=O)CCCCCNC(=O)c1cc(NC(=O)C(C)(COCC(=O)NCCOCCOC2OC(CO)C(O)C(O)C2NC(C)=O)COCC(=O)NCCOCCOC2OC(CO)C(O)C(O)C2NC(C)=O)cc(NC(=O)C(C)(COCC(=O)NCCOCCOC2OC(CO)C(O)C(O)C2NC(C)=O)COCC(=O)NCCOCCOC2OC(CO)C(O)C(O)C2NC(C)=O)c1 ; *OCCOCCNC(=O)NC(CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O)C(=O)NC(COCCC(=O)NCCOCCOC1OC(CO)C(O)C(O)C1NC(C)=O)COCCC(=O)NCCOCCOC1OC(CO)C(O)C(O)C1NC(C)=O]

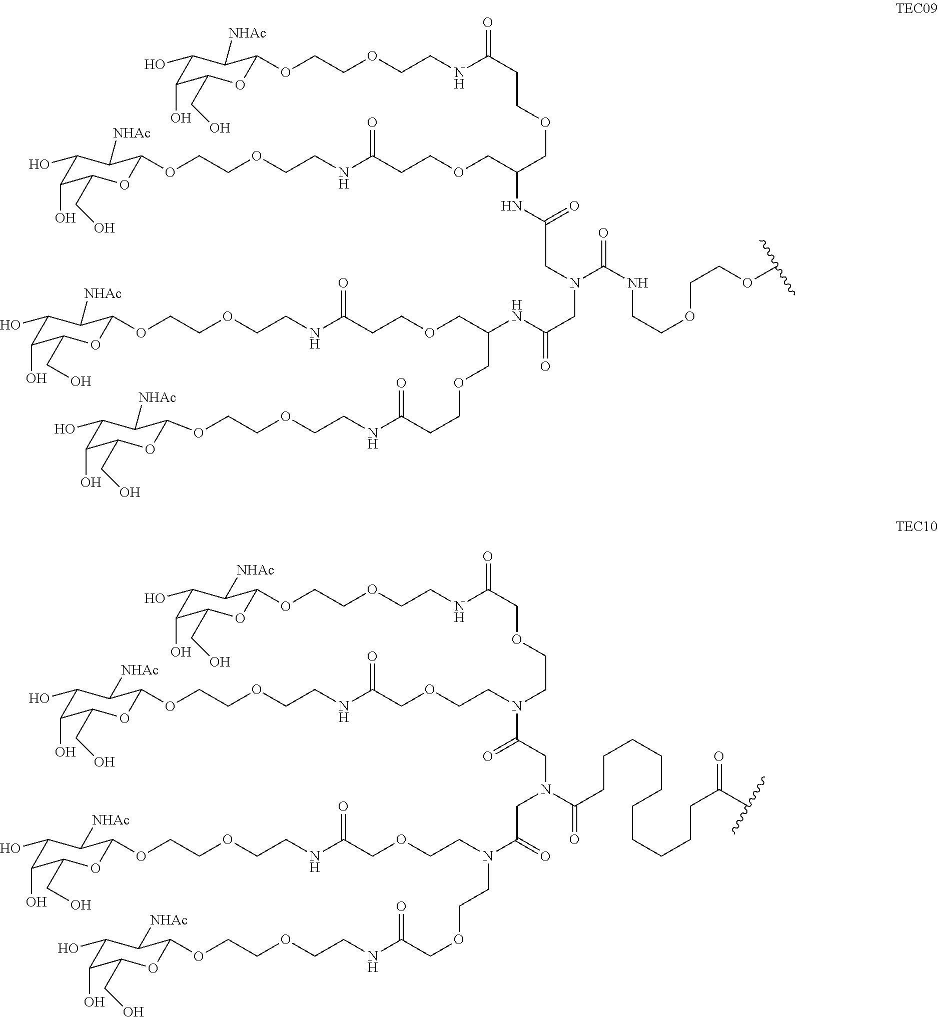 [SMILES: *OCCOCCNC(=O)N(CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O)CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCCCCCCCCCC(=O)N(CC(=O)N(CCOCC(=O)NCCOCCOC1OC(CO)C(O)C(O)C1NC(C)=O)CCOCC(=O)NCCOCCOC1OC(CO)C(O)C(O)C1NC(C)=O)CC(=O)N(CCOCC(=O)NCCOCCOC1OC(CO)C(O)C(O)C1NC(C)=O)CCOCC(=O)NCCOCCOC1OC(CO)C(O)C(O)C1NC(C)=O]

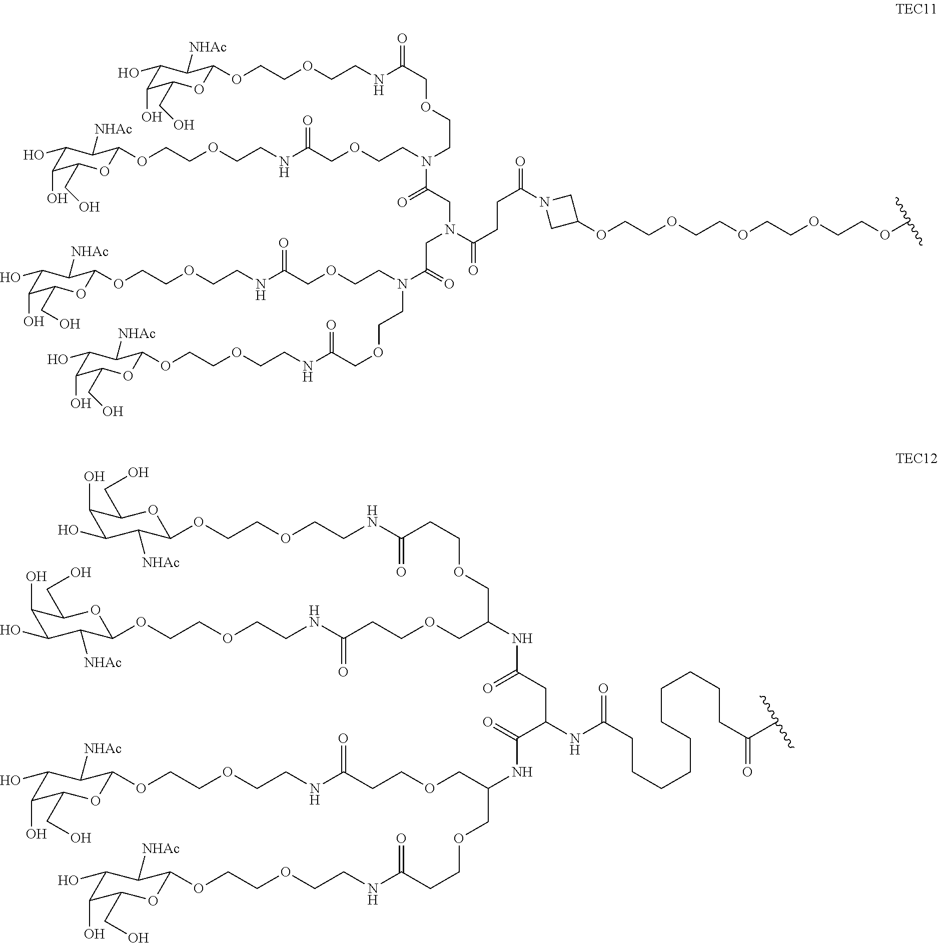 [SMILES: *OCCOCCOCCOCCOC1CN(C(=O)CCC(=O)N(CC(=O)N(CCOCC(=O)NCCOCCOC2OC(CO)C(O)C(O)C2NC(C)=O)CCOCC(=O)NCCOCCOC2OC(CO)C(O)C(O)C2NC(C)=O)CC(=O)N(CCOCC(=O)NCCOCCOC2OC(CO)C(O)C(O)C2NC(C)=O)CCOCC(=O)NCCOCCOC2OC(CO)C(O)C(O)C2NC(C)=O)C1 ; *C(=O)CCCCCCCCCCC(=O)NC(CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O)C(=O)NC(COCCC(=O)NCCOCCOC1OC(CO)C(O)C(O)C1NC(C)=O)COCCC(=O)NCCOCCOC1OC(CO)C(O)C(O)C1NC(C)=O]

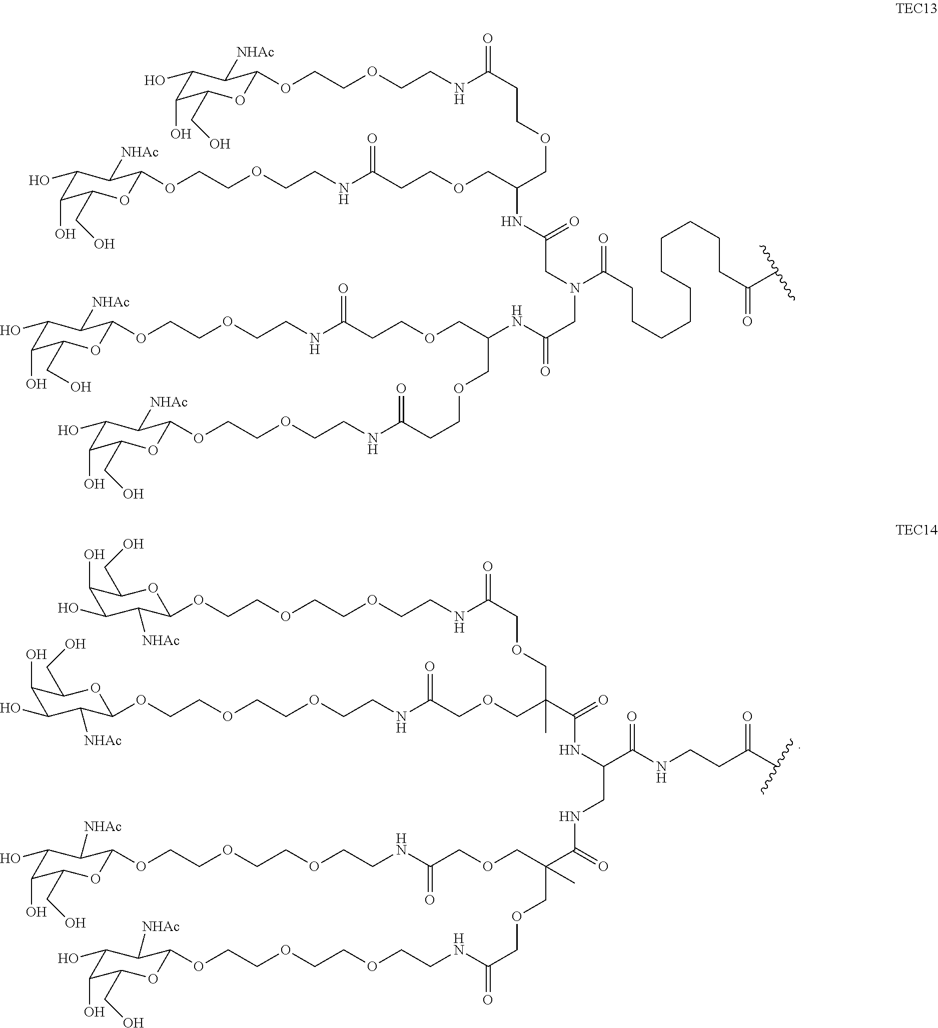 [SMILES: *C(=O)CCCCCCCCCCC(=O)N(CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O)CC(=O)NC(COCCC(=O)NCCOCCOC1OC(CO)C(O)C(O)C1NC(C)=O)COCCC(=O)NCCOCCOC1OC(CO)C(O)C(O)C1NC(C)=O ; *C(=O)CCNC(=O)C(CNC(=O)C(C)(COCC(=O)NCCOCCOCCOC1OC(CO)C(O)C(O)C1NC(C)=O)COCC(=O)NCCOCCOCCOC1OC(CO)C(O)C(O)C1NC(C)=O)NC(=O)C(C)(COCC(=O)NCCOCCOCCOC1OC(CO)C(O)C(O)C1NC(C)=O)COCC(=O)NCCOCCOCCOC1OC(CO)C(O)C(O)C1NC(C)=O]

15. An activated/modified ligand having an activation/modification group, wherein, the activated/modified ligand having an activation/modification group is obtained by activating/modifying the ligand of claim 1, and the activation/modification group includes but is not limited to: 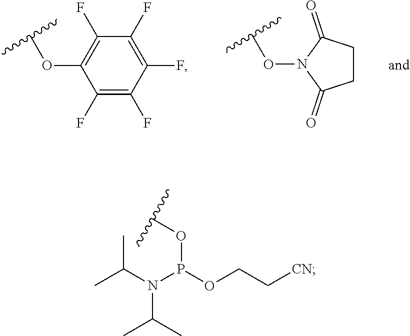 [SMILES: *Oc1c(F)c(F)c(F)c(F)c1F ; *ON1C(=O)CCC1=O ; *OP(OCCC#N)N(C(C)C)C(C)C]
and
the activation/modification occurs at:
(1) residue position 5 of B terminus or residue position 7 of L terminus of the ligand of Formula I; or
(2) residue position 7 of B terminus or residue position 9 of L terminus of the ligand of Formula II.

16. Use of the ligand of claim 1 in linking a functional compound.

17. (canceled)

18. A conjugate formed by coupling the ligand of claim 1 with a nucleic acid or a fluorescent compound, wherein:
the conjugate formed by coupling the ligand of Formula II with a nucleic acid or a fluorescent compound has a structure of Formula IV: 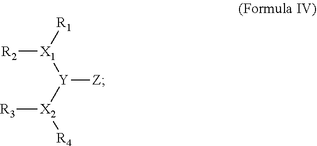
in Formula III and/or Formula IV, Z is the nucleic acid or the fluorescent compound;
the conjugate has a bond, including, but not limited to an amide bond, a phosphodiester bond and a phosphorothioate bond, between Y and Z.

19. (canceled)

20. (canceled)

21. A method for preparing the ligand of Formula VI, 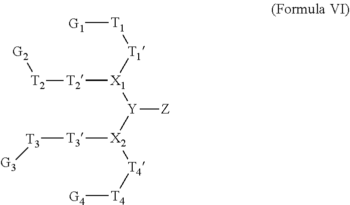
comprising the steps of:
(1) synthesis of a chain with sugar group comprising 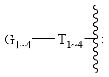
a glycoside comprising a chain of T₁-T₄ linked to position 1 of glycosyl and a protective group on saccharide hydroxyl is obtained by subjecting saccharide G₁-G₄ without a protective group to protection reaction (generally acetylation) to obtain a saccharide having a protective group on saccharide hydroxyl, which is then subjected to glycosylation reaction with a chain of T₁-T₄ having a protective group at one end and a chemically reactive group at the other end;
(2) synthesis of a chain with sugar group comprising 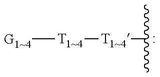
the saccharide chain comprising 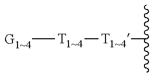
is obtained by subjecting the saccharide chain comprising 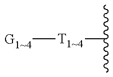
to deprotection reaction at T₁-T₄ terminus and then to condensation or substitution reaction with a compound comprising T₁′-T₄′; the compound comprising T₁′-T₄′ may be deprotected first and then react with T₁-T₄;
(3) synthesis of a compound comprising 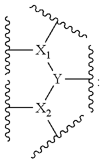
where a₁-a₄ are present, a compound comprising 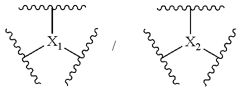
is firstly subjected to protection reaction to obtain a compound comprising 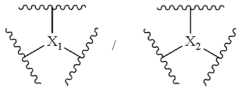
in which a₁-a₄ have a protective group, which is then subjected to condensation or substitution reaction with a compound comprising 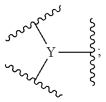
a₁-a₄ may not be protected, and the compound comprising 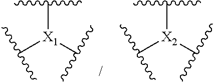
is directly linked to the compound comprising 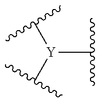
by condensation or substitution reaction; if 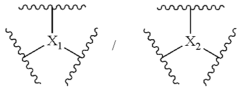
and/or 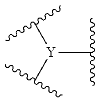
have a protective group at residue position 5/6, the linking reaction is conducted after the protective group is removed; where only a₁ and a₂ are absent, or where only a₃ and a₄ are absent, or where a₁-a₄ are all absent, the compound comprising 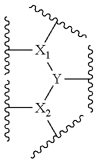
is synthesized by following the above-described method with a₁-a₄ protection/deprotection involved or not;
(4) synthesis of a compound comprising 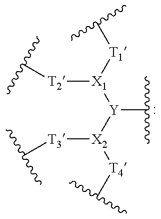
the compound comprising 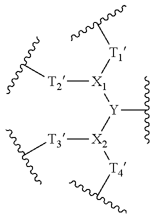
is obtained by subjecting the compound comprising 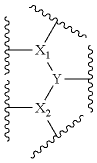
to addition, condensation or substitution reaction with a chain or precursor compound comprising T₁′-T₄′, or alternatively by subjecting the compound comprising 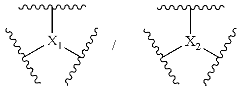
to addition, condensation or substitution reaction with the chain or precursor compound comprising T₁′-T₄′ to obtain a compound comprising 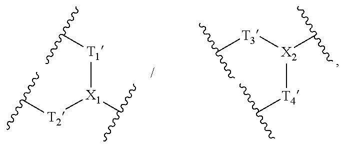
which is then subjected to condensation or substitution reaction with a compound comprising 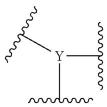
at residue position 5/6;
(5) synthesis of a compound comprising 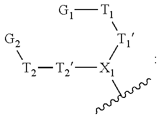
the compound comprising 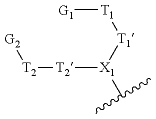
is obtained by subjecting the compound comprising 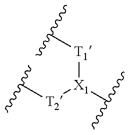
to deprotection reaction at T₁′-T₂′ terminus, and then to condensation or substitution reaction with the saccharide chain comprising 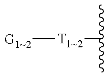
with protected glycosyl and unprotected T₁-T₂ terminus, or alternatively by subjecting a compound comprising 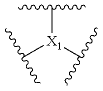
to condensation or substitution reaction with the saccharide chain comprising 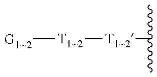
with protected glycosyl and unprotected T₁′-T₂′ terminus;
(6) synthesis of a compound comprising 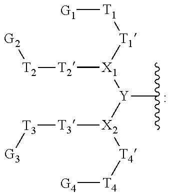
the compound comprising 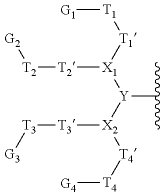
is obtained by subjecting the saccharide chain comprising 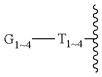
and the compound comprising 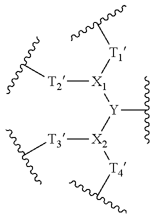
to deprotection reaction to remove their respective reactive groups, and then to condensation or substitution reaction with each other at residue positions 1-4, or alternatively by subjecting the saccharide chain comprising 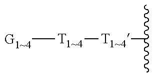
to reaction with the compound comprising 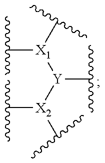
(7) introduction of L: a ligand compound having a naked chemically reactive group at L terminus is obtained by subjecting a L-comprising compound to condensation or substitution reaction with B at terminal residue position 7 in any of steps (3)-(6), with or without final removal of the protecting group from L terminus;
(8) introduction of linker K between any two of the abovementioned linked moieties: linker K is introduced either prior to or during the linkage of the two moieties at a specified position; and
optionally, (9) modification of the ligand: the modified ligand is obtained by modifying the ligand having a chemically reactive group at L terminus with by an active ester or phosphoramidite.
